(19) United States
(12) Patent Application Publication
(10) Pub. No.: US 2006/0177837 A1
(43) Pub. Date: Aug. 10, 2006

(54) Systems and methods for identifying diagnostic indicators

(76) Inventors: Ivan Borozan, Toronto (CA); Limin Chen, Toronto (CA); Aled M. Edwards, Toronto (CA); Elizabeth J. Heathcote, Toronto (CA); Ian D. McGilvray, Toronto (CA)

Correspondence Address: JONES DAY, 222 EAST 41ST ST, NEW YORK, NY, 10017, US

(21) Appl. No.: 11/204,186

(22) Filed: Aug. 15, 2005

Related U.S. Application Data
(60) Provisional application No. 60/601,227, filed on Aug. 13, 2004.

(51) Int. Cl. C12Q 1/68; G06F 19/00

(52) U.S. Cl. 435/6; 702/20

(57) ABSTRACT

Systems and methods are provided for predicting patient response to a therapy regimen for a liver disease or a disease that is treatable with an immunomodulatory disease therapy using gene expression classifiers. Systems and methods for screening for modulators of target gene expression are also provided. Systems and methods for developing therapeutics against one or more of the proteins coded for by genes of the present invention are also provided. Systems and methods for predicting a patient response to a regimen of pegylated interferon alpha and ribavirin in a therapy for hepatitis C viral infection are also provided.

### CROSS-REFERENCE TO RELATED APPLICATIONS

This application claims benefit, under 35 U.S.C. §119(e), of U.S. Provisional Patent Application No. 60/601,227 filed on Aug. 13, 2004, which is incorporated herein, by reference, in its entirety.

### 1 FIELD OF THE INVENTION

The present invention relates to methods for predicting patient response to a therapy regimen for a liver disease or a disease that is treatable with an immunomodulatory disease therapy using gene expression classifiers. The invention also relates to methods for screening for modulators of target gene expression. The present invention also provides methods for developing therapeutics against one or more of the proteins coded for by genes of the present invention.

### 2 BACKGROUND OF THE INVENTION

The therapy regimens for some diseases that are treatable with an immunomodulatory disease therapy are quite costly and have serious side-effects, and time-consuming. It can be some time before the results of the therapy can be ascertained, and if the therapy is ineffective, some time has elapsed before the patient can commence an alternative therapy regimen. It would be advantageous to be able to predict a patient's response to a therapy regimen before time and costs have been invested. There are presently different tests for patient response to therapy regimens currently available. However, these standard tests do not probe the molecular basis for a patient's non-responsiveness to a given therapy regimen for the diseases, and therefore can be somewhat inaccurate.

In a particular example, more than 3 million North Americans and more than 170 million people worldwide are infected chronically with HCV (see National Institutes of Health—National Institutes of Health Consensus Development Conference Statement Management of Hepatitis C. Hepatology 2002; 36, 5 Suppl 1: S3-20; and Poynard et al., 2003, Lancet. 362:2095-100, each of which are hereby incorporated by reference in its entirety) Currently there is no vaccine or small molecule therapy for this chronic disease, which can lead to serious liver disease and cancer. The most effective treatment is pegylated interferon alpha plus ribavirin (PegIFN/rib), which is associated with morbid side effects, a variable cure rate and high costs (NIH 2002). Although it is likely that the interaction of the virus with hepatic microenvironments creates a cellular state that is non-responsive to treatment (see Girard et al., 2002, Virology 295:272-83; Ghosh et al., 2003, Virology 306: 51-9; and Naganuma et al., 2000, J Virol. 74:8744-50, each of which is hereby incorporated by reference in its entirety), the molecular mechanisms leading to this state are not known and it is not possible to predict treatment outcomes prior to initiation of therapy. Viral and host factors both play a role: for example, infection with HCV genotypes 1 and 4 is associated with at best a 60% response rate, and increasing degrees of hepatic fibrosis are associated with poorer response rates (NIH). Mutations in viral (NS5A, NS5B) and host (MxA, OAS, PKR) proteins can enhance (NS5A, NSSB) or partially inhibit (MxA) the response to IFN-based treatment (Nishiguchi et al., 2001, Hepatology 33: 241-7; Watanabe et al., 2001, J Infect Dis. 183:1195-203; Murashima et al., 2000, J Med Virol. 62:185-90; Knapp et al., 2003, Genes Immun. 4:411-9; and Suzuki et al., 2004, J Viral Hepat. 11:271-6, each of which is incorporated by reference in its entirety). Increased MxA protein in hepatic biopsies is associated with poorer responses to treatment (MacQuillan et al., 2000, J Med Virol. 68:197-205, which is hereby incorporated by reference in its entirety). While these studies are intriguing the heterogeneity of viral and host phenotypes makes it very unlikely that any single factor will accurately predict the cellular response to treatment.

The ultimate response to treatment can only be gauged after PegIFN/rib has been initiated. It is currently recommended that patients undergo at least a twelve week course of combination therapy and then be assessed for an antiviral response. An early viral response (EVR, 2-log decrease in baseline HCV RNA titers) is indicative of the eventual outcome, though only with 60-90% accuracy (NIH 2002). However, the 3-month regimen is asso-ciated with maximum morbid side effects and is expensive. (see see National Institutes of Health—National Institutes of Health Consensus Development Conference Statement Management of Hepatitis C. Hepatology 2002;36, 5 Suppl 1: S3-20; and Fried, 2002, Hepatology 36:S237-S244, each of which is hereby incorporated by reference in its entirety.

In an exemplary embodiment, the hepatic gene expression profiles of 15 nonresponder (NR) and 16 responder (R) patients was compared to liver tissue from 20 normal livers in order to identify any liver-specific characteristics that might influence responses to treatment. All of the HCV biopsies are taken prior to initiation of treatment with PegIFN/rib as part of the patient work up to decide on suitability for antiviral therapy. Applicants observed a distinct profile that accurately classified patient samples by their eventual responder/non-responder status.

### 3 SUMMARY OF THE INVENTION

The present invention provides a method of determining responsiveness to a therapy for a disease in a subject, the method comprising: applying an abundance value for each product in a plurality of products to a model, wherein the abundance value for all or a portion of the products in the plurality of products is obtained by measurement of a biological sample from the subject, and the plurality of products comprises a respective product of each of at least four different genes set forth in table 1; wherein a first result of the applying is deemed to indicate that the subject is responsive to the therapy for the disease, and a second result of the applying is deemed to indicate that the subject is nonresponsive to the therapy for the disease, and wherein either (i) the therapy is a liver disease therapy and the disease is a liver disease, or (ii) the therapy is a immunomodulatory disease therapy and the disease is a disease treatable with an immunomodulatory disease therapy.

Each product in the plurality of products can be an abundance value for an RNA transcript of a gene set forth in Table 1 in the biological sample. Each product in the plurality of products can be an abundance value for a protein encoded by a gene set forth in Table 1 in the biological sample. The therapy may be a liver disease therapy for a liver disease, or the therapy is a immunomodulatory disease therapy and the disease is a disease treatable with an immunomodulatory disease therapy. The model may be a clustering algorithm, a neural network, a regression model, linear discriminant analysis, quadratic discriminant analysis, principal component analysis, a support vector machine, a decision tree, or a nearest neighbor analysis, or any combination of models. The training subjects used in the models may comprise at least two training subjects, or between two and one thousand training subjects.

In different aspects of the present invention, the plurality of products may consist of respective products of a maximum of one hundred genes, fifty genes, twenty-five genes, fifteen genes, ten genes, or eight genes. The plurality of products may consist of respective products of all of the genes set forth in Table 1, between four and forty genes set forth in Table 1, four and twenty genes set forth in Table 1, or between four and eight genes set forth in Table 1.

In one aspect of the present invention, the plurality of products comprises a product of one or more of the group consisting of SEQ ID NO: 1, SEQ ID NO: 3, SEQ ID NO. 5, SEQ ID NO: 7, and SEQ ID NO: 9. In another aspect of the present invention, the plurality of products comprises a product of one or more of the group consisting of SEQ ID NO: 2, SEQ ID NO: 4, SEQ ID NO. 6, SEQ ID NO: 8, and SEQ ID NO: 10. In still other aspects of the present invention, the plurality of products consists of products of OAS3, G1P3, DUSP1, IFIT1, MX1, G1P2, LAP3, cig5, LGP1, USP18, RPS28, CEB1, RPLP2, STXBP5, ETEF1, OAS2, ATF5, and PI3KAP1, respectively, or of a product of IFIT1, OAS2, DUSP1, ATF5, LGP1, RPS28, USP18, and STXBP5, respectively.

In different embodiments of the present invention, the subject is human, a mouse, a rat, a monkey, a hamster, a sheep, a cow, a pig, a horse, a cat or a dog.

In yet another aspect of the present invention, the method may further comprise a step of determining the abundance value for each product in the plurality of products prior to the step (a). The determining may comprise hybridizing a polynucleotide encoding the product under conditions of high stringency to nucleotides of the genes set forth in Table 1, or hybridizing a nucleotide sequence under conditions of high stringency to a polynucleotide that is complementary to nucleotides of the genes. The determining may comprise hybridizing a polynucleotide encoding the product under conditions of moderate stringency to nucleotides of the genes set forth in Table 1, or hybridizing a nucleotide sequence under conditions of moderate stringency to a polynucleotide that is complementary to nucleotides of the genes.

In still another aspect of the invention, the disease therapy comprises administration of human interferon to the subject, where the human interferon may be human interferon alpha or human interferon beta.

In a specific embodiment, the disease is hepatitis C. In another embodiment, the disease is an immune-related disease, such as, but not limited to, multiple sclerosis, idiopathic pulmonary fibrosis, Guillain-Barre Syndrome, adult systemic mastocytosis, ulcerative colitis, Crohn's disease, hepatitis C associated cryoglobulinemia, or HTLV-1 associated myelopathy. In yet another embodiment, the disease is caused by a viral infection of the subject, or a bacterial disease caused by a bacterium. The bacterium may be cryptococcal meningitis or Tuberculosis.

In yet another embodiment, the disease is a neoplastic disease, diabetic retinopathy or Peyronie's disease. In yet other embodiments, the disease is renal cell carcinoma, hepatocellular carcinoma, a malignant carcinoid tumor, a neuroendocrine tumor, lymphoma, acute leukemia, chronic leukemia, chronic myelogenous leukemia, urothelial cancer, prostate cancer, penile cancer, nasopharyngeal cancer, pancreatic cancer, gastric cancer, cervical cancer, colorectal cancer, small cell lung cancer, non small cell lung cancer, malignant mesothelioma, or breast cancer.

The present invention also provides a computer program product comprising a computer readable storage medium and a computer program mechanism embedded therein, the computer program mechanism comprising: a data analysis module for determining a responsiveness to an disease therapy in a subject for a disease, wherein either (i) the therapy is a liver disease therapy and the disease is a liver disease, or (ii) the therapy is an immunomodulatory disease therapy and the disease is a disease treatable with an immunomodulatory disease therapy, the data analysis module comprising: instructions for applying an abundance of each product in a plurality of products to a model, wherein the abundance of all or a portion of the products in the plurality of products is obtained by measurement of a biological sample from the subject, and the plurality of products comprises a respective product of each of at least four different genes set forth in table 1; wherein a first result of the instructions for applying is deemed to indicate that the subject is responsive to the disease therapy for the disease, and a second result of the instructions for applying is deemed to indicate that the subject is not responsive to the disease therapy for the disease.

The present invention also provides a computer comprising: a central processing unit; a memory, coupled to the central processing unit, the memory storing a data analysis module for determining a responsiveness to a disease therapy in a subject for a disease, wherein either (i) the therapy is a liver disease therapy and the disease is a liver disease, or (ii) the therapy is an immunomodulatory disease therapy and the disease is a disease treatable with an immunomodulatory disease therapy, the data analysis module comprising: instructions for applying an abundance of each product in a plurality of products to a model, wherein the abundance of all or a portion of the products in the plurality of products is obtained by measurement of a biological sample from the subject, and the plurality of products comprises a respective product of each of at least four different genes set forth in table 1; wherein a first result of the instructions for applying is deemed to indicate that the subject is responsive to the disease therapy for the disease, and a second result of the instructions for applying is deemed to indicate that the subject is not responsive to the disease therapy for the disease.

In yet another aspect, the present invention provides a method for identifying a candidate molecule for use as a liver disease therapy agent or an immunomodulatory disease therapy agent, comprising: (a) contacting a cell, or recombinantly expressing within the cell, a test molecule; (b) determining whether the RNA expression or protein expression in the cell of at least one open reading frame is changed in step (a) relative to the expression of the open reading frame in the absence of the test molecule, each the open reading frame being regulated by a promoter native to a gene in Table 1 or a homolog of a gene in Table 1, wherein the RNA expression or protein expression of the at least one open reading frame is changed, the test molecule is identified as a candidate molecule for use as a liver disease therapy agent or an immunomodulatory disease therapy agent.

In a related embodiment, step (b) may comprise determining whether the RNA expression or protein expression of the at least one open reading frame is lowered in step (a) relative to the expression of the open reading frame in the absence of the candidate molecule wherein at least one open reading frame is regulated by a promoter native to SEQ ID NO: 10. In other embodiments, step (b) may comprise determining whether the RNA expression or protein expression of the at least one open reading frame is lowered in step (a) relative to the expression of the open reading frame in the absence of the candidate molecule wherein at least one open reading frame is regulated by a promoter native to ISG15. In yet other embodiments, step (b) may comprise determining whether RNA expression is changed, whether protein expression is changed, or whether RNA or protein expression of at least two of the open reading frames is changed.

In another related embodiment, step (a) may comprise contacting the cell with the candidate molecule, where step (a) is carried out in a liquid high throughput-like assay. In yet another embodiment, the cell comprises a promoter region of at least one gene selected from the group consisting of SEQ ID NO: 1, SEQ ID NO: 3, SEQ ID NO: 5, SEQ ID NO: 7, SEQ ID NO: 9, and homologs of each of the foregoing, each promoter region being operably linked to a marker gene; and where step (b) comprise determining whether the RNA expression or protein expression of the marker gene(s) is changed in step (a) relative to the expression of the marker gene in the absence of the candidate molecule. The marker gene may green fluorescent protein, red fluorescent protein, blue fluorescent protein, luciferase, LEU2, LYS2, ADE2, TRP1, CAN1, CYH2, GUS, CUP1, or chloramphenicol acetyl transferase.

In still another aspect, the present invention provides a method for identifying a candidate molecule for use as a liver disease therapy agent or an immunomodulatory disease therapy agent, comprising determining whether a test molecule specifically binds to (a) a first polypeptide, the amino acid sequence of which comprises SEQ ID NO: 2, SEQ ID NO: 4, SEQ ID NO: 6, SEQ ID NO: 8, or SEQ ID NO: 10; or (b) a second polypeptide that comprises a homolog of SEQ ID NO: 2, SEQ ID NO: 4, SEQ ID NO: 6, SEQ ID NO: 8, or SEQ ID NO: 10; or (c) a third polypeptide that comprises the protein product of a polynucleotide wherein the polynucleotide hybridizes under conditions of high stringency to a nucleic acid consisting of SEQ ID NO: 1, SEQ ID NO: 3, SEQ ID NO: 5, SEQ ID NO: 7, or SEQ ID NO: 9 or the complements of SEQ ID NO: 1, SEQ ID NO: 3, SEQ ID NO: 5, SEQ ID NO: 7, or SEQ ID NO: 9, wherein the determining comprises contacting the polypeptide of (a), (b) or (c) above with the test molecule under conditions suitable for binding, and detecting specific binding of the test molecule to the soluble polypeptide, wherein when specific binding is detected, the test molecule is identified as a candidate molecule for use as a liver disease therapy agent or an immunomodulatory disease therapy agent. The specific binding of the test molecule to the polypeptide may be detected by gel filtration, an affinity column, or a modulation of an enzymatic activity of the polypeptide.

The present invention also provides a method of administering a liver disease therapy or an immunomodulatory disease therapy comprising administering to a subject in which the treatment is desired a therapeutically effective amount of a compound that modulates in the subject an abundance or an activity of a protein comprising a sequence selected from the group consisting of SEQ ID NO: 2, SEQ ID NO: 4, SEQ ID NO: 6, SEQ ID NO: 8, SEQ ID NO: 10 and homologs of each of the foregoing. The subject may be human, a mouse, a rat, a monkey, a hamster, a sheep, a cow, a pig, a horse, a cat or a dog. In a specific embodiment, the compound antagonizes an activity of a protein comprising SEQ ID NO: 10 in the subject.

The present invention also method for identifying a candidate molecule for use as a liver disease therapy agent or an immunomodulatory disease therapy agent, comprising: contacting a cell, or recombinantly expressing within the cell, a test molecule, and determining whether the abundance or activity of a protein comprising SEQ ID NO: 2, SEQ ID NO: 4, SEQ ID NO: 6, SEQ ID NO: 8, or SEQ ID NO: 10 in the cell is changed relative to the abundance or activity, respectively, of the protein in the absence of the test molecule, wherein when the abundance or activity of the protein is changed, the test molecule is identified as a candidate molecule for use as a liver disease therapy agent or an immunomodulatory disease therapy agent.

In still another aspect, the present invention provides a method for identifying a liver disease therapy agent or an immunomodulatory disease therapy agent, comprising: (i) contacting a polypeptide with a test molecule, wherein the polypeptide is: (a) a first polypeptide, the amino acid sequence of which comprises SEQ ID NO: 2, SEQ ID NO: 4, SEQ ID NO: 6, SEQ ID NO: 8, or SEQ ID NO: 10; or (b) a second polypeptide that comprises a homolog of SEQ ID NO: 2, SEQ ID NO: 4, SEQ ID NO: 6, SEQ ID NO: 8, or SEQ ID NO: 10; or (c) a third polypeptide that comprises the protein product of a polynucleotide wherein the polynucleotide hybridizes under conditions of high stringency to a nucleic acid consisting of SEQ ID NO: 1, SEQ ID NO: 3, SEQ ID NO: 5, SEQ ID NO: 7, or SEQ ID NO: 9 or the complements of SEQ ID NO: 1, SEQ ID NO: 3, SEQ ID NO: 5, SEQ ID NO: 7, or SEQ ID NO: 9; and (ii) determining whether the test molecule modulates the biological activity of the polypeptide relative to the biological activity of the polypeptide in the absence of the test molecule, wherein when the abundance or activity of the polypeptide is changed, the test molecule is identified as a candidate molecule for use as a liver disease therapy agent or an immunomodulatory disease therapy agent.

The present invention provides a computer system comprising: a central processing unit; and a memory, coupled to the central processing unit, the memory storing (a) a sequence of one or more genes or a sequence of a polypeptide encoded by the one or more genes, wherein the one or more genes are selected from the group consisting of G1P2/ISG15/IFI-15, G1P3/IFI-6-16, OAS3, RPLP2, CEB1, VIPERIN/C1G5, PI3KAP1, MX1, LAP3, ETEF1, IFIT1I/IFI56, OAS2, DUSP1, ATF5, LGP-1, RPS28, USP18/UBP43, and STXBP5; (b) one or more computer programs, wherein the computer programs comprise instructions for executing at least one supervised classifier analysis technique; and (c) instructions for outputting a predicted response of a subject to a regimen of pegylated interferon alpha (hereafter PegIFNα) and ribavirin in a therapy for hepatitis C viral infection.

The present invention provides a method for predicting the response of a subject to a regimen of PegIFNα and ribavirin in a therapy for a hepatitis C viral infection, the method comprising: (a) determining the expression levels of the following genes in a tissue sample (e.g., liver, blood, any bodily fluid, peripheral mononuclear blood cells, any tissue, lymphocytes, a biopsy, etc.) from the subject: G1P2/ISG15/IFI-15, G1P3/IFI-6-16, OAS3, RPLP2, CEB1, VIPERIN/CIG5, PI3KAP1, MX1, LAP3, ETEF1, IFIT1/IFI56, OAS2, DUSP1, ATF5, LGP-1, RPS28, USP18/UBP43, and STXBP5; (b) comparing the levels of expression in (a) to a corresponding control sample from a subject not having a hepatitis C viral infection; and (c) predicting that the subject will be nonresponsive to a regimen of PegIFNα and ribavirin in a therapy for hepatitis C if there is an increase in the expression levels of G1P2/ISG15/IFI-15, G1P3/IFI-6-16, OAS3, RPLP2, CEB1, VIPERIN/CIG5, PI3KAP1, MX1, LAP3, IFIT1/IFI56, OAS2, DUSP1, ATF5, LGP-1, RPS28, and USP18/UBP43 in (a) relative to the expression levels of the genes in the control sample, and if there is a decrease in the expression levels of ETEF1 and STXBP5 in (a) relative to the expression levels of the genes in the control sample.

The present invention also provides a method for predicting the response of a subject to a regimen of PegIFNα and ribavirin in a therapy for a hepatitis C viral infection, the method comprising: (a) determining the expression levels of the following genes in a tissue sample (e.g., liver, blood, any bodily fluid, any tissue, a biopsy, peripheral mononuclear blood cells, lymphocytes, etc.) from the subject: IFIT1/IFI56, OAS2, DUSP1, ATF5, LGP-1, RPS28, USP18/UBP43, and STXBP5; (b) comparing the levels of expression in (a) to a corresponding control sample from a subject not having a hepatitis C viral infection; and (c) predicting that the subject will be nonresponsive to a regimen of PegIFNα and ribavirin in a therapy for a hepatitis C viral infection if there is an increase in the expression levels of IFIT1/IFI56, OAS2, DUSP1, ATF5, LGP-1, RPS28, and USP18/UBP43 in (a) relative to the expression levels of the genes in the control sample, and if there is a decrease in the expression levels of STXBP5 in (a) relative to the expression levels of STXBP5 in the control sample.

The present invention also provides a method for predicting the response of a subject to a regimen of PegIFNα and ribavirin in a therapy for a hepatitis C viral infection, the method comprising: (a) determining the expression levels of at least one of the following genes in a tissue sample (e.g., liver, blood, any bodily fluid, any tissue, a biopsy, peripheral mononuclear blood cells, lymphocytes, etc.) from the subject: G1P2/ISG15/IFI-15, G1P3/IFI-6-16, OAS3, RPLP2, CEB1, VIPERIN/CIG5, PI3KAP1, MX1, LAP3, ETEF1, IFIT1/IFI56, OAS2, DUSP1, ATF5, LGP-1, RPS28, USP18/UBP43, and STXBP5; (b) comparing the levels of expression in (a) to a corresponding control sample from a subject not having a hepatitis C viral infection; and (c) predicting that the subject will be nonresponsive to a regimen of PegIFNα and ribavirin in a therapy for the hepatitis C viral infection if there is an increase in the expression levels of G1P2/ISG15/IFI-15, G1P3/IFI-6-16, OAS3, RPLP2, CEB1, VIPERIN/CIG5, PI3KAP1, MX1, LAP3, IFIT1/IFI56, OAS2, DUSP1, ATF5, LGP-1, RPS28, and USP18/UBP43 in (a) relative to the expression levels of the genes in the control sample, and if there is a decrease in the expression levels of ETEF1 and STXBP5 in (a) relative to the expression levels of the genes in the control sample.

The present invention also provides a method for predicting the response of a subject to a regimen of PegIFNα and ribavirin in a therapy for a hepatitis C viral infection, the method comprising: (a) determining the expression levels of at least one of the following genes in a tissue sample (e.g., liver, blood, any bodily fluid, any tissue, a biopsy, peripheral mononuclear blood cells, lymphocytes, etc.) from the subject: IFIT1/IFI56, OAS2, DUSP1, ATF5, LGP-1, RPS28, USP18/UBP43, and STXBP5; (b) comparing the levels of expression in (a) to a corresponding control sample from a subject not having a hepatitis C viral infection; and (c) predicting that the subject will be nonresponsive to a regimen of PegIFNα and ribavirin in a therapy for hepatitis C if there is an increase in the expression levels of IFIT1I/IFI56, OAS2, DUSP1, ATF5, LGP-1, RPS28, and USP18/UBP43 in (a) relative to the expression levels in the genes in the control sample, and if there is a decrease in the expression levels of STXBP5 in (a) relative to the expression levels in the genes in the control sample.

In another aspect, the present invention provides a method for predicting the response of a subject to a regimen of PegIFNα and ribavirin in a therapy for a hepatitis C viral infection, the method comprising: (a) determining the expression levels of two or more of the following genes in a tissue (e.g., liver, blood, any bodily fluid, any tissue, a biopsy, peripheral mononuclear blood cells, lymphocytes, etc.) sample from the subject: G1P2/ISG15/IFI-15, G1P3/IFI-6-16, OAS3, RPLP2, CEB1, VIPERIN/CIG5, PI3KAP1, MX1, LAP3, ETEF1, IFIT1/IFI56, OAS2, DUSP1, ATF5, LGP-1, RPS28, USP18/UBP43, and STXBP5; (b) comparing the levels of expression in (a) to a corresponding control sample from a subject not having a hepatitis C viral infection; and (c) predicting that a subject will be nonresponsive to a regimen of PegIFNα and ribavirin in a therapy for hepatitis C if there is an increase in the expression levels of G1P2/ISG15/IFI-15, G1P3/IFI-6-16, OAS3, RPLP2, CEB1, VIPERIN/CIG5, PI3KAP1, MX1, LAP3, IFIT1/IFI56, OAS2, DUSP1, ATF5, LGP-1, RPS28, and USP18/UBP43 in (a) relative to the expression levels of the genes in the control sample, and if there is a decrease in the expression levels of ETEF1 and STXBP5 in (a) relative to the expression levels of the genes in the control sample.

In another aspect, the present invention provides a method for predicting the response of a subject to a regimen of PegIFNα and ribavirin in a therapy for a hepatitis C viral infection, the method comprising: (a) determining the expression levels of two or more of the following genes in a tissue sample (e.g., liver, blood, any bodily fluid, any tissue, a biopsy, peripheral mononuclear blood cells, lymphocytes, etc.) from the subject: IFIT1/IFI56, OAS2, DUSP1, ATF5, LGP-1, RPS28, USP18/UBP43, and STXBP5; (b) comparing the levels of expression in (a) to a corresponding control sample from a subject not having a hepatitis C viral infection; and (c) predicting that a subject will be nonresponsive to a regimen of PegIFNα and ribavirin in a therapy for hepatitis C if there is an increase in the expression levels of IFIT1/IFI56, OAS2, DUSP1, ATF5, LGP-1, RPS28, and USP18/UBP43 in (a) relative to the expression levels in the genes in the control sample, and if there is a decrease in the expression levels of STXBP5 in (a) relative to the expression levels in the genes in the control sample.

In yet another aspect, the present invention provides a method for predicting the response of a subject to a regimen of PegIFNα and ribavirin in a therapy for a hepatitis C viral infection, the method comprising: (a) determining the expression levels of at least 1 of the following genes in a tissue sample (e.g., liver, blood, any bodily fluid, any tissue, a biopsy, peripheral mononuclear blood cells, lymphocytes, etc.) from the subject: IFI-6-16 (G1P3), LAP3 (luecine aminopeptidase 3) CIG5 (Viperin) and LGP1 (d11lgp1e-like); (b) comparing the levels of expression in (a) to a corresponding control sample from a subject not infected with a hepatitis C viral infection; and (c) predicting that the subject will be nonresponsive to a regimen of PegIFNα and ribavirin in a therapy for hepatitis C if there is an increase in the expression levels of the genes in (a) relative to the expression levels of the genes in the control sample.

In still another aspect, the present invention provides a method of determining responsiveness to a regimen of PegIFNα and ribavirin for a hepatitis C viral infection in a subject, the method comprising: applying an abundance value for each product in a plurality of products to a model, wherein the abundance value for all or a portion of the products in the plurality of products is obtained by measurement of a liver sample from the subject, and the plurality of products comprises a respective product of each of at least four different genes set forth in table 1; wherein a first result of the applying is deemed to indicate that the subject is responsive to the PegIFNα plus ribavirin therapy for the hepatitis C viral infection, and a second result of the applying is deemed to indicate that the subject is nonresponsive to the PegIFNα plus ribavirin therapy for the hepatitis C viral infection.

The present invention also provides a computer program product for use in conjunction with a computer system, the computer program product comprising a computer readable storage medium, the computer readable storage medium comprising a sequence of one or more genes or a sequence of a polypeptide encoded by the one or more genes, wherein the one or more genes is G1P2/ISG15/IFI-15, G1P3/IFI-6-16, OAS3, RPLP2, CEB1, VIPERIN/CIG5, PI3KAP1, MX1, LAP3, ETEF1, IFIT1/IFI56, OAS2, DUSP1, ATF5, LGP-1, RPS28, USP18/UBP43, STXBP5 or some combination thereof, and instructions for outputting a predicted response of a subject to a regimen of PegIFNα and ribavirin in a therapy for hepatitis C viral infection.

3.1 Terminology

As used herein, the term “analog” in the context of proteinaceous agent (e.g., proteins, polypeptides, peptides, and antibodies) refers to a proteinaceous agent that possesses a similar or identical function as a second proteinaceous agent but does not necessarily comprise a similar or identical amino acid sequence of the second proteinaceous agent, or possess a similar or identical structure of the second proteinaceous agent. A proteinaceous agent that has a similar amino acid sequence refers to a second proteinaceous agent that satisfies at least one of the following: (a) a proteinaceous agent having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a second proteinaceous agent; (b) a proteinaceous agent encoded by a nucleotide sequence that hybridizes under stringent conditions to a nucleotide sequence encoding a second proteinaceous agent of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, or at least 150 contiguous amino acid residues; and (c) a proteinaceous agent encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a second proteinaceous agent. A proteinaceous agent with similar structure to a second proteinaceous agent refers to a proteinaceous agent that has a similar secondary, tertiary or quaternary structure to the second proteinaceous agent. The structure of a proteinaceous agent can be determined by methods known to those skilled in the art, including but not limited to, peptide sequencing, X-ray crystallography, nuclear magnetic resonance, circular dichroism, and crystallographic electron microscopy.

As used herein, the term “analog” in the context of a non-proteinaceous analog refers to a second organic or inorganic molecule which possess a similar or identical function as a first organic or inorganic molecule and is structurally similar to the first organic or inorganic molecule.

As used herein, the terms “compound” and “agent” are used interchangeably.

As used herein, the term “derivative” in the context of proteinaceous agent (e.g., proteins, polypeptides, peptides, and antibodies) refers to a proteinaceous agent that comprises an amino acid sequence which has been altered by the introduction of amino acid residue substitutions, deletions, and/or additions. The term “derivative” as used herein also refers to a proteinaceous agent which has been modified, i.e., by the covalent attachment of any type of molecule to the proteinaceous agent. For example, but not by way of limitation, an antibody may be modified, e.g., by glycosylation, acetylation, pegylation, phosphorylation, amidation, derivatization by known protecting/blocking groups, proteolytic cleavage, linkage to a cellular ligand or other protein, etc. A derivative of a proteinaceous agent may be produced by chemical modifications using techniques known to those of skill in the art, including, but not limited to specific chemical cleavage, acetylation, formylation, metabolic synthesis of tunicamycin, etc. Further, a derivative of a proteinaceous agent may contain one or more non-classical amino acids. A derivative of a proteinaceous agent possesses a similar or identical function as the proteinaceous agent from which it was derived.

As used herein, the term “derivative” in the context of a non-proteinaceous derivative refers to a second organic or inorganic molecule that is formed based upon the structure of a first organic or inorganic molecule. A derivative of an organic molecule includes, but is not limited to, a molecule modified, e.g., by the addition or deletion of a hydroxyl, methyl, ethyl, carboxyl or amine group. An organic molecule may also be esterified, alkylated and/or phosphorylated.

As used herein, the term “diagnosis” refers to a process of determining an individual's predicted response to a therapy regimen to a disease that is treatable with an immunomodulatory disease therapy or a therapy regimen to a liver disease. In this context, “diagnosis” refers to a process whereby one determines whether an individual is expected to be responsive to a liver disease therapy regimen or a therapy regimen for a disease that is treatable with an immunomodulatory disease therapy (“responder”) or is expected not to be responsive to the therapy regimen (“non-responder”) while minimizing the likelihood that the individual is improperly predicted to be responsive to a liver disease therapy regimen or a therapy regimen for a disease that is treatable with an immunomodulatory disease therapy (“responder”) or improperly predicted not to be responsive to the therapy regimen (“non-responder”). For example, in the case of a hepatitis C viral infection, a subject is designated as a non-responder, or non-responsive, if the HCV RNA is detectable at the end of therapy, as a responder, or responsive, after achieving a sustained viral response (SVR) if both end-of-treatment and 6 months follow-up HCV RNA was undetectable, and as a relapser if the HCV RNA was undetectable at the end of treatment but subsequently became detectable at the 6 months follow-up.

As used herein, the term “disease treatable with an immunomodulatory disease” refers to any disease which can be treated using a modulator of the immune system, such as an interferon-treated disease.

As used herein, the term “effective amount” refers to the amount of a compound which is sufficient to reduce or ameliorate the progression, severity and/or duration of a liver disease or a disease that is treatable with an immunomodulatory disease therapy, or one or more symptoms thereof, prevent the development, recurrence or onset of a liver disease or a disease that is treatable with an immunomodulatory disease therapy or one or more symptoms thereof, prevent the advancement of a liver disease or a disease that is treatable with an immunomodulatory disease therapy or one or more symptoms thereof, or enhance or improve the prophylactic or therapeutic effect(s) of another therapy.

As used herein, the term “fragment” refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of another polypeptide or a protein. In a specific embodiment, a fragment of a protein or polypeptide retains at least one function of the protein or polypeptide. In another embodiment, a fragment of a protein or polypeptide retains at least two, three, four, or five functions of the protein or polypeptide. Preferably, a fragment of an antibody retains the ability to immunospecifically bind to an antigen.

As used herein, the term “fusion protein” refers to a polypeptide that comprises an amino acid sequence of a first protein or polypeptide or functional fragment, analog or derivative thereof, and an amino acid sequence of a heterologous protein, polypeptide, or peptide (i.e., a second protein or polypeptide or fragment, analog or derivative thereof different than the first protein or fragment, analog or derivative thereof). In one embodiment, a fusion protein comprises a prophylactic or therapeutic agent fused to a heterologous protein, polypeptide or peptide. In accordance with this embodiment, the heterologous protein, polypeptide or peptide may or may not be a different type of prophylactic or therapeutic agent.

As used herein, the term “hybridizes under stringent conditions” describes conditions for hybridization and washing under which nucleotide sequences at least 30% (preferably, 35%, 40%, 45%, 50%, 55%, 60%, 65%, 70%, 75%, 80%, 85%, 90% or 98%) identical to each other typically remain hybridized to each other. Such stringent conditions are known to those skilled in the art and can be found in Current Protocols in Molecular Biology, John Wiley & Sons, N.Y. (1989), 6.3.1-6.3.6. In one, non-limiting example stringent hybridization conditions are hybridization at 6× sodium chloride/sodium citrate (SSC) at about 45° C., followed by one or more washes in 0.1×SSC, 0.2% SDS at about 68° C. In a preferred, non-limiting example stringent hybridization conditions are hybridization in 6×SSC at about 45° C., followed by one or more washes in 0.2.×.SSC, 0.1% SDS at 50-65° C. (i.e., one or more washes at 50° C., 55° C., 60° C. or 65° C.). It is understood that the nucleic acids of the invention do not include nucleic acid molecules that hybridize under these conditions solely to a nucleotide sequence consisting of only A or T nucleotides.

As used herein, the term “immunospecifically binds to an antigen” and analogous terms refer to peptides, polypeptides, proteins, fusion proteins and antibodies or fragments thereof that specifically bind to an antigen or a fragment and do not specifically bind to other antigens. A peptide, polypeptide, protein, or antibody that immunospecifically binds to an antigen may bind to other peptides, polypeptides, or proteins with lower affinity as determined by, e.g., immunoassays, BlAcore, or other assays known in the art. Antibodies or fragments that immunospecifically bind to an antigen may cross-reactive with related antigens. Preferably, antibodies or antibody fragments that immunospecifically bind to an antigen do not cross-react with other antigens.

As used herein, “specific binding” refers to refers to binding between molecules that is detectable over background binding, and is not non-specific. The molecule is still capable of binding to other molecules.

As used herein, the terms “manage”, “managing” and “management” refer to the beneficial effects that a subject derives from a therapy (e.g., a prophylactic or therapeutic agent) which does not result in a cure of a liver disease or a disease that is treatable with an immunomodulatory disease therapy. In certain embodiments, a subject is administered one or more therapies to “manage” a liver disease or a disease that is treatable with an immunomodulatory disease therapy so as to prevent the progression or worsening of the liver disease or the disease that is treatable with an immunomodulatory disease therapy.

As used herein, the terms “non-responsive” and “refractory” describe patients treated with a currently available therapy (e.g., prophylactic or therapeutic agent) for a liver disease or a disease that is treatable with an immunomodulatory disease therapy, which is not clinically adequate to relieve one or more symptoms associated therewith. Typically, such patients suffer from severe, persistently active disease and require additional therapy to ameliorate the symptoms associated with the liver disease or the disease that is treatable with an immunomodulatory disease therapy.

As used herein, “normal” refers to an individual who has not shown any symptoms of a liver disease or a disease that is treatable with an immunomodulatory disease therapy or has not been diagnosed with a liver disease or a disease that is treatable with an immunomodulatory disease therapy. “Normal”, according to the invention, also refers to a sample taken from normal individuals within 14 hours post-mortem. A normal liver tissue sample, for example, refers to the whole or a piece of liver tissue retrieved within 14 hours post-mortem from an individual who was not diagnosed with a liver disease or a disease that is treatable with an immunomodulatory disease therapy and whose corpse does not show any symptoms of a liver disease or a disease that is treatable with an immunomodulatory disease therapy at the time of tissue removal. In alternative embodiments of the invention, the “normal” liver tissue sample is retrieved less than 14 hours post-mortem, e.g., within 13 hours, 12 hours, 11 hours, 10 hours, 9 hours, 8 hours, 7 hours, 6 hours, 5 hours, 4 hours, 3 hours, 2 hours, or 1 hour post-mortem. In one embodiment of the invention, the “normal” liver tissue sample is retrieved 14 hours post-mortem and the integrity of mRNA samples extracted is confirmed.

To determine the “percent identity” of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (e.g., percent identity equals number of identical overlapping positions/total number of positions times one hundred percent). In one embodiment, the two sequences are the same length.

The determination of “percent identity” between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score equal to 100, wordlength equal to twelve to obtain nucleotide sequences homologous to a nucleic acid molecules of the present invention. BLAST protein searches can be performed with the XBLAST program parameters set, e.g., to score-50, wordlength equal to three to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

A particularly useful BLAST program for determining sequence identity is the WU-BLAST-2 program that is described by Altschul et al., Methods in Enzymology, 266:460-480 (1996); http://blast.wustl/edu/blast/REACRCE.html. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity. A percent amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In one embodiment of the invention, percent (%) nucleic acid sequence identity is defined as the percentage of nucleotide residues in a candidate sequence that are identical with the nucleotide residues of the sequence. A preferred method of computing sequence identity utilizes the BLASTN module of WU-BLAST-2 set to the default parameters, with overlap span and overlap fraction set to 1 and 0.125, respectively. The alignment may include the introduction of gaps in the sequences to be aligned. The percentage of homology is determined based on the number of homologous nucleosides in relation to the total number of nucleosides.

As used herein, the term “population” in the context of subjects refers to two or more, preferably 5 or more, 10 or more, 25 or more, 50 or more, 100 or more, 150 or more, 200 or more, 250 or more, 300 or more, or 500 or more subjects.

As used herein, the terms “purified” and “isolated” in the context of a compound other than a nucleic acid molecule or proteinaceous agent, e.g., a compound identified in accordance with the method of the invention, refer to a compound that is substantially free of chemical precursors or other chemicals when chemically synthesized. In a specific embodiment, the compound is 60%, preferably 65%, 70%, 75%, 80%, 85%, 90%, or 99% free of other, different compounds. In a preferred embodiment, a compound identified in accordance with the methods of the invention is purified.

As used herein, the terms “purified” and “isolated” in the context of a nucleic acid molecule refer to a nucleic acid molecule which is separated from other nucleic acid molecules which are present in the natural source of the nucleic acid molecule. Moreover, a “purified” nucleic acid molecule, such as a cDNA molecule, can be substantially free of other cellular material, or culture medium when produced by recombinant techniques, or substantially free of chemical precursors or other chemicals when chemically synthesized. In a preferred embodiment, a nucleic acid molecule is purified.

As used herein, the terms “purified” and “isolated” in the context of a proteinaceous agent (e.g., a peptide, polypeptide, protein or antibody) refer to a proteinaceous agent which is substantially free of cellular material or contaminating proteins from the cell or tissue source from which it is derived, or substantially free of chemical precursors or other chemicals when chemically synthesized. The language “substantially free of cellular material” includes preparations of a proteinaceous agent in which the proteinaceous agent is separated from cellular components of the cells from which it is isolated or recombinantly produced. Thus, a proteinaceous agent that is substantially free of cellular material includes preparations of a proteinaceous agent having less than about 30%, 20%, 10%, or 5% (by dry weight) of heterologous proteinaceous agent (e.g., protein, polypeptide, peptide, or antibody; also referred to as a “contaminating protein”). When the proteinaceous agent is recombinantly produced, it is also preferably substantially free of culture medium, i.e., culture medium represents less than about 20%, 10%, or 5% of the volume of the protein preparation. When the proteinaceous agent is produced by chemical synthesis, it is preferably substantially free of chemical precursors or other chemicals, i.e., it is separated from chemical precursors or other chemicals which are involved in the synthesis of the proteinaceous agent. Accordingly, such preparations of a proteinaceous agent have less than about 30%, 20%, 10%, 5% (by dry weight) of chemical precursors or compounds other than the proteinaceous agent of interest. Preferably, proteinaceous agents disclosed herein are isolated.

As used herein, the terms “therapeutic agent” and “therapeutic agents” refer to any compound(s) which can be used in the treatment, management or amelioration of a liver disease or a disease that is treatable with an immunomodulatory disease therapy or one or more symptoms thereof. In certain embodiments, the term “therapeutic agent” refers to a compound identified in the screening assays described herein. In other embodiments, the term “therapeutic agent” refers to an agent other than a compound identified in the screening assays described herein which is known to be useful for, or has been or is currently being used to treat, manage or ameliorate a liver disease or a disease that is treatable with an immunomodulatory disease therapy or one or more symptoms thereof.

As used herein, the term “therapeutically effective amount” refers to that amount of a therapy (e.g., a therapeutic agent) sufficient to result in the amelioration of a liver disease or a disease that is treatable with an immunomodulatory disease therapy or one or more symptoms thereof, prevent advancement of a liver disease or a disease that is treatable with an immunomodulatory disease therapy, cause regression of a liver disease or a disease that is treatable with an immunomodulatory disease therapy, or to enhance or improve the therapeutic effect(s) of another therapy (e.g., therapeutic agent). In a specific embodiment, a therapeutically effective amount refers to the amount of a therapy (e.g., a therapeutic agent) that reduces liver disease activity, or activity of the disease that is treatable with an immunomodulatory disease therapy, or viral load in the case of a viral infection. Preferably, a therapeutically effective of a therapy (e.g., a therapeutic agent) reduces the swelling of the joint by at least 5%, preferably at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% relative to a control such as phosphate buffered saline (“PBS”).

As used herein, the terms “treat”, “treatment” and “treating” refer to the reduction or amelioration of the progression, severity and/or duration of a liver disease or a disease that is treatable with an immunomodulatory disease therapy or one or more symptoms thereof resulting from the administration of one or more compounds identified in accordance the methods of the invention, or a combination of one or more compounds identified in accordance with the invention and another therapy.

### 4 BRIEF DESCRIPTION OF THE DRAWINGS

FIG. 1 shows an exemplary computer system for use in the methods of the present invention.

FIGS. 2A and 2B illustrate exemplary steps of the method in accordance with one embodiment of the invention.

FIG. 3 shows a plot of the PCR verification for the indicated genes for samples from four responders to a therapy for a genotype 1 hepatitis C viral (HCV) infection, as compared to four genotype 1 HCV non-responder samples and three normal liver samples.

FIG. 4 shows the results of a hierarchical cluster analysis restricted to 18 discriminant genes present in 31 subjects, which includes responders and non-responders.

FIG. 5A shows the results of hierarchical cluster analysis of samples from 31 subjects using a classifier set of 8 genes. FIG. 5B shows the results of nearest neighbor analysis, linear discriminant analysis and principal component analysis of samples from 31 subjects using the classifier set of 8 genes.

FIG. 6A shows the results of hierarchical cluster analysis of samples from only the subjects having a genotype 1 HCV infection, using a classifier set of genes. FIG. 6B shows the results of nearest neighbor analysis, linear discriminant analysis and principal component analysis of samples from only the subjects having a genotype 1 HCV infection, using a classifier set of genes.

FIGS. 7A and 7B show the gene (SEQ ID NO:1) and protein (SEQ ID NO:2) sequences, respectively, of CIG5/Viperin.

FIGS. 8A and 8B show the gene (SEQ ID NO:3) and protein (SEQ ID NO:4) sequences, respectively, of LGP1.

FIGS. 9A and 9B show the gene (SEQ ID NO:5) and protein (SEQ ID NO:6) sequences, respectively, of interferon, alpha-inducible protein (clone IFI-6-16).

FIGS. 10A and 10B show the gene (SEQ ID NO:7) and protein (SEQ ID NO:8) sequences, respectively, of human leucine aminopeptidase 3 (LAP3).

FIGS. 11A and 11B show the gene (SEQ ID NO:9) and protein (SEQ ID NO:10) sequences, respectively, of ubiquitin specific protease 18 (USP18).

FIG. 12 shows a log 2(R) vs log 2(G) plot with a fitted line from a simple linear regression of log₂(R) on log 2(G).

FIG. 13 shows four M vs. A plots of a non-normalized data set with fitted lowess curves.

FIG. 14 shows four M vs. A plots of the normalized data set with fitted lowess curves.

FIG. 15 shows boxplots of 31 non-normalized arrays.

FIG. 16 shows boxplots of 31 normalized arrays.

FIG. 17 shows an exemplary plot of the misclassification error rate versus k obtained using the knn.cv( ) function (nearest-neighbor classifier function) for an estimated gene combination set.

### 5 DETAILED DESCRIPTION OF THE INVENTION

A large proportion of patients do not respond to liver disease therapy regimens, or therapy regimens for diseases that may be treatable with an immunomodulatory disease therapy, for reasons that are unclear. In fact, some of the most effective standard therapies for a liver disease, or a disease that is treatable with an immunomodulatory disease therapy, are completely ineffective for some patients, even while exposing them to unpleasant, and often debilitating, side-effects. Representative liver diseases and diseases that are treatable with an immunomodulatory disease therapy are provided in Section 5.8, below. In addition, many of the standard therapies can be extremely costly and time consuming to implement. A method for predicting a patient's response to a given liver disease therapy regimen or a therapy regimen for a disease that is treatable with an immunomodulatory disease therapy could be used to tailor a treatment regimen that would be more likely to succeed, and thereby reduce the instances of treatment failure or patient relapse. Accordingly, the present invention provides a systems and methods for predicting a patient's response to given liver disease therapy regimens or therapy regimens for diseases that is treatable with an immunomodulatory disease therapy. The invention also provides systems and methods for determining the molecular basis for the lack of effectiveness to standard therapies by certain patients. The present invention also provides systems and methods for identifying genes that, in combination, discriminate between responders and non-responders to the liver disease therapy regimen or the therapy regimen for a disease that is treatable with an immunomodulatory disease therapy. In addition to the significant diagnostic and prognostic benefit, such combinations of genes shed light on the molecular basis of liver disease treatment regimen resistance or resistance to the therapy regimen for the disease that is treatable with an immunomodulatory disease therapy.

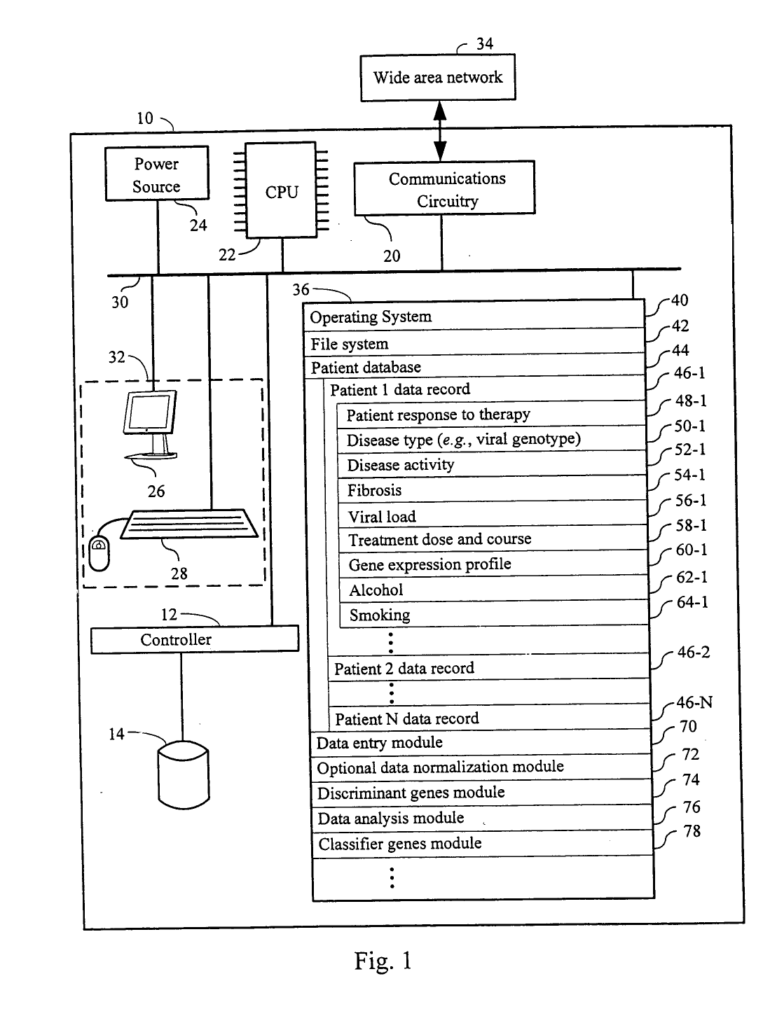

FIG. 1 details an exemplary system for use in the methods of the present invention. The system is preferably a computer system 10 having:

a central processing unit 22;

a main non-volatile storage unit 14, for example a hard disk drive, for storing software and data, the storage unit 14 controlled by storage controller 12;

a system memory 36, preferably high speed random-access memory (RAM), for storing system control programs, data, and application programs, comprising programs and data loaded from non-volatile storage unit 14; system memory 36 may also include read-only memory (ROM);

a user interface 32, comprising one or more input devices (e.g., keyboard 28) and a display 26 or other output device;

a network interface card 20 for connecting to any wired or wireless communication network 34 (e.g., a wide area network such as the Internet);

an internal bus 30 for interconnecting the aforementioned elements of the system; and

a power source 24 to power the aforementioned elements.

Operation of computer 10 is controlled primarily by operating system 40, which is executed by central processing unit 22. Operating system 40 can be stored in system memory 36. In a typical implementation, system memory 36 includes:

an operating system 40;

a file system 42 for controlling access to the various files and data structures used by the present invention;

one or more patient databases 44 for storing patient data;

a data entry module 70 for inputting information into database 44;

an optional data normalization module 72 for optionally normalizing microarray data;

a discriminant genes module 74 that stores information about the set of discriminant genes that differentially express in responders and non-responders to a liver disease therapy regimen or a therapy regimen for a disease that is treatable with an immunomodulatory disease therapy;

a data analysis module 76 for performing classification algorithms; and

a classifier genes module 78 comprising information about the classifier genes that classify patients based on their predicted response to liver disease therapy regimens or therapy regimens for a disease that is treatable with an immunomodulatory disease therapy.

As illustrated in FIG. 1, computer 10 comprises patient database 44. Database 44 can be any form of data storage system including, but not limited to, a flat file, a relational database (SQL), and an on-line analytical processing (OLAP) database (MDX and/or variants thereof). In some specific embodiments, database 44 is a hierarchical OLAP cube. In some specific embodiments, database 44 comprises a star schema that is not stored as a cube but has dimension tables that define hierarchy. Still further, in some embodiments, database 44 has hierarchy that is not explicitly broken out in the underlying database or database schema (e.g., dimension tables are not hierarchically arranged). In some embodiments, patient database 44 is a single database that includes patient data. In other embodiments, patient database 44 in fact comprises a plurality of databases that may or may not all be hosted by the same computer 10. In such embodiments, some component data structures of patient database 44 are stored on computer systems that are not illustrated by FIG. 1 but that are addressable by wide area network 34. Section 5.27 describes exemplary architectures for patient database 44.

In some embodiments, patient database 44 includes records 46 for 10 or more subjects. In some embodiments, patient database 44 includes records 46 for 10 and 100 subjects. In still other embodiments, patient database 44 includes records 46 for between 100 and 500, between 500 and 1000, or more than 1000 subjects. Information about each subject 46 in patient database 44 includes age, sex, whether they smoke or not 64, alcoholic consumption 62, disease activity, treatment dose and course 58, compliance to therapy or dose reduction, and where applicable, baseline viral load 56, disease type 50 (e.g., viral genotype), hepatic fibrosis (i.e., liver scarring) 54, therapy compliance, and dose reduction.

In some embodiments, database 44 and related software modules illustrated in FIG. 1 (e.g. modules 70, 72, 74, 76, and 78) illustrated in FIG. 1 are on a single computer (computer 10) and in other embodiments database 44 and related software modules illustrated in FIG. 1 are hosted by several computers (not shown). In fact, any arrangement of database 44 and the modules illustrated in FIG. 1 on one or more computers is within the scope of the present invention so long as these components are addressable with respect to each other across network 34 or by other electronic means. Thus, the present invention fully encompasses a broad array of computer systems.

5.1 Predicting Clinical Response to Liver Disease Therapy Regimens or Immunomodulatory Disease Therapy Regimens Based on Gene Expression Profiles

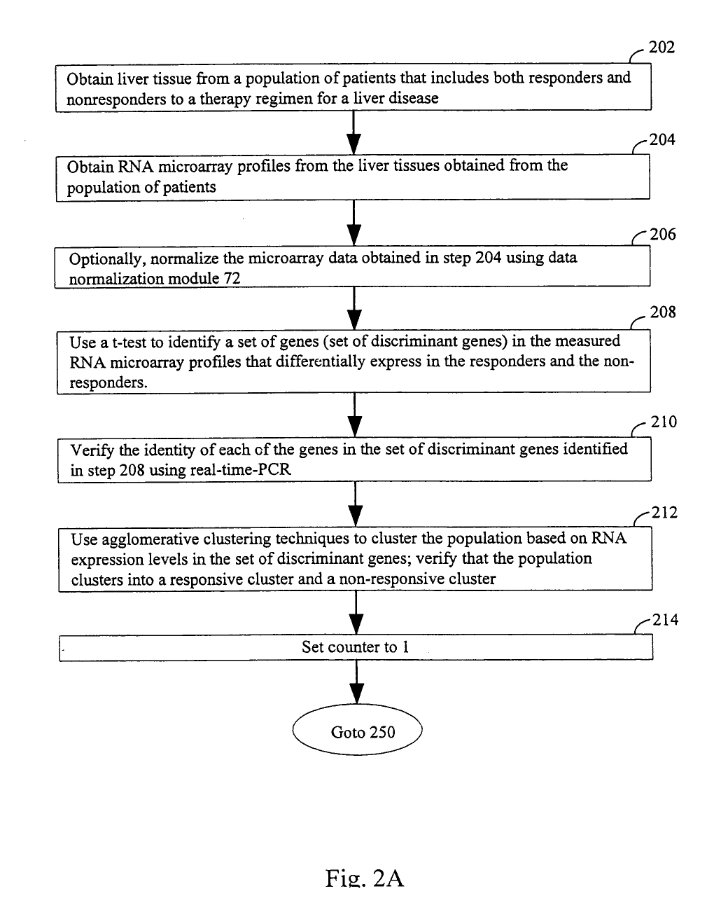

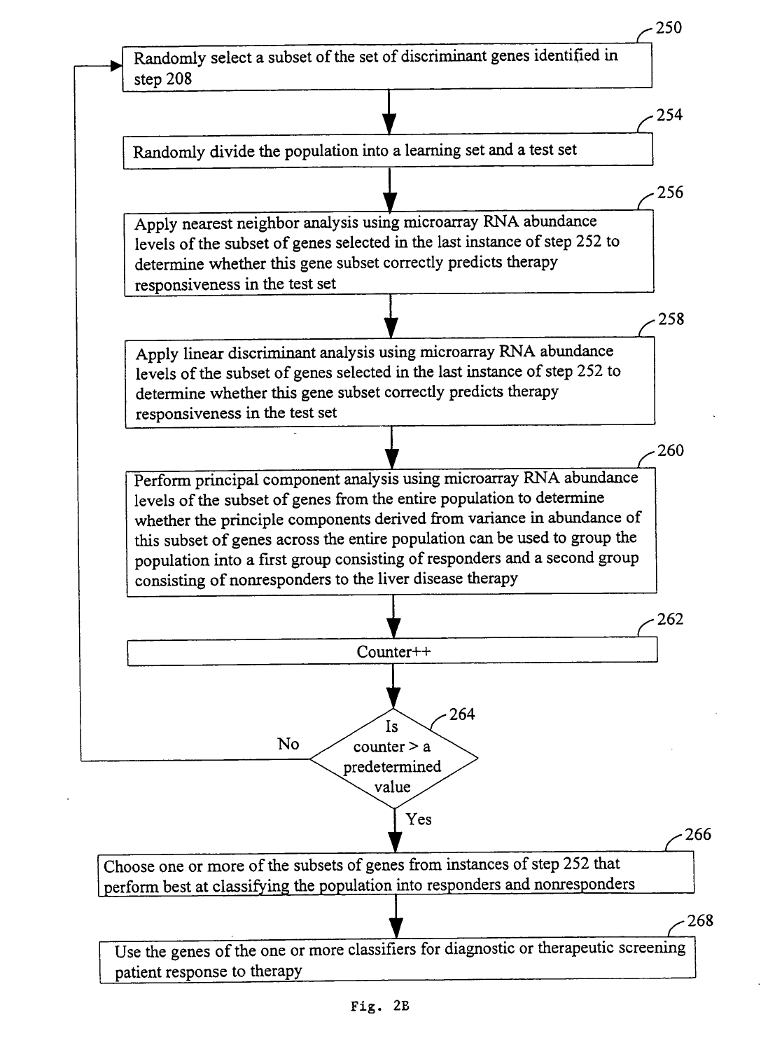

This section describes methods of the present invention for identifying a set of discriminant genes from which one or more sets of classifier genes can be identified. A set of classifier genes is a subset of the set of discriminant genes which can be used to predict a patient's response to a given liver disease therapy regimen or a therapy regimen for a disease that is treatable with an immunomodulatory disease therapy. Exemplary steps in accordance with one embodiment of the invention are illustrated in FIG. 2. While this section is directed to gene expression, it will be appreciated that protein abundance levels of the genes described in this section and referenced in Table 1 could be used instead of, or in addition to, gene expression levels in order to construct discriminators (sets of genes or gene products from those defined in Table 1) that predict a patient's response to a given liver disease therapy regimen or a therapy regimen for a disease that is treatable with an immunomodulatory disease therapy. The method disclosed in FIG. 2 can be conceptualized as having three parts. In the first part, steps 202-212, a population of subjects is used that includes subjects that respond to a treatment regimen (“responders”) and subjects that do not respond to a treatment regimen (“nonresponders”). A set of discriminant genes are identified that differentially express between the responders and the non-responders. In the second part, steps 250-266, a set of classifier genes is derived from the set of discriminant genes. The set of classifier genes is identified from among the set of discriminant genes by identify those genes that perform best at classifying the responders and non-responders. In the third part of the exemplary method, step 268, the set of classifier genes are used for the diagnostic or therapeutic screening of a patient that is not in the initial population. Thus, in step 268, the set of classifier genes is used to determine, in advance of treatment, whether a patient is likely to respond (be a “responder”) or not (be a “nonresponder”) to a given therapy. A more detailed description of the method is presented below.

In part one, steps 202-212 provide a method for identifying a set of discriminant genes that discriminate between responders and non-responders to a liver disease therapy regimen or a therapy regimen for a disease that is treatable with an immunomodulatory disease therapy based on differential gene expression levels between the responders and non-responders. An initial test or trial population was used for identifying the set of discriminant genes. Liver biopsies were taken from the subjects in the trial population prior to initiation of a liver disease therapy regimen or a therapy regimen for a disease that is treatable with an immunomodulatory disease therapy. On completion of the therapy regimen, the subjects in the trial population were tested for responsiveness to the therapy regimen, e.g., whether or not the patient exhibits the desired response conditions to the liver disease therapy regimen or the therapy regimen for a disease that is treatable with an immunomodulatory disease therapy. For example, responsiveness to therapy would mean no detectable viral RNA in the blood in the case of a chronic hepatitis C viral infection. The tests could be performed immediately after completion of the therapy regimen, within a week of completion, or one month, two months, six months or more after completion of the therapy regimen. Based on the test results, the subjects who were responsive to therapy were assigned to a group responder group, and those non-responsive to a non-responder group. The gene expression levels derived from the liver biopsies taken prior to therapy were analyzed relative to the assignment of subject in the population to the responder or non-responder group in order to identify the set of discriminant genes, as described in greater detail below with reference to FIG. 2.

Step 202.

In step 202, a biological sample (e.g., liver, blood, any bodily fluid, any tissue, a biopsy, peripheral mononuclear blood cells, lymphocytes, etc.) was obtained from a patient population that includes both responders and non-responders to a liver disease therapy regimen or a therapy regimen for a disease that is treatable with an immunomodulatory disease therapy. In some embodiments, a tissue (e.g., liver, blood, any bodily fluid, any tissue, a biopsy, peripheral mononuclear blood cells, lymphocytes, etc.) is obtained from 10 or more subjects. In some embodiments, tissue is obtained from between 10 and 100 subjects. In still other embodiments, tissue is obtained from between 100 and 500, between 500 and 1000, or more than 1000 subjects. In some embodiments, certain information about each subject in the patient population is stored in appropriate data fields (e.g., fields 48 through 64 of FIG. 1) in step 202.

Step 204.

In step 204, DNA microarray data was obtained from the tissues of subjects in the population defined in step 202. The DNA microarray data provides expression levels of a plurality of genes expressed in the liver biopsies. In some embodiments, the microarray data was measured as described in Section 5.6. In some embodiments, the gene microarray data from each subject was stored in patient database 44 in fields 60.

Step 206.

In some embodiments, the microarray data obtained in step 204 was normalized using normalization module 72 (see FIG. 1). In other embodiments, the normalization step is optional, and can be omitted. Examples of normalization routines are found in Section 5.5.

Step 208.

In step 208, a t-test was used to identify a set of discriminant genes in the measured DNA microarray profiles that differentially express in the responders and non-responders to the liver disease therapy regimen or a therapy regimen for a disease that is treatable with an immunomodulatory disease therapy. The gene expression levels determined from the liver biopsies or responders and non-responders was compared to identify the set of discriminant genes that is altered between the responders and non-responders. This alteration can be either a relative up-regulation or down-regulation of gene in the non-responders as compared to the responders. For example a gene belongs in the set of discriminant genes if it tends to be expressed at an expression level in the set of responders that is statistically different than the expression level of the same gene in the set on nonresponders. Preferably, the gene expression in the set of discriminant genes can be measured in the samples from all subjects. However, this is not an absolute requirement. Minimally, what is needed to determine whether a gene belongs in the set of discriminant genes is for their to be enough measurements of the gene expression in subjects that are responders to a liver therapy regimen and subjects that are nonresponders to a liver therapy regimen so that a determination can be made as to whether the gene is differentially expressed in the two classes of subjects. In some embodiments, this requirement two or more measurements of the gene among subjects that are responders to a liver disease therapy regimen or a therapy regimen for a disease that is treatable with an immunomodulatory disease therapy, and two or more measurements of the gene among subjects that are not responders to the liver disease therapy regimen or the therapy regimen for a disease that is treatable with an immunomodulatory disease therapy.

A t-test was used to determine whether there is a statistically significant difference between the expression levels between the responders and non-responders in the population identified in step 202. A description of an exemplary t-test that can be used in the present invention is provided in Section 5.3. In some embodiments, the t-test is performed by data analysis module 76. In a preferred embodiment, the difference in the expression levels of a gene in the set of discriminant genes between the responders and non-responders is characterized by a p-value of less than 0.01. More preferably, the difference in the expression levels of a gene in the set of discriminant genes between the responders and non-responders is characterized by a p-value of less than 0.005.

Step 210.

In step 210, the identity of each of the genes in the set of discriminant genes identified in step 208 was verified using real-time-PCR (RT-PCR). Section 5.6.2 provides a description of RT-PCR methods. Given that gene expression differences detected in microarray profiles may not always be reliable or reproducible, real-time PCR serves to independently quantify the gene expression levels first measured using the microarray data. The RT-PCR expression levels were then used in the t-test described in step 208 to verify that the genes first identified as discriminating in step 208 (based upon the microarray data) still discriminate between the responders and the nonresponders of step 202 when RT-PCR data is used. If the t-test results based upon the RT-PCR data were inconsistent with the microarray results for a given gene in the set of discriminant genes, that particular gene was eliminated from the set of discriminant genes.

Step 212.

A hierarchical cluster analysis was performed in step 212 in order to test the differences in the population based on the gene expression levels of the set of discriminant genes identified in step 210. Section 5.4.1 describes unsupervised classification schemes that can be performed by data analysis module 76 in step 212. In a preferred embodiment, the unsupervised hierarchical cluster analysis is an agglomerative clustering technique. In such an embodiment, the expression values for the set of discriminant genes identified in step 208 used to cluster the population identified in step 202. For example, consider the case in which ten molecular markers are selected in step 208 as the set of discriminant genes. Each member m of the population of step 202 will have expression values for each of the ten molecular markers. Such values from a member m in the population define the vector:

| | | | | | | | | | |
|---|---|---|---|---|---|---|---|---|---|
| X₁ₘ | X₂ₘ | X₃ₘ | X₄ₘ | X₅ₘ | X₆ₘ | X₇ₘ | X₈ₘ | X₉ₘ | X₁₀ₘ |

where Xᵢₘ is the expression level of the i^{th} molecular marker in organism m. If there are m organisms in the population identified in step 202, selection of i molecular markers in step 208 will define m vectors. Note that the methods of the present invention do not require that the expression value of every single gene in the set of discriminant genes be represented in every single vector m. In other words, data from an organism in which one of the i^{th} genes is not found can still be used for clustering. In such instances, the missing expression value is assigned either a “zero” or some other normalized value. In some embodiments, prior to clustering, the gene expression values are normalized to have a mean value of zero and unit variance.

Those members of the population of step 202 that exhibit similar expression patterns across the population will tend to cluster together. The set of discriminating genes is considered to be suitable set for use in developing a classifier in this aspect of the invention when the vectors cluster into the two trait groups found in the training population: responders and nonresponders.

Step 214.

In step 214, a counter is set to 1.

In part two, steps 250-266 provide a method of determining a gene subset of the set of discriminant genes that accurately differentiates between non-responders and responders to a given liver disease therapy regimen or a therapy regimen for a disease that is treatable with an immunomodulatory disease therapy. The one or more subsets of genes that accurately classifies the population of step 202 into non-responders and responders are collectively referred to as classifier genes. A random subset of the set of discriminant genes from step 208 was selected and tested for its ability to accurately classify the therapy responsiveness of the subjects in the trial population into responders and nonresponders. Steps 250-260 can be performed any number of times in order to identify one or more sets of classifier genes.

Step 250.

In step 250, a subset of the set of discriminant genes was selected at random to test for its ability to accurately classify the population of step 202 into a responder group and a non-responder group. The subset of discriminant genes can include any subcombination of the set of discriminant genes of step 208. Examples of such subcombinations included random combinations of 4, 6, 8, 10, 12, 14, 16 or more genes in the set of discriminant genes of step 208. Since different gene combinations will have different predictive abilities, each subset is tested for its ability to correctly classify the trial population of step 202 into responders and nonresponders.

Steps 254-256.

At least one supervised classifier analysis technique was performed by module 76 to determine whether the selected subset of genes correctly predicts therapy responsiveness. Supervised classifier analysis techniques are described in Section 5.4.2. In step 254, the trial population of step 254 was first randomly divided into two separate sets: a learning set and a test set. The learning set was grouped into a responder set and a non-responder set according to therapy responsiveness. In some embodiments, the division of the population of step 202 in a given instance of step 254 proceeds as follows. Gene expression data on p genes for n mRNA samples can be summarized by an n times p matrix X=(xᵢⱼ) where xᵢⱼ denotes the expression level of gene (variable) j in mRNA sample (observation) i. When mRNA samples belong to known classes the data for each observation consist of a gene expression profile xᵢ=(xᵢₗ, . . . , xᵢₚ) and a class label yᵢ, e.g., of predictor variable xᵢ and response yᵢ. Let K define a set of classes yᵢ then nₖ denote the number of observations belonging to class k.

Let LS denote a learning set of gene expression profiles selected in the last instance of step 250 LS={(xₗ,yₗ) , . . . , (xₙₗ,yₙₗ)} of known class labels {yₗ, . . ,yₙ} (here n=2 and consists of responders and nonresponders) and let T={xₗ, . . . ,xₙ} denote the test set of observations xᵢ. The predictor set of known classes (e.g., the learning set LS) can be used to predict the class for each observation xᵢ in the test set T.

In step 256, a nearest-neighbor analysis was performed. Such an analysis requires the division of the population into a learning set and a test set that was performed in step 254. The learning set LS was used as neighbors as detailed in Section 5.4.2.1. Then, a misclassification rate was computed. In typical embodiments, steps 254 and 256 were repeated several times for a given subset of the set of discriminant genes and the misclassification rate from each of these cycles of steps 254 and 256 is determined by summing the misclassification rate from each of the cycles and then dividing by the number of cycles that were performed. For example, in some embodiments, steps 254 and 256 were repeated 1,000 times for a given subcombination of the set of discriminant genes. Each cycle produced an error rate. The error rates were summed and divided by 1000 in order to obtain the overall error rate for the subcombination of genes selected in the last instance of step 250.

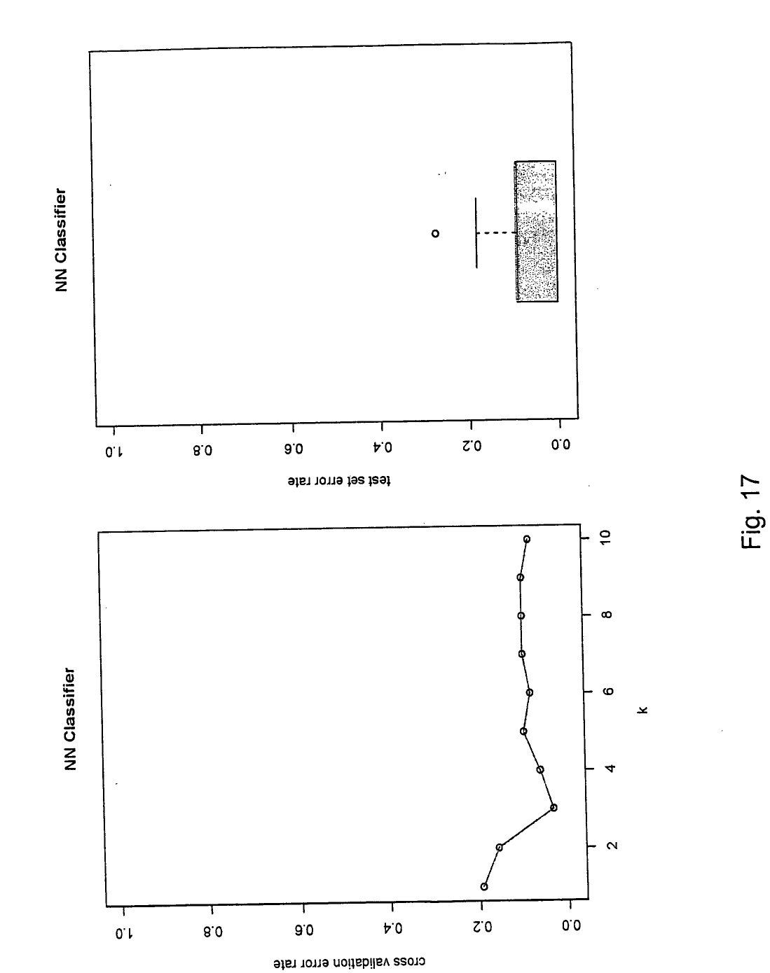

In some embodiments, the misclassification rate was calculated using a k-nearest neighbor cross-validation classification function knn.cv( ). See, e.g., Mardia, K. V., J. T. Kent, and J. M. Bibby, “Multivariate Analysis, London: Academic Press (1979); Venables, W. N. and B. D. Ripley, “Modern Applied Statistics with S-PLUS,” Springer-Verlag (1997); and Venables, W. N. and Ripley, B. D., “Modern Applied Statistics with S,” 4^{th} edition, Springer, 2002. Subsets of genes with the lowest misclassification rate were selected and then gene combinations which performed best in both the unsupervised and supervised analyses are also selected. FIG. 17 shows an exemplary plot of the misclassification error rate versus k obtained using the knn.cv( ) function for an estimated gene combination set.

The misclassification error rate of a classifier can be estimated using a 2:1 sampling scheme. For each run the data set X is randomly divided into a learning set and test set.

In a specific embodiment, the learning set contains two thirds of the data set, while the test set contains one third of the data set. Then a predictor set of eight genes with p values <0.0001 and folds>=|1.5| was selected from the learning set and applied to the test set.

The misclassification rate is calculated in each run over r=94 runs in some mbodiments. The estimated error rate for the subset of genes is then given by

$$< E >= \frac{1}{r} \sum \frac{r}{r = 1} {E}_{r} = 0.21$$

as shown in FIG. 17.

Step 258.

In step 258, a linear discriminant analysis (LDA) was performed. LDA attempts to classify a subject into one of two categories based on certain object properties. In other words, LDA tests whether object attributes measured in an experiment predict categorization of the objects. LDA typically requires continuous independent variables and a dichotomous categorical dependent variable. In the present invention, the expression values for the genes selected in the last instance of step 250 across the population of step 202 serve as the requisite continuous independent variables. The trait subgroup classification of each of the members of the training population serves as the dichotomous categorical dependent variable.

LDA seeks the linear combination of variables that maximizes the ratio of between-group variance and within-group variance by using the grouping information. Implicitly, the linear weights used by LDA depend on how the expression of a gene across the population of step 202 separates in the two groups (e.g., the responder and the nonresponder group) and how this gene expression correlates with the expression of other genes. In some embodiments of step 258, LDA was applied to the N members in the population of step 202 by the K molecular markers in the combination of genes selected in the last instance of step 250. Then, the linear discriminant of each member of the learning set was plotted. Ideally, those members of the training population representing a first trait subgroup (e.g., the responders) will cluster into one range of linear discriminant values (e.g., negative) and those member of the training population representing a second trait subgroup (e.g., the nonresponders) will cluster into a second range of linear discriminant values (e.g., positive). The LDA is considered more successful when the separation between the clusters of discriminant values is larger. For more information on linear discriminant analysis, see Duda, Pattern Classification, Second Edition, 2001, John Wiley & Sons, Inc; and Hastie, 2001, The Elements of Statistical Learning, Springer, New York; Venables & Ripley, 1997, Modern Applied Statistics with s-plus, Springer, N.Y., which is hereby incorporated by reference in its entirety. More information on how LDA is computed in one embodiment of the present invention is found in Section 5.4.2.2.

Step 260.

In step 260, a principal component analysis was performed using the microarray RNA abundance levels of the subset of genes from the entire population of step 202 to determine whether the principal components derived from variance in abundance of the subset of genes across the entire population of step 202 can be used to group the trial population into a first group consisting of responders and a second group consisting of non-responders to the liver disease therapy regimen or a therapy regimen for a disease that is treatable with an immunomodulatory disease therapy. More information on principal component analysis is provided in Section 5.4.1.3.

Step 262.

In step 262, the counter from step 214 was advanced by one after each iteration of the selection and evaluation process for a subset of genes in the set of discrimant genes.

Step 264.

In step 264, a determination was made as to whether the loop defined by steps 250-264 has been computed a predetermined number of times. If so, (264-Yes) process control continued to step 266. If not (264-No), process control returned to step 250 where a new subset of the set of discriminant genes of step 208 is selected. In principle, steps 250-264 can be performed any number of times in order to identify one or more subsets of classifier genes. In some embodiments, steps 250-264 are repeated up to 1,000, 10,000, 25,000, 50,000 or more times.

Step 266.

In step 266, one or more of the subsets of discriminant genes (classifier genes) were chosen that (i) had the lowest misclassification rate, as judged by the k-nearest neighbor cross-validation classification, and that (ii) performed best in both the principal component analysis and the linear discriminant analysis. In some embodiments, a single set of classifier genes was identified for its predictive ability to accurately classify the trial population.

Step 268.

In part three, step 268, the one or more sets of classifier genes identified in step 266 were used for diagnostic or therapeutic screening of a patient response to a therapy regimen for a liver disease or an immunomodulatory disease therapy regimen. Given the method provided in FIGS. 2A and 2B, and the description of each stage of the method provided above, any one, two, four or more of the discriminant genes or classifier genes identified in steps 208, 210 or 266 could be used to discriminate between responders and non-responders to a therapy for a liver disease or a disease that is treatable with an immunomodulatory disease therapy. Therefore, any one, two, four or more of the genes and gene products identified in steps 208, 210 or 266 are useful for diagnosing a disease, such as any of the diseases listed in Section 5.8. Generally, naturally occurring, e.g., non-recombinant, protein and RNA can be used for the purposes of diagnosis and prognosis. Additionally, any one, two, four or more of the genes and gene products identified in steps 208, 210 or 266 are useful for predicting a subject's resistance or non-resistance to a therapy regimen for these diseases. Moreover, modulators of the activity or abundance levels of the genes and gene products identified in steps 208, 210 or 266 are useful in treating a disease, such as any of the diseases listed in Section 5.8. Also, modulators of the genes and gene products identified in steps 208, 210 or 266 are useful in treating a disease, such as any of the diseases listed in Section 5.8. In a specific embodiment, the diseases are treatable with an immunomodulatory disease therapy, such as the interferon-treated diseases listed in Section 5.8.2.

Any of the genes identified in steps 208, 210 or 266 can be used in accordance with step 268 for diagnostic and therapeutic screening of a patient response to a therapy regimen for a disease. Also, any number or combination of the genes identified in steps 208, 210 or 266 can form a set of classifier genes for responsiveness to a therapy regimen for a disease. A subset or sub-combination of the genes identified in steps 208, 210 or 266 forming a set of classifier genes can consist of 2, 4, 6, 8 or more of the genes. A subset or sub-combination of the genes identified in steps 208, 210 or 266 forming a set of classifier genes can comprise 1, 2, 4, 6, 8 or more of the genes. In some embodiments, the set of genes used to discriminate between responders and non-responders consists of no more than 50 genes. In other embodiments, the set of genes used to discriminate between responders and non-responders consists of no more than 40, 25, 15, 10 or 8 genes of the genes identified in steps 208, 210 or 266. In specific embodiments, a plurality of products consists of the respective products of a maximum of 100, 50, 40, 25, 15, 10 or 8 genes, and optionally, at least of 100, 50, 40, 25, 15, 10, 8, 4 or 2 of the genes.

In some embodiments, expression levels from a test subject are used in a nearest neighbor analysis. Recall that several possible subcombinations of the set of discriminant genes of step 208 were tested in iterations of loop 250-264. For each of these subcombinations, a nearest neighbor analysis, a linear discriminant analysis, and a principal component analysis was developed. Therefore, for the set of classifier genes selected in step 266, there exists suitable models for nearest neighbor analysis, linear discriminant analysis, and principal component analysis based upon the training population of step 202. These models can be used to classify a new subject as either responsive or nonresponsive. For instance the expression levels of the set of classifier genes selected in step 266 can be measured from a liver biopsy of the subject and used to classify the subject as a responder or a nonresponder using the trained nearest neighbor model of step 256. Alternatively, or additionally, the expression levels of the set of classifier genes selected in step 266 can be measured from a liver biopsy of the subject and used to classify the subject as a responder or a nonresponder using the linear discriminant analysis model of step 258. Alternatively, or additionally, the expression levels of the set of classifier genes selected in step 266 can be measured from a liver biopsy of the subject and used to classify the subject as a responder or a nonresponder using the principal component analysis model of step 260.

In fact, any set of classifier genes from the set of discriminant genes can be used in a classification technique to classify subjects as nonresponders. Such classification techniques include the four that were described in conjunction with FIG. 2 (clustering, nearest neighbor analysis, linear discriminant analysis, and principal component analysis). However, the invention is not so limited. Any form of pattern classification technique and/or statistical technique known in the art that can classify a subject into two classifications can be used. Exemplary additional techniques that can be used to classify subjects into responders and nonresponders using subsets of the set of discriminating genes are described in Section 5.28 below.

The present invention further contemplates that each gene in the set of discriminant genes of step 266 can individually be screened in order to identify compounds useful in the treatment of a liver disease or a disease that is treatable with an immunomodulatory disease therapy, such as the diseases listed in Section 5.8. Such methods are disclosed in Sections 5.9 through 25, below. Compounds identified using the methods of Sections 5.9 through 25 can be used as diagnostics as disclosed in Section 5.26.

5.1.1 Classifying Responders and Non-Responders to Hepatitis C Viral Infection Therapy

PegIFN plus ribavirin (PegIFN/rib) treatment is the most effective treatment for chronic Hepatitis C viral infection (HCV), and is increasingly used despite unpleasant side effects and high costs. However, a large proportion of patients do not respond to therapy for reasons that are unclear. It would therefore be advantageous to be able to predict a patient's response to the treatment before initiation of a treatment regimen. Accordingly, one aspect of the present invention provides a method for identifying a set of discriminant genes (and from this one or more sets of classifier genes) that can be used for predicting a patient's response to a therapy regimen for a hepatitis C viral infection. In addition, it would be advantageous to be able to use gene expression profiling to determine a molecular basis for treatment failure, and as a result be able to provide alternative treatments for the patient. Accordingly, another aspect of the present invention provides a method for determining the molecular basis for treatment failure. This section presents a non-limiting example of the practice of the methods of the invention for identifying discriminant and classifier genes for patient response to a PegIFN/rib treatment regimen for HCV using a trial population of 31 subjects.

In step 202 of FIG. 2A, needle liver biopsies were taken by protocol prior to therapy from a trial population of patients. The data was entered into patient database 44 for each subject in the trial population is presented in Table 4. The patients in this study were well-matched for most clinical variables with the exception of viral genotype and sex. There were no significant differences between the subjects in the responder group (R) and non-responder group (NR) when compared for age, baseline viral load, disease activity, hepatic fibrosis, compliance to therapy or dose reduction. The liver disease type, e.g., HCV of genotype 1, 2, 3 or 6, was also entered into patient database 44. Table 4 shows that infection with genotype 1 had the highest failure rate with therapy in the trial population, in that all NR patients were infected with HCV genotype 1. The data in Table 4 is presented as mean±standard deviation (SD).

Where data is presented in fractions, the denominator represents the number of patients for whom full data was available. Statistics are either Welch t-test or chi-square analysis. The number of patients who receive at least 80% of the dose of Peg/IFN/rib for at least 80% of the time is also recorded in database 44 over the course of therapy.

In accordance with steps 204-208, gene expression levels were determined for the subjects in the trial population and compared to identify a set of discriminant genes. A 19000 gene microarray was employed to compare hepatic gene expression profiles from liver biopsies taken on the 31 subjects (15 NR and 16 R) prior to treatment with PegIFN/rib in order to determine which hepatic genes discriminate between HCV infection of responders and non-responders.

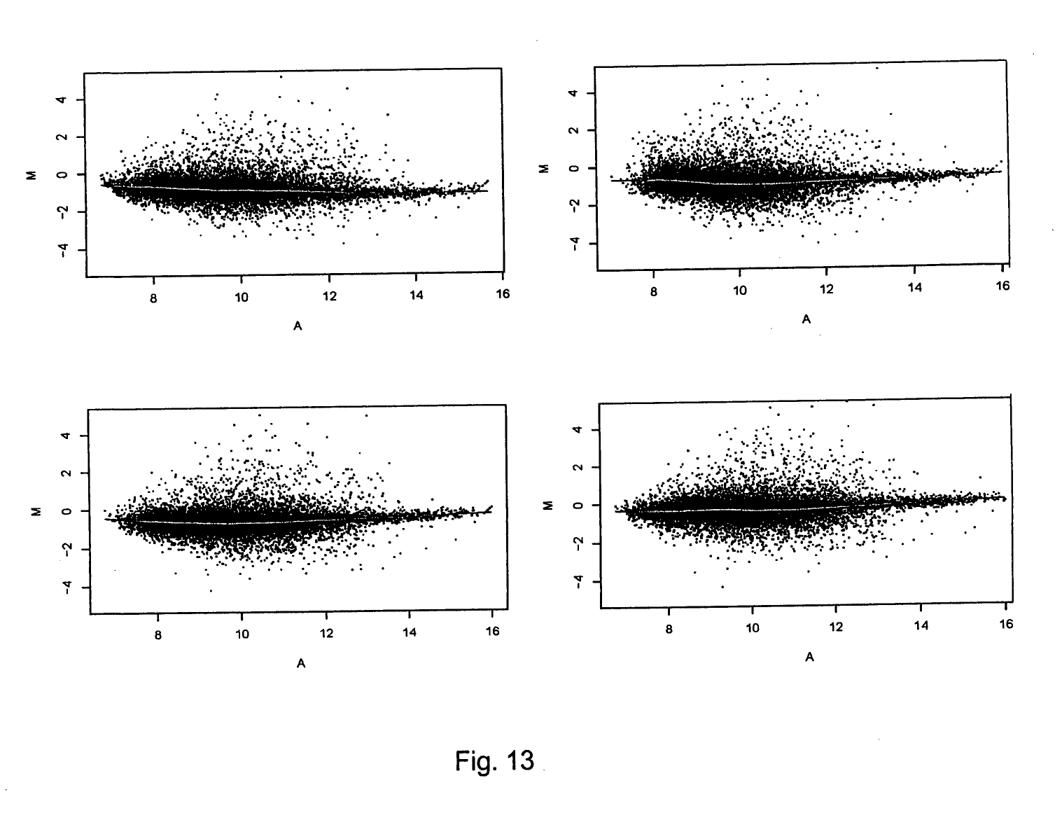

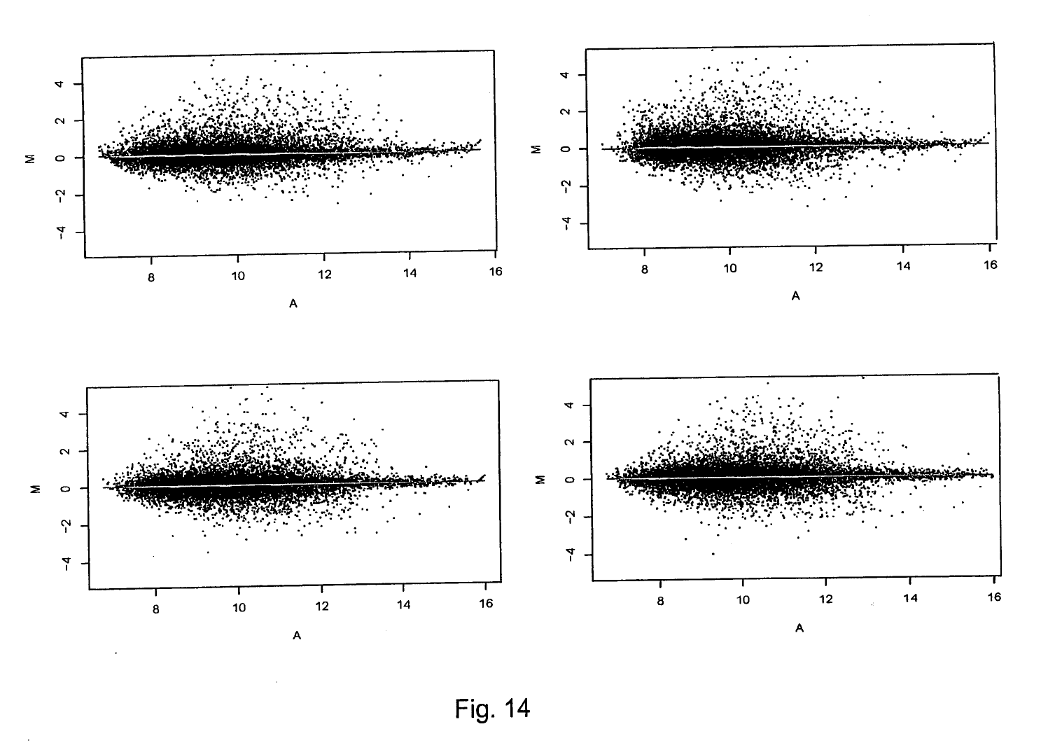

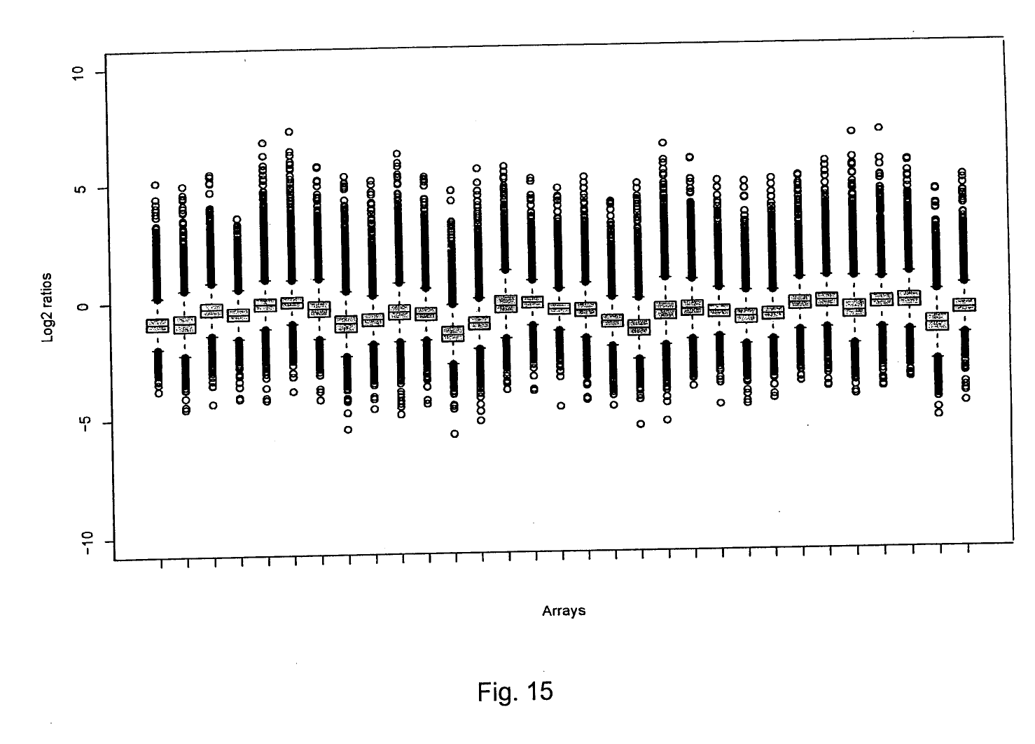

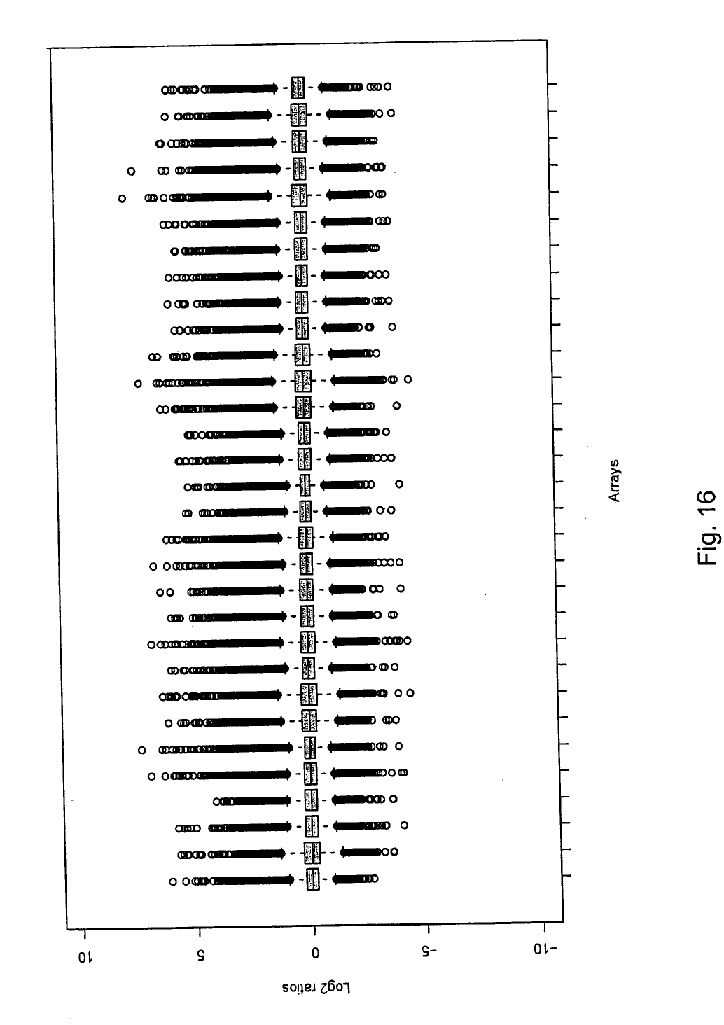

In a specific embodiment, the data was normalized using data normalization module 72 prior to the step of comparing. FIG. 13 shows four M vs. A plots of the non normalized data set with fitted lowess curves, while FIG. 14 shows four M vs. A plots of the normalized data set with fitted lowess curves, as described in Section 5.5. FIG. 15 shows boxplots of the 31 arrays which have been normalized using the intensity dependent normalization method. In this example, the differences in scales are not large enough as to scale the log₂ ratios between the arrays. FIG. 16 shows boxplots of 31 non-normalized and normalized arrays. In other embodiments, the normalization routine is omitted.

The change from baseline, uninfected hepatic expression, is assessed by comparing the expression levels of genes found to be significantly altered between NR and R liver tissue to that found in biopsies from 20 normal livers using a t-test, in accordance with step 208. Preferably the expression level differs consistently between NR and R liver tissue and does not correlated to any obvious clinical parameter. In a specific embodiment, the t-test is performed by data analysis module 76 using a multtest( ) package, as described in greater detail in Section 5.3. A total of forty genes, listed in Table 1, were identified whose gene expression level could be both measured in 75% of more of the samples and differed between the R and NR groups with a p-value of 0.05, and which could be used to discriminate between the groups. The GenBank Accession number is provided for each gene in Table 1 (NCBI GenBank Database: http://www.ncbi.nlm.nih.gov/entrez/query.fcgi?db=Nucleotide).

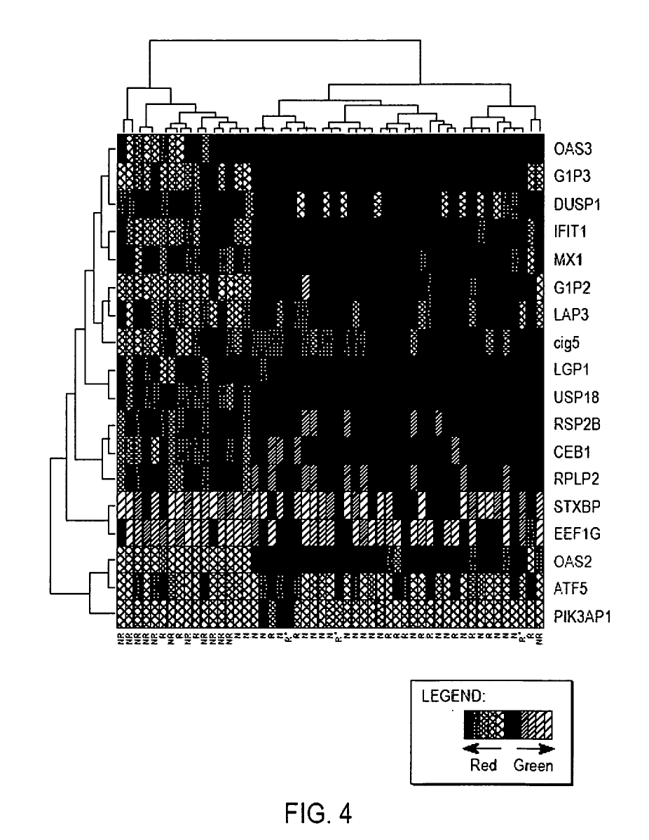

In a specific embodiment, two, four, six or more of the forty genes of Table 1 can be used as a set of classifier genes. Of the forty genes listed in Table 1, a total of 18 discriminant genes, listed in Table 5, are identified whose gene expression level could be both measured in all samples and differed between the R and NR groups with a p-value of less than 0.005. Most of the difference between NR and sustained virologic response (SVR) samples is a relative up-regulation of genes in NR tissue. When comparing only the genes that discriminate between R and NR liver, R gene expression profiles actually co-cluster with normal liver (FIG. 4). If the analysis is performed with a p-value of 0.01, then a larger number of candidate discriminant genes are found, including regucalcin gene promotor region related protein (RGPR). Table 2 lists candidate discriminant genes for a p-value of 0.01 for all genotypes, including genotypes 1, 2, 3, and 6, while Table 3 lists candidate discriminant genes for genotype 1 samples only.

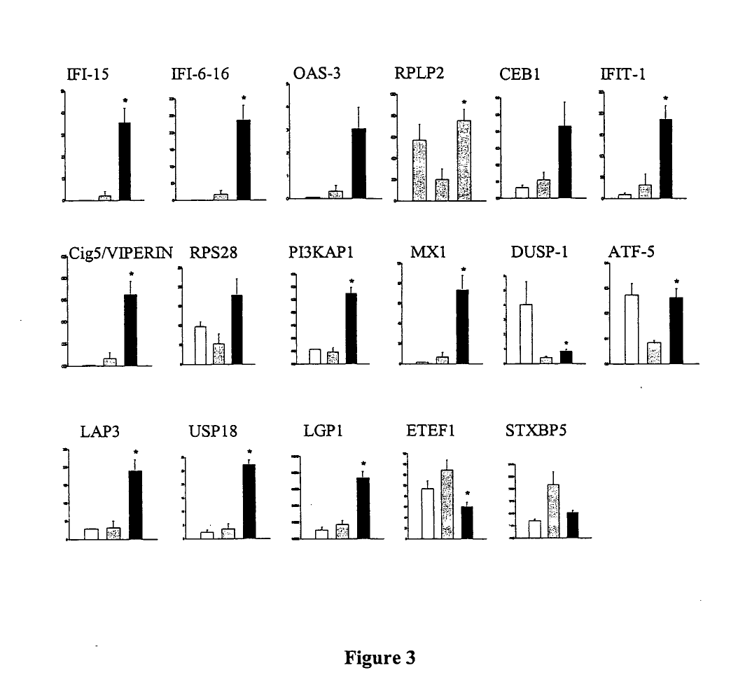

Gene expression differences detected in microarray studies does not always prove reproducible. Therefore, in accordance with step 210, the identity of the 18 discriminant genes identified using microarrays is independently verified using real-time PCR. Real-time PCR also independently quantifies the differences suggested by the DNA microarray. A list of the primers that can be used for the real-time PCR of each of 18 discriminant genes is provided in Table 7. FIG. 3 shows a plot of the PCR verification for the indicated 18 genes for four genotype 1 R samples, as compared to four genotype 1 NR samples and three normal liver samples. Preferably, these differences are maintained regardless of the genotype of the samples chosen for quantitative PCR.

In accordance with step 212, an unsupervised hierarchical cluster analysis was performed in order to test for differences in hepatic gene expression profiles between normal and infected liver tissue. This analysis is limited to the 18 genes found to be statistically different between NR and R liver tissue, and compared normal, NR and R liver tissue. FIG. 4 shows the results of a hierarchical cluster analysis restricted to the 18 discriminant genes present in the 31 subjects. Red denotes an increase and green a decrease when compared to the reference RNA pool. The asterisk denotes the subjects who relapsed following treatment with IFN α/ribavirin. Normal liver tissue was found to co-cluster with patients who responded to treatment, while all NR samples form part of a discrete cluster. As predicted from the results of Table 5, the cluster analysis clearly segregated all NR samples in one family, with all but 2 R samples and all normal liver samples segregated in another large cluster. The results of the real-time PCR verified the identity of 18 discriminant genes for responders and non-responders to a PegIFNα plus ribavirin (PegIFN/rib) treatment for a hepatitis C viral infection as the following: G1P2/ISG15/IFI-15, G1P3/IFI-6-16, OAS3, RPLP2, CEB1, VIPERIN/CIG5, PI3KAP1, MX1, LAP3, ETEF1, IFIT1/IFI56, OAS2, DUSP1, ATF5, LGP-1, RPS28, USP18/UBP43, and STXBP5.

In another aspect of the methods of the invention, a subset of the set of discriminant genes, the classifier genes, was identified in accordance with steps 250-266. A set of classifier genes can include two, four, six, eight or more of the discriminant genes. Since hierarchical clustering in general is not robust and is sensitive to small changes in the data, which then can produce very different results, one or more supervised classification analyses is performed to identify the classifier genes. The unsupervised hierarchical cluster analysis is highly suggestive of a consistent difference between NR and R samples. This form of analysis was supplemented with other forms of analysis as described below.

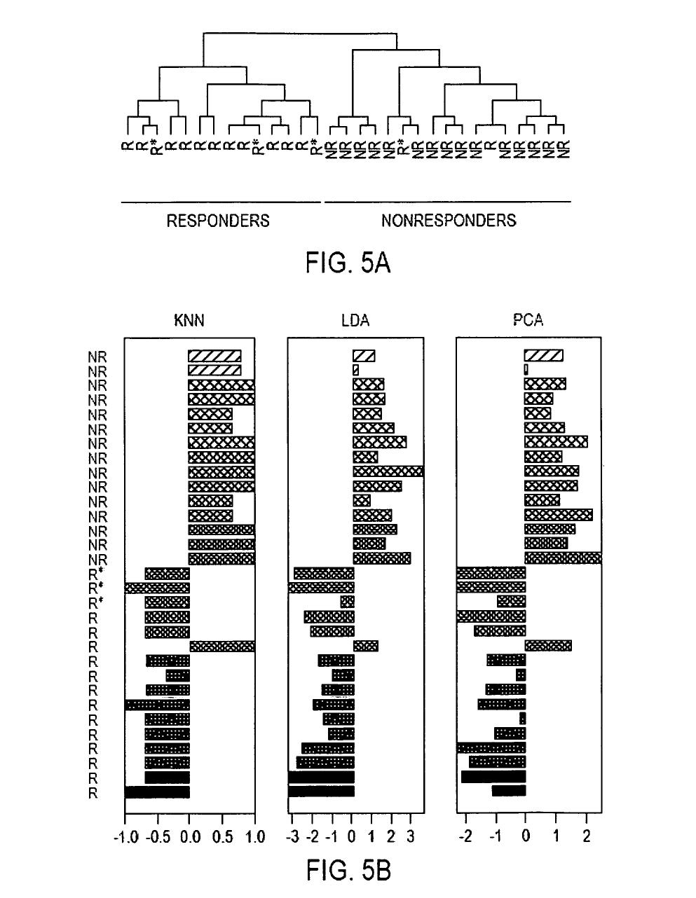

Since different gene combinations have different predictive abilities, randomly selected combinations of the discriminant genes are assessed for their ability to correctly classify the 31 NR and R samples. In order to determine whether the discriminant genes can be used to predict treatment response, both nearest-neighbors analysis (KNN) and linear discriminants analysis (LDA) are performed on the subset of discriminant genes. FIG. 17 shows boxplots of an HCV data test set error rates from 94 runs for a sampling scheme for a nearest neighbor classifier built using 8 preselected genes, with two thirds of the population placed in the learning set and one third in the test set. The results of the supervised classification analyses were then corroborated using principal component analysis. The set of classifiers genes for patient response to a PegIFN/rib treatment regimen for HCV with the highest overall classification accuracy was G1P2, ATF5, IFIT1, MX1, USP18/UPB43, DUSP1, CEB1, and RPS28. FIG. 5A shows the results of hierarchical cluster analysis of all samples using the eight classifier genes for all subjects. FIG. 5B shows the results of nearest neighbor analysis, linear discriminant analysis and principal component analysis of all subjects using the eight classifier genes. In both figures, an asterisk denotes treatment relapsers. Using this predictive gene subset both KNN and LDA classifier analyses accurately identified 30 of 31 samples, while the PCA analysis clearly separated R and NR samples into two distinct groups (FIG. 5B).

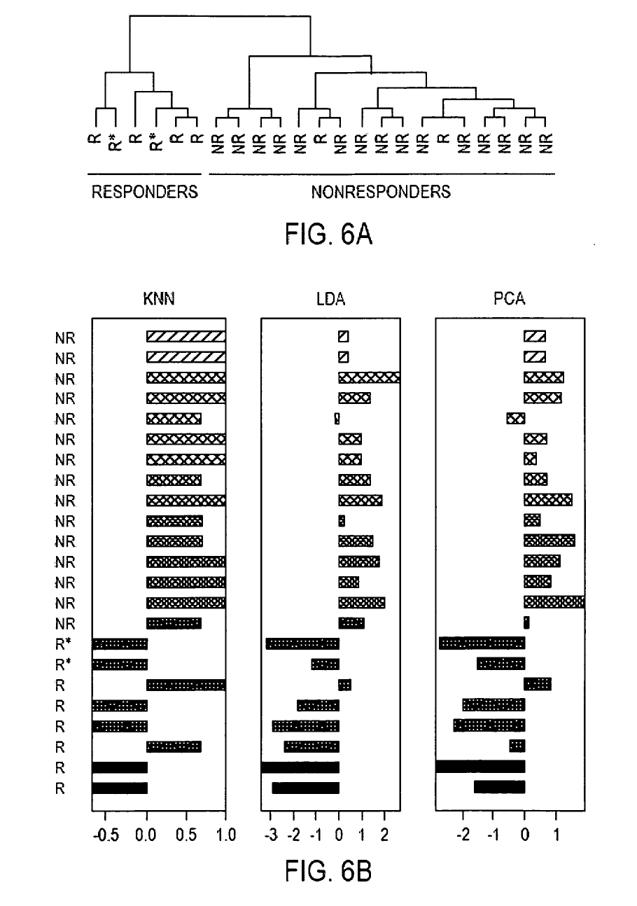

The classifier genes are seen to predict 30/31 outcomes in the cohort of 31 patients with chronic HCV. However, since genotype 1 patients are the least likely to respond to treatment (and in fact formed the entire NR arm of the cohort), the classifier genes are also examined for ability to predict the response of the 23 genotype 1 subjects in the trial population. As shown in Table 6, among the patients infected with genotype 1, there were no significant differences in age, sex, baseline viral load, disease activity, hepatic fibrosis, treatment compliance or PegIFN/rib dose reduction in the genotype 1 NR and R patients. FIG. 6A shows the results of hierarchical cluster analysis of the genotype 1 samples only, using the eight classifier genes for all subjects. FIG. 6B shows the results of nearest neighbor analysis, linear discriminant analysis and principal component analysis of the genotype 1 subjects using the eight classifier genes. The classifier genes were shown to correctly classify 21/23 samples using nearest-neighbors and linear discriminants analysis, while principal components analysis clearly created two distinct clusters (FIG. 6).

The mathematical models used in the exemplary embodiment of an PegIFN/rib therapy regimen for a hepatitis C viral infection mathematical model include clustering, principal component analysis, nearest neighbor analysis, and linear discriminant analysis. However, other classification schemes or mathematical model that can be used in other embodiments of the invention include regression models, neural networks, quadratic discriminant analysis, support vector machines, decision trees, evolutionary methods, random subspace methods or other algorithms. Those of skill in the art recognize these and other classification scheme or mathematical model which are applicable to the methods of the present invention.

The identity of the differentially regulated genes also suggests a mechanism for resistance to treatment. The non-responders are characterized by a general up-regulation of interferon-responsive genes, both in comparison to R and to normal liver tissue. Therefore, in another aspect of the invention, hepatic gene expression profiling identified consistent molecular differences in subjects who subsequently fail PegIFN/rib treatment: the upregulation of a specific set of IFN-responsive genes in NR livers translates to non-response to exogenous therapy. In accordance with another aspect of the present invention, the identified discriminant and classifier genes is used in predicting clinical responses to treatment in step 268 of FIG. 2B. Subjects in the non-responder and responder groups are found to differ fundamentally in their innate interferon response to HCV infection. The profile of patients responding to treatment is found to be more similar to uninfected samples. The major contributor to the difference is an up-regulation of gene expression in NR liver. HCV infection of NR patients is associated with a consistent alteration in local hepatic gene expression not found following HCV infection of patients who will subsequently respond to treatment. Many of the discriminant genes are IFN-responsive, suggesting that the NR patients have adopted a different, yet characteristic, equilibrium in their host-virus immune response. In a further aspect, the invention provides therapeutic approaches that modify the host immune response, which may increase the efficacy of the interferon treatment. The present invention takes advantage of these differences in gene expression levels to provide novel aspects of HCV pathogenesis. These differences also form the basis for the predictive subset of classifier genes that can be used to predict treatment responses prior to initiation of PegIFN/rib therapy.

As described above, the methods of the present invention can be performed on a relatively small trial population, e.g., 30 subjects or less. In fact, an accurate set of classifier genes can be developed from even smaller patient numbers. When expression profiles from the first five nonresponders and seven responders in the exemplary trial population were compared, the seven genes that were most statistically different between these two groups accurately predicted 17 of the 19 subsequent outcomes (accrued on a prospective basis). Two of the seven genes were included in the set of 8 classifier genes, namely USP18 and IFIT1. This finding argues that the difference between NR and R liver gene expression profiles is highly consistent and therefore can form the basis for an accurate prediction system. Therefore, in other embodiments the trial population includes less then 30 subjects. In alternate embodiments, the trial population includes 40, 50, 100 or more subjects.

If validated prospectively on 42 additional samples the predictor set is 100% accurate in predicting the responders (specificity=100%) while its sensitivity is estimated to be 69% also its positive predictive value (PPV) is calculated to be 1 while its negative predictive value (NPV) is calculated to be 0.39. The predictor set is also 69% curate in predicting the non responders (specificity=69%) while its sensitivity is estimated to be 100% also its positive predictive value (PPV) is calculated to be 0.39 while its negative predictive value (NPV) is calculated to be 1.

Once identified, the classifier genes are broadly applicable. The methods of the invention define non-responder status at a molecular level, e.g., when compared to normal liver tissue, the principal difference between NR and R liver biopsies is found to be an altered expression of genes in NR tissue. The difference in gene expression profiles could not be explained by differences in local inflammation alone, since R and NR subjects in the trial population were well-matched in terms of viral load, disease activity and hepatic fibrosis. The practice of the methods of the present invention shows that HCV infection of NR patients affects a fundamentally different response than does HCV infection of R patients. The method of the present invention is found to be a better predictor of response to therapy than the standard clinical predictors.

A recent report compared 5NR and 10R liver biopsies with a 200 ISG gene microarray (see, Daiba et al., 2004, Biochem Biophys Res Commun. 315: 1088-96, which is hereby incorporated by reference in its entirety). In this study, liver biopsies were collected over an 8-year period from two institutions, treatment regimens differed, and the NR profile was characterized by a marked down-regulation of gene expression. However, the set of discriminant genes and classifier genes of the present invention were not identified as important in discriminating NR and R patients in this analysis, even though the 19,000 gene array used in the exemplary embodiment of the invention contains many of the genes in the 200 ISG gene microarray. Additionally, the present invention is the first to comprehensively investigate the basis of PegIFN/rib nonresponder status using gene expression profiling. Also, the present invention is the first to identify the set of discriminant genes and classifier genes for predicting response to PegIFN/rib treatment for HCV.

Any of the genes listed in Table 1 can be used in accordance with step 268 for diagnostic and therapeutic screening of a patient response to a therapy regimen for a disease. Also, any number or combination of the genes listed in Table 1 can form a set of classifier genes for responsiveness to a therapy regimen for a disease. A subset or sub-combination of the genes listed in Table 1 forming a set of classifier genes can consist of 2, 4, 6, 8 or more of the genes. A subset or sub-combination of the genes listed in Table 1 forming a set of classifier genes can comprise 1, 2, 4, 6, 8 or more of the genes. In an exemplary embodiment, the estimated error rate for a classifier consisting of one gene (G1P2) was 10% by cross validation using only the training set of 31 samples. When the one gene classifier was applied to different sample set of 18 subjects in all the error rate becomes 28%, as opposed to 22% using an 8 gene classifier set. In another exemplary embodiment, the estimated error rate for a classifier consisting of two genes (OAS3 and ATF5) was 8% by cross validation, using only the training set of 31 samples. When the two gene classifier was applied to a different sample set of 18 subjects in all the error rate becomes 28%, as opposed to 22% using an eight gene classifier set. In some embodiments, the set of genes used to discriminate between responders and non-responders comprises no more than 50 genes. In other embodiments, set of genes used to discriminate between responders and non-responders comprises no more than 40, 25, 15, 10 or 8 genes of the genes set forth in Table 1. In specific embodiments, a plurality of products consists of the respective products of a maximum of all, 25, 15, 10 or 8 genes set forth in Table 1, and optionally, at least 15, 10, 8, 4 or 2 of the genes set forth in Table 1.

Given the process in FIGS. 2A and 2B and the description provided above, any one, two four or more of the genes listed in Table 1 could be used to discriminate between responders and non-responders to a therapy regimen to a liver disease or a disease that is treatable with an immunomodulatory disease therapy. Therefore, the genes and gene products of Table 1 are useful for diagnosing a disease, such as any of the diseases listed in Section 5.8. Additionally, any one, two, four or more of the genes and gene products listed in Table 1 are useful for predicting a subject's resistance or non-resistance to a therapy regimen for these diseases. Moreover, modulators of the activity or abundance levels of the genes and gene products listed in Table 1 are useful in treating a disease, such as any of the diseases listed in Section 5.8. Also, modulators of the genes and gene products listed in Table 1 are useful in treating a disease, such as any of the diseases listed in Section 5.8. In a specific embodiment, the diseases are treatable with an immunomodulatory disease therapy, such as the interferon-treated diseases listed in Section 5.8.2.

5.1.2 Target Genes

As described above, the present invention provides a set of discriminant genes for use in discriminating and predicting response to PegIFN/rib treatment for HCV. The set of discriminant genes are listed in Table 1. Further, a set of 8 classifier genes in the set of discriminant genes are described. Other groups have performed studies on one or more of the discriminant genes and classifier genes. For example, polymorphisms of OAS have been weakly linked to self-limited HCV infection (Knapp 2003), and polymorphisms of Mx1 have been weakly linked to response status (Knapp 2003). Hepatic mRNA levels for OAS, Mx1, and G1P2 are increased in chronic HCV but none, alone, have been linked to treatment outcome (see, MacQuillan et al., 2003, J Med Virol. 70:219-27, which is hereby incorporated by reference in its entirety). Many of the others are ISGs with antiviral activity, and are consistent with an alteration in IFN-responsiveness being linked to treatment non-response. The genes that are not directly IFN-responsive may play roles in cellular pathways important for IFN responses (PI3AP1, DUSP1) (see, Rani et al., 2002, J Biol Chem. 277:38456-61; and Duong et al., 2004, Gastroenterology 126:263-77, which is hereby incorporated by reference in its entirety), and are involved in inflammatory cell activation and maturation (LAP) (see, Beninga, 1998, J Biol Chem. 273:18734-42; and Verhoeckx et al., 2004, Proteomics 4: 1014-28, each of which is hereby incorporated by reference in its entirety). The composition of the classifier gene set was found to be unrelated to confounding clinical factors, such as viral load, degree of fibrosis and age. In order to determine if the expression of any of the individual genes was correlated to any clinical factor, multivariate analyses was performed to determine the effect of each of these factors on the expression levels of each gene. The expression of USP18 was significantly affected by the degree of fibrosis (data not shown), but none of the other 17 discriminant genes are linked to any of the clinical factors.

Two genes in the classifier gene set, IG15 and USP18/UBP43, are noteworthy because they belong to a new, and potentially very important, interferon regulatory pathway. Both genes are expressed more highly in NR compared with R liver tissue. ISG15 is a ubiquitin-like protein which is thought to be important to innate immune functions (see, Kim and Zhang, 2003, Biochem Biophys Res Commun 307: 431-4, which is hereby incorporated by reference in its entirety). The USP18/UBP43 protease specifically removes ISG15 from ISG15-modified proteins (see, Malakhov et al., 2002, J Biol Chem 277: 9976-81, which is hereby incorporated by reference in its entirety); loss of USP18 in mice leads to IFN hypersensitivity (Malaknova 2003). It is intriguing that these two genes, linked biochemically, appear in the set of 18 genes (out of 19,000) that differ between NR and R patients. The finding that both USP18 and ISG15 are expressed more highly in NR compared with R liver tissue also suggests that this pathway may be important for the altered response to IFN treatment seen in NR patients, and potentially that inhibitors of this pathway may have therapeutic relevance in HCV infection, and perhaps even in other viral diseases.

The present invention also provides target genes whose gene expression levels can be used as predictors of response to PegIFN/rib treatment for HCV. In preferred embodiment, the present invention provides for measuring the expression levels of IFI-6-16, LAP3, CIG5 and LGP1 genes at the protein and/or RNA level as a predictor of response to PegIFN/rib treatment for HCV. The gene expression levels IFI-6-16, LAP3, CIG5, LGP1, and USP18 genes is found to be up-regulated in non-responsers to PegIFN/rib treatment for HCV.

5.1.2.1 CIG5/VIPERIN

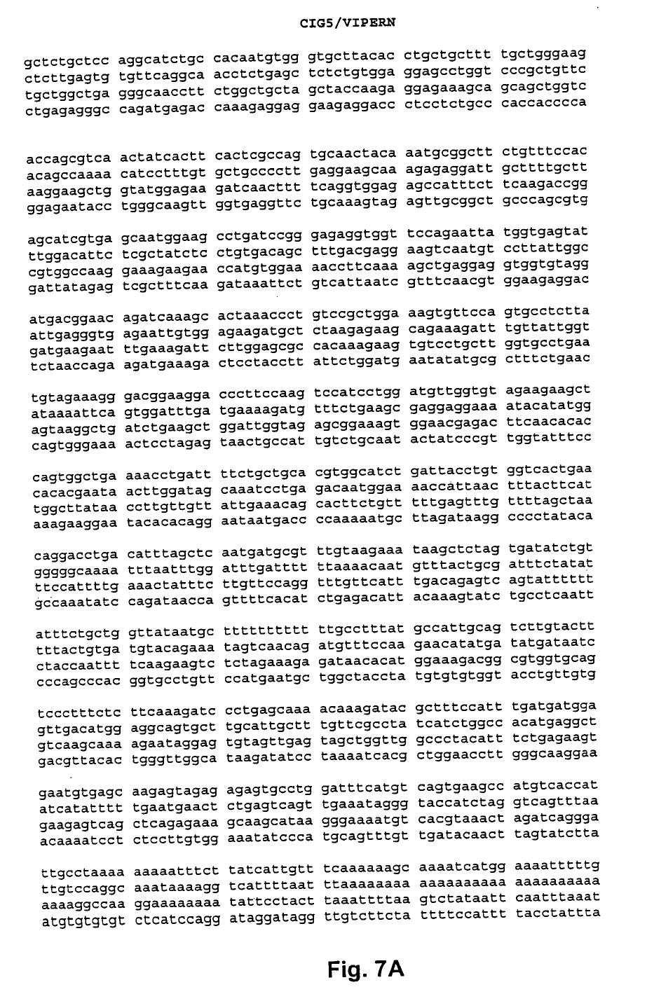

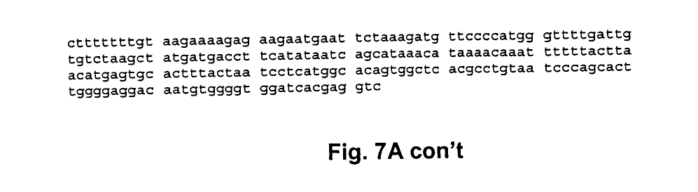

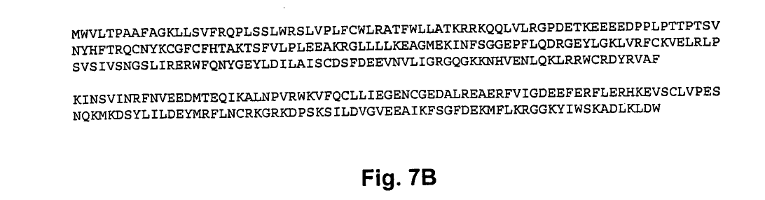

The CIG5/Viperin (VIG1, CIG %) gene was identified as an IFN induced gene hat contributes to an antiviral immune response in Gomez, D., Ph.D. Dissertation, State University of New York at Stony Brook (2003). Alternative names given to the CIG5/Viperin gene are VIG1 and CIG %. The gene (SEQ ID NO:1) and protein (SEQ ID NO:2) sequences of CIG5 are shown in FIGS. 7A and 7B, respectively. The interferon (IFN) family of cytokines functions in the mediation of cellular immunity and development. IFNs exert changes in cells through the activation of signaling pathways that ultimately result in new gene expression. Also, IFN induced expression of antiviral genes is an essential component of the innate immune response. The Gomez thesis assessed the regulated expression of CIG5/Viperin in response to IFN and Newcastle disease virus. There have also been a few studies of the CIG5 RNA and protein induction by a human cytomegalovirus infection. See, e.g., Zhu et al. “Use of differential display analysis to assess the effect of human cytomegalovirus infection on the accumulation of cellular RNAs: induction of interferon-responsive RNAs,” Proc. Natl. Acad. Sci. U.S.A., vol. 94, pp. 13985-13990 (1997). Chin et al., “Viperin (cig5), an IFN-inducible antiviral protein directly induced by human cytomegalovirus,” Proc. Natl. Acad. Sci. U.S.A., vol. 99, 2461 (2002). Homologs of CIG5/Viperin in other species, including mice, rats, monkeys, hamsters, sheep, cows, pigs, horses, cats and dogs, are also encompasses within the scope of the present invention.

5.1.2.2 LGP1

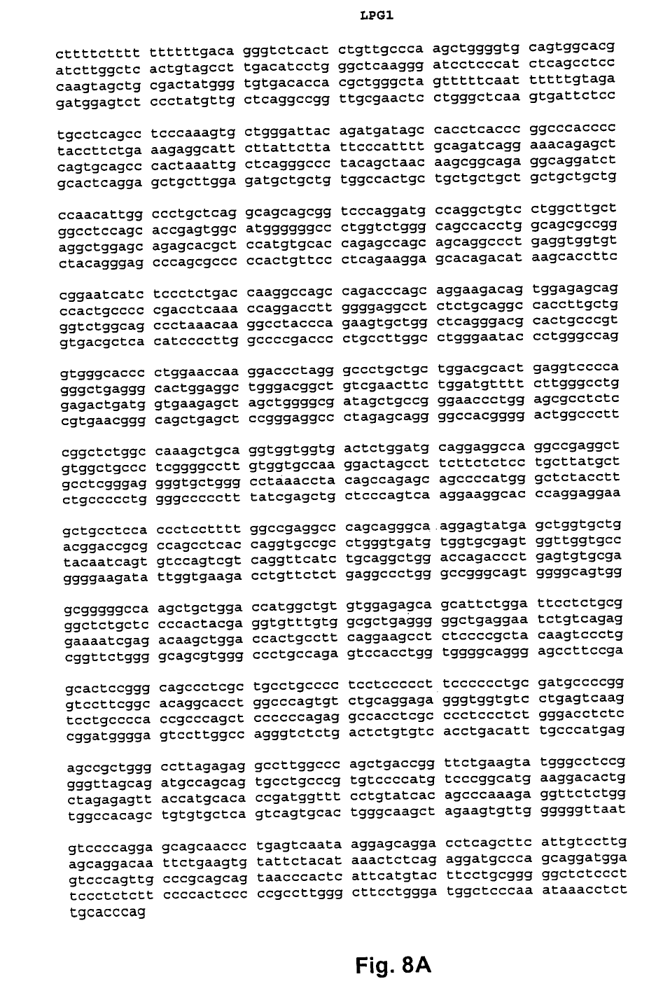

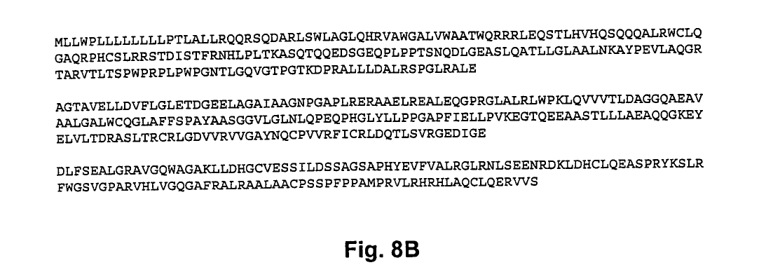

The gene (SEQ ID NO:3) and protein (SEQ ID NO:4) sequences of LGP1 (D11lgp1e-like) are shown in FIGS. 8A and 8B, respectively. An alternative name given to the LGP1 gene is d11Lgp1. Human LGP1 consists of 532 and 530 amino acids in mouse and human, respectively (88% similarity). A region in the carboxy-terminal half of LGP1 has limited homology with Arabidopsis thaliana GH3-like proteins. In a study to identify additional genes in the Stat3/5 locus that may participate in normal and neoplastic development of the mammary gland, Cui et al. cloned and sequenced 500 kb and searched for genes preferentially expressed in mammary tissue. Cui, Y. et al., “The Stat3/5 locus encodes novel endoplasmic reticulum and helicase-like proteins that are preferentially expressed in normal and neoplastic mammary tissue,” Genomics 78 (3):129-134 (2001). Cui et al. cloned D11Lgp1 and D11Lgp2, both of which are most highly expressed in normal mammary tissue and mammary tumors from several transgenic mouse models. Immunofluorescence studies demonstrated that LGP1 is located in the nuclear envelope and the endoplasmic reticulum. Homologs of LGP1 in other species, including mice, rats, monkeys, hamsters, sheep, cows, pigs, horses, cats and dogs, are also encompasses within the scope of the present invention.

5.1.2.3 IFI-6-16

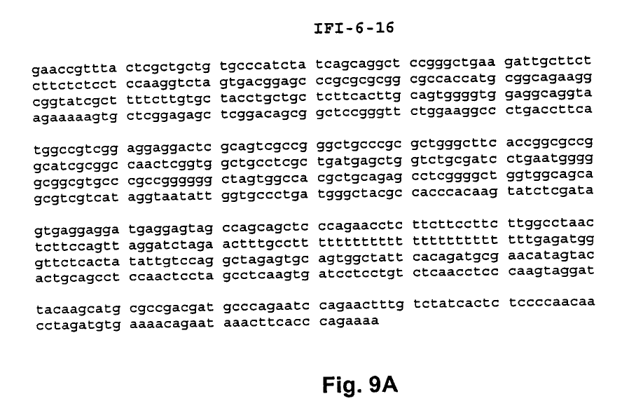

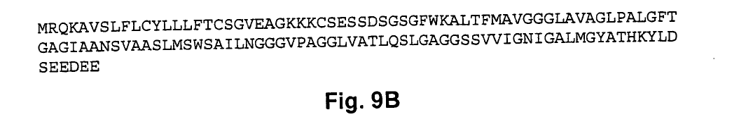

IFN-alpha has been observed to induce a number of responsive genes in HCV replicon cells. Alternative names given to the IFI-6-16 gene are 6-16, G1P3 and IFI616. Zhu, H. et al., “Gene expression associated with interferon alpha antiviral activity in an HCV replicon cell line,” Hepatology 37 (5): 1180-1188 (2003). IFI-6-16 (interferon, alpha-inducible protein (clone IFI-6-16), G1P3) was found to enhance IFN-alpha antiviral efficacy. The gene (SEQ ID NO:5) and protein (SEQ ID NO:6) sequences of IFI-6-16 are shown in FIGS. 9A and 9B, respectively. The up-regulation of IFI-6-16 has been observed after ribavirin antiviral treatment for the respiratory syncytial virus (RSV). For example, Zhang et al. studied the high-density microarrays to investigate the hypothesis that ribavirin modifies the virus-induced epithelial genomic response to replicating virus for the RSV. Zhang et al., “Ribavirin treatment up-regulates antiviral gene expression via the interferon-stimulated response element in respiratory syncytial virus-infected epithelial cells,” Journal of Virology 77 (10): 5933-5947 (2003). The study investigated the mechanism for up-regulation of the IFN-signaling pathway, where an enhanced expression of IFI 6-16 transcript was independently reproduced by Northern blot analysis. The study found that ribavirin potentiates virus-induced IFN-stimulated response element signaling to enhance the expression of antiviral IFN-stimulated response genes. Homologs of IFI-6-16 in other species, including mice, rats, monkeys, hamsters, sheep, cows, pigs, horses, cats and dogs, are also encompasses within the scope of the present invention.

5.1.2.4 LAP3

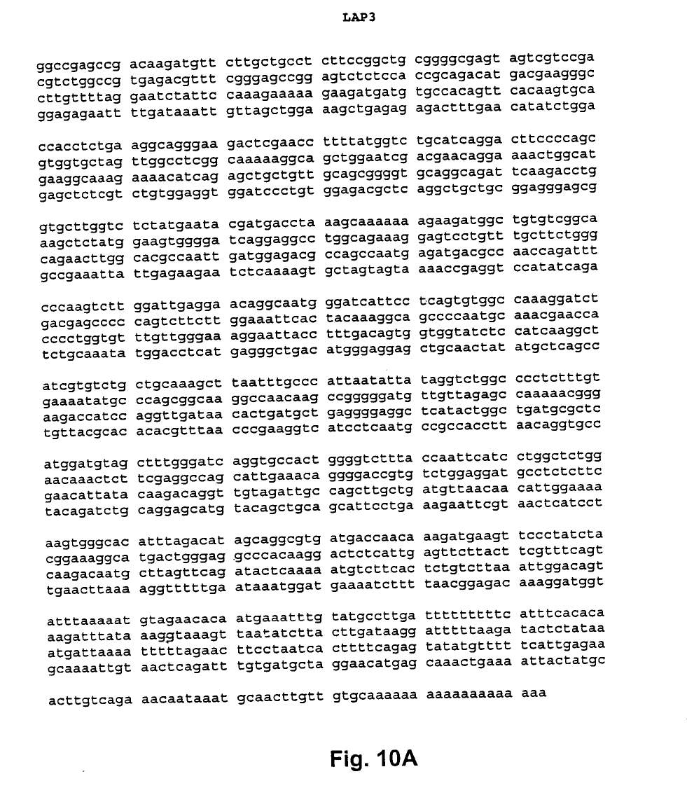

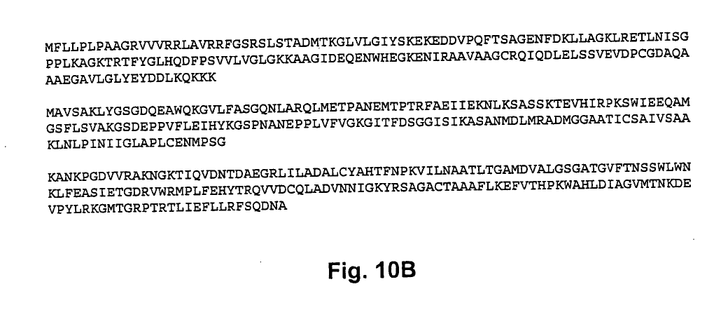

FIGS. 10A and 10B show the gene (SEQ ID NO:7) and protein (SEQ ID NO:8) sequences of human leucine aminopeptidase 3 (LAP3), respectively. Alternative names given to the LAP3 gene are luecine aminopeptidase 3 and LAPEP. Tsunogake et al. conducted an in vitro study of the effects of three aminopeptidase inhibitors on the production of various kinds of cytokines from normal human peripheral blood mononuclear cells (PB-MNC) and a human clonal stromal cell line. Tsunogake S. et al., “Effect of aminopeptidase inhibitors on the production of various cytokines by peripheral blood mononuclear cells and stromal cells and on stem cell factor gene expression in stromal cells: Comparison of ubenimex with its stereoisomers,” Journal of Immunotherapy 10/2: 41-47 (1994). Tsunogake et al. found that the stimulatory effects of the inhibitor ubenimex on cytokine production was exerted through inhibition of leucine aminopeptidase. Homologs of LAP3 in other species, including mice, rats, monkeys, hamsters, sheep, cows, pigs, horses, cats and dogs, are also encompasses within the scope of the present invention.

Leucine aminopeptidase is over-expressed in patients that do not respond to treatment. Using the methods of the present invention, LAP inhibitors can be identified using biochemical assays, such as those described by Grant and colleagues using fluorogenic substrates. Representative inhibitors that might prove efficacious include those described by Kafarski and colleagues. See, for example, Grant S K, Sklar J G, Cummings R T., Development of novel assays for proteolytic enzymes using rhodamine-based fluorogenic substrates, 2002, J Biomol Screen. 7, p. 531-40; and Grembecka J, Mucha A, Cierpicki T, Kafarski P., The most potent organophosphorus inhibitors of leucine aminopeptidase. Structure-based design, chemistry, and activity, J Med Chem. Jun. 19, 2003; 46(13):2641-55, which is hereby incorporated by reference in its entirety.

5.1.2.5 USP18

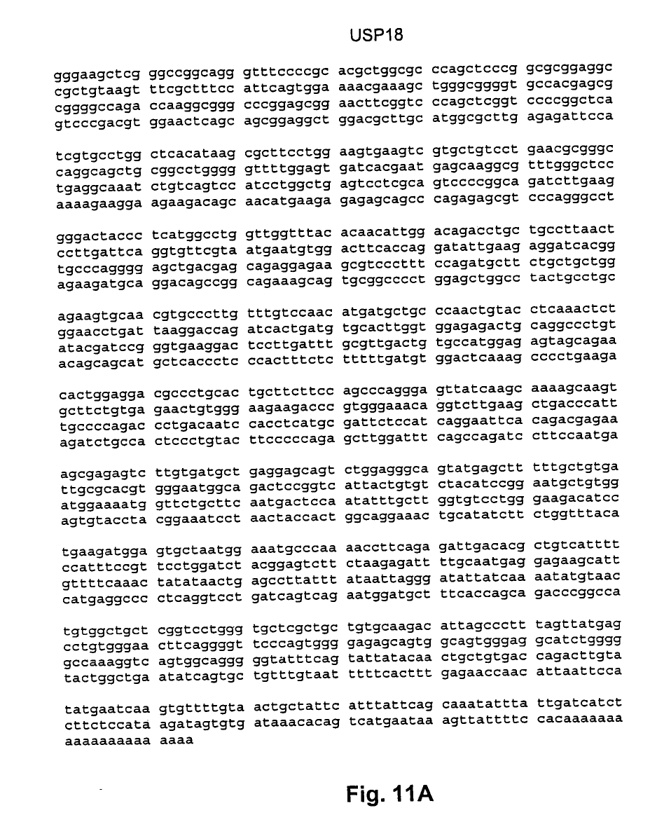

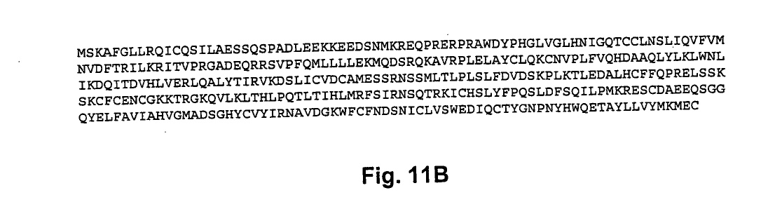

Ubiquitin specific protease 18 (USP18) is a protease that removes the ubiquitin-like protein (ISG-15) from proteins. The enzyme has been shown to cleave proteins in vitro. Alternative names given to USP18 are UBP43 and ISG43. The gene (SEQ ID NO:9) and protein (SEQ ID NO:10) sequences of USP18 are shown in FIGS. 11A and 11B, respectively. Inhibitors of USP18 function could be identified in vivo by assaying for cleavage of a ISG15-USP18 fusion protein expressed in E coli, according to Malakhov M P, et al., “UBP43 (USP18) specifically removes ISG15 from conjugated proteins,” J Biol. Chem. 277(12):9976-81 (2002). Alternatively, the activity of USP18 could be tested by the release of a radio-labeled, or fluorescently-labelled ISG15 proteins from a PEST sequence. Malakhov M P, et al., “UBP43 (USP18) specifically removes ISG15 from conjugated proteins,” J Biol. Chem. 277(12):9976-81 (2002). USP18 could also be screened for small molecules that bind the protein, using any of a number of assays, for example differential scanning calorimetry. See also Kim K I et al., “ISG15, not just another ubiquitin-like protein,” Biochem Biophys Res Commun. August 1;307(3):431-4 (2003); Malakhova O A et al., “Protein ISGylation modulates the JAK-STAT signaling pathway,” Genes Dev. 17(4):455-60 (2003); Ritchie K J, et al., “Dysregulation of protein modification by ISG15 results in brain cell injury,” Genes Dev. 16(17):2207-12 (2002); Malakhova O, et al., “Lipopolysaccharide activates the expression of ISG15-specific protease UBP43 via interferon regulatory factor 3,” J Biol Chem. 277(17):14703-11 (2002); Malakhov M P, et al., “UBP43 (USP18) specifically removes ISG15 from conjugated proteins,” J Biol Chem. 277(12):9976-81 (2002); Liu L Q, et al., “A novel ubiquitin-specific protease, UBP43, cloned from leukemia fusion protein AML1-ETO-expressing mice, functions in hematopoietic cell differentiation,” Mol Cell Biol. (4):3029-38 (1888); Malakhova O A, et al., “Protein ISGylation modulates the JAK-STAT signaling pathway,” Genes Dev. 17(4):455-60 (2003); Schwer H, et al., “Cloning and characterization of a novel human ubiquitin-specific protease, a homologue of murine UBP43 (Usp18),” Genomics 65(1):44-52 (2000); and Nakaya T, et al., “Gene induction pathways mediated by distinct IRFs during viral infection,” Biochem Biophys Res Commun. 283(5):1150-6 (2001).

Homologs of USP18 in other species, including mice, rats, monkeys, hamsters, sheep, cows, pigs, horses, cats and dogs, are also encompasses within the scope of the present invention.

5.1.3 Hepatitis C Virus Assay

Randall G, et al. developed a hepatitis C virus cell culture replication system. Randall G, et al., “Hepatitis C virus cell culture replication systems: their potential use for the development of antiviral therapies,” Curr. Opin. Infect. Dis. (6):743-7 (2001). The absence of an efficient cell culture system and an accessible small animal model to study hepatitis C virus replication and pathogenesis were major obstacles to the development of effective antiviral therapies. Studies of surrogate model systems, either related viruses or chimeric viruses containing part of the hepatitis C virus genome, gave insight into hepatitis C virus replication, in addition to being a powerful tool for drug discovery. The development of an efficient system for the initiation of replication in cell culture provided a viable screen for inhibitors of hepatitis C virus replication. It also advanced the ultimate goal of an infectious cell culture system for hepatitis C virus.

To test the role of any gene for HCV viral replication, the replication of the HCV genome could be monitored in cell culture in the presence or absence of a silencing RNA (RNAi) for the corresponding gene of interest.

5.1.4 Specimen Sources

Unless otherwise indicated herein, any biological sample or any biological sample from an organ inflicted with the disease, e.g., liver tissue sample, pancreatic tissue sample, or blood sample, etc., obtained from any subject may be used in accordance with the methods of the invention. In a specific embodiment, the biological sample is a blood sample from a subject with a liver disease or a disease treatable with an immunomodulatory disease therapy. Examples of subjects from which such a sample may be obtained and utilized in accordance with the methods of the invention include, but are not limited to, asymptomatic subjects, subjects manifesting or exhibiting 1, 2, 3, 4 or more symptoms of the liver disease or the disease that is treatable with an immunomodulatory disease therapy (“the disease”), subjects clinically diagnosed as having the disease, subjects predisposed to the disease (e.g., subjects with a family history of the disease, subjects with a genetic predisposition to the disease, and subjects that lead a lifestyle that predisposes them to the disease or increases the likelihood of contracting the disease), subjects suspected of having the disease, subjects undergoing a therapy for the disease, subjects with the disease and at least one other condition (e.g., subjects with 2, 3, 4, 5 or more conditions), subjects not undergoing a therapy for the disease, subjects determined by a medical practitioner (e.g., a physician) to be healthy or free of the disease (i.e., normal), subjects that have been cured of the disease, subjects that are managing their disease, and subjects that have not been diagnosed with the disease. In a specific embodiment, the subjects from which a sample may be obtained and utilized have mild, marked, moderate or severe liver disease or disease that is treatable with an immunomodulatory disease therapy.

5.2 Measured Signals

The present invention provides systems and methods for manipulating and analyzing measured signals, e.g., measured intensity signals obtained in a microarray gene expression experiment. For example, the measured signals can represent measurements of the abundances or activities of cellular constituents in a cell or organism; or measurements of the responses of cellular constituents in a living cell or organism to a perturbation to the living cell or organism. As used herein, the term “cellular constituent” comprises individual genes, proteins, mRNA expressing a gene, a cDNA, a cRNA, and/or any other variable cellular component or protein activities, degree of protein modification (e.g., phosphorylation), for example, that is typically measured in a biological experiment by those skilled in the art. Furthermore, the term “cellular constituents” comprises biological molecules that are secreted by a cell including, but not limited to, hormones, matrix metalloproteinases, and blood serum proteins (e.g., granulocyte colony stimulating factor, human growth hormone, etc.). Such measured intensity signals permit analysis of data using traditional statistical methods, e.g., ANOVA and regression analysis (e.g., to determine statistical significance of measured data).

The measured signals can be obtained by both single-channel measurement and two-channel measurement. As used herein, a “single-channel measurement” refers broadly to where measurements of cellular constituents are made on a single sample (e.g., a sample prepared from a living cell or organism having been subjected to a given condition) in a single experimental reaction, whereas a “two-channel measurement” refers to where measurements of cellular constituents are made distinguishably and concurrently on two different samples (e.g., two samples prepared from cells or organisms, each having been separately subjected to a given condition) in the same experimental reaction. The cells or organisms from which the two samples in a two-channel experiment are derived can be subjected to the same condition or different conditions. The expression “same experimental reaction” means in the same reaction mixture, for example, by contacting with the same reagents in the same composition at the same time (e.g., using the same microarray for nucleic acid hybridization to measure mRNA, cDNA or amplified RNA; or the same antibody array to measure protein levels). In this disclosure, a measurement in a “same-vs.-same” experiment is referenced. As used herein, such a measurement refers to either a two-channel measurement performed in an experiment in which the two samples are derived from cells or organism having been subjected to the same condition or a measurement obtained in two single-channel measurements performed separately with two samples which are derived from cells or organisms having been subjected to the same condition.

While the experiment design is described in terms of using measured signals obtained from a microarray experiment, it will be clear to a person of ordinary skill in the art that the signals measured in many other kinds of experiments, eg., signals measured in a protein array experiment, an ELISA assay, or signals measured in a 2D protein gel experiment, are also applicable to the invention.

5.2.1 Biological State and Expression Profiles

The state of a cell or other biological sample is represented by cellular constituents (any measurable biological variables) as defined in Section 5.2.1. 1, infra. Those cellular constituents vary in response to perturbations such as time or dosage, or under different conditions. The measured signals can be measurements of such cellular constituents or measurements of responses of cellular constituents.

5.2.1.1 Biological State

As used herein, the term “biological sample” is broadly defined to include any cell, tissue, organ or multicellular organism. A biological sample can be derived, for example, from cell or tissue cultures in vitro. Alternatively, a biological sample can be derived from a living organism. In preferred embodiments, the biological sample comprises a living cell or organism.

The state of a biological sample can be measured by the content, activities or structures of its cellular constituents. The state of a biological sample, as used herein, is taken from the state of a collection of cellular constituents, which are sufficient to characterize the cell or organism for an intended purpose including, but not limited to characterizing the effects of a drug or other perturbation. The term “cellular constituent” is also broadly defined in this disclosure to encompass any kind of measurable biological variable. The measurements and/or observations made on the state of these constituents can be of their abundances (e.g., amounts or concentrations in a biological sample) e.g., of mRNA or proteins, or their activities, or their states of modification (e.g., phosphorylation), or other measurements relevant to the biology of a biological sample. In various embodiments, this invention includes making such measurements and/or observations on different collections of cellular constituents. These different collections of cellular constituents are also called herein aspects of the biological state of a biological sample.

This invention is also adaptable, where relevant, to “mixed” aspects of the biological state of a biological sample in which measurements of different aspects of the biological state of a biological sample are combined. For example, in one mixed aspect, the abundances of certain RNA species and of certain protein species, are combined with measurements of the activities of certain other protein species. Further, it will be appreciated from the following that this invention is also adaptable to other aspects of the biological state of the biological sample that are measurable.

The biological state of a biological sample (e.g., a cell or cell culture) is represented by a profile of some number of cellular constituents. Such a profile of cellular constituents can be represented by a vector S, where Sᵢ is the level of the i'th cellular constituent, for example, the transcript level of gene i, or alternatively, the abundance or activity level of protein i.

In some embodiments, cellular constituents are measured as continuous variables. For example, transcriptional rates are typically measured as number of molecules synthesized per unit of time. Transcriptional rate may also be measured as percentage of a control rate. However, in some other embodiments, cellular constituents may be measured as categorical variables. For example, transcriptional rates may be measured as either “on” or “off”, where the value “on” indicates a transcriptional rate above a predetermined threshold and value “off” indicates a transcriptional rate below that threshold.

In preferred embodiments, the measured signals are measured in a microarray gene expression experiment. In other preferred embodiments, the measured signals are measured in an ELISA assay, a protein array experiment or a 2D gel protein experiment.

In one embodiment, the measured signals are signals obtained in a microarray experiment in which two spots or probes on a microarray are used for obtaining each measured signal, one comprising the targeted nucleotide sequence, e.g., the target probe, e.g., a perfect-match probe, and the other comprising a reference sequence, e.g., a reference probe, e.g., a mutated mismatch probe. The RP probe is used as a negative control, e.g., to remove undesired effects from non-specific hybridization. In one embodiment, the measured signal obtained in such a manner is defined as the difference between the intensities of the target probe and reference probe. In preferred embodiments, a multiple slide, two channel indirect cDNA design is used. Each mRNA sample is reverse transcribed into cDNA and then co-hybridized with a common reference sample on a glass slide. Use of the common reference sample approach allows for a comparison of gene expression levels across arrays. Thus, all comparisons of interest are indirect in the sense that the difference in mRNA transcript abundance between two or more classes of test samples is relative to a common reference. The relative mRNA transcript abundance between the test and the reference samples is determined by the fluorescent intensity measurement of the red (Cy5) labeled test and green (Cy3) labeled reference samples (Cy3 and Cy5 are the most commonly used cyanine dyes). The main reason for an indirect (as opposed to direct) design is the scarcity of control samples (or normal liver samples) which could be used as reference samples

5.2.1.2 Biological Responses and Expression Profiles

The responses of a biological sample to a perturbation, e.g., under a condition, such as the application of a drug, one of the factors in an experiment design, can be measured by observing the changes in the biological state of the biological sample. For example, the responses of a biological sample can be responses of a living cell or organism to a perturbation, e.g., application of a drug, a genetic mutation, an environmental change, and so on, to the living cell or organism. A response profile is a collection of changes of cellular constituents. In the experiment design, the response profile of a biological sample (e.g., a cell or cell culture) to the perturbation m can be represented by a vector v^{(m)}, where vᵢ^{m} is the amplitude of response of cellular constituent i under the perturbation m. Each vᵢ^{m} is then the value assigned to one of the levels of a factor of the experiment design. In some particularly preferred embodiments of this invention, the biological response to the application of a drug, a drug candidate or any other perturbation, is measured by the induced change in the transcript level of at least 2 genes, more preferably more than 5 genes, most preferably more than 10 genes, and possibly more than 100 genes and more than 1,000 genes.

In another preferred embodiment of the invention, the biological response to the application of a drug, a drug candidate or any other perturbation, is measured by the induced change in the expression levels of a plurality of exons in at least 2 genes, more preferably more than 5 genes, most preferably more than 10 genes, and possibly more than 100 genes and more than 1,000 genes. In some embodiments of the invention, the response is simply the difference between biological variables before and after perturbation. In some preferred embodiments, the response is defined as the ratio of cellular constituents before and after a perturbation is applied.

5.3 t-Test Analysis

A t-test can be performed by data analysis module 76 to identify differentially expressed genes in the measured microarray profiles. The t-test assesses whether the means of two groups are statistically different from each other. The t-test can be used, for example, to identify those cellular constituents that have significantly different mean abundances in the set of responders and nonresponders. See, for example, Smith, 1991, Statistical Reasoning, Allyn and Bacon, Needham Heights, Mass., pp. 361-365. The t-test is represented by the following formula:

$$t = \frac{{\overline{X}}_{T} - {\overline{X}}_{C}}{\sqrt{\frac{{var}_{T}}{{n}_{T}} + \frac{{var}_{C}}{{n}_{C}}}}$$

where,

the numerator is the numerator is the difference between the mean level of a given cellular constituent in a first group (T) and a second group (C); and
var_{T} is the variance (square of the deviation) in the level of the given gene in group T;
var_{C} is the variance (square of the deviation) in the level of the given gene in group C;
n_{T} is the number of organisms in group T; and
n_{C} is the number of organisms in group C.

The t-value will be positive if the first mean is larger than the second and negative if it is smaller. The significance of any t-value is determined by looking up the value in a table of significance to test whether the ratio is large enough to say that the difference between the groups is not likely to have been a chance finding. To test the significance, a risk level (called the alpha level or p) is set. In some embodiments of the present invention, p is set at 0.05. This means that the five times out of a hundred there would be a statistically significant difference between the means even if there was none (i.e., by “chance”). In some embodiments, p is set at 0.025, 0.01 or 0.005.

Further, to test significance, the number of degrees of freedom (df) for the test need to be determined. In the t-test, the degrees of freedom is the sum of the persons in both groups (T and C) minus 2. Given p, the df, and the t-value, it is possible to look the t-value up in a standard table of significance (see, for example, Table III of Fisher and Yates, Statistical Tables for Biological, Agricultural, and Medical Research, Longman Group Ltd., London) to determine whether the t-value is small enough to be significant. Another method that can be performed by data analysis module 76 is the paired t-test. The paired t-test assesses whether the means of two groups are statistically different from each other. The paired t-test is generally used when measurements are taken from the same organism before and after some perturbation, such as before and after a liver disease therapy regimen or a therapy regimen for a disease that is treatable with an immunomodulatory disease therapy. For example, the paired t-test can be used to determine the significance of a difference in blood pressure before and after administration of a compound that affects blood pressure. The paired t-test is represented by the following formula:

$$t = \frac{\overline{d}}{\frac{{S}_{d}}{\sqrt{n}}}$$

where,
the numerator is the paired sample mean;
S_{d} is the paired sample deviation; and
n is the number of pairs considered.

In a specific embodiment, the t-test is performed by data analysis module 76 using a multtest( ) package, which includes an estimation of adjusted p-values by permutation, if there is concern arising from multiple hypothesis testing. See, e.g., Dudoit, 2003, Statistical Science 18, p. 71-103. The differential gene expressions between the two groups are identified by computing the two-sample Welch t-statistics. The normalized gene expression data is an n H p matrix X′ of log₂ ratios of n rows (genes) and p=p₁+p₂ columns (samples) (for example, p=31 for p₁=16 responders and p₂=15 non-responders). Different patients in each respective class are considered as biological replicates of the same condition. For each gene j the t-statistics between the two groups p₁ (responders) and p₂ (nonresponders) is obtained by the t-test formula given above, where X_{T} and X_{C} denote the average expression level of gene j in the n_{C}=p₁ responder group and the n_{T}=p₂ non-responder group, respectively, and var_{C} and var_{T} denote the sample variances of gene j expression level in the two groups.

5.4 Classification Schemes

The present invention employs a number of classification schemes, which are performed by data analysis module 76. A few representative classification schemes are present in this section. In some embodiments the classification scheme is a supervised classification scheme whereas in other embodiments the classification scheme is unsupervised. Supervised classification schemes in accordance with the present invent use techniques that include, but are not limited to, linear discriminant analysis and nearest neighbor analysis. Unsupervised classification schemes in accordance with the present invention include, but are not limited to, agglomerative cluster analysis and principal component analysis.

5.4.1 Unsupervised Classification Schemes

An unsupervised analysis can be defined as a method which seeks to determine structures in data without use of a training set. Embodiments of an unsupervised classification scheme include a hierarchical cluster analysis and principal component analysis. An unsupervised classification scheme can be used to test for differences in gene expression profiles between normal liver tissue and diseased liver tissue or to corroborate the results of a supervised classification scheme (described in Section 5.4.2, below).

5.4.1.1 Clustering Techniques

In some embodiments, clustering is used in step 212 to cluster the population based on RNA expression levels (or RT-PCR levels) in the set of discriminant genes identified in steps 208 and 210 to verify that the population clusters into a responsive cluster and a non-responsive cluster. Clustering is described on pages 211-256 of Duda and Hart, Pattern Classification and Scene Analysis, 1973, John Wiley & Sons, Inc., New York (“Duda”). As described in Section 6.7 of Duda, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined.

Similarity measures are discussed in Section 6.7 of Duda, where it is stated that one way to begin a clustering investigation is to define a distance function and to compute the matrix of distances between all pairs of samples in a dataset. If distance is a good measure of similarity, then the distance between samples in the same cluster will be significantly less than the distance between samples in different clusters. However, as stated on page 215 of Duda, clustering does not require the use of a distance metric. For example, a nonmetric similarity function s(x, x′) can be used to compare two vectors x and x′. Conventionally, s(x, x′) is a symmetric function whose value is large when x and x′ are somehow “similar”. An example of a nonmetric similarity function s(x, x′) is provided on page 216 of Duda.

Once a method for measuring “similarity” or “disimilarity” between points in a dataset has been selected, clustering requires a criterion function that measures the clustering quality of any partition of the data. Partitions of the data set that extremize the criterion function are used to cluster the data. See page 217 of Duda. Criterion functions are discussed in Section 6.8 of Duda.

More recently, Duda et al., Pattern Classification, 2^{nd} edition, John Wiley & Sons, Inc. New York, has been published. Pages 537-563 describe clustering in detail. More information on clustering techniques can be found in Kaufman and Rousseeuw, 1990, Finding Groups in Data: An Introduction to Cluster Analysis, Wiley, New York, N.Y.; Everitt, 1993, Cluster analysis (3d ed.), Wiley, New York, N.Y.; and Backer, 1995, Computer-Assisted Reasoning in Cluster Analysis, Prentice Hall, Upper Saddle River, N.J. A specific example of a clustering technique that can be performed in the methods of the invention is agglomerative clustering.

5.4.1.2 Agglomerative Clustering

Agglomerative (bottom-up clustering) procedures start with n singleton clusters and form a sequence of partitions by successively merging clusters. The major steps in agglomerative clustering are contained in the following procedure, where c is the desired number of final clusters, Dᵢ and Dⱼ are clusters, xᵢ is an element, and there are n such elements:

| | |
|---|---|
| 1 | begin initialize c, ĉ [symbol] n, Dᵢ [symbol] {xᵢ}, i = 1, ..., n |
| 2 | do ĉ [symbol] ĉ −1 |
| 3 | find nearest clusters, say, Dᵢ and Dⱼ |
| 4 | merge Dᵢ and Dⱼ |
| 5 | until c = ĉ |
| 6 | return c clusters |
| 7 | end |

In this algorithm, the terminology a←b assigns to variable a the new value b. As described, the procedure terminates when the specified number of clusters has been obtained and returns the clusters as a set of points. A key point in this algorithm is how to measure the distance between two clusters Dᵢ and Dⱼ. The method used to define the distance between clusters Dᵢ and Dⱼ defines the type of agglomerative clustering technique used. Representative techniques include the nearest-neighbor algorithm, farthest-neighbor algorithm, the average linkage algorithm, the centroid algorithm, and the sum-of-squares algorithm. The agglomerative clustering can use, for example, a nearest neighbor algorithm, a farthest-neighbor algorithm, an average linkage algorithm, a centroid algorithm, or a sum of squares algorithm.

In a specific embodiment, the agglomerative clustering function hclust( ) from the R package mva is used to cluster the data into the responder and non-responder groups. See, e.g., Becker et al., “The New S Language,” Wadsworth and Brooks/Cole (S version) (1988); Everitt, B, “Cluster Analysis,” London: Heinemann Educ. Books. (1974); Hartigan, J. A, “Clustering Algorithms,” New York: Wiley (1975); Sneath, P. H. A. and R. R. Sokal, “Numerical Taxonomy,” San Francisco:Freeman (1973); Anderberg, 1973, Cluster Analysis for Applications, Academic Press, New York; and Gordon, 1999, Classification, 2^{nd} ed., Chapman and Hall, CRC, London, each of which is hereby incorporated by reference in its entirety.

The function starts by considering each clustering object as one cluster then iteratively proceed to join the two most similar clusters until there is just a single cluster left. At each stage the distances between clusters are computed using the squared Euclidean distances. The algorithm used in hclust( ) orders subtrees so that the tighter cluster is on the left. To calculate distances between clusters the complete linkage clustering method is used, which groups clusters according to the greatest distance between objects in each cluster. This methods tend to form tight clusters of similar objects and can be sensitive to outliers.

5.4.1.3 Principal Component Analysis

Principal component analysis (PCA) has been proposed to analyze gene expression data. Principal component analysis is a classical technique to reduce the dimensionality of a data set by transforming the data to a new set of variable (principal components) that summarize the features of the data. See, for example, Jolliffe, 1986, Principal Component Analysis, Springer, N.Y. Principal components (PCs) are uncorrelated and are ordered such that the k^{th} PC has the kth largest variance among PCs. The k^{th} PC can be interpreted as the direction that maximizes the variation of the projections of the data points such that it is orthogonal to the first k-1 PCs. The first few PCs capture most of the variation in the data set. In contrast, the last few PCs are often assumed to capture only the residual ‘noise’ in the data.

PCA can also be used to create a model in accordance with the present invention. In such an approach, vectors for the molecular markers selected in the last instance of step 214 can be constructed in the same manner described for clustering above. In fact the set of vectors, where each vector represents the expression values for the select molecular markers from a particular member of the training population, can be considered a matrix. In some embodiments, this matrix is represented in a Free-Wilson method of qualitative binary description of monomers (Kubinyi, 1990, 3D QSAR in drug design theory methods and applications, Pergamon Press, Oxford, pp 589-638), and distributed in a maximally compressed space using PCA so that the first principal component (PC) captures the largest amount of variance information possible, the second principal component (PC) captures the second largest amount of all variance information, and so forth until all variance information in the matrix has been accounted for.

Then, each of the vectors (where each vector represents a member of the training population) is plotted. Many different types of plots are possible. In some embodiments, a one-dimensional plot is made. In this one-dimensional plot, the value for the first principal component from each of the members of the training population is plotted. In this form of plot, the expectation is that members of a first trait subgroup will cluster in one range of first principal component values and members of a second trait subgroup will cluster in a second range of first principal component values.

In one ideal example, the population of step 202 comprises two trait subgroups: “responders” and “nonresponders.” The first principal component is computed using the gene expression values for the genes selected in the last instance of step 250 across the entire population of step 202. Then, each member of the training set is plotted as a function of the value for the first principal component. In this ideal example, those members of the training population in which the first principal component is positive are the “responders” and those members of the training population in which the first principal component is negative are “nonresponders.”

In some embodiments, the members of the training population are plotted against more than one principal component. For example, in some embodiments, the members of the training population are plotted on a two-dimensional plot in which the first dimension is the first principal component and the second dimension is the second principal component. In such a two-dimensional plot, the expectation is that members of each trait subgroup represented in the training population will cluster into discrete groups. For example, a first cluster of members in the two-dimensional plot will represent a the responders and a second cluster of members in the two-dimensional plot will represent the nonresponders.

In some embodiments, principal component analysis is performed by using the R mva package (Anderson, 1973, Cluster Analysis for applications, Academic Press, New York 1973; Gordon, Classification, Second Edition, Chapman and Hall, CRC, 1999.). Principal component analysis is further described in Duda, Pattern Classification, Second Edition, 2001, John Wiley & Sons, Inc.

As in the hierarchical cluster analysis, the principal component analysis method seeks to structure data according to similarities between objects. Briefly, in some embodiments, the method seeks linear combinations among samples with maximal (or minimal) variance. By searching to maximize solutions of the characteristic equation of a covariance matrix Σ, a set of eigenvectors are found with directions along the greatest variability in the data. Thus the first eigenvector (leading to a first linear combination and thus first principal component) corresponds to the largest eigenvalue of Σ, subsequent eigenvectors are in directions of the largest possible variations uncorrelated with those that have been taken before.

Let Σ denote the covariance matrix of data X of n row vectors xₙ=(xₙₗ, . . . , xₙₚ) of p normalized log₂ gene expression levels belonging to a class label yₙ, the method searches to maximize the sample variance of a linear combination x.a of a row vector x with ∥a∥=a^{T}a=1, as:
a^{T}Σ=Λa

where Λ is the diagonal matrix of eigenvalues. The function prcomp( ) from the R package mva can be used for the principal component analysis. See, e.g., Becker, R. A., Chambers, J. M. and Wilks, A. R, “The New S Language,” Wadsworth and Brooks/Cole, (S version) (1988); S. Dudoit, J. Fridlyand, and T. P. Speed, “Comparison of discrimination methods for the classification of tumors using gene expression data,” Journal of the American Statistical Association, Vol. 97, No. 457, p. 77-87; Mardia, K. V., J. T. Kent, and J. M. Bibby, “Multivariate Analysis,” London: Academic Press (1979); Venables, W. N. and B. D. Ripley, “Modern Applied Statistics with S-PLUS,” Springer-Verlag (1997).

5.4.2 Supervised Classification Schemes

Data analysis module 76 also includes instructions for applying a supervised classification scheme to the intensity measurements in the plurality of profiles. Supervised analyses assume a given structure of data and seek to classify samples into known categories. A classification algorithm assigns, or predicts, a class or a category of a given test sample from a set of known data samples, the learning set. Embodiments of a supervised classification scheme include a nearest neighbor classifier and a linear discriminant analysis.

5.4.2.1 Nearest Neighbor Classifier

One of the main tasks of any classification algorithms is to assign (or predict) a class (or a category) of a given test sample from a set of known data samples (the learning set). The nearest neighbor classifier method is chosen many times because of its power and simplicity. For algorithmic details of exemplary algorithms that can be used, see, e.g., Murtagh, F. “Multidimensional Clustering Algorithms”, in COMP-STAT Lectures 4,.Wuerzburg: Physica-Verlag (1985).

Nearest neighbor classifiers are memory-based and require no model to be fit. Given a query point x₀, the k training points x₍ᵣ₎, r, k closest in distance to x₀ are identified and then the point x₀ is classified using the k nearest neighbors. Ties can be broken at random. In some embodiments, Euclidean distance in feature space is used to determine distance as:
d₍ᵢ₎=∥x₍ᵢ₎−xₒ∥.
Typically, when the nearest neighbor algorithm is used, the gene expression data from step 204 (and/or step 210) is standardized to have mean zero and variance 1. In the present invention, the members of the population from step 202 are randomly divided into a training set and a test set. For example, in one embodiment, ⅔ of the members of the training population are placed in the training set and ⅓ of the members of the training population are placed in the test set. The combination of genes selected in the last instance of step 250 represents the feature space into which members of the test set are plotted. Next, the ability of the training set to correctly characterize the members of the test set is computed. In some embodiments, nearest neighbor computation is performed several times for a given combination of genes using a k-nearest neighbour cross validation classification function knn.cv( ). In each iteration, the members of the training population are randomly assigned to the training set and the test set. Then, the classifier quality of the genes is taken as the average of each such iteration of the nearest neighbor computation.

The nearest neighbor rule can be refined to deal with issues of unequal class priors, differential misclassification costs, and feature selection. Many of these refinements involve some form of weighted voting for the neighbors. For more information on nearest neighbor analysis, see Duda, Pattern Classification, Second Edition, 2001, John Wiley & Sons, Inc; and Hastie, 2001, The Elements of Statistical Learning, Springer, N.Y.

5.4.2.2 Linear Discriminant Analysis

Linear discriminant analysis, one of the best established discriminant analysis methods, and was introduced by Fisher (1936). If W and B denote, respectively, the within-class covariance and the between-class covariance matrices, then linear discriminant analysis seeks a linear combination of xa variables so that the ratio a^{T}Ba/a^{T}Wa is maximal. This can be achieved by first scaling the data x so as to obtain an identity matrix for a within-class correlation matrix then proceed to maximize a^{T}Ba (with ∥a∥=1) in order to find a set of eigenvectors with directions along the greatest variability in the data. Each of the eigenvectors gives a unique linear combination called a linear discriminant. The Ida( ) function from the R package MASS can be used to perform a linear discriminant analysis. See, e.g., Becker, R. A., Chambers, J. M. and Wilks, A. R., “The New S Language,” Wadsworth and Brooks/Cole (S version) (1988); S. Dudoit, J. Fridlyand, and T. P. Speed, “Comparison of discrimination methods for the classification of tumors using gene expression data,” Journal of the American Statistical Association, Vol. 97, No. 457, p. 77-87; Mardia, K. V., J. T. Kent, and J. M. Bibby, “Multivariate Analysis,” London: Academic Press (1979); Venables, W. N. and B. D. Ripley, “Modern Applied Statistics with S-PLUS,” Springer-Verlag (1997); and Venables, W. N. and Ripley, B. D., “Modern Applied Statistics with S,” Fourth edition. (2002).

5.5 Exemplary Normalization Routines

Optionally, a number of different normalization protocols can be performed by data normalization module 72 to normalize cellular constituent abundance data. Some such normalization protocols are described in this section. Typically, the normalization comprises normalizing the expression level measurement of each gene in a plurality of genes that is expressed by patient. Many of the normalization protocols described in this section are used to normalize microarray data. It will be appreciated that there are many other suitable normalization protocols that may be used in accordance with the present invention. All such protocols are within the scope of the present invention. Many of the normalization protocols found in this section are found in publicly available software, such as Microarray Explorer (Image Processing Section, Laboratory of Experimental and Computational Biology, National Cancer Institute, Frederick, Md. 21702, USA).

One normalization protocol is Z-score of intensity. In this protocol, raw expression intensities are normalized by the (mean intensity)/(standard deviation) of raw intensities for all spots in a sample. For microarray data, the Z-score of intensity method normalizes each hybridized sample by the mean and standard deviation of the raw intensities for all of the spots in that sample. The mean intensity mnIᵢ and the standard deviation sdIᵢ are computed for the raw intensity of control genes. It is useful for standardizing the mean (to 0.0) and the range of data between hybridized samples to about −3.0 to +3.0. When using the Z-score, the Z differences (Z_{diff}) are computed rather than ratios. The Z-score intensity (Z-scoreᵢⱼ) for intensity Iᵢⱼ for probe i (hybridization probe, protein, or other binding entity) and spot j is computed as:
Z-scoreᵢⱼ=(Iᵢⱼ−mnIᵢ)/sdIᵢ,
and
Zdiffⱼ(x,y)=Z-scoreₓⱼ−Z-score_{yj}

where x represents the x channel and y represents the y channel.

Another normalization protocol is the median intensity normalization protocol in which the raw intensities for all spots in each sample are normalized by the median of the raw intensities. For microarray data, the median intensity normalization method normalizes each hybridized sample by the median of the raw intensities of control genes (medianIᵢ) for all of the spots in that sample. Thus, upon normalization by the median intensity normalization method, the raw intensity Iᵢⱼ for probe i and spot j, has the value Imᵢⱼ where,
Imᵢⱼ=(Iᵢⱼ/medianIᵢ).

Another normalization protocol is the log median intensity protocol. In this protocol, raw expression intensities are normalized by the log of the median scaled raw intensities of representative spots for all spots in the sample. For microarray data, the log median intensity method normalizes each hybridized sample by the log of median scaled raw intensities of control genes (medianIᵢ) for all of the spots in that sample. As used herein, control genes are a set of genes that have reproducible accurately measured expression values. The value 1.0 is added to the intensity value to avoid taking the log(0.0) when intensity has zero value. Upon normalization by the median intensity normalization method, the raw intensity Iᵢⱼ for probe i and spot j, has the value Imᵢⱼ here,
Imᵢⱼ=log(1.0+(Iᵢⱼ/medianIᵢ)).

Yet another normalization protocol is the Z-score standard deviation log of intensity protocol. In this protocol, raw expression intensities are normalized by the mean log intensity (mnLIᵢ) and standard deviation log intensity (sdLIᵢ). For microarray data, the mean log intensity and the standard deviation log intensity is computed for the log of raw intensity of control genes. Then, the Z-score intensity Z log Sᵢⱼ for probe i and spot j is:
Z log Sᵢⱼ=(log(Iᵢⱼ)−mnLIᵢ)/sdLIᵢ.

Still another normalization protocol is the Z-score mean absolute deviation of log intensity protocol. In this protocol, raw expression intensities are normalized by the Z-score of the log intensity using the equation (log(intensity)-mean logarithm)/standard deviation logarithm. For microarray data, the Z-score mean absolute deviation of log intensity protocol normalizes each bound sample by the mean and mean absolute deviation of the logs of the raw intensities for all of the spots in the sample. The mean log intensity mnLIᵢ and the mean absolute deviation log intensity madLIᵢ are computed for the log of raw intensity of control genes. Then, the Z-score intensity Z log Aᵢⱼ for probe i and spot j is:
Z log Aᵢⱼ=(log(Iᵢⱼ)−mnLIᵢ)/madLIᵢ.

Another normalization protocol is the user normalization gene set protocol. In this protocol, raw expression intensities are normalized by the sum of the genes in a user defined gene set in each sample. This method is useful if a subset of genes has been determined to have relatively constant expression across a set of samples. Yet another normalization protocol is the calibration DNA gene set protocol in which each sample is normalized by the sum of calibration DNA genes. As used herein, calibration DNA genes are genes that produce reproducible expression values that are accurately measured. Such genes tend to have the same expression values on each of several different microarrays. The algorithm is the same as user normalization gene set protocol described above, but the set is predefined as the genes flagged as calibration DNA.

Yet another normalization protocol is the ratio median intensity correction protocol. This protocol is useful in embodiments in which a two-color fluorescence labeling and detection scheme is used. See, for example, section 5.6.1, infra. In the case where the two fluors in a two-color fluorescence labeling and detection scheme are Cy3 and Cy5, measurements are normalized by multiplying the ratio (Cy3/Cy5) by medianCy5/medianCy3 intensities. If background correction is enabled, measurements are normalized by multiplying the ratio (Cy3/Cy5) by (medianCy5-medianBkgdCy5)/(medianCy3-medianBkgdCy3) where medianBkgd means median background levels.

In some embodiments, intensity background correction is used to normalize measurements. The background intensity data from a spot quantification programs may be used to correct spot intensity. Background may be specified as either a global value or on a per-spot basis. If the array images have low background, then intensity background correction may not be necessary.

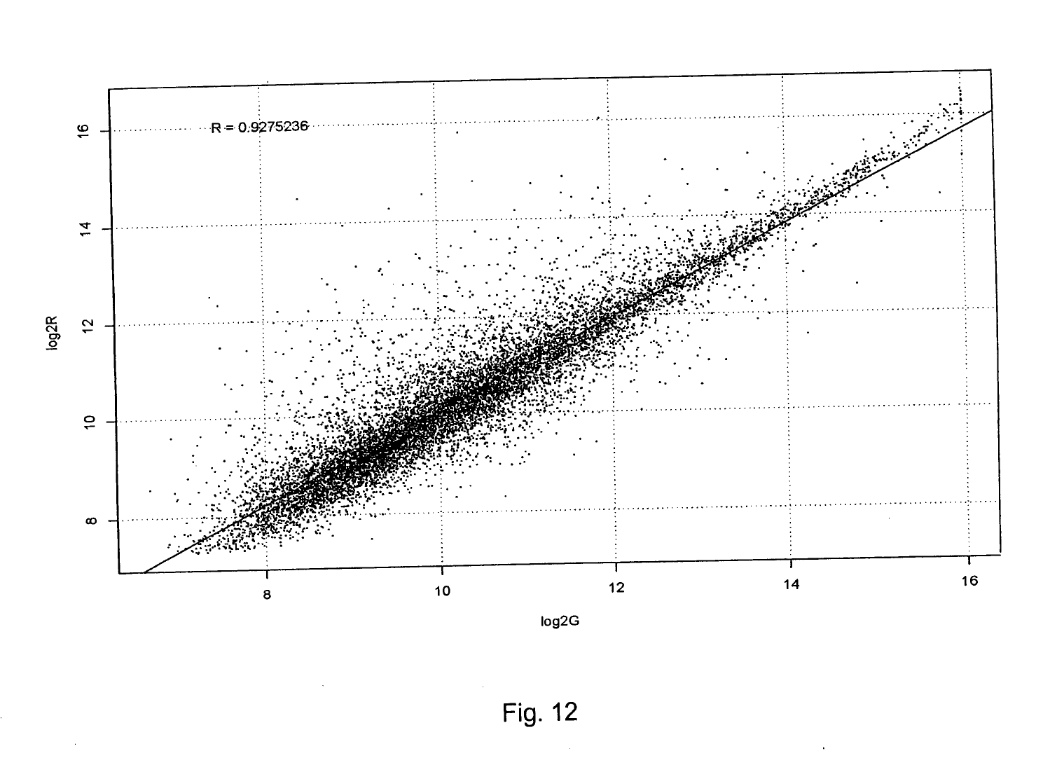

An intensity dependent normalization can be implemented in R, a language and environment for statistical computing and graphics, since most arrays present a non linear dependence between the M=log₂(R/G) and the overall intensity A=log₂\sqrt{(RG)}. For example, FIG. 12 shows a log₂(R) vs log₂(G) plot with a fitted line from a simple linear regression of log₂(R) on log₂(G). In a specific embodiment, the normalization method uses a lowess( ) scatter plot smoother which can be applied to all or a subgroup of probes on the array. The lowess( ) function is fitted to each M vs A plot and the adjusted M′ⱼ=Mⱼ−Mⱼ (lowess) values for each probe j are computed. The adjusted probe intensities are then given by R=2^{(Aj+(M′j/2))} and G=^{2(Aj-(M′j/2))}. For lowess( ), see, e.g., Becker et al., “The New S Language,” Wadsworth and Brooks/Cole (S version),1988; Ripley, 1996, Pattern Recognition and Neural Networks, Cambridge University Press; and Cleveland, 1979, J. Amer. Statist. Assoc. 74, 829:836, each of which is hereby incorporated by reference in its entirety. The normalization method can be applied assuming that (i) only a small proportion of genes is expected to vary between the reference and test samples

$$( i . e . , \frac{1}{j} \sum \frac{j}{1} {M}_{j} \approx 0 )$$

(ii) that the expression levels of up and down regulated genes is symmetric. Any array can be eliminated from further study if it shows significant defects and poor linear correlation coefficients (R<0.90), which is calculated by fitting a simple linear regression of normalized log₂(R) on log₂(G).

5.6 Transcriptional State Measurements

This section provides some exemplary methods for measuring the expression level of genes, which are one type of cellular constituent. One of skill in the art will appreciate that this invention is not limited to the following specific methods for measuring the expression level of genes in each organism in a plurality of organisms.

5.6.1 Transcript Assay Using Microarrays

The techniques described in this section are particularly useful for the determination of the expression state or the transcriptional state of a cell or cell type or any other cell sample by monitoring expression profiles. These techniques include the provision of polynucleotide probe arrays that can be used to provide simultaneous determination of the expression levels of a plurality of genes. These techniques further provide methods for designing and making such polynucleotide probe arrays.

The expression level of a nucleotide sequence in a gene can be measured by any high throughput techniques. However measured, the result is either the absolute or relative amounts of transcripts or response data, including but not limited to values representing abundances or abundance ratios. Preferably, measurement of the expression profile is made by hybridization to transcript arrays, which are described in this subsection. In one embodiment, “transcript arrays” or “profiling arrays” are used. Transcript arrays can be employed for analyzing the expression profile in a cell sample and especially for measuring the expression profile of a cell sample of a particular tissue type or developmental state or exposed to a drug of interest.

In one embodiment, a molecular profile is an expression profile that is obtained by hybridizing detectably labeled polynucleotides representing the nucleotide sequences in mRNA transcripts present in a cell (e.g., fluorescently labeled cDNA synthesized from total cell mRNA) to a microarray. A microarray is an array of positionally-addressable binding (e.g., hybridization) sites on a support for representing many of the nucleotide sequences in the genome of a cell or organism, preferably most or almost all of the genes. Each of such binding sites consists of polynucleotide probes bound to the predetermined region on the support. Microarrays can be made in a number of ways, of which several are described herein below. However produced, microarrays share certain characteristics. The arrays are reproducible, allowing multiple copies of a given array to be produced and easily compared with each other. Preferably, the microarrays are made from materials that are stable under binding (e.g., nucleic acid hybridization) conditions. Microarrays are preferably small, e.g., between 1 cm² and 25 cm², preferably 1 to 3 cm². However, both larger and smaller arrays are also contemplated and may be preferable, e.g., for simultaneously evaluating a very large number or very small number of different probes.

Preferably, a given binding site or unique set of binding sites in the microarray will specifically bind (e.g., hybridize) to a nucleotide sequence in a single gene from a cell or organism (e.g., to exon of a specific mRNA or a specific cDNA derived therefrom).

In some embodiments, the microarray is a first edition Human HuFL6800 (6800 elements) or a second edition HuU95A (12,000 elements) GeneChip. The HuFL6800 chip contains probes corresponding to 5000 named genes (based on the National Center for Biotechnology Information UniGene Build 139, as provided by Affymetrix, Santa Clara, Calif.), whereas the HuU95A contains more than 12,000 probe sets corresponding to 8900 names genes (UniGene Build 139).

The microarrays used can include one or more test probes, each of which has a polynucleotide sequence that is complementary to a subsequence of RNA or DNA to be detected. Each probe typically has a different nucleic acid sequence, and the position of each probe on the solid surface of the array is usually known. Indeed, the microarrays are preferably addressable arrays, more preferably positionally addressable arrays. Each probe of the array is preferably located at a known, predetermined position on the solid support so that the identity (e.g., the sequence) of each probe can be determined from its position on the array (e.g., on the support or surface). In some embodiments, the arrays are ordered arrays.

Preferably, the density of probes on a microarray or a set of microarrays is 100 different (e.g., non-identical) probes per 1 cm² or higher. More preferably, a microarray used in the methods of the invention will have at least 550 probes per 1 cm², at least 1,000 probes per 1 cm², at least 1,500 probes per 1 cm² or at least 2,000 probes per 1 cm². In a particularly preferred embodiment, the microarray is a high density array, preferably having a density of at least 2,500 different probes per 1 cm². The microarrays used in the invention therefore preferably contain at least 2,500, at least 5,000, at least 10,000, at least 15,000, at least 20,000, at least 25,000, at least 50,000 or at least 55,000 different (e.g., non-identical) probes.

In one embodiment, the microarray is an array (e.g., a matrix) in which each position represents a discrete binding site for a nucleotide sequence of a transcript encoded by a gene (e.g., for an exon of an mRNA or a cDNA derived therefrom). The collection of binding sites on a microarray contains sets of binding sites for a plurality of genes. For example, in various embodiments, the microarrays of the invention can comprise binding sites for products encoded by fewer than 50% of the genes in the genome of an organism. Alternatively, the microarrays of the invention can have binding sites for the products encoded by at least 50%, at least 75%, at least 85%, at least 90%, at least 95%, at least 99% or 100% of the genes in the genome of an organism. In other embodiments, the microarrays of the invention can having binding sites for products encoded by fewer than 50%, by at least 50%, by at least 75%, by at least 85%, by at least 90%, by at least 95%, by at least 99% or by 100% of the genes expressed by a cell of an organism. The binding site can be a DNA or DNA analog to which a particular RNA can specifically hybridize. The DNA or DNA analog can be, e.g., a synthetic oligomer or a gene fragment, e.g. corresponding to an exon.

In some embodiments of the present invention, a gene or an exon in a gene is represented in the profiling arrays by a set of binding sites comprising probes with different polynucleotides that are complementary to different sequence segments of the gene or the exon. Such polynucleotides are preferably of the length of 15 to 200 bases, more preferably of the length of 20 to 100 bases, most preferably 40-60 bases. Each probe sequence can also comprise linker sequences in addition to the sequence that is complementary to its target sequence. As used herein, a linker sequence is a sequence between the sequence that is complementary to its target sequence and the surface of support. For example, in preferred embodiments, the profiling arrays of the invention comprise one probe specific to each target gene or exon. However, if desired, the profiling arrays can contain at least 2, 5, 10, 100, or 1000 or more probes specific to some target genes or exons. For example, the array can contain probes tiled across the sequence of the longest mRNA isoform of a gene at single base steps.

In specific embodiments of the invention, when an exon has alternative spliced variants, a set of polynucleotide probes of successive overlapping sequences, e.g., tiled sequences, across the genomic region containing the longest variant of an exon can be included in the exon profiling arrays. The set of polynucleotide probes can comprise successive overlapping sequences at steps of a predetermined base intervals, e.g. at steps of 1, 5, or 10 base intervals, span, or are tiled across, the mRNA containing the longest variant. Such sets of probes therefore can be used to scan the genomic region containing all variants of an exon to determine the expressed variant or variants of the exon to determine the expressed variant or variants of the exon. Alternatively or additionally, a set of polynucleotide probes comprising exon specific probes and/or variant junction probes can be included in the exon profiling array. As used herein, a variant junction probe refers to a probe specific to the junction region of the particular exon variant and the neighboring exon. In some cases, the probe set contains variant junction probes specifically hybridizable to each of all different splice junction sequences of the exon. In other cases, the probe set contains exon specific probes specifically hybridizable to the common sequences in all different variants of the exon, and/or variant junction probes specifically hybridizable to the different splice junction sequences of the exon.

In some cases, an exon is represented in the exon profiling arrays by a probe comprising a polynucleotide that is complementary to the full length exon. In such instances, an exon is represented by a single binding site on the profiling arrays. In some preferred cases, an exon is represented by one or more binding sites on the profiling arrays, each of the binding sites comprising a probe with a polynucleotide sequence that is complementary to an RNA fragment that is a substantial portion of the target exon. The lengths of such probes are normally between 15-600 bases, preferably between 20-200 bases, more preferably between 30-100 bases, and most preferably between 40-80 bases. The average length of an exon is about 200 bases in some embodiments of the present invention (see, e.g., Lewin, Genes V, Oxford University Press, Oxford, 1994). A probe of length of 40-80 allows more specific binding of the exon than a probe of shorter length, thereby increasing the specificity of the probe to the target exon. For certain genes, one or more targeted exons can have sequence lengths less than 40-80 bases. In such cases, if probes with sequences longer than the target exons are to be used, it can be desirable to design probes comprising sequences that include the entire target exon flanked by sequences from the adjacent constitutively splice exon or exons such that the probe sequences are complementary to the corresponding sequence segments in the mRNAs. Using flanking sequences from adjacent constitutively spliced exon or exons rather than the genomic flanking sequences, e.g., intron sequences, permits comparable hybridization stringency with other probes of the same length. Preferably, the flanking sequences used are from the adjacent constitutively spliced exon or exons that are not involved in any alternative pathways. More preferably, the flanking sequences used do not comprise a significant portion of the sequence of the adjacent exon or exons so that cross-hybridization can be minimized. In some embodiments, when a target exon that is shorter than the desired probe length is involved in alternative splicing, probes comprising flanking sequences in different alternatively spliced mRNAs are designed so that expression level of the exon expressed in different alternatively spliced mRNAs can be measured.

In some instances, when alternative splicing pathways and/or exon duplication in separate genes are to be distinguished, the DNA array or set of arrays can also comprise probes that are complementary to sequences spanning the junction regions of two adjacent exons. Preferably, such probes comprise sequences from the two exons which are not substantially overlapped with probes for each individual exons so that cross hybridization can be minimized. Probes that comprise sequences from more than one exons are useful in distinguishing alternative splicing pathways and/or expression of duplicated exons in separate genes if the exons occurs in one or more alternative spliced mRNAs and/or one or more separated genes that contain the duplicated exons but not in other alternatively spliced mRNAs and/or other genes that contain the duplicated exons. Alternatively, for duplicate exons in separate genes, if the exons from different genes show substantial difference in sequence homology, it is preferable to include probes that are different so that the exons from different genes can be distinguished.

It will be apparent to one skilled in the art that any of the probe schemes, supra, can be combined on the same profiling array and/or on different arrays within the same set of profiling arrays so that a more accurate determination of the expression profile for a plurality of genes can be accomplished. It will also be apparent to one skilled in the art that the different probe schemes can also be used for different levels of accuracies in profiling. For example, a profiling array or array set comprising a small set of probes for each exon can be used to determine the relevant genes and/or RNA splicing pathways under certain specific conditions. An array or array set comprising larger sets of probes for the exons that are of interest is then used to more accurately determine the exon expression profile under such specific conditions. Other DNA array strategies that allow more advantageous use of different probe schemes are also encompassed.

Preferably, the microarrays used in the invention have binding sites (e.g., probes) for sets of exons for one or more genes relevant to the action of a drug of interest or in a biological pathway of interest. As discussed above, a “gene” is identified as a portion of DNA that is transcribed by RNA polymerase, which may include a 5N untranslated region (“UTR”), introns, exons and a 3N UTR. The number of genes in a genome can be estimated from the number of mRNAs expressed by the cell or organism, or by extrapolation of a well characterized portion of the genome. When the genome of the organism of interest has been sequenced, the number of ORFs can be determined and mRNA coding regions identified by analysis of the DNA sequence. In preferred embodiments of the invention, an array set comprising, in total, probes for all known or predicted exons in the genome of an organism are provided. As a non-limiting example, the present invention provides an array set comprising one or two probes for all or a portion of the known exons in the human genome.

It will be appreciated that when cDNA complementary to the RNA of a cell is made and hybridized to a microarray under suitable hybridization conditions, the level of hybridization to the site in the array corresponding to an exon of any particular gene will reflect the prevalence in the cell of mRNA or mRNAs containing the exon transcribed from that gene. For example, when detectably labeled (e.g., with a fluorophore) cDNA complementary to the total cellular mRNA is hybridized to a microarray, the site on the array corresponding to an exon of a gene (i.e., capable of specifically binding the product or products of the gene expressing) that is not transcribed or is removed during RNA splicing in the cell will have little or no signal (e.g., fluorescent signal), and an exon of a gene for which the encoded mRNA expressing the exon is prevalent will have a relatively strong signal. The relative abundance of different mRNAs produced from the same gene by alternative splicing is then determined by the signal strength pattern across the whole set of exons monitored for the gene.

In one embodiment, cDNAs from cell samples from two different conditions are hybridized to the binding sites of the microarray using a two-color protocol. In the case of drug responses one cell sample is exposed to a drug and another cell sample of the same type is not exposed to the drug. In the case of pathway responses one cell is exposed to a pathway perturbation and another cell of the same type is not exposed to the pathway perturbation. The cDNA derived from each of the two cell types are differently labeled (e.g., with Cy3 and Cy5) so that they can be distinguished. In one embodiment, for example, cDNA from a cell treated with a drug (or exposed to a pathway perturbation) is synthesized using a fluorescein-labeled dNTP, and cDNA from a second cell, not drug-exposed, is synthesized using a rhodamine-labeled dNTP. When the two cDNAs are mixed and hybridized to the microarray, the relative intensity of signal from each cDNA set is determined for each site on the array, and any relative difference in abundance of a particular exon detected.

In the example described above, the cDNA from the drug-treated (or pathway perturbed) cell will fluoresce green when the fluorophore is stimulated and the cDNA from the untreated cell will fluoresce red. As a result, when the drug treatment has no effect, either directly or indirectly, on the transcription and/or post-transcriptional splicing of a particular gene in a cell, the exon expression patterns will be indistinguishable in both cells and, upon reverse transcription, red-labeled and green-labeled cDNA will be equally prevalent. When hybridized to the microarray, the binding site(s) for that species of RNA will emit wavelengths characteristic of both fluorophores. In contrast, when the drug-exposed cell is treated with a drug that, directly or indirectly, change the transcription and/or post-transcriptional splicing of a particular gene in the cell, the exon expression pattern as represented by ratio of green to red fluorescence for each exon binding site will change. When the drug increases the prevalence of an mRNA, the ratios for each exon expressed in the mRNA will increase, whereas when the drug decreases the prevalence of an mRNA, the ratio for each exons expressed in the mRNA will decrease.

The use of a two-color fluorescence labeling and detection scheme to define alterations in gene expression has been described in connection with detection of mRNAs, e.g., in Shena et al., 1995, Quantitative monitoring of gene expression patterns with a complementary DNA microarray, Science 270:467-470, which is incorporated by reference in its entirety for all purposes. The scheme is equally applicable to labeling and detection of exons. An advantage of using cDNA labeled with two different fluorophores is that a direct and internally controlled comparison of the mRNA or exon expression levels corresponding to each arrayed gene in two cell states can be made, and variations due to minor differences in experimental conditions (e.g., hybridization conditions) will not affect subsequent analyses. However, it will be recognized that it is also possible to use cDNA from a single cell, and compare, for example, the absolute amount of a particular exon in, e.g., a drug-treated or pathway-perturbed cell and an untreated cell. Furthermore, labeling with more than two colors is also contemplated in the present invention. In some embodiments of the invention, at least 5, 10, 20, or 100 dyes of different colors can be used for labeling. Such labeling permits simultaneous hybridizing of the distinguishably labeled cDNA populations to the same array, and thus measuring, and optionally comparing the expression levels of, mRNA molecules derived from more than two samples. Dyes that can be used include, but are not limited to, fluorescein and its derivatives, rhodamine and its derivatives, texas red, 5Ncarboxy-fluorescein (“FMA”), 2N,7N-dimethoxy-4N,5N-dichloro-6-carboxy-fluorescein (“JOE”), N,N,NN,NN-tetramethyl-6-carboxy-rhodamine (“TAMRA”), 6Ncarboxy-X-rhodamine (“ROX”), HEX, TET, IRD40, and IRD41, cyamine dyes, including but are not limited to Cy3, Cy3.5 and Cy5; BODIPY dyes including but are not limited to BODIPY-FL, BODIPY-TR, BODIPY-TMR, BODIPY-630/650, and BODIPY-650/670; and ALEXA dyes, including but are not limited to ALEXA-488, ALEXA-532, ALEXA-546, ALEXA-568, and ALEXA-594; as well as other fluorescent dyes which will be known to those who are skilled in the art.

In some embodiments of the invention, hybridization data are measured at a plurality of different hybridization times so that the evolution of hybridization levels to equilibrium can be determined. In such embodiments, hybridization levels are most preferably measured at hybridization times spanning the range from 0 to in excess of what is required for sampling of the bound polynucleotides (i.e., the probe or probes) by the labeled polynucleotides so that the mixture is close to or substantially reached equilibrium, and duplexes are at concentrations dependent on affinity and abundance rather than diffusion. However, the hybridization times are preferably short enough that irreversible binding interactions between the labeled polynucleotide and the probes and/or the surface do not occur, or are at least limited. For example, in embodiments wherein polynucleotide arrays are used to probe a complex mixture of fragmented polynucleotides, typical hybridization times may be approximately 0-72 hours. Appropriate hybridization times for other embodiments will depend on the particular polynucleotide sequences and probes used, and may be determined by those skilled in the art (see, e.g., Sambrook et al., Eds., 1989, Molecular Cloning: A Laboratory Manual, 2nd ed., Vol. 1-3, Cold Spring Harbor Laboratory, Cold Spring Harbor, N.Y.).

In one embodiment, hybridization levels at different hybridization times are measured separately on different, identical microarrays. For each such measurement, at hybridization time when hybridization level is measured, the microarray is washed briefly, preferably in room temperature in an aqueous solution of high to moderate salt concentration (e.g., 0.5 to 3 M salt concentration) under conditions which retain all bound or hybridized polynucleotides while removing all unbound polynucleotides. The detectable label on the remaining, hybridized polynucleotide molecules on each probe is then measured by a method which is appropriate to the particular labeling method used. The resulted hybridization levels are then combined to form a hybridization curve. In another embodiment, hybridization levels are measured in real time using a single microarray. In this embodiment, the microarray is allowed to hybridize to the sample without interruption and the microarray is interrogated at each hybridization time in a non-invasive manner. In still another embodiment, one can use one array, hybridize for a short time, wash and measure the hybridization level, put back to the same sample, hybridize for another period of time, wash and measure again to get the hybridization time curve.

Preferably, at least two hybridization levels at two different hybridization times are measured, a first one at a hybridization time that is close to the time scale of cross-hybridization equilibrium and a second one measured at a hybridization time that is longer than the first one. The time scale of cross-hybridization equilibrium depends, inter alia, on sample composition and probe sequence and may be determined by one skilled in the art. In preferred embodiments, the first hybridization level is measured at between 1 to 10 hours, whereas the second hybridization time is measured at 2, 4, 6, 10, 12, 16, 18, 48 or 72 times as long as the first hybridization time.

5.6.1.1 Preparing Probes for Microarrays

As noted above, the “probe” to which a particular polynucleotide molecule, such as an exon, specifically hybridizes according to the invention is a complementary polynucleotide sequence. Preferably one or more probes are selected for each target exon. For example, when a minimum number of probes are to be used for the detection of an exon, the probes normally comprise nucleotide sequences greater than 40 bases in length. Alternatively, when a large set of redundant probes is to be used for an exon, the probes normally comprise nucleotide sequences of 40-60 bases. The probes can also comprise sequences complementary to full length exons. The lengths of exons can range from less than 50 bases to more than 200 bases. Therefore, when a probe length longer than exon is to be used, it is preferable to augment the exon sequence with adjacent constitutively spliced exon sequences such that the probe sequence is complementary to the continuous mRNA fragment that contains the target exon. This will allow comparable hybridization stringency among the probes of an exon profiling array. It will be understood that each probe sequence may also comprise linker sequences in addition to the sequence that is complementary to its target sequence.

The probes may comprise DNA or DNA “mimics” (e.g., derivatives and analogues) corresponding to a portion of each exon of each gene in an organism's genome. In one embodiment, the probes of the microarray are complementary RNA or RNA mimics. DNA mimics are polymers composed of subunits capable of specific, Watson-Crick-like hybridization with DNA, or of specific hybridization with RNA. The nucleic acids can be modified at the base moiety, at the sugar moiety, or at the phosphate backbone. Exemplary DNA mimics include, e.g., phosphorothioates. DNA can be obtained, e.g., by polymerase chain reaction (PCR) amplification of exon segments from genomic DNA, cDNA (e.g., by RT-PCR), or cloned sequences. PCR primers are preferably chosen based on known sequence of the exons or cDNA that result in amplification of unique fragments (i.e., fragments that do not share more than 10 bases of contiguous identical sequence with any other fragment on the microarray). Computer programs that are well known in the art are useful in the design of primers with the required specificity and optimal amplification properties, such as Oligo version 5.0 (National Biosciences). Typically each probe on the microarray will be between 20 bases and 600 bases, and usually between 30 and 200 bases in length. PCR methods are well known in the art, and are described, for example, in Innis et al., eds., 1990, PCR Protocols: A Guide to Methods and Applications, Academic Press Inc., San Diego, Calif. It will be apparent to one skilled in the art that controlled robotic systems are useful for isolating and amplifying nucleic acids.

An alternative, preferred means for generating the polynucleotide probes of the microarray is by synthesis of synthetic polynucleotides or oligonucleotides, e.g., using N-phosphonate or phosphoramidite chemistries (Froehler et al., 1986, Nucleic Acid Res. 14:5399-5407; McBride et al., 1983, Tetrahedron Lett. 24:246-248). Synthetic sequences are typically between 15 and 600 bases in length, more typically between 20 and 100 bases, most preferably between 40 and 70 bases in length. In some embodiments, synthetic nucleic acids include non-natural bases, such as, but by no means limited to, inosine. As noted above, nucleic acid analogues may be used as binding sites for hybridization. An example of a suitable nucleic acid analogue is peptide nucleic acid (see, e.g., Egholm et al., 1993, Nature 363:566-568; and U.S. Pat. No. 5,539,083).

In alternative embodiments, the hybridization sites (e.g., the probes) are made from plasmid or phage clones of genes, cDNAs (e.g., expressed sequence tags), or inserts therefrom (Nguyen et al., 1995, Genomics 29:207-209).

5.6.1.2 Attaching Nucleic Acids to the Solid Surface

Preformed polynucleotide probes can be deposited on a support to form the array. Alternatively, polynucleotide probes can be synthesized directly on the support to form the array. The probes are attached to a solid support or surface, which may be made, e.g., from glass, plastic (e.g., polypropylene, nylon), polyacrylamide, nitrocellulose, gel, or other porous or nonporous material.

A preferred method for attaching the nucleic acids to a surface is by printing on glass plates, as is described generally by Schena et al, 1995, Science 270:467-470. This method is especially useful for preparing microarrays of cDNA (See also, DeRisi et al, 1996, Nature Genetics 14:457-460; Shalon et al., 1996, Genome Res. 6:639-645; and Schena et al., 1995, Proc. Natl. Acad. Sci. U.S.A. 93:10539-11286).

A second preferred method for making microarrays is by making high-density polynucleotide arrays. Techniques are known for producing arrays containing thousands of oligonucleotides complementary to defined sequences, at defined locations on a surface using photolithographic techniques for synthesis in situ (see, Fodor et al., 1991, Science 251:767-773; Pease et al., 1994, Proc. Natl. Acad. Sci. U.S.A. 91:5022-5026; Lockhart et al., 1996, Nature Biotechnology 14:1675; U.S. Pat. Nos. 5,578,832; 5,556,752; and 5,510,270) or other methods for rapid synthesis and deposition of defined oligonucleotides (Blanchard et al., Biosensors & Bioelectronics 11:687-690). When these methods are used, oligonucleotides (e.g., 60-mers) of known sequence are synthesized directly on a surface such as a derivatized glass slide. The array produced can be redundant, with several polynucleotide molecules per exon.

Other methods for making microarrays, e.g., by masking (Maskos and Southern, 1992, Nucl. Acids. Res. 20:1679-1684), may also be used. In principle, and as noted supra, any type of array, for example, dot blots on a nylon hybridization membrane (see Sambrook et al., supra) could be used. However, as will be recognized by those skilled in the art, very small arrays will frequently be preferred because hybridization volumes will be smaller.

In a particularly preferred embodiment, microarrays of the invention are manufactured by means of an ink jet printing device for oligonucleotide synthesis, e.g., using the methods and systems described by Blanchard in International Patent Publication No. WO 98/41531, published Sep. 24, 1998; Blanchard et al., 1996, Biosensors and Bioelectronics 11:687-690; Blanchard, 1998, in Synthetic DNA Arrays in Genetic Engineering, Vol. 20, J. K. Setlow, Ed., Plenum Press, New York at pages 15 111-123; and U.S. Pat. No.6,028,189 to Blanchard. Specifically, the polynucleotide probes in such microarrays are preferably synthesized in arrays, e.g., on a glass slide, by serially depositing individual nucleotide bases in “microdroplets” of a high surface tension solvent such as propylene carbonate. The microdroplets have small volumes (e.g., 100 pL or less, more preferably 50 pL or less) and are separated from each other on the microarray (e.g., by hydrophobic domains) to form circular surface tension wells which define the locations of the array elements (i.e., the different probes). Polynucleotide probes are normally attached to the surface covalently at the 3N end of the polynucleotide. Alternatively, polynucleotide probes can be attached to the surface covalently at the SN end of the polynucleotide (see for example, Blanchard, 1998, in Synthetic DNA Arrays in Genetic Engineering, Vol. 20, J. K. Setlow, Ed., Plenum Press, New York at pages 111-123).

5.6.1.3 Target Polynucleotide Molecules

Target polynucleotides that can be analyzed by the methods and compositions of the invention include RNA molecules such as, but by no means limited to, messenger RNA (mRNA) molecules, ribosomal RNA (rRNA) molecules, cRNA molecules (i.e., RNA molecules prepared from cDNA molecules that are transcribed in vivo) and fragments thereof. Target polynucleotides that can also be analyzed by the methods of the present invention include, but are not limited to DNA molecules such as genomic DNA molecules, cDNA molecules, and fragments thereof including oligonucleotides, ESTs, STSs, etc.

The target polynucleotides can be from any source. For example, the target polynucleotide molecules can be naturally occurring nucleic acid molecules such as genomic or extragenomic DNA molecules isolated from a patient, or RNA molecules, such as mRNA molecules, isolated from a patient. Alternatively, the polynucleotide molecules can be synthesized, including, e.g., nucleic acid molecules synthesized enzymatically in vivo or in vitro, such as cDNA molecules, or polynucleotide molecules synthesized by PCR, RNA molecules synthesized by in vitro transcription, etc. The sample of target polynucleotides can comprise, e.g., molecules of DNA, RNA, or copolymers of DNA and RNA. In preferred embodiments, the target polynucleotides of the invention will correspond to particular genes or to particular gene transcripts (e.g., to particular mRNA sequences expressed in cells or to particular cDNA sequences derived from such mRNA sequences). However, in many embodiments, the target polynucleotides can correspond to particular fragments of a gene transcript. For example, the target polynucleotides may correspond to different exons of the same gene, e.g., so that different splice variants of the gene can be detected and/or analyzed.

In preferred embodiments, the target polynucleotides to be analyzed are prepared in vitro from nucleic acids extracted from cells. For example, in one embodiment, RNA is extracted from cells (e.g., total cellular RNA, poly(A)⁺ messenger RNA, fraction thereof) and messenger RNA is purified from the total extracted RNA. Methods for preparing total and poly(A)⁺ RNA are well known in the art, and are described generally, e.g., in Sambrook et al., supra. In one embodiment, RNA is extracted from cells of the various types of interest in this invention using guanidinium thiocyanate lysis followed by CsCl centrifugation and an oligo dT purification (Chirgwin et al., 1979, Biochemistry 18:5294-5299). In another embodiment, RNA is extracted from cells using guanidinium thiocyanate lysis followed by purification on RNeasy columns (Qiagen). cDNA is then synthesized from the purified mRNA using, e.g., oligo-dT or random primers. In preferred embodiments, the target polynucleotides are cRNA prepared from purified messenger RNA extracted from cells. As used herein, cRNA is defined here as RNA complementary to the source RNA. The extracted RNAs are amplified using a process in which doubled-stranded cDNAs are synthesized from the RNAs using a primer linked to an RNA polymerase promoter in a direction capable of directing transcription of anti-sense RNA. Anti-sense RNAs or cRNAs are then transcribed from the second strand of the double-stranded cDNAs using an RNA polymerase (see, e.g., U.S. Pat. Nos. 5,891,636, 5,716,785; 5,545,522 and 6,132,997; see also, U.S. Pat. No. 6,271,002, and U.S. Provisional Patent Application Ser. No. 60/253,641, filed on Nov. 28, 2000, by Ziman et al.). Both oligo-dT primers (U.S. Pat. Nos. 5,545,522 and 6,132,997) or random primers (U.S. Provisional Patent Application Ser. No. 60/253,641, filed on Nov. 28, 2000, by Ziman et al.) that contain an RNA polymerase promoter or complement thereof can be used. Preferably, the target polynucleotides are short and/or fragmented polynucleotide molecules that are representative of the original nucleic acid population of the cell.

The target polynucleotides to be analyzed by the methods of the invention are preferably detectably labeled. For example, cDNA can be labeled directly, e.g., with nucleotide analogs, or indirectly, e.g., by making a second, labeled cDNA strand using the first strand as a template. Alternatively, the double-stranded cDNA can be transcribed into cRNA and labeled.

Preferably, the detectable label is a fluorescent label, e.g., by incorporation of nucleotide analogs. Other labels suitable for use in the present invention include, but are not limited to, biotin, imminobiotin, antigens, cofactors, dinitrophenol, lipoic acid, olefinic compounds, detectable polypeptides, electron rich molecules, enzymes capable of generating a detectable signal by action upon a substrate, and radioactive isotopes. Preferred radioactive isotopes include ³²P, ³⁵S, ¹⁴C, ¹⁵N and 125I. Fluorescent molecules suitable for the present invention include, but are not limited to, fluorescein and its derivatives, rhodamine and its derivatives, texas red, 5Ncarboxy-fluorescein (“FMA”), 2N,7N-dimethoxy-4N,5N-dichloro-6-carboxy-fluorescein (“JOE”), N,N,NN,NN-tetramethyl-6-carboxy-rhodamine (“TAMRA”), 6Ncarboxy-X-rhodamine (“ROX”), HEX, TET, IRD40, and IRD41. Fluorescent molecules that are suitable for the invention further include: cyamine dyes, including by not limited to Cy3, Cy3.5 and Cy5; BODIPY dyes including but not limited to BODIPY-FL, BODIPY-TR, BODIPY-TMR, BODIPY-630/650, and BODIPY-650/670; and ALEXA dyes, including but not limited to ALEXA-488, ALEXA-532, ALEXA-546, ALEXA-568, and ALEXA-594; as well as other fluorescent dyes which will be known to those who are skilled in the art. Electron rich indicator molecules suitable for the present invention include, but are not limited to, ferritin, hemocyanin, and colloidal gold. Alternatively, in less preferred embodiments the target polynucleotides may be labeled by specifically complexing a first group to the polynucleotide. A second group, covalently linked to an indicator molecules and which has an affinity for the first group, can be used to indirectly detect the target polynucleotide. In such an embodiment, compounds suitable for use as a first group include, but are not limited to, biotin and iminobiotin. Compounds suitable for use as a second group include, but are not limited to, avidin and streptavidin.

5.6.1.4 Hybridization to Microarrays

As described supra, nucleic acid hybridization and wash conditions are chosen so that the polynucleotide molecules to be analyzed by the invention (referred to herein as the “target polynucleotide molecules) specifically bind or specifically hybridize to the complementary polynucleotide sequences of the array, preferably to a specific array site, wherein its complementary DNA is located.

Arrays containing double-stranded probe DNA situated thereon are preferably subjected to denaturing conditions to render the DNA single-stranded prior to contacting with the target polynucleotide molecules. Arrays containing single-stranded probe DNA (e.g., synthetic oligodeoxyribonucleic acids) may need to be denatured prior to contacting with the target polynucleotide molecules, e.g., to remove hairpins or dimers which form due to self complementary sequences.

Optimal hybridization conditions will depend on the length (e.g., oligomer versus polynucleotide greater than 200 bases) and type (e.g., RNA, or DNA) of probe and target nucleic acids. General parameters for specific (e.g., stringent) hybridization conditions for nucleic acids are described in Sambrook et al., (supra), and in Ausubel et al., 1987, Current Protocols in Molecular Biology, Greene Publishing and Wiley-Interscience, New York. When the cDNA microarrays of Schena et al. are used, typical hybridization conditions are hybridization in 5×SSC plus 0.2% SDS at 65° C. for four hours, followed by washes at 25° C. in low stringency wash buffer (1×SSC plus 0.2% SDS), followed by 10 minutes at 25° C. in higher stringency wash buffer (0.1×SSC plus 0.2% SDS) (Shena et al., 1996, Proc. Natl. Acad. Sci. U.S.A. 93:10614). Useful hybridization conditions are also provided in, e.g., Tijessen, 1993, Hybridization with Nucleic Acid Probes, Elsevier Science Publishers B. V. and Kricka, 1992, Nonisotopic DNA Probe Techniques, Academic Press, San Diego, Calif.

Particularly preferred hybridization conditions for use with the screening and/or signaling chips of the present invention include hybridization at a temperature at or near the mean melting temperature of the probes (e.g., within 5° C., more preferably within 2° C.) in 1 M NaCl, 50 mM MES buffer (pH 6.5), 0.5% sodium Sarcosine and 30% formamide.

5.6.1.5 Signal Detection and Data Analysis

It will be appreciated that when target sequences, e.g., cDNA or cRNA, complementary to the RNA of a cell is made and hybridized to a microarray under suitable hybridization conditions, the level of hybridization to the site in the array corresponding to an exon of any particular gene will reflect the prevalence in the cell of mRNA or mRNAs containing the exon transcribed from that gene. For example, when detectably labeled (e.g., with a fluorophore) cDNA complementary to the total cellular mRNA is hybridized to a microarray, the site on the array corresponding to an exon of a gene (e.g., capable of specifically binding the product or products of the gene expressing) that is not transcribed or is removed during RNA splicing in the cell will have little or no signal (e.g., fluorescent signal), and an exon of a gene for which the encoded mRNA expressing the exon is prevalent will have a relatively strong signal. The relative abundance of different mRNAs produced from the same gene by alternative splicing is then determined by the signal strength pattern across the whole set of exons monitored for the gene.

In preferred embodiments, target sequences, e.g., cDNAs or cRNAs, from two different cells are hybridized to the binding sites of the microarray. In the case of drug responses one cell sample is exposed to a drug and another cell sample of the same type is not exposed to the drug. In the case of pathway responses one cell is exposed to a pathway perturbation and another cell of the same type is not exposed to the pathway perturbation. The cDNA or cRNA derived from each of the two cell types are differently labeled so that they can be distinguished. In one embodiment, for example, cDNA from a cell treated with a drug (or exposed to a pathway perturbation) is synthesized using a fluorescein-labeled dNTP, and cDNA from a second cell, not drug-exposed, is synthesized using a rhodamine-labeled dNTP. When the two cDNAs are mixed and hybridized to the microarray, the relative intensity of signal from each cDNA set is determined for each site on the array, and any relative difference in abundance of a particular exon detected.

In the example described above, the cDNA from the drug-treated (or pathway perturbed) cell will fluoresce green when the fluorophore is stimulated and the cDNA from the untreated cell will fluoresce red. As a result, when the drug treatment has no effect, either directly or indirectly, on the transcription and/or post-transcriptional splicing of a particular gene in a cell, the exon expression patterns will be indistinguishable in both cells and, upon reverse transcription, red-labeled and green-labeled cDNA will be equally prevalent. When hybridized to the microarray, the binding site(s) for that species of RNA will emit wavelengths characteristic of both fluorophores. In contrast, when the drug-exposed cell is treated with a drug that, directly or indirectly, changes the transcription and/or post-transcriptional splicing of a particular gene in the cell, the exon expression pattern as represented by ratio of green to red fluorescence for each exon binding site will change. When the drug increases the prevalence of an mRNA, the ratios for each exon expressed in the mRNA will increase, whereas when the drug decreases the prevalence of an mRNA, the ratio for each exons expressed in the mRNA will decrease.

The use of a two-color fluorescence labeling and detection scheme to define alterations in gene expression has been described in connection with detection of mRNAs, e.g., in Shena et al., 1995, Science 270:467-470, which is incorporated by reference in its entirety for all purposes. The scheme is equally applicable to labeling and detection of exons. An advantage of using target sequences, e.g., cDNAs or cRNAs, labeled with two different fluorophores is that a direct and internally controlled comparison of the mRNA or exon expression levels corresponding to each arrayed gene in two cell states can be made, and variations due to minor differences in experimental conditions (e.g., hybridization conditions) will not affect subsequent analyses. However, it will be recognized that it is also possible to use cDNA from a single cell, and compare, for example, the absolute amount of a particular exon in, e.g., a drug-treated or pathway-perturbed cell and an untreated cell.

When fluorescently labeled probes are used, the fluorescence emissions at each site of a transcript array can be, preferably, detected by scanning confocal laser microscopy. In one embodiment, a separate scan, using the appropriate excitation line, is carried out for each of the two fluorophores used. Alternatively, a laser can be used that allows simultaneous specimen illumination at wavelengths specific to the two fluorophores and emissions from the two fluorophores can be analyzed simultaneously (see Shalon et al., 1996, Genome Res. 6:639-645). In a preferred embodiment, the arrays are scanned with a laser fluorescence scanner with a computer controlled X-Y stage and a microscope objective. Sequential excitation of the two fluorophores is achieved with a multi-line, mixed gas laser, and the emitted light is split by wavelength and detected with two photomultiplier tubes. Such fluorescence laser scanning devices are described, e.g., in Schena et al., 1996, Genome Res. 6:639-645. Alternatively, the fiber-optic bundle described by Ferguson et al., 1996, Nature Biotech. 14:1681-1684, can be used to monitor mRNA abundance levels at a large number of sites simultaneously.

Signals are recorded and, in a preferred embodiment, analyzed by computer. In one embodiment, the scanned image is despeckled using a graphics program (e.g., Hijaak Graphics Suite) and then analyzed using an image gridding program that creates a spreadsheet of the average hybridization at each wavelength at each site. If necessary, an experimentally determined correction for “cross talk” (or overlap) between the channels for the two fluors can be made. For any particular hybridization site on the transcript array, a ratio of the emission of the two fluorophores can be calculated. The ratio is independent of the absolute expression level of the cognate gene, but is useful for genes whose expression is significantly modulated by drug administration, gene deletion, or any other tested event.

According to the method of the invention, the relative abundance of an mRNA and/or an exon expressed in an mRNA in two cells or cell lines is scored as perturbed (e.g., the abundance is different in the two sources of mRNA tested) or as not perturbed (e.g., the relative abundance is the same). As used herein, a difference between the two sources of RNA of at least a factor of 25% (e.g., RNA is 25% more abundant in one source than in the other source), more usually 50%, even more often by a factor of 2 (e.g., twice as abundant), 3 (three times as abundant), or 5 (five times as abundant) is scored as a perturbation. Present detection methods allow reliable detection of differences of an order of 1.5 fold to 3-fold.

It is, however, also advantageous to determine the magnitude of the relative difference in abundances for an mRNA and/or an exon expressed in an mRNA in two cells or in two cell lines. This can be carried out, as noted above, by calculating the ratio of the emission of the two fluorophores used for differential labeling, or by analogous methods that will be readily apparent to those of skill in the art.

5.6.2 RT-PCR

In one embodiment, the level of gene expression can be measured by amplifying RNA from a sample using reverse transcription (RT) in combination with the polymerase chain reaction (PCR). In accordance with this embodiment, the reverse transcription may be quantitative.

Total RNA, or mRNA from a sample is used as a template, and a primer specific to the transcribed portion of the gene(s) is used to initiate reverse transcription. Methods of reverse transcribing RNA into cDNA are well known and described in Sambrook et al., 1989, supra. Primer design can be accomplished utilizing commercially available software (e.g., Primer Designer 1.0, Scientific Sofware etc.). The product of the reverse transcription is subsequently used as a template for PCR.

PCR provides a method for rapidly amplifying a particular nucleic acid sequence by using multiple cycles of DNA replication catalyzed by a thermostable, DNA-dependent DNA polymerase to amplify the target sequence of interest. PCR requires the presence of a nucleic acid to be amplified, two single-stranded oligonucleotide primers flanking the sequence to be amplified, a DNA polymerase, deoxyribonucleoside triphosphates, a buffer and salts. The method of PCR is well known in the art. PCR, is performed as described in Mullis and Faloona, 1987, Methods Enzymol., 155: 335, which is incorporated herein by reference.

PCR is performed using template DNA or cDNA (at least 1 fg; more usefully, 1-1000 ng) and at least 25 pmol of oligonucleotide primers. A typical reaction mixture includes: 2 μl of DNA, 25 pmol of oligonucleotide primer, 2.5 μl of 10 M PCR buffer 1 (Perkin-Elmer, Foster City, Calif.), 0.4 μl of 1.25 μM dNTP, 0.15 μl (or 2.5 units) of Taq DNA polymerase (Perkin Elmer, Foster City, Calif.) and deionized water to a total volume of 25 μl. Mineral oil is overlaid and the PCR is performed using a programmable thermal cycler.

The length and temperature of each step of a PCR cycle, as well as the number of cycles, are adjusted according to the stringency requirements in effect. Annealing temperature and timing are determined both by the efficiency with which a primer is expected to anneal to a template and the degree of mismatch that is to be tolerated. The ability to optimize the stringency of primer annealing conditions is well within the knowledge of one of moderate skill in the art. An annealing temperature of between 30° C. and 72° C. is used. Initial denaturation of the template molecules normally occurs at between 92° C. and 99° C. for 4 minutes, followed by 20-40 cycles consisting of denaturation (94-99° C. for 15 seconds to 1 minute), annealing (temperature determined as discussed above; 1-2 minutes), and extension (72° C. for 1 minute). The final extension step is generally carried out for 4 minutes at 72° C., and may be followed by an indefinite (0-24 hour) step at 4° C.

QRT-PCR, which is quantitative in nature, can also be performed to provide a quantitative measure of gene expression levels. In QRT-PCR reverse transcription and PCR can be performed in two steps, or reverse transcription combined with PCR can be performed concurrently. One of these techniques, for which there are commercially available kits such as Taqman® (Perkin Elmer, Foster City, Calif.), is performed with a transcript-specific antisense probe. This probe is specific for the PCR product (e.g. a nucleic acid fragment derived from a gene) and is prepared with a quencher and fluorescent reporter probe complexed to the 5 end of the oligonucleotide. Different fluorescent markers are attached to different reporters, allowing for measurement of two products in one reaction. When Taq DNA polymerase is activated, it cleaves off the fluorescent reporters of the probe bound to the template by virtue of its 5 -to-3 exonuclease activity. In the absence of the quenchers, the reporters now fluoresce. The color change in the reporters is proportional to the amount of each specific product and is measured by a fluorometer; therefore, the amount of each color is measured and the PCR product is quantified. The PCR reactions are performed in 96 well plates so that samples derived from many individuals are processed and measured simultaneously. The Taqman® system has the additional advantage of not requiring gel electrophoresis and allows for quantification when used with a standard curve.

A second technique useful for detecting PCR products quantitatively without is to use an intercalating dye such as the commercially available QuantiTect™ SYBR® Green PCR (Qiagen, Valencia Calif.). RT-PCR is performed using SYBR® green as a fluorescent label which is incorporated into the PCR product during the PCR stage and produces a flourescense proportional to the amount of PCR product.

Both Taqman® and QuantiTect™ SYBR® systems can be used subsequent to reverse transcription of RNA. Reverse transcription can either be performed in the same reaction mixture as the PCR step (one-step protocol) or reverse transcription can be performed first prior to amplification utilizing PCR (two-step protocol).

Additionally, other systems to quantitatively measure mRNA expression products are known including Molecular Beacons® which uses a probe having a fluorescent molecule and a quencher molecule, the probe capable of forming a hairpin structure such that when in the hairpin form, the fluorescence molecule is quenched, and when hybridized the flourescense increases giving a quantitative measurement of gene expression.

Additional techniques to quantitatively measure RNA expression include, but are not limited to, polymerase chain reaction, ligase chain reaction, Qbeta replicase (see, e.g., International Application No. PCT/US87/00880), isothermal amplification method (see, e.g., Walker et al. (1992) PNAS 89:382-396), strand displacement amplification (SDA), repair chain reaction, Asymmetric Quantitative PCR (see, e.g., U.S. Publication No. US200330134307A1) and the multiplex microsphere bead assay described in Fuja et al., 2004, Journal of Biotechnology 108:193-205.

The level of gene expression can be measured by amplifying RNA from a sample using transcription based amplification systems (TAS), including nucleic acid sequence amplification (NASBA) and 3SR. See, e.g., Kwoh et al (1989) PNAS USA 86:1173; International Publication No. WO 88/10315; and U.S. Pat. No. 6,329,179. In NASBA, the nucleic acids may be prepared for amplification using conventional phenol/chloroform extraction, heat denaturation, treatment with lysis buffer and minispin columns for isolation of DNA and RNA or guanidinium chloride extraction of RNA. These amplification techniques involve annealing a primer that has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by a polymerase such as T7 or SP6. In an isothermal cyclic reaction, the RNA's are reverse transcribed into double stranded DNA, and transcribed once with a polymerase such as T7 or SP6. The resulting products, whether truncated or complete, indicate target specific sequences.

Several techniques may be used to separate amplification products. For example, amplification products may be separated by agarose, agarose-acrylamide or polyacrylamide gel electrophoresis using conventional methods. See Sambrook et al., 1989. Several techniques for detecting PCR products quantitatively without electrophoresis may also be used according to the invention (see for example PCR Protocols, A Guide to Methods and Applications, Innis et al., Academic Press, Inc. N.Y., (1990)). For example, chromatographic techniques may be employed to effect separation. There are many kinds of chromatography which may be used in the present invention: adsorption, partition, ion-exchange and molecular sieve, HPLC, and many specialized techniques for using them including column, paper, thin-layer and gas chromatography (Freifelder, Physical Biochemistry Applications to Biochemistry and Molecular Biology, 2nd ed., Wm. Freeman and Co., New York, N.Y., 1982).

Another example of a separation methodology is done by covalently labeling the oligonucleotide primers used in a PCR reaction with various types of small molecule ligands. In one such separation, a different ligand is present on each oligonucleotide. A molecule, perhaps an antibody or avidin if the ligand is biotin, that specifically binds to one of the ligands is used to coat the surface of a plate such as a 96 well ELISA plate. Upon application of the PCR reactions to the surface of such a prepared plate, the PCR products are bound with specificity to the surface. After washing the plate to remove unbound reagents, a solution containing a second molecule that binds to the first ligand is added. This second molecule is linked to some kind of reporter system. The second molecule only binds to the plate if a PCR product has been produced whereby both oligonucleotide primers are incorporated into the final PCR products. The amount of the PCR product is then detected and quantified in a commercial plate reader much as ELISA reactions are detected and quantified. An ELISA-like system such as the one described here has been developed by the Raggio Italgene company under the C-Track trade name.

Amplification products must be visualized in order to confirm amplification of the nucleic acid sequences of interest. One typical visualization method involves staining of a gel with ethidium bromide and visualization under UV light. Alternatively, if the amplification products are integrally labeled with radio- or fluorometrically-labeled nucleotides, the amplification products may then be exposed to x-ray film or visualized under the appropriate stimulating spectra, following separation.

In one embodiment, visualization is achieved indirectly. Following separation of amplification products, a labeled, nucleic acid probe is brought into contact with the amplified nucleic acid sequence of interest. The probe preferably is conjugated to a chromophore but may be radiolabeled. In another embodiment, the probe is conjugated to a binding partner, such as an antibody or biotin, where the other member of the binding pair carries a detectable moiety.

In another embodiment, detection is by Southern blotting and hybridization with a labeled probe. The techniques involved in Southern blotting are well known to those of skill in the art and may be found in many standard books on molecular protocols. See Sambrook et al., 1989. Briefly, amplification products are separated by gel electrophoresis. The gel is then contacted with a membrane, such as nitrocellulose, permitting transfer of the nucleic acid and non-covalent binding. Subsequently, the membrane is incubated with a chromophore-conjugated probe that is capable of hybridizing with a target amplification product. Detection is by exposure of the membrane to x-ray film or ion-emitting detection devices.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

5.6.3 Nuclease Protection Assays

Nuclease protection assays (including both ribonuclease protection assays and S1 nuclease assays) can be used to detect and quantitate specific mRNAs. In nuclease protection assays, an antisense probe (labeled with, e.g., radiolabeled or nonisotopic) hybridizes in solution to an RNA sample. Following hybridization, single-stranded, unhybridized probe and RNA are degraded by nucleases. An acrylamide gel is used to separate the remaining protected fragments. Typically, solution hybridization is more efficient than membrane-based hybridization, and it can accommodate up to 100 μg of sample RNA, compared with the 20-30 μg maximum of blot hybridizations.

The ribonuclease protection assay, which is the most common type of nuclease protection assay, requires the use of RNA probes. Oligonucleotides and other single-stranded DNA probes can only be used in assays containing S1 nuclease. The single-stranded, antisense probe must typically be completely homologous to target RNA to prevent cleavage of the probe:target hybrid by nuclease.

5.6.4 Northern Blot Assay

A standard Northern blot assay can be used to ascertain an RNA transcript size, identify alternatively spliced RNA transcripts, and the relative amounts of RNA (in particular, mRNA) in a sample, in accordance with conventional Northern hybridization techniques known to those persons of ordinary skill in the art. In Northern blots, RNA samples are first separated by size via electrophoresis in an agarose gel under denaturing conditions. The RNA is then transferred to a membrane, crosslinked and hybridized with a labeled probe. Nonisotopic or high specific activity radiolabeled probes can be used including random-primed, nick-translated, or PCR-generated DNA probes, in vitro transcribed RNA probes, and oligonucleotides.

Additionally, sequences with only partial homology (e.g., cDNA from a different species or genomic DNA fragments that might contain an exon) may be used as probes. The labeled probe, e.g., a radiolabelled cDNA, either containing the full-length, single stranded DNA or a fragment of that DNA sequence may be at least 20, at least 30, at least 50, or at least 100 consecutive nucleotides in length. The probe can be labeled by any of the many different methods known to those skilled in this art. The labels most commonly employed for these studies are radioactive elements, enzymes, chemicals that fluoresce when exposed to ultraviolet light, and others. A number of fluorescent materials are known and can be utilized as labels. These include, but are not limited to, fluorescein, rhodamine, auramine, Texas Red, AMCA blue and Lucifer Yellow. A particular detecting material is anti-rabbit antibody prepared in goats and conjugated with fluorescein through an isothiocyanate. Proteins can also be labeled with a radioactive element or with an enzyme. The radioactive label can be detected by any of the currently available counting procedures. Non-limiting examples of isotopes include ³H, ¹⁴C, ³²P, ³⁵S, ³⁶Cl, ⁵¹Cr, ⁵⁷Co, ⁵⁸Co, ⁵⁹Fe, 90Y, ¹²⁵I, ¹³¹I, and ¹⁸⁶Rc. Enzyme labels are likewise useful, and can be detected by any of the presently utilized colorimetric, spectrophotometric, fluorospectrophotometric, amperometric or gasometric techniques. The enzyme is conjugated to the selected particle by reaction with bridging molecules such as carbodiimides, diisocyanates, glutaraldehyde and the like. Any enzymes known to one of skill in the art can be utilized. Examples of such enzymes include, but are not limited to, peroxidase, beta-D-galactosidase, urease, glucose oxidase plus peroxidase and alkaline phosphatase. U.S. Pat. Nos. 3,654,090, 3,850,752, and 4,016,043 are referred to by way of example for their disclosure of alternate labeling material and methods.

5.6.5 Other Methods of Transcriptional State Measurement

The transcriptional state of cellular constituent in a biological specimen can be measured by other gene expression technologies known in the art. Several such technologies produce pools of restriction fragments of limited complexity for electrophoretic analysis, such as methods combining double restriction enzyme digestion with phasing primers (see, e.g., European Patent 0 534858 A1, filed Sep. 24, 1992, by Zabeau et al.), or methods selecting restriction fragments with sites closest to a defined mRNA end (see, e.g., Prashar et al., 1996, Proc. Natl. Acad. Sci. USA 93:659-663). Other methods statistically sample cDNA pools, such as by sequencing sufficient bases (e.g., 20-50 bases) in each of multiple cDNAs to identify each cDNA, or by sequencing short tags (e.g., 9-10 bases) that are generated at known positions relative to a defined mRNA end (see, e.g., Velculescu, 1995, Science 270:484-487).

5.7 Measurement of Other Aspects of the Biological State

In various embodiments of the present invention, aspects of the biological state other than the transcriptional state, such as the translational state, the activity state, or mixed aspects can be measured. Thus, in such embodiments, cellular constituent data used in a molecular profile can include translational state measurements or even protein expression measurements. Details of embodiments in which aspects of the biological state other than the transcriptional state are described in this section.

5.7.1 Translational State Measurements

Measurement of the translational state can be performed according to several methods. For example, whole genome monitoring of protein (e.g., the “proteome,”) can be carried out by constructing a microarray in which binding sites comprise immobilized, preferably monoclonal, antibodies specific to a plurality of protein species encoded by the cell genome. Preferably, antibodies are present for a substantial fraction of the encoded proteins, or at least for those proteins relevant to the action of a drug of interest. Methods for making monoclonal antibodies are well known (see, e.g., Harlow and Lane, 1988, Antibodies: A Laboratory Manual, Cold Spring Harbor, N.Y., which is incorporated in its entirety for all purposes). In one embodiment, monoclonal antibodies are raised against synthetic peptide fragments designed based on genomic sequence of the cell. With such an antibody array, proteins from the cell are contacted to the array and their binding is assayed with assays known in the art.

Alternatively, proteins can be separated by two-dimensional gel electrophoresis systems. Two-dimensional gel electrophoresis is well-known in the art and typically involves iso-electric focusing along a first dimension followed by SDS-PAGE electrophoresis along a second dimension. See, e.g., Hames et al., 1990, Gel Electrophoresis of Proteins: A Practical Approach, IRL Press, New York; Shevchenko et al., 1996, Proc. Natl. Acad. Sci. USA 93:1440-1445; Sagliocco et al., 1996, Yeast 12:1519-1533; Lander, 1996, Science 274:536-539. The resulting electropherograms can be analyzed by numerous techniques, including mass spectrometric techniques, Western blotting and immunoblot analysis using polyclonal and monoclonal antibodies, and internal and N-terminal micro-sequencing. Using these techniques, it is possible to identify a substantial fraction of all the proteins produced under given physiological conditions, including in cells (e.g., in yeast) exposed to a drug, or in cells modified by, e.g., deletion or over-expression of a specific gene.

5.7.2 Protein Detection

Standard techniques can also be utilized for determining the amount of the protein or proteins of interest present in a sample. For example, standard techniques can be employed using, e.g., immunoassays such as, for example, Western blot, immunoprecipitation followed by sodium dodecyl sulfate polyacrylamide gel electrophoresis (SDS-PAGE), immunocytochemistry, and the like to determine the amount of the protein or proteins of interest present in a sample. A preferred agent for detecting a protein of interest is an antibody capable of binding to a protein of interest, preferably an antibody with a detectable label.

For such detection methods, protein from the sample to be analyzed can easily be isolated using techniques which are well known to those of skill in the art. Protein isolation methods can, for example, be such as those described in Harlow and Lane (Harlow, E. and Lane, D., 1988, “Antibodies: A Laboratory Manual”, Cold Spring Harbor Laboratory Press, Cold Spring Harbor, N.Y.).

Preferred methods for the detection of the protein or proteins of interest involve their detection via interaction with a protein-specific antibody. For example, antibodies directed a protein of interest can be utilized as described herein. Antibodies can be generated utilizing standard techniques well known to those of skill in the art. See, e.g., Section 5.5.1 of this application and Section 5.2 of U.S. Publication No.20040018200 for a more detailed discussion of such antibody generation techniques, which is incorporated herein by reference. Briefly, such antibodies can be polyclonal, or more preferably, monoclonal. An intact antibody, or an antibody fragment ( e.g., Fab or F(ab′)₂) can, for example, be used. Preferably, the antibody is a human or humanized antibody.

For example, antibodies, or fragments of antibodies, specific for a protein of interest can be used to quantitatively or qualitatively detect the presence of the protein. This can be accomplished, for example, by immunofluorescence techniques. Antibodies (or fragments thereof) can, additionally, be employed histologically, as in immunofluorescence or immunoelectron microscopy, for in situ detection of a protein of interest. In situ detection can be accomplished by removing a histological specimen (e.g., a biopsy specimen) from a patient, and applying thereto a labeled antibody thereto that is directed to a particular protein. The antibody (or fragment) is preferably applied by overlaying the labeled antibody (or fragment) onto a biological sample. Through the use of such a procedure, it is possible to determine not only the presence of the protein of interest, but also its distribution, its presence in lymphocytes within the sample. A wide variety of well-known histological methods (such as staining procedures) can be utilized in order to achieve such in situ detection.

Immunoassays for a protein of interest typically comprise incubating a biological sample of a detectably labeled antibody capable of identifying a protein of interest, and detecting the bound antibody by any of a number of techniques well-known in the art. As discussed in more detail, below, the term “labeled” can refer to direct labeling of the antibody via, e.g., coupling (i.e., physically linking) a detectable substance to the antibody, and can also refer to indirect labeling of the antibody by reactivity with another reagent that is directly labeled. Examples of indirect labeling include detection of a primary antibody using a fluorescently labeled secondary antibody.

The biological sample can be brought in contact with and immobilized onto a solid phase support or carrier such as nitrocellulose, or other solid support which is capable of immobilizing cells, cell particles or soluble proteins. The support can then be washed with suitable buffers followed by treatment with the detectably labeled fingerprint gene-specific antibody. The solid phase support can then be washed with the buffer a second time to remove unbound antibody. The amount of bound label on solid support can then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material can have virtually any possible structural configuration so long as the coupled molecule is capable of binding to an antigen or antibody. Thus, the support configuration can be spherical, as in a bead, or cylindrical, as in the inside surface of a test tube, or the external surface of a rod. Alternatively, the surface can be flat such as a sheet, test strip, etc. Preferred supports include polystyrene beads. Those skilled in the art will know many other suitable carriers for binding antibody or antigen, or will be able to ascertain the same by use of routine experimentation.

One of the ways in which a protein-specific antibody can be detectably labeled is by linking the same to an enzyme and use in an enzyme immunoassay (EIA) (Voller, A., “The Enzyme Linked Immunosorbent Assay (ELISA)”, 1978, Diagnostic Horizons 2:1-7, Microbiological Associates Quarterly Publication, Walkersville, Md.); Voller, A. et al., 1978, J. Clin. Pathol. 31:507-520; Butler, J. E., 1981, Meth. Enzymol. 73:482-523; Maggio, E. (ed.), 1980, Enzyme Immunoassay, CRC Press, Boca Raton, Fla.; Ishikawa, E. et al., (eds.), 1981, Enzyme Immunoassay, Kgaku Shoin, Tokyo). The enzyme which is bound to the antibody will react with an appropriate substrate, preferably a chromogenic substrate, in such a manner as to produce a chemical moiety which can be detected, for example, by spectrophotometric, fluorimetric or by visual means. Enzymes which can be used to detectably label the antibody include, but are not limited to, malate dehydrogenase, staphylococcal nuclease, delta-5-steroid isomerase, yeast alcohol dehydrogenase, alpha-glycerophosphate, dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholinesterase. The detection can be accomplished by colorimetric methods which employ a chromogenic substrate for the enzyme. Detection can also be accomplished by visual comparison of the extent of enzymatic reaction of a substrate in comparison with similarly prepared standards.

Detection can also be accomplished using any of a variety of other immunoassays. For example, by radioactively labeling the antibodies or antibody fragments, it is possible to detect a protein of interest through the use of a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope (e.g., ¹²⁵I, ¹³¹I, ³⁵S or ³H) can be detected by such means as the use of a gamma counter or a scintillation counter or by autoradiography.

It is also possible to label the antibody with a fluorescent compound. When the fluorescently labeled antibody is exposed to light of the proper wavelength, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

The antibody can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA).

The antibody also can be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound can be used to label the antibody of the present invention. Bioluminescence is a type of chemiluminescence found in biological systems in, which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

The protein can also be detected by monitoring its catalytic activity, if the protein is an enzyme. The protein can also be detected using coupled enzymatic assays.

5.8 Diseases

5.8.1 Liver Diseases

Disorders of the liver, referred to herein as a “liver disease” include, but are not limited to, hepatic injury; non-alcoholic fatty liver disease; jaundice and cholestasis, such as bilirubin and bile formation; hepatic failure and cirrhosis, such as cirrhosis, portal hypertension, including ascites, portosystemic shunts, and splenomegaly; infectious disorders, such as viral hepatitis, including hepatitis A-E infection and infection by other hepatitis viruses, clinicopathologic syndromes, such as the carrier state, asymptomatic infection, acute viral hepatitis, chronic viral hepatitis, and fulminant hepatitis; autoimmune hepatitis; drug- and toxin-induced liver disease, such as alcoholic liver disease; inborn errors of metabolism and pediatric liver disease, such as hemochromatosis, Wilson disease, α₁-antitrypsin deficiency, and neonatal hepatitis; intrahepatic biliary tract disease, such as secondary biliary cirrhosis, primary biliary cirrhosis, primary sclerosing cholangitis, and anomalies of the biliary tree; circulatory disorders, such as impaired blood flow into the liver, including hepatic artery compromise and portal vein obstruction and thrombosis, impaired blood flow through the liver, including passive congestion and centrilobular necrosis and peliosis hepatis, hepatic vein outflow obstruction, including hepatic vein thrombosis (Budd-Chiari syndrome) and veno-occlusive disease; hepatic disease associated with pregnancy, such as preeclampsia and eclampsia, acute fatty liver of pregnancy, and intrehepatic cholestasis of pregnancy; hepatic complications of organ or bone marrow transplantation, such as drug toxicity after bone marrow transplantation, graft-versus-host disease and liver rejection, and nonimmunologic damage to liver allografts; tumors and tumorous conditions, such as nodular hyperplasias, adenomas, and malignant tumors, including primary carcinoma of the liver and metastatic tumors.

5.8.2 Disease that are Treatable with an Immunomodulatory Disease Therapy

The present invention is also applicable to diseases that are treatable with an immunomodulatory disease therapy, such interferon-treated diseases, including, but not limited to, immune-mediated diseases, bacterial and viral infectious diseases, and neoplastic diseases. Immune-mediated diseases include, but are not limited to, multiple sclerosis, idiopathic pulmonary fibrosis, Guillain-Barre Syndrome, adult systemic mastocytosis, ulcerative colitis, Crohn's disease, hepatitis C associated cryoglobulinemia, HTLV-1 associated myelopathy (tropical spastic paraparesis). Essentially any virus would be potentially IFN-sensitive. A list of viral infectious diseases include, but are not limited to, hepatitis C, hepatitis B, fulminant viral hepatitis, cytomegalovirus, papillomavirus, severe acute respiratory syndrome (SARS)/coronavirus, Epstein-Barr virus (EBV), Japanese encephalitis, West Nile Virus, viral myocarditis, and human immunodeficiency virus (HIV). Bacterial infectious diseases include, but are not limited to, cryptococcal meningitis and tuberculosis. IFN has been broadly used, sometimes in combination with other agents, as an immunomodulatory agent in the treatment of localized or metastatic diseases. Neoplastic diseases include, but are not limited to, multiple melanoma, renal cell carcinoma, hepatocellular carcinoma (hepatoma), malignant carcinoid tumours, neuroendocrine tumors, lymphoma, acute leukemia, chronic leukemia (particularly chronic myelogenous leukemia), urothelial cancer, prostate cancer, penile cancer, nasopharyngeal cancer, pancreatic cancer, gastric cancer, cervical cancer, colorectal cancer, small cell lung cancer, non-small cell lung cancer, malignant mesothelioma, and breast cancer. Other interferon-treated diseases include, but are not limited to, diabetic retinopathy and Peyronie's disease (erectile dysfunction).

In some embodiments, any of the following diseased can be diagnosed and or treated using the systems and methods of the present invention: hepatitis A virus, hepatitis B virus, hepatitis C virus, human papilloma virus, human immunodeficiency virus, respiratory syncitial virus, human adenovirus, fowl adenovirus 1, African swine fever virus, lymphocytic choriomeningitis virus, ippy virus, lassa virus, equine arteritis virus, human astrovirus 1, autographa californica nucleopolyhedrovirus, plodia interpunctella granulovirus, commelina yellow mottle virus, rice tungro bacilliform virus, mushroom bacilliform virus, infectious pancreatic necrosis virus, infectious bursal disease virus, drosophila x virus, alfalfa mosaic virus, tobacco streak virus, brome mosaic virus, cucumber mosaic virus, apple stem grooving virus, carnation latent virus, cauliflower mosaic virus, chicken anemia virus, beet yellows virus, cowpea mosaic virus, tobacco ringspot virus, avian infectious bronchitis virus, alteromonas phage pm2, pseudomonas phage phi6, hepatitis delta virus, carnation ringspot virus, red clover necrotic mosaic virus, sweet clover necrotic mosaic virus, pea enation mosaic virus, ebola virus zair, soil-borne wheat mosaic virus, beet necrotic yellow vein virus, sulfobolus virus 1, maize streak virus, beet curly top virus, bean golden mosaic virus, duck hepatitis B virus, human herpesvirus, human herpesvirus, ateline herpesvirus 2, barley stripe mosaic virus, cryphonectria hypovirus 1-ep713, raspberry bushy dwarf virus, acholeplasma phage 151, chilo iridescent virus, goldfish virus 1, enterobacteria phage ms2, enterobacteria phage qbeta, thermoproteus virus 1, maize chlorotic mottle virus, maize rayado fino virus, coliphage phix174, spiromicrovirus, spiroplasma phage, bdellomicrovirus, bdellovibrio phage, chlamydiamicrovirus, chlamydia phage 1, coliphage t4, tobacco necrosis virus, nodamura virus, influenzavirus a, influenzavirus C, thogoto virus, rabbit (shope) papillomavirus, human parainfluenza virus, measles virus, rubulavirus, mumps virus, human respiratory syncytial virus, gaeumannomyces graminis virus, penicillium chrysogenum virus, white clover cryptic virus, white clover cryptic virus 2, minute mice virus, adeno-associated virus, junonia coenia densovirus, bombyx mori virus, aedes aegypti densovirus, 1-paramecium bursaria chlorella nc64a virus, paramecium bursaria chlorella virus, 2-paramecium bursaria chlorella pbi virus, 3-hydra viridis chlorella virus, human poliovirus 1, human rhinovirus 1A, hepatovirus, encephalomyocarditis virus, foot-and-mouth disease virus, acholeplasma phage 12, coliphage t7, campoletis sonorensis virus, cotesia melanoscela virus, potato virus X, potato virus Y, ryegrass mosaic virus, barley yellow mosaic virus, fowlpox virus, sheep pox virus, swinepox virus, molluscum contagiosum virus, yaba monkey tumor virus, entomopoxvirus A, melolontha melolontha entomopoxvirus, amsacta moorei entomopoxvirus, chironomus luridus entomopoxvirus, reovirus 3, epizootic hemarrhogic disease virus 1, or simian rotavirus SA11.

In particular, lymphocytic choriomeningitis virus can be treated using the methods of the present invention. On Jun. 2, 2005, Reuters Health reported that four transplant recipients in the United States became infected with lymphocytic choriomeningitis virus (LCMV), which is normally carried by rodents, after receiving organs from a single donor infected with the virus, according to researchers from the Centers for Disease Control and Prevention. LCMV seldom causes problems for healthy individuals, but in immunesuppressed patients such as transplant recipients, infection can be serious and even fatal. Currently, there are no effective pre-transplant tests for screening organ or tissue donors for LCMV infection. The present invention will address the need for such a test.

5.9 Methods for Detecting Changes in Gene Expression or Protein Expression

This invention provides several methods for detecting changes in gene expression or protein expression, including but not limited to the expression of SEQ ID NO: 1, SEQ ID NO: 3, SEQ ID NO: 5, SEQ ID NO: 7, and SEQ ID NO: 9, homologs of each of the foregoing, and marker genes operably linked to each of the forgoing. Assays for changes in gene expression are well known in the art (see, e.g., PCT Publication No. WO 96/34099, published Oct. 31, 1996, which is incorporated by reference herein in its entirety). Such assays can be performed in vitro using transformed cell lines, immortalized cell lines, or recombinant cell lines.

The RNA expression or protein expression of an open reading frame (which may be of a marker gene or may be of a gene referenced in Section 5.1.2), regulated by a promoter native to the gene referenced in Section 5.1.2 can be measured by measuring the amount or abundance of the RNA (as RNA or cDNA) or protein. In particular, the assays may detect the presence of increased or decreased expression of a gene referenced in Section 5.1.2 (e.g., SEQ ID NO: 1, SEQ ID NO: 3, SEQ ID NO: 5, SEQ ID NO: 7, and SEQ ID NO: 9) on the basis of increased or decreased mRNA expression (using, e.g., nucleic acid probes), increased or decreased levels of protein products (using, e.g., antibodies thereto), or increased or decreased levels of expression of a marker gene (e.g., green fluorescent protein “GFP”) operably linked to the 5 promoter region in a recombinant construct. A protein product of a gene is a protein coded by the gene.

The present invention envisions monitoring changes in gene expression (e.g., a gene referenced in Section 5.1.2) or marker gene expression by any expression analysis technique known to one of skill in the art, including but not limited to, differential display, serial analysis of gene expression (SAGE), nucleic acid array technology, oligonucleotide array technology, GeneChip expression analysis, dot blot hybridization, northern blot hybridization, QRT-PCR, subtractive hybridization, protein chip arrays, Western blot, immunoprecipitation followed by SDS PAGE, immunocytochemistry, proteome analysis and mass-spectrometry of two-dimensional protein gels.

Methods of gene expression profiling to measure changes in gene expression are well-known in the art, as exemplified by the following references describing subtractive hybridization (Wang and Brown, 1991, Proc. Natl. Acad. Sci. U.S.A. 88:11505-11509), differential display (Liang and Pardee, 1992, Science 257:967-971), SAGE (Velculescu et al., 1995, Science 270:484-487), proteome analysis (Humphery-Smith et al., 1997, Electrophoresis 18:1217-1242; Dainese et al., 1997, Electrophoresis 18:432-442), and hybridization-based methods employing nucleic acid arrays (Heller et al., 1997, Proc. Natl. Acad. Sci. U.S.A. 94:2150-2155; Lashkari et al., 1997, Proc. Natl. Acad. Sci. U.S.A. 94:13057-13062; Wodicka et al., 1997, Nature Biotechnol. 15:1259-1267). Microarray technology is described in more detail below.

In one series of embodiments, various expression analysis techniques can be used to identify molecules that affect expression of a gene referenced in Section 5.1.2 or marker gene expression, by comparing a cell line expressing a gene disclosed in Section 5.1.2 (e.g. SEQ ID NO: 1, SEQ ID NO: 3, SEQ ID NO: 5, SEQ ID NO: 7, and SEQ ID NO: 9) or a marker gene under the control of a gene promoter sequence in the absence of a test molecule to a cell line expressing the same gene or marker gene under the control of the same promoter sequence in the presence of the test molecule. In a preferred embodiment, expression analysis techniques are used to identify a molecule that upregulates a gene referenced in Section 5.1.2 or upregulates marker gene expression upon treatment of a cell with the molecule.

5.10 Methods for Monitoring Reporter Gene Expression of a Gene of the Present Invention

5.10.1 Heterologous Reporter Gene Construct

In a preferred embodiment, the cell being assayed for reporter gene expression contains a fusion construct of at least one transcriptional promoter region for a gene disclosed in Section 5.1.2 (e.g., SEQ ID NO: 1, SEQ ID NO: 3, SEQ ID NO: 5, SEQ ID NO: 7, and SEQ ID NO: 9) (also referred to herein as the test gene), or homologs of the foregoing, each operably linked to a marker gene expressing a detectable and/or selectable product. Increased expression of a marker gene operably linked to a gene promoter indicates increased expression of the test gene.

The marker gene is a sequence encoding a detectable or selectable marker, the expression of which is regulated by at least one gene promoter region in the heterologous construct used in the present invention. Preferably, the assay is carried out in the absence of background levels of marker gene expression (e.g., in a cell that is mutant or otherwise lacking in the marker gene). If not already lacking in endogenous marker gene activity, cells mutant in the marker gene may be selected by known methods, or the cells can be made mutant in the marker gene by known gene-disruption methods prior to introducing the marker gene (Rothstein, 1983, Meth. Enzymol. 101:202-211).

A marker gene of the invention can be any gene that encodes a detectable and/or selectable product. The detectable marker can be any molecule that can give rise to a detectable signal, e.g., a fluorescent protein or a protein that can be readily visualized or that is recognizable by a specific antibody or that gives rise enzymatically to a signal. The selectable marker can be any molecule that can be selected for its expression, e.g., which gives cells a selective advantage over cells not having the selectable marker under appropriate (selective) conditions. In preferred aspects, the selectable marker is an essential nutrient in which the cell in which the interaction assay occurs is mutant or otherwise lacks or is deficient, and the selection medium lacks such nutrient. In one embodiment, one type of marker gene is used to detect gene expression. In another embodiment, more than one type of marker gene is used to detect gene expression.

Preferred marker genes include but are not limited to, green fluorescent protein (GFP) (Cubitt et al., 1995, Trends Biochem. Sci. 20:448-455), red fluorescent protein, blue fluorescent protein, luciferase, LEU2, LYS2, ADE2, TRP1, CAN1, CYH2, GUS, CUP1 or chloramphenicol acetyl transferase (CAT). Other marker genes include, but are not limited to, URA3, HIS3 and/or the lacZ genes (see e.g., Rose and Botstein, 1983, Meth. Enzymol. 101:167-180) operably linked to GAL4 DNA-binding domain recognition elements. Alam and Cook disclose non-limiting examples of detectable marker genes that can be operably linked to a glucan synthase pathway reporter gene promoter region (Alam and Cook, 1990, Anal. Biochem. 188:245-254).

In a preferred embodiment, more than one different marker gene is used to detect transcriptional activation, e.g., one encoding a detectable marker, and one or more encoding one or more different selectable marker(s), or e.g., different detectable markers. Expression of the marker genes can be detected and/or selected for by techniques known in the art (see e.g. U.S. Pat. Nos. 6,057,101 and 6,083,693).

Methods to construct a suitable reporter construct are disclosed herein by way of illustration and not limitation and any other methods known in the art can also be used. In a preferred embodiment, the reporter gene construct is a chimeric reporter construct comprising a marker gene that is transcribed under the control of a gene promoter sequence comprising all or a portion of a promoter region of SEQ ID NO: 1, SEQ ID NO: 3, SEQ ID NO: 5, SEQ ID NO: 7, and SEQ ID NO: 9. If not already a part of the DNA sequence, the translation initiation codon, ATG, is provided in the correct reading frame upstream of the DNA sequence.

Vectors comprising all or portions of the gene sequences of SEQ ID NO: 1, SEQ ID NO: 3, SEQ ID NO: 5, SEQ ID NO: 7, and SEQ ID NO: 9 useful in the construction of recombinant reporter gene constructs and cells are provided. The vectors of this invention also include those vectors comprising DNA sequences that hybridize under stringent conditions to SEQ ID NO: 1, SEQ ID NO: 3, SEQ ID NO: 5, SEQ ID NO: 7, and SEQ ID NO: 9 gene sequences, and conservatively modified variations thereof.

The vectors of this invention may be present in transformed or transfected cells, cell lysates, or in partially purified or substantially pure forms. DNA vectors may contain a means for amplifying the copy number of the gene of interest, stabilizing sequences, or alternatively may be designed to favor directed or non-directed integration into the host cell genome.

Given the strategies described herein, one of skill in the art can construct a variety of vectors and nucleic acid molecules comprising functionally equivalent nucleic acids. DNA cloning and sequencing methods are well known to those of skill in the art and are described in an assortment of laboratory manuals, including Sambrook et al., 1989, supra; and Ausubel et al., 2002 Supplement.

Transformation and other methods of introducing nucleic acids into a host cell (e.g., transfection, electroporation, liposome delivery, membrane fusion techniques, high velocity DNA-coated pellets, viral infection and protoplast fusion) can be accomplished by a variety of methods that are well known in the art (see, for instance, Ausubel, supra, and Sambrook, supra). S. cerevisiae cells of the invention can be transformed or transfected with an expression vector, such as a plasmid, a cosmid, or the like, wherein the expression vector comprises the DNA of interest. Alternatively, the cells can be infected by a viral expression vector comprising the DNA or RNA of interest.

Particular details of the transfection and expression of nucleic acid sequences are well documented and are understood by those of skill in the art. Further details on the various technical aspects of each of the steps used in recombinant production of foreign genes in expression systems can be found in a number of texts and laboratory manuals in the art (see, e.g., Ausubel et al., 2002, herein incorporated by reference).

5.10.2 Other Methods for Monitoring Reporter Gene Expression

In accordance with the present invention, reporter gene expression can be monitored at the RNA or the protein level. In a specific embodiment, molecules that affect reporter gene expression can be identified by detecting differences in the level of marker protein expressed by cells contacted with a test molecule versus the level of marker protein expressed by cells in the absence of the test molecule.

Protein expression can be monitored using a variety of methods that are well known to those of skill in the art. For example, protein chips or protein microarrays (e.g., ProteinChip™, Ciphergen Biosystem) and two-dimensional electrophoresis (see e.g., U.S. Pat. No. 6,064,754) can be utilized to monitor protein expression levels. As used herein “two-dimensional electrophoresis”) (2D-electrophoresis) means a technique comprising isoelectric focusing, followed by denaturing electrophoresis, generating a two-dimensional gel (2D-gel) containing a plurality of proteins. Any protocol for 2D-electrophoresis known to one of ordinary skill in the art can be used to analyze protein expression by the reporter genes of the invention. For example, 2D electrophoresis can be performed according to the methods described in O'Farrell, 1975, J. Biol. Chem. 250: 4007-4021.

Liquid High Throughput-Like Assay. In a preferred embodiment, a liquid high throughput-like assay is used to determine the protein expression level of a reporter gene. The following exemplary, but not limiting, assay may be used:

A reporter construct is transformed into a cell strain. Cultures from solid media plates are used to innoculate liquid cultures in Casamino Acids media or an equivalent media. This liquid culture is grown and then diluted in Casamino Acids media or an equivalent media.

A test molecule is selected for the assay, preferably but not necessarily along with a negative control molecule. The test molecule and negative control molecule are separately added to an assay plate containing multiple wells and serially diluted (e.g., 1 to 2) into Casamino Acids media plus DMSO in sequential columns, so that each plate contains a range of concentrations of each drug. If a negative control is being used, one column of each plate may be used as a “no drug” control, containing only Casamino Acids media plus DMSO. The skilled artisan will note that different assay plates can be used, such as those with 96, 384 or 1536 well format.

An aliquot of liquid reporter strain is added to each well of the serial dilution plates from above and mixed. The assay plates are then incubated. After incubation the assay plates are analyzed for detectable marker gene product. In a preferred embodiment, the assay plates are imaged in a Molecular Dynamics Fluorimager SI to measure the fluorescence from the GFP reporters.

The results are then analyzed, as described above. If the drug is an inhibitor of the gene product (e.g., an inhibitor of e.g. SEQ ID NO: 1, SEQ ID NO: 3, SEQ ID NO: 5, SEQ ID NO: 7, and SEQ ID NO: 9) the reporter will show increases in fluorescence for the higher drug concentrations versus the lower drug concentrations and/or the no drug controls.

5.10.3 Specific Embodiments

One embodiment of the present invention provides a method for determining whether a candidate molecule affects the gene expression level of the target genes identified by the methods of the present invention and/or a biological function of one or more target gene products identified by the methods of the present invention. In step (a) of the method, a cell from the organism is contacted with the candidate molecule. Alternatively, the candidate molecule is recombinantly expressed within the cell. In step (b) of the method, a determination is made as to whether the RNA expression or protein expression in the cell of at least one open reading frame is changed in step (a) relative to the expression of the open reading frame in the absence of the candidate molecule, where each open reading frame is regulated by a promoter native to a nucleic acid sequence selected from the group consisting of SEQ ID NO: 1, SEQ ID NO: 3, SEQ ID NO: 5, SEQ ID NO: 7, and SEQ ID NO: 9 and homologs (e.g., orthologs, and paralogs) of each of the foregoing.

The candidate molecule affects the gene expression level of the target genes identified by the methods of the present invention and/or a biological function of one or more target gene products identified by the methods of the present invention when the RNA expression or protein expression of the at least one open reading frame is changed. The candidate molecule does not affect the gene expression level of the target genes identified by the methods of the present invention and/or a biological function of one or more target gene products identified by the methods of the present invention when the RNA expression or protein expression of the at least one open reading frame is unchanged.

In some embodiments, the candidate molecule affects the gene expression level of the target genes identified by the methods of the present invention and/or a biological function of one or more target gene products identified by the methods of the present invention when a cell from the organism that is contacted with the candidate molecule exhibits a lower expression level of a protein sequence in the group consisting of SEQ ID NO: 2, SEQ ID NO: 4, SEQ ID NO: 6, SEQ ID NO: 8, and SEQ ID NO: 10 relative to a cell from the organism that is not contacted with the candidate molecule.

In some embodiments step (b) comprises determining whether RNA expression is changed. In some embodiments, step (b) comprises determining whether protein expression is changed. In some embodiments, step (b) comprises determining whether RNA or protein expression of at least two of the open reading frames is changed. In some embodiments, step (a) comprises contacting the cell with the candidate molecule and step (a) is carried out in a liquid high throughput-like assay.

In some embodiments, the cell comprises a promoter region of at least one gene selected from the group consisting of SEQ ID NO: 1, SEQ ID NO: 3, SEQ ID NO: 5, SEQ ID NO: 7, and SEQ ID NO: 9 and homologs of each of the foregoing, each promoter region being operably linked to a marker gene. Further, in such embodiments, step (b) comprises determining whether the RNA expression or protein expression of the marker gene(s) is changed in step (a) relative to the expression of the marker gene in the absence of the candidate molecule. In some embodiments, the marker gene is selected from the group consisting of green fluorescent protein, red fluorescent protein, blue fluorescent protein, luciferase, LEU2, LYS2, ADE2, TRP1, CAN1, CYH2, GUS, CUP1 and chloramphenicol acetyl transferase.

Another aspect of the invention provides a method of identifying a molecule that specifically binds to a ligand selected from the group consisting of (i) a protein encoded by a gene selected from the group consisting of SEQ ID NO: 1, SEQ ID NO: 3, SEQ ID NO: 5, SEQ ID NO: 7, and SEQ ID NO: 9. The method comprises (a) contacting the ligand with one or more candidate molecules under conditions conducive to binding between the ligand and the candidate molecules; and (b) identifying a molecule within the one or more candidate molecules that binds to the ligand.

5.11 Method of Treating a Liver Disease or a Disease that is Treatable with an Immunomodulatory Disease Therapy

One aspect of the invention provides a method of treating a liver disease or a disease that is treatable with an immunomodulatory disease therapy. The method comprises administering to a subject in which treatment is desired a therapeutically effective amount of a molecule that inhibits a function of one or more of the group consisting of SEQ ID NO: 1, SEQ ID NO: 3, SEQ ID NO: 5, SEQ ID NO: 7, and SEQ ID NO: 9, and homologs (e.g., orthologs and paralogs) thereof.

In some embodiments, the subject is human. In some embodiments, the molecule that inhibits a function of one or more of the group consisting of SEQ ID NO: 1, SEQ ID NO: 3, SEQ ID NO: 5, SEQ ID NO: 7, and SEQ ID NO: 9 and homologs (e.g., orthologs and paralogs) thereof, is selected from the group consisting of an antibody that binds to one of SEQ ID NO: 1, SEQ ID NO: 3, SEQ ID NO: 5, SEQ ID NO: 7, and SEQ ID NO: 9, and homologs thereof, or a fragment or derivative therefof.

Another aspect of the invention provides a method of treating a liver disease or a disease that is treatable with an immunomodulatory disease therapy. The method comprises administering to a subject in which treatment is desired a therapeutically effective amount of a molecule that enhances a function of one or more of the group consisting of SEQ ID NO: 1, SEQ ID NO: 3, SEQ ID NO: 5, SEQ ID NO: 7, and SEQ ID NO: 9 and homologs thereof. In some embodiments, the subject is human.

Yet another aspect of the invention provides a method of diagnosing a liver disease or a disease that is treatable with an immunomodulatory disease therapy or the predisposition to the liver disease or the disease that is treatable with an immunomodulatory disease therapy, where the liver disease or disease that is treatable with an immunomodulatory disease therapy is characterized by an aberrant level of one of SEQ ID NO: 1, SEQ ID NO: 3, SEQ ID NO: 5, SEQ ID NO: 7, and SEQ ID NO: 9 (or homologs thereof) in a subject. The method comprises measuring the level of any one of SEQ ID NO: 1, SEQ ID NO: 3, SEQ ID NO: 5, SEQ ID NO: 7, and SEQ ID NO: 9 (or homologs thereof) in a sample derived from the subject, in which an increase or decrease in the level of one of SEQ ID NO: 1, SEQ ID NO: 3, SEQ ID NO: 5, SEQ ID NO: 7, and SEQ ID NO: 9 (or homologs thereof) in the sample, relative to the level of one of said SEQ ID NO: 1, SEQ ID NO: 3, SEQ ID NO: 5, SEQ ID NO: 7, and SEQ ID NO: 9 (or homologs thereof) found in an analogous sample not having the liver disease or the disease that is treatable with an immunomodulatory disease therapy, indicates the presence of the liver disease or the disease that is treatable with an immunomodulatory disease therapy in the subject.

Still another aspect of the invention provides a method of diagnosing or screening for the presence of or predisposition for developing a liver disease or a disease that is treatable with an immunomodulatory disease therapy in a subject comprising detecting one or more mutations in at least one of SEQ ID NO: 1, SEQ ID NO: 3, SEQ ID NO: 5, SEQ ID NO: 7, and SEQ ID NO: 9 (or homologs thereof) in a sample derived from the subject, in which the presence of the one or more mutations indicates the presence of the liver disease or disorder or a predisposition for developing the liver disease or disease that is treatable with an immunomodulatory disease therapy.

5.12 Transgenic Animals

The invention also provides animal models. Transgenic animals that have incorporated and express a constitutively-functional gene related to a liver disease or a disease that is treatable with an immunomodulatory disease therapy have use as animal models of liver diseases and diseases that are treatable with an immunomodulatory disease therapy. Such animals can be used to screen for or test molecules for the ability to prevent such liver diseases and diseases that are treatable with an immunomodulatory disease therapy. In one embodiment, animal models for liver diseases and diseases that are treatable with an immunomodulatory disease therapy is provided. Such animals can be initially produced by promoting homologous recombination between a gene related to a liver disease or a disease that is treatable with an immunomodulatory disease therapy (e.g. SEQ ID NO: 1, SEQ ID NO: 3, SEQ ID NO: 5, SEQ ID NO: 7, and SEQ ID NO: 9, and homologs thereof) in its chromosome and an exogenous gene related to a liver disease or a disease that is treatable with an immunomodulatory disease therapy that has been rendered biologically inactive. Preferably the sequence inserted is a heterologous sequence, e.g., an antibiotic resistance gene. In a preferred aspect, this homologous recombination is carried out by transforming embryo-derived stem (ES) cells with a vector containing an insertionally inactivated gene, where the active gene encodes a particular gene related to a liver disease or a disease that is treatable with an immunomodulatory disease therapy, such that homologous recombination occurs; the ES cells are then injected into a blastocyst, and the blastocyst is implanted into a foster mother, followed by the birth of the chimeric animal, also called a “knockout animal,” in which a gene related to a liver disease or a disease that is treatable with an immunomodulatory disease therapy has been inactivated (see Capecchi, 1989, Science 244: 1288-1292). The chimeric animal can be bred to produce additional knockout animals. Chimeric animals can be and are preferably non-human mammals such as mice, hamsters, sheep, pigs, cattle, etc. In a specific embodiment, a knockout mouse is produced.

Such knockout animals are expected to develop or be predisposed to developing liver diseases or diseases that are treatable with an immunomodulatory disease therapy and thus can have use as animal models of such liver diseases and diseases that are treatable with an immunomodulatory disease therapy, e.g., to screen for or test molecules for the ability to promote activation or proliferation and thus treat or prevent such liver diseases or diseases that are treatable with an immunomodulatory disease therapy.

In a different embodiment of the invention, transgenic animals that have incorporated and express a constitutively-functional gene related to a liver disease or a disease that is treatable with an immunomodulatory disease therapy have use as animal models of liver diseases and diseases that are treatable with an immunomodulatory disease therapy, involving in T-cell overactivation, or in which T cell activation is desired.

In particular, each transgenic line expressing a particular key gene under the control of the regulatory sequences of a characterizing gene is created by the introduction, for example by pronuclear injection, of a vector containing the transgene into a founder animal, such that the transgene is transmitted to offspring in the line. The transgene preferably randomly integrates into the genome of the founder but in specific embodiments can be introduced by directed homologous recombination. In a preferred embodiment, the transgene is present at a location on the chromosome other than the site of the endogenous characterizing gene. In a preferred embodiment, homologous recombination in bacteria is used for target-directed insertion of the key gene sequence into the genomic DNA for all or a portion of the characterizing gene, including sufficient characterizing gene regulatory sequences to promote expression of the characterizing gene in its endogenous expression pattern. In a preferred embodiment, the characterizing gene sequences are on a bacterial artificial chromosome (BAC). In specific embodiments, the key gene coding sequences are inserted as a 5 fusion with the characterizing gene coding sequence such that the key gene coding sequences are inserted in frame and directly 3 from the initiation codon for the characterizing gene coding sequences. In another embodiment, the key gene coding sequences are inserted into the 3 untranslated region (UTR) of the characterizing gene and, preferably, have their own internal ribosome entry sequence (IRES).

The vector (preferably a BAC) comprising the key gene coding sequences and characterizing gene sequences is then introduced into the genome of a potential founder animal to generate a line of transgenic animals. Potential founder animals can be screened for the selective expression of the key gene sequence in the population of cells characterized by expression of the endogenous characterizing gene. Transgenic animals that exhibit appropriate expression (e.g., detectable expression of the key gene product having the same expression pattern within the animal as the endogenous characterizing gene) are selected as founders for a line of transgenic animals.

One aspect of the invention provides a recombinant non-human animal that is the product of a process comprising introducing a nucleic acid encoding at least a domain of one of SEQ ID NO: 1, SEQ ID NO: 3, SEQ ID NO: 5, SEQ ID NO: 7, and SEQ ID NO: 9 (or homologs thereof) into the non-human animal.

5.13 Screening for Gene Agonists and Antagonists

The genes and gene products referenced in Section 5.1.2 can be used to prepare protein for screening by methods that are routine and well known in the art (see, e.g., Sambrook et al., 2001, Molecular Cloning, A Laboratory Manual, Third Edition, Cold Spring Harbor Laboratory Press, N.Y.; and Ausubel et al., 1989, Current Protocols in Molecular Biology, Green Publishing Associates and Wiley Interscience, N.Y., both of which are hereby incorporated by reference in their entireties).

For example, using any of the gene sequences referenced in Section 5.1.2 (e.g., SEQ ID NO: 1, SEQ ID NO: 3, SEQ ID NO: 5, SEQ ID NO: 7, and SEQ ID NO: 9) oligonucleotide primers for PCR amplification can be designed. PCR amplification is then used to amplify specifically the obesity related protein coding sequence, which can be cloned into an appropriate expression vector using routine techniques. That vector can then be introduced into bacterial or cultured eukaryotic cells (e.g., cultured mammalian cells, insect cells, etc.) such that the gene product is expressed in the bacterial or cultured cell. The gene product can then be isolated from the bacterial or eukaryotic cell culture.

By way of example, diversity libraries, such as random or combinatorial peptide or nonpeptide libraries, can be screened for molecules that specifically bind to and/or modulate the function of the gene product. Many libraries are known in the art that can be used, e.g., chemically synthesized libraries, recombinant (e.g., phage display libraries), and in vitro translation-based libraries.

Examples of chemically synthesized libraries are described in Fodor et al., 1991, Science 251:767-773; Houghten et al., 1991, Nature 354:84-86; Lam et al., 1991, Nature 354:82-84; Medynski, 1994, Bio/Technology 12:709-710; Gallop et al., 1994, J. Medicinal Chemistry 37:1233-1251; Ohlmeyer et al., 1993, Proc. Natl. Acad. Sci. USA 90:10922-10926; Erb et al., 1994, Proc. Natl. Acad. Sci. USA 91:11422-11426; Houghten et al., 1992, Biotechniques 13:412; Jayawickreme et al., 1994, Proc. Natl. Acad. Sci. USA 91:1614-1618; Salmon et al., 1993, Proc. Natl. Acad. Sci. USA 90:11708-11712; PCT Publication No. WO 93/20242; and Brenner and Lerner, 1992, Proc. Natl. Acad. Sci. USA 89:5381-5383.

Examples of phage display libraries are described in Scott and Smith, 1990, Science 249:386-390; Devlin et al., 1990, Science, 249:404-406; Christian, R. B., et al., 1992, J. Mol. Biol. 227:711-718; Lenstra, 1992, J. Immunol. Meth. 152:149-157; Kay et al., 1993, Gene 128:59-65; and PCT Publication No. WO 94/18318 dated Aug. 18, 1994. In vitro translation-based libraries include but are not limited to those described in PCT Publication No. WO 91/05058 dated Apr. 18, 1991; and Mattheakis et al., 1994, Proc. Natl. Acad. Sci. USA 91:9022-9026.

By way of examples of nonpeptide libraries, a benzodiazepine library (see e.g., Bunin et al., 1994, Proc. Natl. Acad. Sci. USA 91:4708-4712) can be adapted for use. Peptoid libraries (Simon et al., 1992, Proc. Natl. Acad. Sci. USA 89:9367-9371) can also be used. Another example of a library that can be used, in which the amide functionalities in peptides have been permethylated to generate a chemically transformed combinatorial library, is described by Ostresh et al. (1994, Proc. Natl. Acad. Sci. USA 91:11138-11142).

Screening the libraries can be accomplished by any of a variety of commonly known methods. See, e.g., the following references, which disclose screening of peptide libraries: Parmley and Smith, 1989, Adv. Exp. Med. Biol. 251:215-218; Scott and Smith, 1990, Science 249:386-390; Fowlkes et al., 1992; BioTechniques 13:422-427; Oldenburg et al., 1992, Proc. Natl. Acad. Sci. USA 89:5393-5397; Yu et al., 1994, Cell. 76:933-945; Staudt et al., 1988, Science 241:577-580; Bock et al., 1992, Nature 355:564-566; Tuerk et al., 1992, Proc. Natl. Acad. Sci. USA 89:6988-6992; Ellington et al., 1992, Nature 355:850-852; U.S. Pat. No. 5,096,815, U.S. Pat. No. 5,223,409, and U.S. Pat. No. 5,198,346, all to Ladner et al.; Rebar and Pabo, 1993, Science 263:671-673; and PCT Publication No. WO 94/18318.

In a specific embodiments, screening can be carried out by contacting the library members with a gene product referenced in Section 5.1.2 (or nucleic acid or derivative) immobilized on a solid phase and harvesting those library members that bind to the protein (or nucleic acid or derivative). Examples of such screening methods, termed “panning” techniques, are described by way of example in Parmley and Smith, 1988, Gene 73:305-318; Fowlkes et al., 1992, BioTechniques 13:422-427; PCT Publication No. WO 94/18318; and in references cited hereinabove.

In another embodiment, the two-hybrid system for selecting interacting proteins in yeast (Fields and Song, 1989, Nature 340:245-246; Chien et al., 1991, Proc. Natl. Acad. Sci. USA 88:9578-9582) can be used to identify molecules that specifically bind to a gene product referenced in Section 5.1.2 or a derivative of such gene product.

5.14 Low Stringency Conditions

The invention also relates to nucleic acids hybridizable to or complementary to all or a portion of the nucleic acid sequences referenced in Section 5.1.2 under conditions of low stringency. By way of example and not limitation, procedures using such conditions of low stringency are as follows (see also Shilo and Weinberg, 1981, Proc. Natl. Acad. Sci. U.S.A. 78:6789-6792): filters containing DNA are pretreated for 6 hours at 40° C. in a solution containing 35% formamide, 5×SSC, 50 mM Tris-HCl (pH 7.5), 5 mM EDTA, 0.1% PVP, 0.1% Ficoll, 1% BSA, and 500 mg/ml denatured salmon sperm DNA. Hybridizations are carried out in the same solution with the following modifications: 0.02% PVP, 0.02% Ficoll, 0.2% BSA, 100 mg g/ml salmon sperm DNA, 10% (wt/vol) dextran sulfate, and 5-20×106 cpm 32P-labeled probe is used. Filters are incubated in hybridization mixture for 18-20 hours at 40° C., and then washed for 1.5 hours at 55° C. in a solution containing 2×SSC, 25 mM Tris-HCl (pH 7.4), 5 mM EDTA, and 0.1% SDS. The wash solution is replaced with fresh solution and incubated an additional 1.5 hours at 60° C. Filters are blotted dry and exposed for autoradiography. If necessary, filters are washed for a third time at 65-68° C. and re-exposed to film. Other conditions of low stringency that can be used are well known in the art (e.g., as employed for cross-species hybridizations).

5.15 High Stringency Conditions

The invention also relates to nucleic acids hybridizable to or complementary to all or a portion of the nucleic acid sequences referenced in Section 5.1.2 under conditions of high stringency. By way of example and not limitation, procedures using such conditions of high stringency are as follows: prehybridization of filters containing DNA is carried out for 8 hours to overnight at 65° C. in buffer composed of 6×SSC, 50 mM Tris-HCl (pH 7.5), 1 mM EDTA, 0.02% PVP, 0.02% Ficoll, 0.02% BSA, and 500 mg/ml denatured salmon sperm DNA. Filters are hybridized for 48 hours at 65° C. in prehybridization mixture containing 100 mg/ml denatured salmon sperm DNA and 5-20×106 cpm of 32P-labeled probe. Washing of filters is done at 37° C. for one hour in a solution containing 2×SSC, 0.01% PVP, 0.01% Ficoll, and 0.01% BSA. This is followed by a wash in 0.1×SSC at 50° C. for 45 minutes before autoradiography. Other conditions of high stringency that may be used are well known in the art.

5.16 Moderate Stringency Conditions

In another specific embodiment, the invention relates to nucleic acids hybridizable to or complementary to all or a portion of the nucleic acid sequences referenced in Section 5.1.2 under conditions of moderate stringency. As used herein, conditions of moderate stringency, as known to those having ordinary skill in the art, and as defined by Sambrook et al., Molecular Cloning. A Laboratory Manual, 2^{nd} Ed. Vol. 1, pp. 1.101-104, Cold Spring Harbor Laboratory Press, 1989), include use of a prewashing solution for the nitrocellulose filters 5×SSC, 0.5% SDS, 1.0 mM EDTA (pH 8.0), hybridization conditions of 50 percent formamide, 6×SSC at 42° C. (or other similar hybridization solution, or Stark's solution, in 50% formamide at 42° C.), and washing conditions of about 60° C., 0.5×SSC, 0.1% SDS. See also, Ausubel et al., eds., in the Current Protocols in Molecular Biology series of laboratory technique manuals,® 1987-1997, Current Protocols,® 1994-1997, John Wiley and Sons, Inc.). The skilled artisan will recognize that the temperature, salt concentration, and chaotrope composition of hybridization and wash solutions can be adjusted as necessary according to factors such as the length and nucleotide base composition of the probe.

5.17 Derivative and Antisense Nucleic Acids

Nucleic acids encoding derivatives of gene sequences referenced in Section 5.1.2 (e.g., SEQ ID NO: 1, SEQ ID NO: 3, SEQ ID NO: 5, SEQ ID NO: 7, and SEQ ID NO: 9) and antisense nucleic acids to such sequence are additionally provided. As is readily apparent, as used herein, a nucleic acid encoding a fragment or portion of a given nucleic acid sequence (e.g. a fragment of SEQ ID NO: 5) shall be construed as referring to a nucleic acid encoding only the recited fragment or portion of the specific nucleic acid and not the other contiguous portions of the nucleic acid as a continuous sequence.

5.18 Gene Product Antibody Production

The antibodies of the invention or fragments thereof can be produced by any method known in the art for the synthesis of antibodies, in particular, by chemical synthesis or preferably, by recombinant expression techniques.

Polyclonal antibodies can be produced by various procedures well known in the art. For example, a gene product of the present invention, as referenced in Section 5.1.2, or an immunogenic or antigenic fragment thereof can be administered to various host animals including, but not limited to, rabbits, mice, rats, etc. to induce the production of sera containing polyclonal antibodies specific for the obesity related gene product. Various adjuvants can be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2^{nd} ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with osteopontin or an immunogenic or antigenic fragment thereof and once an immune response is detected, e.g., antibodies specific for osteopontin are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding the obesity related gene products of the present invention. Ascites fluid, which generally contains high levels of antibodies, can be generated by immunizing mice with positive hybridoma clones.

Accordingly, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with a gene product referenced in Section 5.1.2 or an immunogenic or antigenic fragment thereof with myeloma cells and then screening the hybridomas resulting from the fusion for hybridoma clones that secrete an antibody able to bind to the subject gene product referenced in Section 5.1.2.

Antibody fragments that recognize specific epitopes can be generated by any technique known to those of skill in the art. For example, Fab and F(ab′)2 fragments of the invention can be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles that carry the polynucleotide sequences encoding them. In particular, DNA sequences encoding VH and VL domains are amplified from animal cDNA libraries (e.g., human or murine cDNA libraries of lymphoid tissues). The DNA encoding the VH and VL domains are recombined together with a scFv linker by PCR and cloned into a phagemid vector (e.g., p CANTAB 6 or pComb 3 HSS). The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to an antigen of interest can be selected or identified with antigen, e.g., using labeled antigen or antigen bound or captured to a solid surface or bead. Examples of phage display methods that can be used to make the antibodies of the present invention include those disclosed in Brinkman et at., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT application No. PCT/GB91/O1 134; PCT publications WO 90/02809; WO 91/10737; WO 92/01047; WO 92/18619; WO 93/1 1236; WO 95/15982; WO 95/20401; WO97/13844; and U.S. Pat. Nos. 5,698,426; 5,223,409; 5,403,484; 5,580,717; 5,427,908; 5,750,753; 5,821,047; 5,571,698; 5,427,908; 5,516,637; 5,780,225; 5,658,727; 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in PCT publication WO 92/22324; Mullinax et al., 1992, BioTechniques 12(6):864-869; and Sawai et al., 1995, AJRI 34:26-34; and Better et al., 1988, Science 240:1041-1043 (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lamba constant regions. Preferably, the vectors for expressing the VH or VL domains comprise an EF-1α promoter, a secretion signal, a cloning site for the variable domain, constant domains, and a selection marker such as neomycin. The VH and VL domains can also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it can be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and PCT publications WO 98/46645, WO 98/50433, WO 98/24893, WO98/16654, WO 96/34096, WO 96/33735, and WO 91/10741; each of which is incorporated herein by reference in its entirety.

Human antibodies can also be produced using transgenic mice that are incapable of expressing functional endogenous immunoglobulins, but which can express human immunoglobulin genes. For example, the human heavy and light chain immunoglobulin gene complexes can be introduced randomly or by homologous recombination into mouse embryonic stem cells. Alternatively, the human variable region, constant region, and diversity region can be introduced into mouse embryonic stem cells in addition to the human heavy and light chain genes. The mouse heavy and light chain immunoglobulin genes can be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring that express human antibodies. The transgenic mice are immunized in the normal fashion with a selected antigen, e.g., all or a portion of a polypeptide of interest. Monoclonal antibodies directed against the antigen can be obtained from the immunized transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., PCT publications WO 98/24893; WO 96/34096; WO 96/33735; U.S. Pat. Nos. 5,413,923; 5,625,126; 5,633,425; 5,569,825; 5,661,016; 5,545,806; 5,814,318; and 5,939,598, which are incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above.

A chimeric antibody is a molecule in which different portions of the antibody are derived from different immunoglobulin molecules such as antibodies having a variable region. derived from a human antibody and a non-human immunoglobulin constant region. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; U.S. Pat. Nos. 5,807,715; 4,816,567; and 4,816,397, which are incorporated herein by reference in their entirety. Chimeric antibodies comprising one or more CDRs from human species and framework regions from a non-human immunoglobulin molecule can be produced using a variety of techniques known in the art including, for example, CDR-grafting (EP 239,400; PCT publication WO 91/09967; U.S. Pat. Nos. 5,225,539; 5,530,101; and 5,585,089), veneering or resurfacing (EP 592,106; EP 519,596; Padlan, 1991, Molecular Immunology 28(4/5):489-498; Studnicka et al., 1994, Protein Engineering 7(6):805-814; Roguska et al., 1994, PNAS 91:969-973), and chain shuffling (U.S. Pat. No. 5,565,332).

Further, the antibodies of the invention can, in turn, be utilized to generate anti-idiotype antibodies that “mimic” one or more of the obesity related gene products of the present invention using techniques well known to those skilled in the art. (See, e.g., Greenspan & Bona, 1989, FASEB J. 7:437-444; and Nissinoff, 1991, J. Immunol. 147:2429-2438).

5.19 Polynucleotides Encoding a Gene Product Antibody

The invention provides polynucleotides comprising a nucleotide sequence encoding an antibody of the invention or a fragment thereof. The invention also encompasses polynucleotides that hybridize under high stringency, intermediate or lower stringency hybridization conditions, e.g., as defined supra, to polynucleotides that encode an antibody of the invention.

The polynucleotides can be obtained, and the nucleotide sequence of the polynucleotides determined, by any method known in the art. Nucleotide sequences encoding these antibodies can be determined using any nucleic acid sequencing method known in the art. Such a polynucleotide encoding the antibody can be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody can be generated from nucleic acid from a suitable source. If a clone containing a nucleic acid encoding a particular antibody is not available, but the sequence of the antibody molecule is known, a nucleic acid encoding the immunoglobulin can be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody of the invention) by PCR amplification using synthetic primers hybridizable to the 3 and 5 ends of the sequence or by cloning using an oligonucleotide probe specific for the particular gene sequence to identify, e.g., a cDNA clone from a cDNA library that encodes the antibody. Amplified nucleic acids generated by PCR can then be cloned into replicable cloning vectors using any method well known in the art.

Once the nucleotide sequence of the antibody is determined, the nucleotide sequence of the antibody can be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2^{nd} Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

5.20 Recombinant Expression of an Antibody to a Gene Product of Interest

Recombinant expression of an antibody of the invention, derivative or analog thereof, (e.g., a heavy or light chain of an antibody of the invention or a portion thereof or a single chain antibody of the invention), requires construction of an expression vector containing a polynucleotide that encodes the antibody. Once a polynucleotide encoding an antibody molecule or a heavy or light chain of an antibody, or portion thereof (preferably, but not necessarily, containing the heavy or light chain variable domain), of the invention has been obtained, the vector for the production of the antibody molecule can be produced by recombinant DNA technology using techniques well known in the art. Thus, methods for preparing a protein by expressing a polynucleotide containing an antibody encoding nucleotide sequences are described herein. Methods that are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a portion thereof, or a heavy or light chain CDR, operably linked to a promoter. Such vectors can include the nucleotide sequence encoding the constant region of the antibody molecule (see, e.g., PCT Publication WO 86/05807; PCT Publication WO 89/01036; and U.S. Pat. No. 5,122,464) and the variable domain of the antibody can be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or portion thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems can be utilized to express the antibody molecules of the invention. Such host-expression systems represent vehicles by which the coding sequences of interest can be produced and subsequently purified, but also represent cells that may, when transformed or transfected with the appropriate nucleotide coding sequences, express an antibody molecule of the invention in situ. These include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; Cockett et al., 1990, Bio/Technology 8:2).

In bacterial systems, a number of expression vectors can be advantageously selected depending upon the use intended for the antibody molecule being expressed. For example, when a large quantity of such a protein is to be produced, for the generation of pharmaceutical compositions of an antibody molecule, vectors that direct the expression of high levels of fusion protein products that are readily purified can be desirable. Such vectors include, but are not limited to, the E. coli expression vector pUR278 (Ruther et al., 1983, EMBO 12:1791), in which the antibody coding sequence can be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes in some instances. The virus grows in Spodoptera frugiperda cells. The antibody coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the antibody coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bittner et al., 1987, Methods in Enzymol. 153:51-544).

In addition, a host cell strain can be chosen that modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product can be used. Such mammalian host cells include but are not limited to CH0, VERY, BHK, Hela, COS, MDCK, 293, 3T3, W138, and in particular, breast cancer cell lines such as, for example, BT483, Hs578T, HTB2, BT20 and T47D, and normal mammary gland cell line such as, for example, CRL7030 and HsS78Bst.

For long-term, high-yield production of recombinant proteins, stable expression is preferred. For example, cell lines that stably express the antibody molecule can be engineered. Rather than using expression vectors that contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines that express the antibody molecule. Such engineered cell lines can be particularly useful in screening and evaluation of compositions that interact directly or indirectly with the antibody molecule.

A number of selection systems can be used including, but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; May, 1993, TIB TECH 11(5):155-2 15); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase. See, for example, Crouse et al., 1983, Mol. Cell. Biol. 3:257.

The host cell can be co-transfected with two expression vectors of the invention, the first vector encoding a heavy chain derived polypeptide and the second vector encoding a light chain derived polypeptide. The two vectors can contain identical selectable markers that enable equal expression of heavy and light chain polypeptides. Alternatively, a single vector may be used that encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2 197). The coding sequences for the heavy and light chains may comprise cDNA or genomic DNA.

Once an antibody molecule of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

5.21 Anti-Sense Nucleic Acids

The function of the genes referenced in Section 5.1.2 can be inhibited by use of antisense nucleic acids. The present invention provides the therapeutic or prophylactic use of nucleic acids of at least six nucleotides in length that are antisense to a gene or cDNA encoding an obesity related gene product referenced in Section 5.1.2, or portions thereof. An “antisense” nucleic acid as used herein refers to a nucleic acid capable of hybridizing to a portion of a nucleic acid referenced in Section 5.1.2 (preferably mRNA, e.g., the sequence of SEQ ID NO: 1, SEQ ID NO: 3, SEQ ID NO: 5, SEQ ID NO: 7, and SEQ ID NO: 9) by virtue of some sequence complementarity. The antisense nucleic acid can be complementary to a coding and/or noncoding region of an obesity related mRNA.

The antisense nucleic acids can be oligonucleotides that are double-stranded or single-stranded RNA or DNA or a modification or derivative thereof, which can be directly administered to a cell, or which can be produced intracellularly by transcription of exogenous, introduced sequences.

The antisense nucleic acids are of at least six nucleotides and are preferably oligonucleotides (ranging from 6 to about 200 oligonucleotides). In specific aspects, the oligonucleotide is at least 10 nucleotides, at least 15 nucleotides, at least 100 nucleotides, or at least 200 nucleotides. The oligonucleotides can be DNA or RNA or chimeric mixtures or derivatives or modified versions thereof, single-stranded or double-stranded. The oligonucleotide can be modified at the base moiety, sugar moiety, or phosphate backbone. The oligonucleotide can include other appending groups such as peptides, or agents facilitating transport across the cell membrane (see, e.g., Letsinger et al., 1989, Proc. Natl. Acad. Sci. U.S.A. 86: 6553-6556; Lemaitre et al., 1987, Proc. Natl. Acad. Sci. 84: 648-652; PCT Publication No. WO 88/09810, published Dec. 15, 1988) or blood-brain barrier (see, e.g., PCT Publication No. WO 89/10134, published Apr. 25, 1988), hybridization-triggered cleavage agents (see, e.g., Krol et al., 1988, BioTechniques 6: 958-976) or intercalating agents (see, e.g., Zon, 1988, Pharm. Res. 5: 539-549).

In a preferred aspect of the invention, the antisense oligonucleotide is provided, preferably as single-stranded DNA. The oligonucleotide can be modified at any position on its structure with constituents generally known in the art. The antisense oligonucleotides can comprise at least one modified base moiety that is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5 -methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacefic acid (v), 5-methyl-2-thiouracil, 3-(3-amino-3-N-2-carboxypropyl)uracil, and 2,6-diaminopurine.

In another embodiment, the oligonucleotide comprises at least one modified sugar moiety selected from the group including, but not limited to, arabinose, 2-fluoroarabinose, xylulose, and hexose.

In yet another embodiment, the oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, a formacetal, or analogs thereof.

In yet another embodiment, the oligonucleotide is an α-anomeric oligonucleotide. An α-anomeric oligonucleotide forms specific double-stranded hybrids with complementary RNA in which, contrary to the usual β-units, the strands run parallel to each other (Gautier et al., 1987, Nucl. Acids Res. 15: 6625-6641).

The oligonucleotide can be conjugated to another molecule, e.g., a peptide, hybridization triggered cross-linking agent, transport agent, hybridization-triggered cleavage agent, etc.

Oligonucleotides may be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al. (1988, Nucl. Acids Res. 16: 3209), methylphosphonate oligonucleotides can be prepared by use of controlled pore glass polymer supports (Sarin et al., 1988, Proc. Natl. Acad. Sci. U.S.A. 85: 7448-7451), etc.

In a specific embodiment, the antisense oligonucleotides comprise catalytic RNAs, or ribozymes (see, e.g., PCT International Publication WO 90/11364, published Oct. 4, 1990; Sarver et al., 1990, Science 247: 1222-1225). In another embodiment, the oligonucleotide is a 2 -0-methylribonucleotide (Inoue et al., 1987, Nucl. Acids Res. 15: 6131-6148), or a chimeric RNA-DNA analog (Inoue et al., 1987, FEBS Lett. 215: 327-330).

In an alternative embodiment, antisense nucleic acids are produced intracellularly by transcription from an exogenous sequence. For example, a vector can be introduced in vivo such that it is taken up by a cell, within which cell the vector or a portion thereof is transcribed, producing an antisense nucleic acid (RNA) of the invention. Such a vector would contain a sequence encoding an antisense nucleic acid. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequences encoding the antisense RNAs can be by any promoter known in the art to act in mammalian, preferably human, cells. Such promoters can be inducible or constitutive. Such promoters include, but are not limited to, the SV40 early promoter region (Bernoist and Chambon, 1981, Nature 290: 304-310), the promoter contained in the 3 long terminal repeat of Rous sarcoma virus (Yamamoto et al., 1980, Cell 22: 787-797), the herpes thymidine kinase promoter (Wagner et al., 1981, Proc. Natl. Acad. Sci. U.S.A. 78: 1441-1445), the regulatory sequences of the metallothionein gene (Brinster et al., 1982, Nature 296: 39-42), etc.

The antisense nucleic acids of the invention comprise a sequence complementary to at least a portion of an RNA transcript of a gene referenced in Section 5.1.2. However, absolute complementarity, although preferred, is not required. A sequence “complementary to at least a portion of an RNA,” as referred to herein, means a sequence having sufficient complementarity to be able to hybridize with the RNA, forming a stable duplex; in the case of double-stranded antisense nucleic acids, a single strand of the duplex DNA can thus be tested, or triplex formation can be assayed. The ability to hybridize will depend on both the degree of complementarity and the length of the antisense nucleic acid.

Generally, the longer the hybridizing nucleic acid, the more base mismatches with an obesity related RNA (target RNA) it may contain and still form a stable duplex (or triplex, as the case may be). One skilled in the art can ascertain a tolerable degree of mismatch by use of standard procedures to determine the melting point of the hybridized complex.

Pharmaceutical compositions of the invention, comprising an effective amount of an antisense nucleic acid in a pharmaceutically acceptable carrier can be administered in therapeutic methods of the invention. The amount of antisense nucleic acid that will be effective in the treatment of a particular disorder or condition will depend on the nature of the disorder or condition, and can be determined by standard clinical techniques. Where possible, it is desirable to determine the antisense cytotoxicity in vitro, and then in useful animal model systems prior to testing and use in humans.

In a specific embodiment, pharmaceutical compositions comprising antisense nucleic acids are administered via liposomes, microparticles, or microcapsules. In various embodiments of the invention, it may be useful to use such compositions to achieve sustained release of antisense nucleic acids. In a specific embodiment, it can be desirable to utilize liposomes targeted via antibodies to specific identifiable central nervous system cell types (Leonetti et al., 1990, Proc. Natl. Acad. Sci. U.S.A. 87:

2448-2451; Renneisen et al., 1990, J. Biol. Chem. 265: 16337-16342).

5.22 RNA Interference

In certain embodiments, an RNA interference (RNAi) molecule is used to decrease the gene expression level. RNA interference (RNAi) is defined as the ability of double-stranded RNA (dsRNA) to suppress the expression of a gene corresponding to its own sequence. RNAi is also called post-transcriptional gene silencing or PTGS. Since the only RNA molecules normally found in the cytoplasm of a cell are molecules of single-stranded mRNA, the cell has enzymes that recognize and cut dsRNA into fragments containing 21-25 base pairs (approximately two turns of a double helix and which are referred to as small interfering RNA or siRNA). The antisense strand of the fragment separates enough from the sense strand so that it hybridizes with the complementary sense sequence on a molecule of endogenous cellular mRNA. This hybridization triggers cutting of the mRNA in the double-stranded region, thus destroying its ability to be translated into a polypeptide. Introducing dsRNA corresponding to a particular gene thus knocks out the cell's own expression of that gene in particular tissues and/or at a chosen time.

Double-stranded (ds) RNA can be used to interfere with gene expression in mammals (Wianny & Zernicka-Goetz, 2000, Nature Cell Biology 2: 70-75; incorporated herein by reference in its entirety). dsRNA is used as inhibitory RNA or RNAi of the function of a gene (e.g., SEQ ID NO:1, SEQ ID NO:3, SEQ ID NO:5, SEQ ID NO:7, and SEQ ID NO:9) to produce a phenotype that is the same as that of a null mutant of the gene (Wianny & Zernicka-Goetz, 2000, Nature Cell Biology 2: 70-75).

RNA interference (RNAi) is a potent method to suppress gene expression in mammalian cells, and has generated much excitement in the scientific community (Couzin, 2002, Science 298:2296-2297; McManus et al., 2002, Nat. Rev. Genet. 3, 737-747; Hannon, G. J., 2002, Nature 418, 244-251; Paddison et al., 2002, Cancer Cell 2, 17-23). RNA interference is conserved throughout evolution, from C. elegans to humans, and is believed to function in protecting cells from invasion by RNA viruses. When a cell is infected by a dsRNA virus, the dsRNA is recognized and targeted for cleavage by an RNaseIII-type enzyme termed Dicer. The Dicer enzyme “dices” the RNA into short duplexes of 2 Int, termed siRNAs or short-interfering RNAs, composed of 19 nt of perfectly paired ribonucleotides with two unpaired nucleotides on the 3′ end of each strand. These short duplexes associate with a multiprotein complex termed RISC, and direct this complex to mRNA transcripts with sequence similarity to the siRNA. As a result, nucleases present in the RISC complex cleave the mRNA transcript, thereby abolishing expression of the gene product. In the case of viral infection, this mechanism would result in destruction of viral transcripts, thus preventing viral synthesis. Since the siRNAs are double-stranded, either strand has the potential to associate with RISC and direct silencing of transcripts with sequence similarity.

Specific gene silencing promises the potential to harness human genome data to elucidate gene function, identify drug targets, and develop more specific therapeutics. Many of these applications assume a high degree of specificity of siRNAs for their intended targets. Cross-hybridization with transcripts containing partial identity to the siRNA sequence may elicit phenotypes reflecting silencing of unintended transcripts in addition to the target gene. This could confound the identification of the gene implicated in the phenotype. Numerous reports in the literature purport the exquisite specificity of siRNAs, suggesting a requirement for near-perfect identity with the siRNA sequence (Elbashir et al., 2001. EMBO J. 20:6877-6888; Tuschl et al., 1999, Genes Dev. 13:3191-3197; Hutvagner et al., Sciencexpress 297:2056-2060). One recent report suggests that perfect sequence complementarity is required for siRNA-targeted transcript cleavage, while partial complementarity will lead to tranlational repression without transcript degradation, in the manner of microRNAs (Hutvagner et al., Sciencexpress 297:2056-2060).

The biological function of small regulatory RNAs, including siRNAs and miRNAs is not well understood. One prevailing question regards the mechanism by which the distinct silencing pathways of these two classes of regulatory RNA are determined. miRNAs are regulatory RNAs expressed from the genome, and are processed from precursor stem-loop structures to produce single-stranded nucleic acids that bind to sequences in the 3′ UTR of the target mRNA (Lee et al., 1993, Cell 75:843-854; Reinhart et al., 2000, Nature 403:901-906; Lee et al., 2001, Science 294:862-864; Lau et al., 2001, Science 294:858-862; Hutvagner et al., 2001, Science 293:834-838). miRNAs bind to transcript sequences with only partial complementarity (Zeng et al., 2002, Molec. Cell 9:1327-1333) and repress translation without affecting steady-state RNA levels (Lee et al., 1993, Cell 75:843-854; Wightman et al., 1993, Cell 75:855-862). Both miRNAs and siRNAs are processed by Dicer and associate with components of the RNA-induced silencing complex (Hutvagner et al., 2001, Science 293:834-838; Grishok et al., 2001, Cell 106: 23-34; Ketting et al., 2001, Genes Dev. 15:2654-2659; Williams et al., 2002, Proc. Natl. Acad. Sci. USA 99:6889-6894; Hammond et al., 2001, Science 293:1146-1150; Mourlatos et al., 2002, Genes Dev. 16:720-728). A recent report (Hutvagner et al., 2002, Sciencexpress 297:2056-2060) hypothesizes that gene regulation through the miRNA pathway versus the siRNA pathway is determined solely by the degree of complementarity to the target transcript. It is speculated that siRNAs with only partial identity to the mRNA target will function in translational repression, similar to an miRNA, rather than triggering RNA degradation.

It has also been shown that siRNA and shRNA can be used to silence genes in vivo. The ability to utilize siRNA and shRNA for gene silencing in vivo has the potential to enable selection and development of siRNAs for therapeutic use. A recent report highlights the potential therapeutic application of siRNAs. Fas-mediated apoptosis is implicated in a broad spectrum of liver diseases, where lives could be saved by inhibiting apoptotic death of hepatocytes. Song (Song et al. 2003, Nat. Medicine 9, 347-351) injected mice intravenously with siRNA targeted to the Fas receptor. The Fas gene was silenced in mouse hepatocytes at the mRNA and protein levels, prevented apoptosis, and protected the mice from hepatitis-induced liver damage. Thus, silencing Fas expression holds therapeutic promise to prevent liver injury by protecting hepatocytes from cytotoxicity. As another example, injected mice intraperitoneally with siRNA targeting TNF-a. Lipopolysaccharide-induced TNF-a gene expression was inhibited, and these mice were protected from sepsis. Collectively, these results suggest that siRNAs can function in vivo, and may hold potential as therapeutic drugs (Sorensen et al., 2003, J. Mol. Biol. 327, 761-766).

Martinez et al. reported that RNA interference can be used to selectively target oncogenic mutations (Martinez et al., 2002, Proc. Natl. Acad. Sci. USA 99:14849-14854). In this report, an siRNA that targets the region of the R248W mutant of p53 containing the point mutation was shown to silence the expression of the mutant p53 but not the wild-type p53.

Wilda et al. reported that an siRNA targeting the M-BCR/ABL fusion mRNA can be used to deplete the M-BCR/ABL mRNA and the M-BRC/ABL oncoprotein in leukemic cells (Wilda et al., 2002, Oncogene 21:5716-5724). However, the report also showed that applying the siRNA in combination with Imatinib, a small-molecule ABL kinase tyrosine inhibitor, to leukemic cells did not further increase in the induction of apoptosis.

U.S. Pat. No. 6,506,559 discloses a RNA interference process for inhibiting expression of a target gene in a cell. The process comprises introducing partially or fully doubled-stranded RNA having a sequence in the duplex region that is identical to a sequence in the target gene into the cell or into the extracellular environment. RNA sequences with insertions, deletions, and single point mutations relative to the target sequence are also found as effective for expression inhibition.

U.S. Patent Application Publication No. US 2002/0086356 discloses RNA interference in a Drosophila in vitro system using RNA segments 21-23 nucleotides (nt) in length. The patent application publication teaches that when these 21-23 nt fragments are purified and added back to Drosophila extracts, they mediate sequence-specific RNA interference in the absence of long dsRNA. The patent application publication also teaches that chemically synthesized oligonucleotides of the same or similar nature can also be used to target specific mRNAs for degradation in mammalian cells.

PCT publication WO 02/44321 discloses that double-stranded RNA (dsRNA) 19-23 nt in length induces sequence-specific post-transcriptional gene silencing in a Drosophila in vitro system. The PCT publication teaches that short interfering RNAs (siRNAs) generated by an RNase IlI-like processing reaction from long dsRNA or chemically synthesized siRNA duplexes with overhanging 3′ ends mediate efficient target RNA cleavage in the lysate, and the cleavage site is located near the center of the region spanned by the guiding siRNA. The PCT publication also provides evidence that the direction of dsRNA processing determines whether sense or antisense target RNA can be cleaved by the produced siRNP complex.

U.S. Patent Application Publication No. US 2002/016216 discloses a method for attenuating expression of a target gene in cultured cells by introducing double stranded RNA (dsRNA) that comprises a nucleotide sequence that hybridizes under stringent conditions to a nucleotide sequence of the target gene into the cells in an amount sufficient to attenuate expression of the target gene.

PCT publication WO 03/006477 discloses engineered RNA precursors that when expressed in a cell are processed by the cell to produce targeted small interfering RNAs (siRNAs) that selectively silence targeted genes (by cleaving specific mRNAs) using the cell's own RNA interference (RNAi) pathway. The PCT publication teaches that by introducing nucleic acid molecules that encode these engineered RNA precursors into cells in vivo with appropriate regulatory sequences, expression of the engineered RNA precursors can be selectively controlled both temporally and spatially, i.e., at particular times and/or in particular tissues, organs, or cells.

5.23 Antisense

The present invention encompasses antisense nucleic acid molecules, i.e., molecules which are complementary to all or part of a sense nucleic acid encoding a gene (e.g., SEQ ID NO:1, SEQ ID NO:3, SEQ ID NO:5, SEQ ID NO:7, and SEQ ID NO:9), e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire coding strand, or to only a portion thereof, e.g., all or part of the protein coding region (or open reading frame). An antisense nucleic acid molecule can be antisense to all or part of a non-coding region of the coding strand of a nucleotide sequence encoding a polypeptide of the invention. The non-coding regions (“5 and 3 untranslated regions”) are the 5 and 3 sequences which flank the coding region and are not translated into amino acids.

An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, β-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, β-D-mannosylqueosine, 5 -methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, e.g., nucleic acid encoding SEQ ID NO:1, SEQ ID NO:3, SEQ ID NO:5, SEQ ID NO:7, and SEQ ID NO:9.

The antisense nucleic acid molecules of the invention are typically administered to a subject or generated in situ such that they hybridize with or bind to cellular mRNA and/or genomic DNA encoding a selected polypeptide of the invention to thereby inhibit expression, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual β-units, the strands run parallel to each other (Gaultier et al., 1987, Nucleic Acids Res. 15:6625). The antisense nucleic acid molecule can also comprise a 2 -o-methylribonucleotide (Inoue et al., 1987, Nucleic Acids Res. 15:6131) or a chimeric RNA-DNA analogue (Inoue et al., 1987, FEBS Lett. 215:327).

5.24 Ribozymes

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes; described in Haselhoff and Gerlach, 1988, Nature 334:585-591) can be used to catalytically cleave mRNA transcripts to thereby inhibit translation of the protein encoded by the mRNA. A ribozyme having specificity for a nucleic acid molecule encoding a gene of interest (e.g., SEQ ID NO: 1, SEQ ID NO:3, SEQ ID NO:5, SEQ ID NO:7, and SEQ ID NO:9) can be designed based upon the nucleotide sequence of the gene (e.g., SEQ ID NO:1, SEQ ID NO:3, SEQ ID NO:5, SEQ ID NO:7, and SEQ ID NO:9). For example, a derivative of a Tetrahymena L-19 IVS RNA can be constructed in which the nucleotide sequence of the active site is complementary to the nucleotide sequence to be cleaved in U.S. Pat. Nos. 4,987,071 and 5,116,742. Alternatively, an mRNA encoding a polypeptide of the invention can be used to select a catalytic RNA having a specific ribonuclease activity from a pool of RNA molecules. See, e.g., Bartel and Szostak, 1993, Science 261:1411.

5.25 Gene Product Analogs, Derivatives and Fragments

The invention further provides methods of modulating the genes referenced in Section 5.1.2 using agonists and promoters of such genes. Agonists include, but are not limited to, active fragments thereof (wherein a fragment is at least 10, 15, 20, 30, 50, 75, 100, or 150 amino acid portion of an obesity related gene product disclosed in Section 6.7.5) and analogs and derivatives thereof, and nucleic acids encoding any of the foregoing.

For recombinant expression of gene products, and fragments, derivatives and analogs thereof, the nucleic acid containing all or a portion of the nucleotide sequence encoding the protein can be inserted into an appropriate expression vector, e.g., a vector that contains the necessary elements for the transcription and translation of the inserted protein coding sequence. In a preferred embodiment, the regulatory elements (e.g., promoter) are heterologous (i.e., not the native gene promoter). Promoters which may be used include but are not limited to the SV40 early promoter (Bernoist and Chambon, 1981, Nature 290: 304-310), the promoter contained in the 3 long terminal repeat of Rous sarcoma virus (Yamamoto et al., 1980, Cell 22: 787-797), the herpes thymidine kinase promoter (Wagner et al., 1981, Proc. Natl. Acad. Sci. USA 78: 1441-1445), the regulatory sequences of the metallothionein gene (Brinster et al., 1982, Nature 296: 39-42); prokaryotic expression vectors such as the β-lactamase promoter (Villa-Kamaroff et al., 1978, Proc. Natl. Acad. Sci. USA 75: 3727-3731) or the tac promoter (DeBoer et al., 1983, Proc. Natl. Acad. Sci. USA 80: 21-25; see also “Useful Proteins from Recombinant Bacteria”: in Scientific American 1980, 242:79-94); plant expression vectors comprising the nopaline synthetase promoter (Herrar-Estrella et al., 1984, Nature 303: 209-213) or the cauliflower mosaic virus 35S RNA promoter (Garder et al., 1981, Nucleic Acids Res. 9:2871), and the promoter of the photosynthetic enzyme ribulose bisphosphate carboxylase (Herrera-Estrella et al., 1984, Nature 310: 115-120); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been utilized in transgenic animals: elastase 1 gene control region which is active in pancreatic acinar cells (Swift et al., 1984, Cell 38: 639-646; Ornitz et al., 1986, Cold Spring Harbor Symp. Quant. Biol. 50: 399-409; MacDonald 1987, Hepatology 7: 425-515); insulin gene control region which is active in pancreatic beta cells (Hanahan et al., 1985, Nature 315: 115-122), immunoglobulin gene control region which is active in lymphoid cells (Grosschedl et al., 1984, Cell 38: 647-658; Adams et al., 1985, Nature 318: 533-538; Alexander et al., 1987, Mol. Cell Biol. 7: 1436-1444), mouse mammary tumor virus control region which is active in testicular, breast, lymphoid and mast cells (Leder et al., 1986, Cell 45: 485-495), albumin gene control region which is active in liver (Pinckert et al., 1987, Genes and Devel. 1: 268-276), alpha-fetoprotein gene control region which is active in liver (Krumlauf et al., 1985, Mol. Cell. Biol. 5: 1639-1648; Hammer et al., 1987, Science 235: 53-58), alpha-1 antitrypsin gene control region which is active in liver (Kelsey et al., 1987, Genes and Devel. 1: 161-171), beta globin gene control region which is active in myeloid cells (Mogram et al., 1985, Nature 315: 338-340; Kollias et al., 1986, Cell 46: 89-94), myelin basic protein gene control region which is active in oligodendrocyte cells of the brain (Readhead et al., 1987, Cell 48: 703-712), myosin light chain-2 gene control region which is active in skeletal muscle (Sani 1985, Nature 314: 283-286), and gonadotrophic releasing hormone gene control region which is active in gonadotrophs of the hypothalamus (Mason et al., 1986, Science 234: 1372-1378).

A variety of host-vector systems can be utilized to express the protein coding sequence. These include, but are not limited to, mammalian cell systems infected with virus (e.g., vaccinia virus, adenovirus, etc.); insect cell systems infected with virus (e.g. baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements can be used.

Once a gene product disclosed in Section 5.1.2, or fragment, derivative or analog thereof has been recombinantly expressed, it can be isolated and purified by standard methods including chromatography (e.g., ion exchange, affinity, and sizing column chromatography), centrifugation, differential solubility, or by any other standard technique for the purification of proteins. An obesity related gene product can also be purified by any standard purification method from natural sources.

Alternatively, an obesity related gene product, analog or derivative thereof of the present invention can be synthesized by standard chemical methods known in the art (e.g., see Hunkapiller et al., 1984, Nature 310:105-111).

Standard techniques known to those of skill in the art can be used to introduce mutations in the nucleotide sequence encoding a molecule of the invention, including, for example, site-directed mutagenesis and PCR-mediated mutagenesis that results in amino acid substitutions. Preferably, the derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In a preferred embodiment, the derivatives have conservative amino acid substitutions are made at one or more predicted non-essential amino acid residues. A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Biological activity can be deduced by identifying known protein motifs. Following mutagenesis, the encoded protein can be expressed and the activity of the protein can be determined.

In a specific embodiment, the gene analog, derivative or fragment thereof is encoded by a nucleotide sequence that hybridizes to the nucleotide sequence of SEQ ID NO: 1, SEQ ID NO: 3, SEQ ID NO: 5, SEQ ID NO: 7, and SEQ ID NO: 9 under stringent conditions, e.g., hybridization to filter-bound DNA in 6× sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions that are known to those of skill in the art (see, for example, Ausubel, F. M. et al., eds., 1989, Current Protocols in Molecular Biology, Vol. 1, Green Publishing Associates, Inc. and John Wiley & Sons, Inc., New York at pages 6.3.1-6.3.6 and 2.10.3).

In another embodiment, the analog, derivative or fragment comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of SEQ ID NO: 1, SEQ ID NO: 3, SEQ ID NO: 5, SEQ ID NO: 7, and SEQ ID NO: 9. Additionally, the nucleic acid sequence can be mutated in vitro or in vivo, to create and/or destroy translation, initiation, and/or termination sequences, or to create variations in coding regions and/or form new restriction endonuclease sites or destroy preexisting ones, to facilitate further in vitro modification. Any technique for mutagenesis known in the art can be used, including, but not limited to, chemical mutagenesis, in vitro site-directed mutagenesis (Hutchinson, C., et al., 1978, J. Biol. Chem 253:6551), use of TAB® linkers (Pharmacia), etc.

Manipulations of the sequence can also be made at the protein level. Included within the scope of the invention are protein fragments or other derivatives or analogs that are differentially modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications can be carried out by known techniques including, but not limited to, specific chemical cleavage by cyanogen bromide, trypsin, chymotrypsin, papain, V8 protease, NaBH₄, acetylation, formylation, oxidation, reduction; metabolic synthesis in the presence of tunicamycin, etc.

In addition, analogs and derivatives of the gene products referenced in Section 5.1.2 can be chemically synthesized. Furthermore, if desired, nonclassical amino acids or chemical amino acid analogs can be introduced as a substitution or addition into such sequences. Non-classical amino acids include but are not limited to the D-isomers of the common amino acids, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosine, citrulline, cysteic acid, t-butylglycine, t-butylalanine, phenylglycine, cyclohexylalanine, β-alanine, fluoro-amino acids, designer amino acids such as β-methyl amino acids, Ca-methyl amino acids, Na-methyl amino acids, and amino acid analogs in general.

Furthermore, the amino acids used to make the analogs and derivatives can be D (dextrorotary), L (levorotary), or some combination of D and L.

In a specific embodiment, the derivative is a chimeric (or fusion) protein comprising a gene product referenced in Section 5.1.2 or fragment thereof (preferably consisting of at least one protein domain or protein structural motif, or at least 15, preferably 20, amino acids of the obesity related protein) joined at its amino- or carboxy-terminus via a peptide bond to an amino acid sequence of a different protein. In one embodiment, such a chimeric protein is produced by recombinant expression of a nucleic acid encoding the protein (comprising an obesity related protein-coding sequence joined in-frame to a coding sequence for a different protein). Such a chimeric product can be made by ligating the appropriate nucleic acid sequences encoding the desired amino acid sequences to each other by methods known in the art, in the proper coding frame, and expressing the chimeric product by methods commonly known in the art. Alternatively, such a chimeric product may be made by protein synthetic techniques, e.g., by use of a peptide synthesizer. Chimeric genes comprising portions of a gene product referenced in Section 5.1.2 (e.g. SEQ ID NO: 1, SEQ ID NO: 3, SEQ ID NO: 5, SEQ ID NO: 7, and SEQ ID NO: 9) fused to any heterologous protein-encoding sequences can be constructed.

5.26 Pharmaceutical Compositions and Methods of Administration

The invention provides methods of treatment, prophylaxis, and amelioration of one or more symptoms associated with obesity by administrating to a subject an effective amount of a modulater of a gene referenced in Section 5.1.2. (e.g. SEQ ID NO: 1, SEQ ID NO: 3, SEQ ID NO: 5, SEQ ID NO: 7, and SEQ ID NO: 9), or a pharmaceutical composition comprising an obesity related gene modulator. In a preferred aspect, the obesity related gene modulator is substantially purified (e.g., substantially free from substances that limit its effect or produce undesired side-effects). The subject is preferably a mammal such as non-primate (e.g., cows, pigs, horses, cats, dogs, rats etc.) and a primate (e.g., monkeys or humans). In a preferred embodiment, the subject is a human.

5.26.1 Delivery Systems

Various delivery systems are known and can be used to administer modulators of the invention or fragment thereof, e.g., encapsulation in liposomes, microparticles, microcapsules, recombinant cells capable of expressing a protein or antibody modulator, receptor-mediated endocytosis (see, e.g., Wu and Wu, 1987, J. Biol. Chem. 262:4429-4432), construction of a nucleic acid as part of a retroviral or other vector, etc. Methods of administering a modulator, or pharmaceutical composition include, but are not limited to, parenteral administration (e.g., intradermal, intramuscular, intraperitoneal, intravenous and subcutaneous), epidural, and mucosal (e.g., intranasal and oral routes). In a specific embodiment, modulators of the present invention or fragments thereof, or pharmaceutical compositions are administered intramuscularly, intravenously, or subcutaneously. The compositions can be administered by any convenient route, for example by infusion or bolus injection, by absorption through epithelial or mucocutaneous linings (e.g., oral mucosa, rectal and intestinal mucosa, etc.) and can be administered together with other biologically active agents.

Administration can be systemic or local. In addition, pulmonary administration can also be employed, e.g., by use of an inhaler or nebulizer, and formulation with an aerosolizing agent. See, e.g., U.S. Pat. Nos. 6,019,968, 5,985,309, 5,934,272, 5,874,064, 5,290,540, and 4,880,078, and PCT Publication No. WO 92/19244. In a preferred embodiment, the pharmaceutical composition is delivered locally to the site of neural tissue damage, e.g., using osmotic or other types of pumps.

5.26.2 Pharmaceutical Compositions

The invention also provides that the pharmaceutical composition is packaged in a hermetically sealed container such as an ampule or sachette indicating the quantity of modulator. In one embodiment, the modulator is supplied as a dry sterilized lyophilized powder or water free concentrate in a hermetically sealed container and can be reconstituted, e.g., with water or saline to the appropriate concentration for administration to a subject. Preferably, the modulator is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, or at least 75 mg. Preferably, the liquid form is supplied in a hermetically sealed container at least 1 mg/ml, more preferably at least 2.5 mg/ml, at least 5 mg/ml, at least 8 mg/ml, at least 10 mg/ml, or at least 25 mg/ml.

In a specific embodiment, it can be desirable to administer the pharmaceutical compositions of the invention locally to the area in need of treatment; this can be achieved by, for example, and not by way of limitation, local infusion, by injection, or by means of an implant, said implant being of a porous, non-porous, or gelatinous material, including membranes, such as sialastic membranes, or fibers. A particularly useful application involves coating, imbedding or derivatizing fibers, such as collagen fibers, protein polymers, etc. with a modulator of the invention. Other useful approaches are described in Otto et at., 1989, J Neuroscience Research 22, 83-91 and Otto and Unsicker, 1990, J Neuroscience 10, 1912-1921, both of which are incorporated herein in their entireties. Preferably, when administering the modulator, care must be taken to use materials to which the modulator does not absorb.

In another embodiment, the composition can be delivered in a vesicle, in particular a liposome (see Langer, 1990, Science 249:1527-1533 1990); Treat et al., 1989, in Liposomes in the Therapy of Infectious Disease and Cancer, Lopez-Berestein and Fidler (eds.), Liss, New York, pp. 353-365; and Lopez-Berestein, ibid., pp. 317-327; see generally ibid.).

In yet another embodiment, the composition can be delivered in a controlled release system. In one embodiment, a pump may be used (see Langer, supra; Sefton, 1987, CRC Crit. Ref. Biomed. Eng. 14:20; Buchwald et al., 1980, Surgery 88:507; Saudek et al., 1989, N. Engl. J. Med. 321-574). In another embodiment, polymeric materials can be used (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. In yet another embodiment, a controlled release system can be placed in proximity of the therapeutic target, i.e., nervous tissue (see, e.g., Goodson, 1984, in Medical Applications of Controlled Release, supra, vol. 2, pp. 115-138). Other controlled release systems are discussed in the review by Langer, 1990, Science 249:1527-1533.

In a specific embodiment, where the composition of the invention is a nucleic acid encoding modulator, the nucleic acid can be administered in vivo to promote expression of its encoded modulator by constructing it as part of an appropriate nucleic acid expression vector and administering it so that it becomes intracellular, e.g., by use of a retroviral vector (see U.S. Pat. No.4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression by homologous recombination.

The pharmaceutical compositions of the invention comprise a prophylactically or therapeutically effective amount of an obesity related gene modulator, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a prophylactically or therapeutically effective amount of the antibody or fragment thereof, preferably in purified form, together with a suitable amount of carrier so as to provide the form for proper administration to the patient. The formulation should suit the mode of administration.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition can also include a solubilizing agent and a local anesthetic such as lignocamne to ease pain at the site of the injection.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc. The amount of the composition delivered is that amount that will be effective in the methods of treatment of the invention.

5.26.3 Gene Therapy

In some embodiments, the compositions are delivered by gene therapy. Gene therapy refers to therapy performed by the administration to a subject of an expressed or expressible nucleic acid. In this embodiment of the invention, the nucleic acids produce their encoded modulator that mediates a therapeutic effect. Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, a composition of the invention comprises nucleic acids encoding a modulator. These nucleic acids are part of an expression vector that expresses the modulator in a suitable host. In particular, such nucleic acids have promoters, preferably heterologous promoters, operably linked to the antibody coding region, the promoter being inducible or constitutive and, optionally, tissue-specific. In another particular embodiment, nucleic acid molecules are used in which the modulator coding sequences and any other desired sequences are flanked by regions that promote homologous recombination at a desired site in the genome, thus providing for intrachromosomal expression of the modulator encoding nucleic acids (Koller and Smithies, 1989, Proc. Natl. Acad. Sci. USA 86:8932-8935; Zijlstra et al., 1989, Nature 342:435-438). In specific embodiments, where the modulator is an antibody, the expressed antibody molecule is a single chain antibody. Alternatively, the nucleic acid sequences include sequences encoding both the heavy and light chains, or fragments thereof, of the antibody.

Delivery of the nucleic acids into a subject can be either direct, in which case the subject is directly exposed to the nucleic acid or nucleic acid-carrying vectors, or indirect, in which case cells are first transformed with the nucleic acids in vitro, then transplanted into the subject. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO92/203 16; WO93/14188, WO 93/20221). Alternatively, the nucleic acid can be introduced intracellularly and incorporated within host cell DNA for expression, by homologous recombination (Koller and Smithies, 1989, Proc. Natl. Acad. Sci. USA 86:8932-8935; and Zijlstra et al., 1989, Nature 342:435-438).

In a specific embodiment, viral vectors that contains nucleic acid sequences encoding an antibody of the invention or fragments thereof are used. For example, a retroviral vector can be used (see Miller et al., 1993, Meth. Enzymol. 217:581-599). These retroviral vectors contain the components necessary for the correct packaging of the viral genome and integration into the host cell DNA. The nucleic acid sequences encoding the antibody to be used in gene therapy are cloned into one or more vectors, which facilitates delivery of the gene into a subject. More detail about retroviral vectors can be found in Boesen et al., 1994, Biotherapy 6:291-302, which describes the use of a retroviral vector to deliver the mdr 1 gene to hematopoietic stem cells in order to make the stem cells more resistant to chemotherapy. Other references illustrating the use of retroviral vectors in gene therapy are Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy and can be targeted to the central nervous system. Adenoviruses have the advantage of being capable of infecting non-dividing cells. Kozarsky and Wilson, 1993, Current Opinion in Genetics and Development 3:499-503 present a review of adenovirus-based gene therapy. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang et al., 1995, Gene Therapy 2:775-783. Adeno-associated virus (AAV) has also been proposed for use in gene therapy (Walsh et al., 1993, Proc. Soc. Exp. Biol. Med. 204:289-300; and U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcellmediated gene transfer, spheroplast fusion, etc. Numerous techniques are known in the art for the introduction of foreign genes into cells (see, e.g., Loeffler and Behr, 1993, Meth. Enzymol. 217:599-618; and Cohen et al., 1993, Meth. Enzymol. 217:618-644) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc. In a preferred embodiment, the cell is a neural cell. In a preferred embodiment, the cell used for gene therapy is autologous to the subject.

In an embodiment in which recombinant cells are used in gene therapy, nucleic acid sequences encoding a modulator are introduced into the cells such that they are expressible by the cells or their progeny, and the recombinant cells are then administered in vivo for therapeutic effect. In a specific embodiment, stem or progenitor cells are used. Any stem and/or progenitor cells that can be isolated and maintained in vitro can potentially be used in accordance with this embodiment of the present invention (see e.g., PCT Publication WO 94/08598; Stemple and Anderson, 1992, Cell 7 1:973-985; Rheinwald, 1980, Meth. Cell Bio. 21A:229; and Pittelkow and Scott, 1986, Mayo Clinic Proc. 61:771). In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

5.27 Exemplary Database Architectures

In some embodiments, patient database 44 (see FIG. 1) is a data warehouse. Data warehouses are typically structured as either relational databases or multidimensional data cubes. In this section, exemplary database 44 has a relational database or a multidimensional data cube architecture are described. For more information on relational databases and multidimensional data cubes, see Berson and Smith, 1997, Data Warehousing, Data Mining and OLAP, McGraw-Hill, New York; Freeze, 2000, Unlocking OLAP with Microsoft SQL Server and Excel 2000, IDG Books Worldwide, Inc., Foster City, Calif.; and Thomson, 1997, OLAP Solutions: Building Multidimensional Information Systems, Wiley Computer Publishing, New York. In addition, it will be appreciated that, in some embodiments, database 44 does not have a formal hierarchical structure.

5.27.1 Data Organization

Databases have typically been used for operational purposes (OLTP), such as order entry, accounting and inventory control. More recently, corporations and scientific projects have been building databases, called data warehouses or large on-line analytical processing (OLAP) databases, explicitly for the purposes of exploration and analysis. The “data warehouse” can be described as a subject-oriented, integrated, time-variant, nonvolatile collection of data in support of management decisions. Data warehouses are built using both relational databases and specialized multidimensional structures called data cubes. In some embodiments database 44 is a datacube or a relational database.

5.27.2 Relational Databases

Relational databases organize data into tables where each row corresponds to a basic entity or fact and each column represents a property of that entity. For example, a table can represent transactions in a bank, where each row corresponds to a single transaction, and each transaction has multiple attributes, such as the transaction amount, the account balance, the bank branch, and the customer. The relational table is referred to as a relation, a row as a tuple, and a column as an attribute or field. The attributes within a relation can be partitioned into two types: dimensions and measures. Dimensions and measures are similar to independent and dependent variables in traditional analysis. For example, the bank branch and the customer would be dimensions, while the account balance would be a measure. A single relational database will often describe many heterogeneous but interrelated entities. For example, a database designed for a restaurant chain might maintain information about employees, products, and sales. The database schema defines the relations in a database, the relationships between those relations, and how the relations model the entities of interest.

5.27.3 Data Cubes

A data warehouse can be constructed as a relational database using either a star or snowflake schema and will provide a conceptual model of a multidimensional data set. Each axis in the corresponding data cube represents a dimension in a relational schema and consists of every possible value for that dimension. For example, an axis corresponding to states would have fifty values, one for each state. Each cell in the data cube corresponds to a unique combination of values for the dimensions. For example, if there are two dimensions, “State” and “Product”, then there would be a cell for every unique combination of the two, e.g., one cell each for (California, Tea), (California, Coffee), (Florida, Tea), (Florida, Coffee), etc. Each cell contains one value per measure of the data cube. So if product production and consumption information is needed, then each cell would contain two values, one for the number of products of each type consumed in that state, and one for the number of products of each type produced in that state. Dimensions within a data warehouse are often augmented with a hierarchical structure. If each dimension has a hierarchical structure, then the data warehouse is not a single data cube but rather a lattice of data cubes.

5.28 Exemplary Pattern Classification Techniques

This subsection describes various pattern classification techniques that can be used in the methods of the present invention in conjunction with the one or more subsets of genes identified in step 266, above, to classify subjects into a class of responders and nonresponders. In many instances, the classifier described in the following subsections are trained using the data obtained for a population in accordance with steps 202-210 of FIG. 2. The techniques described can be used instead of, or in conjunction with the techniques described in other sections, such as, clustering, nearest neighbor analysis, linear discriminant analysis, and principal component analysis.

5.28.1 Regression Models

In some embodiments, a regression model, preferably a logistic regression model is used. Such a regression model includes a coefficient for each of the classifier genes selected in step 266. In such embodiments, the coefficients for the regression model are computed using, for example, a maximum likelihood approach. In such a computation, the expression data measured for the classifier genes (e.g., RT-PCR data) is used. In particular embodiments, gene data from only two trait subgroups (responders and nonresponders) is used and the dependent variable is responsiveness or nonresponsiveness to a liver disease treatment regimen, or a therapy regimen for a disease that is treatable with an immunomodulatory disease therapy, in the subjects for gene express data is available (population of step 202).

In general, the multiple regression equation of interest can be written
Y=α+β₁X₁+β₂X₂+ . . . +βₖXₖ+ε
where Y, the dependent variable, is presence (when Y is positive) or absence (when Y is negative) of the biological feature (e.g., responder, nonresponder). This model says that the dependent variable Y depends on k explanatory variables (the measured characteristic values for the k candidate genes from subjects in the first and second trait subgroups in training data set 44), plus an error term that encompasses various unspecified omitted factors. In the above-identified model, the parameter β₁ gauges the effect of the first explanatory variable X₁ on the dependent variable Y, holding the other explanatory variables constant. Similarly, β₂ gives the effect of the explanatory variable X₂ on Y, holding the remaining explanatory variables constant. In general, in the multiple regression procedure, estimates for βᵢ are obtained by taking into account how uncontrolled changes in other variables influence Y.

Because the dependent variable data is binary, logistical regression can be used. The logistic regression model is a non-linear transformation of the linear regression. The logistic regression model is termed the “logit” model and can be expressed as
ln[p/(1−p)]=α+β₁X₁+β₂X₂+ . . . +βₖXₖ+ε or
[p/(1−p)]=exp^{α} exp^{β}^{₁}^{X}^{₁} exp^{β}^{₂}^{X}^{₂}× . . . ×exp^{β}^{ₖ}^{X}^{ₖ} exp^{ε}
here,

In is the natural logarithm, log^{exp}, where exp=2.71828,

p is the probability that the event Y occurs, p(Y=1),

p/(1−p) is the “odds ratio”,

ln[p/(1−p)] is the log odds ratio, or “logit”, and

all other components of the model are the same as the general regression equation described above. It will be appreciated by those of skill in the art that the term for α and ε can be folded into the same constant. Indeed, in preferred embodiments, a single term is used to represent α and ε. The “logistic” distribution is an S-shaped distribution function. The logit distribution constrains the estimated probabilities (p) to lie between 0 and 1.

In some embodiments of the present invention, the logistic regression model is fit by maximum likelihood estimation (MLE). In other words, the coefficients (e.g., α, β₁, β₂,) are determined by maximum likelihood. A likelihood is a conditional probability (e.g., P(Y|X), the probability of Y given X). The likelihood function (L) measures the probability of observing the particular set of dependent variable values (Y₁, Y₂, . . . , Yₙ) that occur in the sample data set. It is written as the probability of the product of the dependent variables:
L=Prob(Y₁*Y₂ * * * Yₙ)
The higher the likelihood function, the higher the probability of observing the Ys in the sample. MLE involves finding the coefficients (α,β₁,β₂,) that makes the log of the likelihood function (LL<0) as large as possible or −2 times the log of the likelihood function (−2LL) as small as possible. In MLE, some initial estimates of the parameters α,β₁,β₂, are made. Then the likelihood of the data given these parameter estimates is computed. The parameter estimates are improved the likelihood of the data is recalculated. This process is repeated until the parameter estimates do not change much (for example, a change of less than 0.01 or 0.001 in the probability). Examples of logistic regression and fitting logistic logistic regression models are found in Hastie, The Elements of Statistical Learning, Springer, N.Y., 2001, pp. 95-100.

5.28.2 Neural Networks

The present invention is not limited to the use of logistic regression models. In some embodiments, the expression data measured for the classifier genes of step 266 (e.g., RT-PCR data) across the population of step 202 can be used to train a neural network.

A neural network is a two-stage regression or classification model. A neural network has a layered structure that includes a layer of input units (and the bias) connected by a layer of weights to a layer of output units. For regression, the layer of output units typically includes just one output unit. However, neural networks can handle multiple quantitative responses in a seamless fashion.

In multilayer neural networks, there are input units (input layer), hidden units (hidden layer), and output units (output layer). There is, furthermore, a single bias unit that is connected to each unit other than the input units. Neural networks are described in Duda et al., 2001, Pattern Classification, Second Edition, John Wiley & Sons, Inc., New York; and Hastie et al., 2001, The Elements of Statistical Learning, Springer-Verlag, N.Y.

The basic approach to the use of neural networks is to start with an untrained network, present a training pattern to the input layer, and to pass signals through the net and determine the output at the output layer. These outputs are then compared to the target values; any difference corresponds to an error. This error or criterion function is some scalar function of the weights and is minimized when the network outputs match the desired outputs. Thus, the weights are adjusted to reduce this measure of error. For regression, this error can be sum-of-squared errors. For classification, this error can be either squared error or cross-entropy (deviation). See, e.g., Hastie et al., 2001, The Elements of Statistical Learning, Springer-Verlag, N.Y.

Three commonly used training protocols are stochastic, batch, and on-line. In stochastic training, patterns are chosen randomly from the training set and the network weights are updated for each pattern presentation. Multilayer nonlinear networks trained by gradient descent methods such as stochastic back-propagation perform a maximum-likelihood estimation of the weight values in the model defined by the network topology. In batch training, all patterns are presented to the network before learning takes place. Typically, in batch training, several passes are made through the training data. In online training, each pattern is presented once and only once to the net.

In some embodiments, consideration is given to starting values for weights. If the weights are near zero, then the operative part of the sigmoid commonly used in the hidden layer of a neural network (see, e.g., Hastie et al., 2001, The Elements of Statistical Learning, Springer-Verlag, N.Y.) is roughly linear, and hence the neural network collapses into an approximately linear model. In some embodiments, starting values for weights are chosen to be random values near zero. Hence the model starts out nearly linear, and becomes nonlinear as the weights increase. Individual units localize to directions and introduce nonlinearities where needed. Use of exact zero weights leads to zero derivatives and perfect symmetry, and the algorithm never moves. Alternatively, starting with large weights often leads to poor solutions.

Since the scaling of inputs determines the effective scaling of weights in the bottom layer, it can have a large effect on the quality of the final solution. Thus, in some embodiments, at the outset all expression values are standardized to have mean zero and a standard deviation of one. This ensures all inputs are treated equally in the regularization process, and allows one to choose a meaningful range for the random starting weights. With standardization inputs, it is typical to take random uniform weights over the range [−0.7, +0.7].

A recurrent problem in the use of three-layer networks is the optimal number of hidden units to use in the network. The number of inputs and outputs of a three-layer network are determined by the problem to be solved. In the present invention, the number of inputs for a given neural network will equal the number of classifier genes selected in the corresponding instance of step 266. The number of outputs for the neural network will typically be just one. If too many hidden units are used in a neural network, the network will have too many degrees of freedom and is trained too long, there is a danger that the network will overfit the data. If there are too few hidden units, the training set cannot be learned. Generally speaking, however, it is better to have too many hidden units than too few. With too few hidden units, the model might not have enough flexibility to capture the nonlinearities in the date; with too many hidden units, with too many hidden units, the extra weight can be shrunk towards zero if appropriate regularization or pruning, as described below, is used. In typical embodiments, the number of hidden units in somewhere in the range of 5 to 100, with the number increasing with the number of inputs and number of training cases.

One general approach to determining the number of hidden units to use is to apply a regularization approach. In the regularization approach, a new criterion function is constructed that depends not only on the classical training error, but also on classifier complexity. Specifically, the new criterion function penalizes highly complex models; searching for the minimum in this criterion is to balance error on the training set with error on the training set plus a regularization term, which expresses constraints or desirable properties of solutions:
J=Jₚₐₜ+λJ_{reg}.
The parameter λ is adjusted to impose the regularization more or less strongly. In other words, larger values for λ will tend to shrink weights towards zero: typically cross-validation with a validation set is used to estimate λ. This validation set can be obtained by setting aside a random subset of the population measured in step 202 of FIG. 2A. Other forms of penalty have been proposed, for example the weight elimination penalty (see, e.g., Hastie et al., 2001, The Elements of Statistical Learning, Springer-Verlag, N.Y.).

Another approach to determine the number of hidden units to use is to eliminate—prune—weights that are least needed. In one approach, the weights with the smallest magnitude are eliminated (set to zero). Such magnitude-based pruning can work, but is nonoptimal; sometimes weights with small magnitudes are important for learning and training data. In some embodiments, rather than using a magnitude-based pruning approach, Wald statistics are computed. The fundamental idea in Wald Statistics is that they can be used to estimate the importance of a hidden unit (weight) in a model. Then, hidden units having the least importance are eliminated (by setting their input and output weights to zero). Two algorithms in this regard are the Optimal Brain Damage (OBD) and the Optimal Brain Surgeon (OBS) algorithms that use second-order approximation to predict how the training error depends upon a weight, and eliminate the weight that leads to the smallest increase in training error.

Optimal Brain Damage and Optimal Brain Surgeon share the same basic approach of training a network to local minimum error at weight w*, and then pruning a weight that leads to the smallest increase in the training error. The predicted functional increase in the error for a change in full weight vector δw is:

$$δ J = {( \frac{\partial J}{\partial w} )}^{t} \cdot δ w + \frac{1}{2} δ {w}^{t} \cdot \frac{{\partial }^{2} J}{\partial {w}^{2}} \cdot δ w + O ( {‖ δ w ‖}^{3} )$$

where ∂²J/∂w² is the Hessian matrix. The first term vanishes because we are at a local minimum in error; third and higher order terms are ignored. The general solution for minimizing this function given the constraint of deleting one weight is:

$$δ w = - \frac{{w}_{q}}{{[ {H}^{- 1} ]}_{qq}} {H}^{- 1} \cdot {u}_{q} and$$

$${L}_{q} = \frac{1}{2} - \frac{{w}_{q}^{2}}{{[ {H}^{- 1} ]}_{qq}}$$

Here, u_{q} is the unit vector along the qth direction in weight space and L_{q} is approximation to the saliency of the weight q—the increase in training error if weight q is pruned and the other weights updated δw. These equations require the inverse of H. One method to calculate this inverse matrix is to start with a small value, H₀⁻¹=α⁻¹I, where α is a small parameter—effectively a weight constant. Next the matrix is updated with each pattern according to

$${H}_{m + 1}^{- 1} = {H}_{m}^{- 1} - \frac{{H}_{m}^{- 1} {X}_{m + 1} {X}_{m + 1}^{T} {H}_{m}^{- 1}}{\frac{n}{{a}_{m}} + {X}_{m + 1}^{T} {H}_{m}^{- 1} {X}_{m + 1}} \qquad Eqn . 1$$

where the subscripts correspond to the pattern being presented and αₘ decreases with m.

After the full training set has been presented, the inverse Hessian matrix is given by H⁻¹=Hₙ⁻¹. In algorithmic form, the Optimal Brain Surgeon method is:

begin initialize n_{H}, w, θ

train a reasonably large network to minimum error
do compute H⁻¹ by Eqn. 1

${q}^{*} ← arg {min}_{q} {w}_{q}^{2} / ( 2 {⌊ {H}^{- 1} ⌋}_{qq} ) ( saliency {L}_{q} )$
$w ← w - \frac{{w}_{{q}^{*}}}{{[ {H}^{- 1} ]}_{{q}^{*} {q}^{*}}} {H}^{- 1} {e}_{{q}^{*}} ( saliency {L}_{q} )$

until J(w) > θ

return w

end

The Optimal Brain Damage method is computationally simpler because the calculation of the inverse Hessian matrix in line 3 is particularly simple for a diagonal matrix. The above algorithm terminates when the error is greater than a criterion initialized to be θ. Another approach is to change line 6 to terminate when the change in J(w) due to elimination of a weight is greater than some criterion value.

In some embodiments, the back-propagation neural network (see, for example Abdi, 1994, “A nueral network primer”, J. Biol System. 2, 247-283) containing a single hidden layer of ten neurons (ten hidden units) found in EasyNN-Plus version 4.0 g software package (Neural Planner Software Inc.) is used. In one specific example, parameter values within the EasyNN-Plus program were set as follows: learning parameter=0.6, and momentum parameter=0.8. In some embodiments in which the EasyNN-Plus version 4.0 g software package is used, “outlier” samples are identified by performing twenty independently-seeded trials involving 20,000 learning cycles each.

5.28.3 Quadratic Discriminant Analysis

Quadratic discriminant analysis (QDA) takes the same input parameters and returns the same results as LDA. QDA uses quadratic equations, rather than linear equations, to produce results. LDA and QDA are interchangeable, and which to use is a matter of preference and/or availability of software to support the analysis. Logistic regression takes the same input parameters and returns the same results as LDA and QDA.

5.28.4 Support Vector Machines

In some embodiments of the present invention, support vector machines (SVMs) are used in step 268 of FIG. 2. SVMs are a relatively new type of learning algorithm. See, for example, Cristianini and Shawe-Taylor, 2000, An Introduction to Support Vector Machines, Cambridge University Press, Cambridge, Boser et al., 1992, “A training algorithm for optimal margin classifiers,” in Proceedings of the 5^{th} Annual ACM Workshop on Computational Learning Theory, ACM Press, Pittsburgh, Pa., pp. 142-152; Vapnik, 1998, Statistical Learning Theory, Wiley, N.Y. When used for classification, SVMs separate a given set of binary labeled data training data with a hyper-plane that is maximally distance from them. For cases in which no linear separation is possible, SVMs can work in combination with the technique of ‘kernels’, which automatically realizes a non-linear mapping to a feature space. The hyper-plane found by the SVM in feature space corresponds to a non-linear decision boundary in the input space.

In one approach, when a SVM is used, the gene expression data from step 204 and/or step 210 is standardized to have mean zero and unit variance and the members of the training population from step 202 are randomly divided into a training set and a test set. For example, in one embodiment, two thirds of the members of the training population are placed in the training set and one third of the members of the training population are placed in the test set. The expression values across the training set for the combination of genes selected in the last instance of step 266 is used to train the SVM. For more information on SVMs, see Duda, Pattern Classification, Second Edition, 2001, John Wiley & Sons, Inc.; Hastie, 2001, The Elements of Statistical Learning, Springer, N.Y.; and Furey et al., 2000, Bioinformatics 16, 906-914.

5.28.5 Decision Trees

In some embodiments of the present invention, decision trees are implemented in step 268. Decision tree algorithms belong to the class of supervised learning algorithms. The aim of a decision tree is to induce a classifier (a tree) from real-world example data. This tree can be used to classify unseen examples which have not been used to derive the decision tree.

A decision tree is derived from training data. An example contains values for the different attributes and what class the example belongs. In the present invention, the training data is the set of genes selected in the last instance of step 268 across the training population.

The following algorithm describes a decision tree derivation:

```
Tree(Examples,Class,Attributes)
Create a root node
If all Examples have the same Class value, give the root this label
Else if Attributes is empty label the root according to the most common
value
Else begin
  Calculate the information gain for each attribute
  Select the attribute A with highest information gain and make this the
  root attribute
    For each possible value, v, of this attribute
      Add a new branch below the root, corresponding to A = v
      Let Examples(v) be those examples with A = v
      If Examples(v) is empty, make the new branch a leaf node
      labeled with the most common value among Examples
      Else let the new branch be the tree created by
      Tree(Examples(v),Class,Attributes - {A})
end
```

A more detailed description of the calculation of information gain will now be described. If the possible classes vᵢ of the examples have probabilities P(vᵢ) then the information content I of the actual answer is given by:

$$I ( P ( {v}_{1} ) , … , P ( {v}_{n} ) ) = {\sum }_{i = 1}^{n} - P ( {v}_{i} ) {log}_{2} P ( {v}_{i} )$$

The I-value shows how much information we need in order to be able to describe the outcome of a classification for the specific dataset used. Supposing that the dataset contains p positive (e.g. cancer) and n negative (e.g. healthy) examples (e.g. individuals), the information contained in a correct answer is:

$$I ( \frac{p}{p + n} , \frac{n}{p \div n} ) = - \frac{p}{p + n} {log}_{2} \frac{p}{p + n} - \frac{n}{p + n} {log}_{2} \frac{n}{p + n}$$

where log₂ is the logarithm using base two. By testing single attributes the amount of information needed to make a correct classification can be reduced. The remainder for a specific attribute A (e.g. a gene) shows how much the information that is needed can be reduced.

$$Remainder ( A ) = {\sum }_{i = 1}^{v} \frac{{p}_{i} + {n}_{i}}{p + n} I ( \frac{{p}_{i}}{{p}_{i} + {n}_{i}} , \frac{{n}_{i}}{{p}_{i} \div {n}_{i}} )$$

“v” is the number of unique attribute values for attribute A in a certain dataset, “i” is a certain attribute value, “pᵢ” is the number of examples for attribute A where the classification is positive (e.g. cancer), “nᵢ” is the number of examples for attribute A where the classification is negative (e.g. healthy).

The information gain of a specific attribute A is calculated as the difference between the information content for the classes and the remainder of attribute A:

$$Gain ( A ) = I ( \frac{p}{p + n} , \frac{n}{p \div n} ) - Remainder ( A )$$

The information gain is used to evaluate how important the different attributes are for the classification (how well they split up the examples), and the attribute with the highest information.

In general there are a number of different decision tree algorithms, many of which are described in Duda, Pattern Classification, Second Edition, 2001, John Wiley & Sons, Inc. Decision tree algorithms often require consideration of feature processing, impurity measure, stopping criterion, and pruning. Specific decision tree algorithms include, cut are not limited to classification and regression trees (CART), multivariate decision trees, ID3, and C4.5.

In one approach, when a decision tree is used, the gene expression data from step 204 and/or step 210 is standardized to have mean zero and unit variance and the members of the population from step 202 are randomly divided into a training set and a test set. For example, in one embodiment, two thirds of the members of the training population are placed in the training set and one third of the members of the training population are placed in the test set. The expression values, across the training set, for the combination of genes selected in the last instance of step 266 is used to construct the decision tree. Then, the ability for the decision tree to correctly classify members in the test set is determined. In some embodiments, this computation is performed several times for the combination of genes selected in the last instance of step 266. In each iteration of the computation, the members of the training population are randomly assigned to the training set and the test set. Then, the quality of the combination of genes is taken as the average of each such iteration of the decision tree computation.

5.28.6 Evolutionary Methods

Inspired by the process of biological evolution, evolutionary methods of classifier design employ a stochastic search for an optimal classifier. In broad overview, such methods create several classifiers—a population—from the set of genes selected in the last instance of step 266. Each classifier varies somewhat from the other. Next, the classifiers are scored on expression data across the training population. In keeping with the analogy with biological evolution, the resulting (scalar) score is sometimes called the fitness. The classifiers are ranked according to their score and the best classifiers are retained (some portion of the total population of classifiers). Again, in keeping with biological terminology, this is called survival of the fittest. The classifiers are stochastically altered in the next generation—the children or offspring. Some offspring classifiers will have higher scores than their parent in the previous generation, some will have lower scores. The overall process is then repeated for the subsequent generation: The classifiers are scored and the best ones are retained, randomly altered to give yet another generation, and so on. In part, because of the ranking, each generation has, on average, a slightly higher score than the previous one. The process is halted when the single best classifier in a generation has a score that exceeds a desired criterion value. More information on evolutionary methods is found in, for example, Duda, Pattern Classification, Second Edition, 2001, John Wiley & Sons, Inc.

5.28.7 Bagging, Boosting, and the Random Subspace Method

Bagging, boosting and the random subspace method are combining techniques that can be used to improve weak classifiers. These techniques are designed for, and usually applied to, decision trees. In addition, Skurichina and Duin provide evidence to suggest that such techniques can also be useful in linear discriminant analysis.

In bagging, one samples the training set, generating random independent bootstrap replicates, constructs the classifier on each of these, and aggregates them by a simple majority vote in the final decision rule. See, for example, Breiman, 1996, Machine Learning 24, 123-140; and Efron & Tibshirani, An Introduction to Boostrap, Chapman & Hall, New York, 1993.

In boosting, classifiers are constructed on weighted versions of the training set, which are dependent on previous classification results. Initially, all objects have equal weights, and the first classifier is constructed on this data set. Then, weights are changed according to the performance of the classifier. Erroneously classified objects (molecular markers in the data set) get larger weights, and the next classifier is boosted on the reweighted training set. In this way, a sequence of training sets and classifiers is obtained, which is then combined by simple majority voting or by weighted majority voting in the final decision. See, for example, Freund & Schapire, “Experiments with a new boosting algorithm,” Proceedings 13^{th} International Conference on Machine Learning, 1996, 148-156.

To illustrate boosting, consider the case where there are two phenotypic traits exhibited by the population under study, responders and nonresponders. Given a vector of predictor gene X selected in step 266, a classifier G(X) produces a prediction taking one of the type values in the two value set: {extreme phenotype 1, extreme phenotype 2}. The error rate on the training sample is

$$\overline{err} = \frac{1}{N} {\sum }_{i = 1}^{N} I ( {y}_{i} \neq G ( {x}_{i} ) )$$

where N is the number of organisms in the training set (the sum total of the organisms that are either responders or nonresponders). For example, if there are 49 responders and 72 nonresponders under study, N is 121.

A weak classifier is one whose error rate is only slightly better than random guessing. In the boosting algorithm, the weak classification algorithm is repeatedly applied to modified versions of the data, thereby producing a sequence of weak classifiers Gₘ(x), m,=1, 2, . . . , M. The predictions from all of the classifiers in this sequence are then combined through a weighted majority vote to produce the final prediction:

$$G ( x ) = sign ( {\sum }_{m = 1}^{M} {α}_{m} {G}_{m} ( x ) )$$

Here α₁, α₂, . . . , α_{M} are computed by the boosting algorithm and their purpose is to weigh the contribution of each respective Gₘ(x). Their effect is to give higher influence to the more accurate classifiers in the sequence.

The data modifications at each boosting step consist of applying weights w₁, w₂, . . . , wₙ to each of the training observations (xᵢ, yᵢ), i=1, 2, . . . , N. Initially all the weights are set to wᵢ=1/N, so that the first step simply trains the classifier on the data in the usual manner. For each successive iteration m=2, 3, . . . , M the observation weights are individually modified and the classification algorithm is reapplied to the weighted observations. At stem m, those observations that were misclassified by the classifier Gₘ₋₁(x) induced at the previous step have their weights increased, whereas the weights are decreased for those that were classified correctly. Thus as iterations proceed, observations that are difficult to correctly classify receive ever-increasing influence. Each successive classifier is thereby forced to concentrate on those training observations that are missed by previous ones in the sequence.

The exemplary boosting algorithm is summarized as follows:

1. Initialize the observation weights wᵢ=1/N, i=1, 2, . . . , N.

2. For m=1 to M:
(a) Fit a classifier Gₘ(x) to the training set using weights wᵢ.
(b) Compute

$${err}_{m} = \frac{{\sum }_{i = 1}^{N} {w}_{i} I ( {y}_{i} \neq {G}_{m} ( {x}_{i} ) )}{{\sum }_{i = 1}^{N} {w}_{i}}$$
(c) Compute αₘ=log((1-errₘ)/errₘ).
(d) Set wᵢ←wᵢ·exp[αₘ·I(yᵢ≠Gₘ(xᵢ))],i=1, 2, . . . , N.

3. Output

$$G ( x ) = sign ⌊ {\sum }_{m = 1}^{M} {α}_{m} {G}_{m} ( x ) ⌋$$

In the algorithm, the current classifier Gₘ(x) is induced on the weighted observations at line 2a. The resulting weighted error rate is computed at line 2b. Line 2c calculates the weight am given to Gₘ(x) in producing the final classifier G(x) (line 3). The individual weights of each of the observations are updated for the next iteration at line 2d. Observations misclassified by Gₘ(x) have their weights scaled by a factor exp(αₘ), increasing their relative influence for inducing the next classifier Gₘ₊₁(x) in the sequence. In some embodiments, modifications of the Freund and Schapire, 1997, Journal of Computer and System Sciences 55, pp. 119-139, boosting method are used. See, for example, Hasti et al., The Elements of Statistical Learning, 2001, Springer, N.Y., Chapter 10. In some embodiments, boosting or adaptive boosting methods are used.

In some embodiments, modifications of Freund and Schapire, 1997, Journal of Computer and System Sciences 55, pp. 119-139, are used. For example, in some embodiments, feature preselection is performed using a technique such as the nonparametric scoring methods of Park et al., 2002, Pac. Symp. Biocomput. 6, 52-63. Feature preselection is a form of dimensionality reduction in which the genes that discriminate between classifications the best are selected for use in the classifier. Then, the LogitBoost procedure introduced by Friedman et al., 2000, Ann Stat 28, 337-407 is used rather than the boosting procedure of Freund and Schapire. In some embodiments, the boosting and other classification methods of Ben-Dor et al., 2000, Journal of Computational Biology 7, 559-583 are used in the present invention. In some embodiments, the boosting and other classification methods of Freund and Schapire, 1997, Journal of Computer and System Sciences 55, 119-139, are used.

In the random subspace method, classifiers are constructed in random subspaces of the data feature space. These classifiers are usually combined by simple majority voting in the final decision rule. See, for example, Ho, “The Random subspace method for constructing decision forests,” IEEE Trans Pattern Analysis and Machine Intelligence, 1998; 20(8): 832 844.

5.28.8 Other Algorithms

The pattern classification and statistical techniques described above are merely examples of the types of models that can be used to construct a model in step 266 and 268 of FIG. 2. Moreover, combinations of the techniques described above can be used. Some combinations, such as the use of the combination of decision trees and boosting, have been described. However, many other combinations are possible. In addition, in other techniques in the art such as Projection Pursuit and Weighted Voting can be used to construct models in instances of steps 266 and 268.

### 6 EXAMPLES

Examples of the use of the methods of the present invention have been provided in Section 5, above. What follows is additional experimental detail.

Patients with Chronic HCV and Biopsy Specimens. Thirty-one (31) patients with chronic HCV (23 genotype 1, 4 genotype 2, 3 genotype 3, and 1 genotype 6) were seen, treated and followed at University Health Network in the period from October 2001 through May 2004. All treatment-naive patients considering treatment with IFN/rib underwent percutaneous liver biopsy. Baseline viral load determinations were also performed prior to initiation of treatment. The treatment consisted of PegIFN α 2a/2b 80 μg weekly sc and oral ribavirin 800-1200 mg daily (depending on genotype and weight) for 24 weeks (genotype 2 and 3) or 48 weeks (genotype 1 and 6). Quantitative HCV RNA was determined at completion of therapy and six months after.

A patient was designated as NR if the HCV RNA was detectable at the end of therapy, as a relapser HCV RNA was undetectable at the end of treatment but subsequently became detectable at the 6 mo follow-up, and as achieving a sustained viral response (SVR) if both end-of-treatment and 6 months follow-up HCV RNA was undetectable. Compliance was excellent: a single patient discontinued treatment for personal reasons after 16 weeks of treatment. For the purposes of this study, patients were designated as “responders” (R) if the initial HCV RNA was negative; overall, there were 3 relapsers and 13 SVR patients included in the R patient group, and 15 NR patients. Normal liver tissue was biopsied as the first step of 20 right hepatectomy operations performed on living transplant donors. For both HCV-infected and normal liver, portions of each biopsy were promptly immersed in RNAlater (Qiagen), left at −4° C. for 12 hours and then stored at −20° C. pending RNA extraction (see below). All patients gave informed consent for the research protocol, which was approved by the hospital and university Research Ethics Board. All patients were tested for HCV infection; none were positive.

RNA Extraction and Amplification. RNA was extracted from liver biopsies as previously described using Trizol (Invitrogen) (Chen 2003). For amplification, 2 μg of total RNA from each biopsy or from Universal Human Reference RNA (Stratagene) was amplified using the MessageAmp aRNA kit (Ambion), following the manufacturer's instructions. In control experiments we determined that the gene expression profiles from amplified RNA were highly correlated to those developed from non-amplified RNA, with a correlation coefficient of at least 0.85 (data not shown).

cDNA Microarrays. Human single spot (SS-H19K6) microarray chips comprising 19,000 human gene or EST clones were purchased from the UHN Microarray Center (University Health Network, Toronto, Ontario, Canada). Detailed information on the array layout and composition is available at http://www.microarrays.ca/support/glists.html. For each array experiment, 5 μg of aRNA from a given liver biopsy was compared to 5 μg of aRNA from the Universal Human Reference RNA. After reverse transcription with 400U of SuperScript II (Invitrogen), liver cDNA was labeled with Cy5 and reference RNA with Cy3 as previously described (Chen 2003). Hybridization was performed overnight at 37° C. in a humid hybridization chamber containing DIGEasy hybridization buffer (Roche). After 3 washes in 0.1×SSC, arrays were read with a GenePix 4000A (Axon Instruments) laser scanner and quantified with GenePix Pro software (Axon Instruments).

Real-Time PCR. Two-step real-time PCR was performed after reverse transcription (400U SuperScript) of 5 μg of aRNA with 5 μg pd (N)6 Random Hexamer primer (Amersham) in a total volume of 40 μl. A microliter (1 μl) of the reverse transcribed cDNA was then used as a template for real-time PCR quantification, using the QuantiTect SYBR PCR Kit (Qiagen) with 1 μg forward and 1 μg reverse gene-specific primers. Real-time PCR was performed using the DNA Engine Opticon 2 cycler (MJ Research) under the following conditions: 10 min 94° C. activation, 45 (45 sec) cycles denaturation 94° C., 45 sec 56° C. annealing, 1 min 72° C. extension. The relative amounts of mRNA across different samples were compared by normalizing to β-actin. The primers were used for real-time PCR are listed in Table 7.

Statistics. Comparisons between two groups of continuous variables were generally performed using the two-sample Welch t-statistic with the muittest package, which includes an estimation of adjusted p-values by permutation (Dudoit). Where appropriate chi-square analyses were performed.

Clustering and Classifier Analyses. Unsupervised hierachicial clustering and unsupervised principal components analyses were performed using the R mva package (Anderberg 1973, Gordon 1999). Nearest neighbour classifier analyses were performed using the R class package, and linear discriminant analyses were performed with the R MASS package (See, Ripley, 1996, Pattern recognition and neural networks, Cambridge University Press; and Venables and Ripley, 2002, Modern Applied Statistics with S., 4^{th} ed., Springer, each of which is hereby incorporated by reference in its entirety).

### 7 COMPUTER SYSTEMS AND COMPUTER PROGRAM PRODUCTS

The present invention can be implemented as a computer program product that comprises a computer program mechanism embedded in a computer readable storage medium. For instance, the computer program product could contain the program modules shown in FIG. 1. These program modules may be stored on a CD-ROM, DVD, magnetic disk storage product, or any other computer readable data or program storage product. The software modules in the computer program product can also be distributed electronically, via the Internet or otherwise, by transmission of a computer data signal (in which the software modules are embedded) on a carrier wave.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

### 8. REFERENCES CITED

All references cited herein are incorporated herein by reference in their entirety and for all purposes to the same extent as if each individual publication or patent or patent application was specifically and individually indicated to be incorporated by reference in its entirety for all purposes.

TABLE 1

| | | GenBank | | | | | | |
|---|---|---|---|---|---|---|---|---|
| cloneID | Gene name | Accession No. | ratio(nr/svr) | p. value | ratio(nr/normal) | p. value | ratio(svr/normal) | p. value |
| 240733 | metallothionein 1G | NM_005950 | 1.675889 | 0.0427 | 1.037544 | 0.8048 | 0.619101 | 0.0533 |
| 3930678 | eukaryotic translation | BC018857 | 0.65484 | 0.0032 | 0.751038 | 0.0009 | 1.146902 | 0.7341 |
| | elongation factor 1 | | | | | | | |
| | gamma | | | | | | | |
| 127270 | activating transcription | BC005174 | 1.559038 | 0.0046 | 0.963254 | 0.6984 | 0.617851 | 0.0024 |
| | factor 5 | | | | | | | |
| 380876 | serine (or cysteine) | NM_199093 | 1.508247 | 0.0096 | 1.27835 | 0.0109 | 0.847573 | 0.4799 |
| | proteinase inhibitor, | | | | | | | |
| | clade G (C1 inhibitor), | | | | | | | |
| | member 1, | | | | | | | |
| | (angloedema, | | | | | | | |
| | hereditary) | | | | | | | |
| 52905 | dual specificity | BC022463 | 1.560741 | 0.0003 | 0.589062 | 0.002 | 0.377425 | 0.0001 |
| | phosphatase 1 | | | | | | | |
| 108690 | similar to mitochondrial | XM_370619 | 1.658886 | 0.0001 | 2.047791 | 0.0001 | 1.234438 | 0.0246 |
| | carrier protein MGC4399 | | | | | | | |
| 754047 | cyclin-dependent | | 1.877716 | 0.0004 | 2.344404 | 0.0001 | 1.24854 | 0.0278 |
| | kinase (CDC2-like) 11 | | | | | | | |
| 325130 | myxovirus (influenza | BC032602 | 1.578876 | 0.0013 | 1.978349 | 0.0001 | 1.253011 | 0.0394 |
| | virus) resistance 1, | | | | | | | |
| | interferon-inducible | | | | | | | |
| | protein p78 (mouse) | | | | | | | |
| 487534 | leucine | BC065564 | 1.556274 | 0.0003 | 2.103316 | 0.0001 | 1.351507 | 0.0067 |
| | aminopeptidase 3 | | | | | | | |
| 229295 | USP18 | NM_017414 | 1.519708 | 0.0001 | 1.721741 | 0.0001 | 1.132942 | 0.0791 |
| 182425 | regucalcin gene | NM_033127 | 1.833543 | 0.0004 | 1.957702 | 0.0001 | 1.067716 | 0.6299 |
| | promotor region related | | | | | | | |
| | protein | | | | | | | |
| 4734251 | vitronectin (serum | BC005046 | 0.642122 | 0.0366 | 0.966163 | 0.845 | 1.504643 | 0.0184 |
| | spreading factor, | | | | | | | |
| | somatomedin B, | | | | | | | |
| | complement S-protein) | | | | | | | |
| 207215 | major histocompatibility | NG_002397 | 1.549965 | 0.0196 | 2.164933 | 0.0001 | 1.396762 | 0.0452 |
| | complex, class I, B | | | | | | | |
| 324912 | interferon, alpha- | NM_002038 | 2.832964 | 0.0001 | 4.719385 | 0.0001 | 1.665882 | 0.0002 |
| | inducible protein (clone | | | | | | | |
| | IFI-6-16) | | | | | | | |
| 754047 | cyclin-dependent | | 1.877716 | 0.0004 | 2.344404 | 0.0001 | 1.24854 | 0.0278 |
| | kinase (CDC2-like) 11 | | | | | | | |
| 47193 | profilin 2 | BC018049 | 0.649723 | 0.0051 | 0.475485 | 0.0001 | 0.731827 | 0.0228 |
| 37942 | hect domain and | NM_016323 | 2.153414 | 0.0001 | 2.553371 | 0.0001 | 1.185732 | 0.0777 |
| | RLD 5 | | | | | | | |
| 5474956 | ribosomal protein, large | NG_004261 | 2.534145 | 0.0001 | 3.702642 | 0.0001 | 1.461101 | 0.0002 |
| | P2 | | | | | | | |
| 152802 | phospholipase A2, | NM_000300 | 1.920476 | 0.0292 | 3.160705 | 0.0002 | 1.645792 | 0.0975 |
| | group IIA (platelets, | | | | | | | |
| | synovial fluid) | | | | | | | |
| 207669 | D11\|gp1e-like | BC022784 | 1.507536 | 0.0014 | 1.384758 | 0.0094 | 0.918557 | 0.1351 |
| 282007 | Transcribed sequence | P00722 | 1.615911 | 0.0157 | 1.528658 | 0.0086 | 0.946004 | 0.7554 |
| | with strong similarity to | | | | | | | |
| | protein sp: P00722 (E. coli) | | | | | | | |
| | BGAL_ECOLI Beta- | | | | | | | |
| | galactosidase | | | | | | | |
| 324284 | OAS3 | NM_006187 | 2.537255 | 0.0001 | 3.424041 | 0.0001 | 1.349506 | 0.005 |
| 299081 | ribosomal protein, large | NG_004261 | 2.504322 | 0.0001 | 3.26513 | 0.0001 | 1.303798 | 0.0089 |
| | P2 | | | | | | | |
| 485859 | IFRG28 | NM_022147 | 1.800608 | 0.0001 | 2.38847 | 0.0001 | 1.32648 | 0.0083 |
| 5745506 | phosphoinositide-3- | NM_152309 | 1.603736 | 0.005 | 1.663109 | 0.0022 | 1.037021 | 0.8283 |
| | kinase adaptor protein 1 | | | | | | | |
| 149319 | interferon, alpha- | BC009507 | 4.369767 | 0.0001 | 9.694266 | 0.0001 | 2.218486 | 0.0001 |
| | inducible protein (clone | | | | | | | |
| | IFI-15K) | | | | | | | |
| 136508 | OAS2 | BC023637 | 3.800511 | 0.0001 | 6.583685 | 0.0001 | 1.732316 | 0.0009 |
| 4183205 | Fc fragment of IgG | NM_003890 | 1.516997 | 0.0002 | 1.330431 | 0.0025 | 0.877016 | 0.0219 |
| | binding protein | | | | | | | |
| 4338699 | H326 | BC013107 | 1.580091 | 0.0256 | 1.430462 | 0.0512 | 0.905303 | 0.2926 |
| 5745506 | phosphoinositide-3- | NM_152309 | 1.603736 | 0.005 | 1.663109 | 0.0022 | 1.037021 | 0.8283 |
| | kinase adaptor protein 1 | | | | | | | |
| 325364 | interferon-induced | BC007091 | 2.14422 | 0.0001 | 2.834748 | 0.0001 | 1.322041 | 0.0127 |
| | protein with | | | | | | | |
| | tetratricopeptide repeats 1 | | | | | | | |
| 149009 | EST CLONE | | 0.665143 | 0.0001 | 0.768876 | 0.0997 | 1.155956 | 0.4367 |
| 176650 | RPS28 | BC070218 | 1.75452 | 0.0004 | 2.375134 | 0.0001 | 1.353723 | 0.0002 |
| 3894126 | HNRPAB | NM_031266 | 0.613536 | 0.002 | 0.54538 | 0.0004 | 0.888913 | 0.3821 |
| 120600 | viperin | NM_080657 | 1.822573 | 0.0002 | 1.784057 | 0.0001 | 0.978867 | 0.8031 |
| 491243 | chemokine (C—X—C | BC010954 | 1.588481 | 0.0231 | 4.505062 | 0.0001 | 2.836082 | 0.0001 |
| | motif) ligand 10 | | | | | | | |
| 324259 | hypothetical protein | | 0.659259 | 0.0057 | 0.84988 | 0.273 | 1.289144 | 0.1424 |
| 502921 | B aggressive | BC039580 | 1.663659 | 0.0032 | 3.138225 | 0.0001 | 1.886339 | 0.0012 |
| | lymphoma gene | AF307339 | | | | | | |
| | | (short isoform) | | | | | | |
| | | AF307338 | | | | | | |
| | | (long Isoform) | | | | | | |
| 231624 | syntaxin binding protein | NM_139244 | 0.654632 | 0.0034 | 0.959928 | 0.7156 | 1.466362 | 0.0126 |
| | 5 (tomosyn) | | | | | | | |
| 324744 | polymerase I and | BC073759 | 1.615924 | 0.0113 | 2.061478 | 0.0001 | 1.275727 | 0.0649 |
| | transcript release factor | | | | | | | |

TABLE 2

| cloneID | genename | ratio(nr/svr) | p. value | ratio(nr/normal) | p. value | ratio(svr/normal) | p. value |
|---|---|---|---|---|---|---|---|
| 149319 | interferon, alpha-inducible protein (clone IFI-15K) | 4.369767 | 0.0001 | 9.694266 | 0.0001 | 2.218486 | 0.0001 |
| 136508 | OAS2 | 3.800511 | 0.0001 | 6.583685 | 0.0001 | 1.732316 | 0.0009 |
| 324912 | interferon, alpha-inducible protein (clone IFI-6- | 2.832964 | 0.0001 | 4.719385 | 0.0001 | 1.665882 | 0.0002 |
| | 16) | | | | | | |
| 5474956 | ribosomal protein, large P2 | 2.534145 | 0.0001 | 3.702642 | 0.0001 | 1.461101 | 0.0002 |
| 324284 | 2′-5′-oligoadenylate synthetase 3, 100 kDa | 2.537255 | 0.0001 | 3.424041 | 0.0001 | 1.349506 | 0.005 |
| 299081 | ribosomal protein, large P2 | 2.504322 | 0.0001 | 3.26513 | 0.0001 | 1.303798 | 0.0089 |
| 502921 | B aggressive lymphoma gene | 1.663659 | 0.0032 | 3.138225 | 0.0001 | 1.886339 | 0.0012 |
| 325364 | interferon-induced protein with tetratricopeptide | 2.14422 | 0.0001 | 2.834748 | 0.0001 | 1.322041 | 0.0127 |
| | repeats 1 | | | | | | |
| 37942 | cyclin-E binding protein 1 | 2.153414 | 0.0001 | 2.553371 | 0.0001 | 1.185732 | 0.0777 |
| 485859 | IFRG28 | 1.800608 | 0.0001 | 2.38847 | 0.0001 | 1.32648 | 0.0083 |
| 176650 | RPS28 | 1.75452 | 0.0004 | 2.375134 | 0.0001 | 1.353723 | 0.0002 |
| 754047 | cyclin-dependent kinase (CDC2-like) 11 | 1.877716 | 0.0004 | 2.344404 | 0.0001 | 1.24854 | 0.0278 |
| 754047 | cyclin-dependent kinase (CDC2-like) 11 | 1.877716 | 0.0004 | 2.344404 | 0.0001 | 1.24854 | 0.0278 |
| 487534 | leucine aminopeptidase 3 | 1.556274 | 0.0003 | 2.103316 | 0.0001 | 1.351507 | 0.0067 |
| 108690 | NA (EST) | 1.658886 | 0.0001 | 2.047791 | 0.0001 | 1.234438 | 0.0246 |
| 325130 | myxovirus (influenza virus) resistance 1, | 1.578876 | 0.0013 | 1.978349 | 0.0001 | 1.253011 | 0.0394 |
| | interferon-inducible protein p78 (mouse) | | | | | | |
| 182425 | regucalcin gene promotor region related protein | 1.833543 | 0.0004 | 1.957702 | 0.0001 | 1.067716 | 0.6299 |
| 120600 | viperin | 1.822573 | 0.0002 | 1.784057 | 0.0001 | 0.978867 | 0.8031 |
| 229295 | USP18 | 1.519708 | 0.0001 | 1.721741 | 0.0001 | 1.132942 | 0.0791 |
| 5745506 | phosphoinositide-3-kinase adaptor protein 1 | 1.603736 | 0.005 | 1.663109 | 0.0022 | 1.037021 | 0.8283 |
| 5745506 | phosphoinositide-3-kinase adaptor protein 1 | 1.603736 | 0.005 | 1.663109 | 0.0022 | 1.037021 | 0.8283 |
| 207669 | D11\|gp1e-like | 1.507536 | 0.0014 | 1.384758 | 0.0094 | 0.918557 | 0.1351 |
| 4183205 | Fc fragment of IgG binding protein | 1.516997 | 0.0002 | 1.330431 | 0.0025 | 0.877016 | 0.0219 |
| 380876 | serine (or cysteine) proteinase inhibitor, | 1.508247 | 0.0096 | 1.27835 | 0.0109 | 0.847573 | 0.4799 |
| | clade G (C1 inhibitor), member 1, | | | | | | |
| | (angloedema, hereditary) | | | | | | |
| 127270 | activating transcription factor 5 | 1.559038 | 0.0046 | 0.963254 | 0.6984 | 0.617851 | 0.0024 |
| 231624 | syntaxin binding protein 5 (tomosyn) | 0.654632 | 0.0034 | 0.959928 | 0.7156 | 1.466362 | 0.0126 |
| 324259 | NA (EST) | 0.659259 | 0.0057 | 0.84988 | 0.273 | 1.289144 | 0.1424 |
| 149009 | NA (EST) | 0.665143 | 0.0001 | 0.768876 | 0.0997 | 1.155956 | 0.4367 |
| 3930678 | eukaryotic translation elongation factor 1 gamma | 0.65484 | 0.0032 | 0.751038 | 0.0009 | 1.146902 | 0.7341 |
| 52905 | dual specificity phosphatase 1 | 1.560741 | 0.0003 | 0.589062 | 0.002 | 0.377425 | 0.0001 |
| 3894126 | HNRPAB | 0.613536 | 0.002 | 0.54538 | 0.0004 | 0.888913 | 0.3821 |
| 47193 | profilin 2 | 0.649723 | 0.0051 | 0.475485 | 0.0001 | 0.731827 | 0.0228 |

TABLE 3

| cloneID | genename | ratio(nr/svr) | p. value | ratio(nr/normal) | p. value | ratio(svr/normal) | p. value |
|---|---|---|---|---|---|---|---|
| 149319 | interferon, alpha-inducible protein (clone IFI-15K) | 3.617515 | 0.0002 | 9.694266 | 0.0001 | 2.679813 | 0.0003 |
| 136508 | OAS2 | 3.412276 | 0.0007 | 6.583685 | 0.0001 | 1.929412 | 0.0346 |
| 324912 | interferon, alpha-inducible protein (clone IFI-6-16) | 2.719986 | 0.002 | 4.719385 | 0.0001 | 1.735077 | 0.0302 |
| 5474956 | ribosomal protein, large P2 | 2.269657 | 0.001 | 3.702642 | 0.0001 | 1.631367 | 0.0025 |
| 324284 | 2′-5′-oligoadenylate synthetase 3, 100 kDa | 2.37313 | 0.0004 | 3.424041 | 0.0001 | 1.442838 | 0.0056 |
| 299081 | ribosomal protein, large P2 | 2.266478 | 0.0033 | 3.26513 | 0.0001 | 1.440618 | 0.0417 |
| 325364 | interferon-induced protein with | 2.006886 | 0.0024 | 2.834748 | 0.0001 | 1.412511 | 0.0939 |
| | tetratricopeptide repeats 1 | | | | | | |
| 37942 | cyclin-E binding protein 1 | 1.988469 | 0.0011 | 2.553371 | 0.0001 | 1.284089 | 0.1019 |
| 754047 | cyclin-dependent kinase (CDC2-like) 11 | 1.931104 | 0.0056 | 2.344404 | 0.0001 | 1.214023 | 0.1951 |
| 754047 | cyclin-dependent kinase (CDC2-like) 11 | 1.931104 | 0.0056 | 2.344404 | 0.0001 | 1.214023 | 0.1951 |
| 108690 | NA | 1.613572 | 0.0015 | 2.047791 | 0.0001 | 1.269104 | 0.0615 |
| 120600 | viperin | 1.876392 | 0.0035 | 1.784057 | 0.0001 | 0.950791 | 0.711 |
| 487662 | microtubule-associated protein 6 | 1.570995 | 0.0014 | 1.467075 | 0.0003 | 0.933851 | 0.384 |
| 207669 | D11\|gp1e-like | 1.524648 | 0.0015 | 1.384758 | 0.0089 | 0.908248 | 0.0838 |
| 4183205 | Fc fragment of IgG binding protein | 1.594482 | 0.0005 | 1.330431 | 0.0018 | 0.834397 | 0.0217 |
| 743337 | protein phosphatase 2 (formerly 2A), | 1.507574 | 0.0005 | 0.979971 | 0.8397 | 0.650032 | 0.0002 |
| | regulatory subunit A (PR 65), beta isoform | | | | | | |
| 298686 | calcium channel, voltage-dependent, beta 3 subunit | 0.623182 | 0.0063 | 0.826029 | 0.0005 | 1.325501 | 0.0957 |
| 415965 | cleavage and polyadenylation specific | 0.570532 | 0.0078 | 0.767524 | 0.003 | 1.345278 | 0.1203 |
| | factor 1, 160 kDa | | | | | | |
| 47193 | profilin 2 | 0.55155 | 0.0018 | 0.475485 | 0.0001 | 0.862089 | 0.2972 |

TABLE 4

| Variable | NR | R | p |
|---|---|---|---|
| Number | 15 | 16 | |
| Age (yrs) | 46.4 ± 14 | 48.3 ± 10 | 0.6896 |
| Sex (# male) | 7/15 | 13/16 | 0.0443* |
| Genotype 1 | 15/15 | 8/16 | 0.0015* |
| Viral load | 2.4 × 10⁶ ± 3.7 × 10⁶ | 3.8 × 10⁶ ± 4.3 × 10⁶ | 0.3529 |
| (IU/ml) | | | |
| Activity | 1.63 ± 0.44 | 1.81 ± 0.51 | 0.3049 |
| Fibrosis | 2.50 ± 0.84 | 2.65 ± 0.94 | 0.6305 |
| Completed Rx | 14/15 | 16/16 | NS |
| course | | | |
| PegIFN/rib | 14/15 | 12/16 | NS |
| dose >80% | | | |
| Alcohol (10 | 2/12 | 2/13 | NS |
| drinks/week) | | | |
| Smoking | 5/9 | 4/8 | NS |
| (1 ppd) | | | |
| Race (#African | 3/15 | 0/16 | NS |
| American) | | | |

TABLE 5

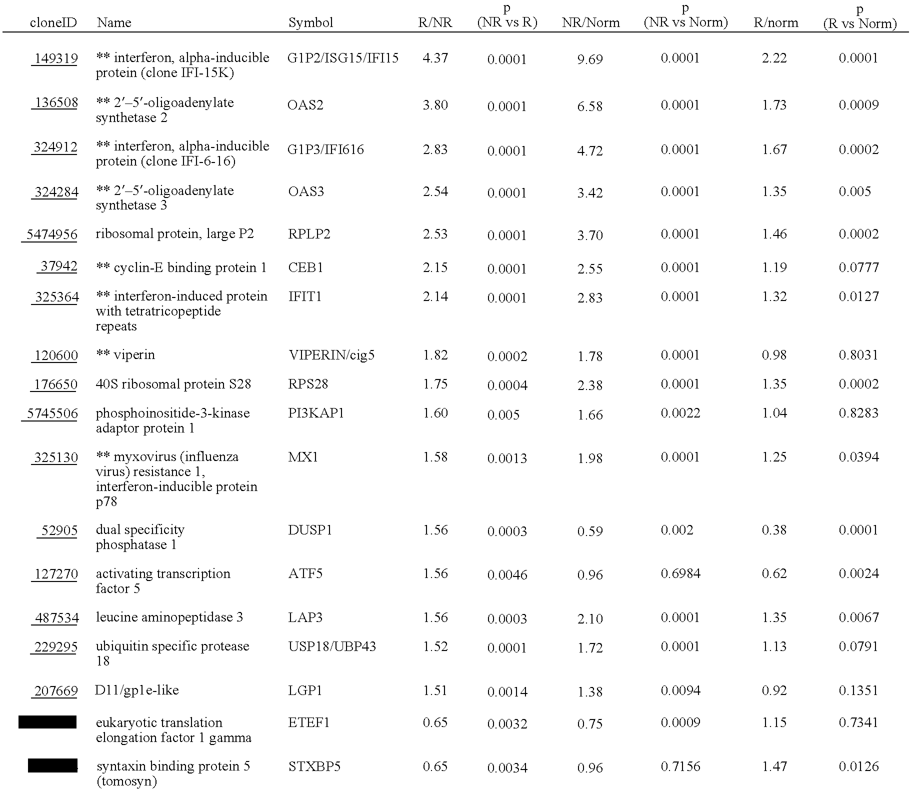

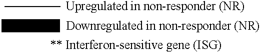

TABLE 6

| Variable | NR | R | p |
|---|---|---|---|
| number | 15 | 8 | |
| Age (yrs) | 50.2 ± 5.1 | 43.9 ± 9.0 | 0.1032 |
| Sex (# | 7/15 | 6/8 | 0.1917 |
| male) | | | |
| Viral load | 2.40 × 10⁶ ± | 4.87 × 10⁶ ± 5.1 × 10⁶ | 0.2597 |
| | 3.7 × 10⁶ | | |
| Activity | 1.63 ± 0.44 | 1.75 ± 0.46 | 0.5681 |
| Fibrosis | 2.50 ± 0.84 | 2.56 ± 0.98 | 0.881 |
| Completed | 13/14 | 7/7 | NS |
| Rx course | | | |
| PegIFN/rib | 14/15 | 7/8 | NS |
| dose >80% | | | |
| Alcohol (10 | 2/12 | 2/5 | NS |
| drinks/wk) | | | |
| Smoking | 5/9 | 3/4 | NS |
| (1 ppd) | | | |

TABLE 7

| | | | | |
|---|---|---|---|---|
| 229295 | CAGACCCTGACAATCCACCT | AGCTCATACTGCCCTCCAGA | 164 | Ubiquitin specific protease 18 |
| | (SEQ ID NO:11) | (SEQ ID NO:29) | | |
| 37942 | GATTGCTGGAGGGAATCAAA | TTGGATTTCCCTTTTTGTGC | 160 | cyclin-E binding protein 1 |
| | (SEQ ID NO:12) | (SEQ ID NO:30) | | |
| 149319 | CGCAGATCACCCAGAAGATT | GCCCTTGTTATTCCTCACCA | 185 | interferon, alpha-inducible protein 1 |
| | (SEQ ID NO:13) | (SEQ ID NO:31) | | |
| 136508 | TCAGCGAGGCCAGTAATCTT | GCAGGACATTCCAAGATGGT | 154 | 2′-5′-oligo adenylate synthetase 2 |
| | (SEQ ID NO:14) | (SEQ ID NO:32) | | |
| 324912 | CTCGCTGATGAGCTGGTCT | ATACTTGTGGGTGGCGTAGC | 148 | interferon, alpha-inducible protein |
| | (SEQ ID NO:15) | (SEQ ID NO:33) | | (clone IF1-6-16) |
| 324284 | GTCAAACCCAAGCCACAAGT | GGGCGAATGTTCACAAAGTT | 110 | 2′-5′-oligoadenylate synthetase 3, |
| | (SEQ ID NO:16) | (SEQ ID NO:34) | | 100 kDa |
| 5474956 | GCTGTAGCCGTCTCTGCTG | AAAAAGGCCAAATCCCATGT | 135 | ribosomal protein, large P2 |
| | (SEQ ID NO:17) | (SEQ ID NO:35) | | |
| 325364 | GCAGCCAAGTTTTACCGAAG | GCCCTATCTGGTGATGCAGT | 109 | interferon-induced protein with |
| | (SEQ ID NO:18) | (SEQ ID NO:36) | | tetratricopeptide repeats 1 |
| 120600 | CTTTTGCTGGGAAGCTCTTG | CAGCTGCTGCTTTCTCCTCT | 331 | viperin |
| | (SEQ ID NO:19) | (SEQ ID NO:37) | | |
| 176650 | CCGTGTGCAGCCTATCAAG | TTTACATTGCGGATGATGGA | 129 | RPS28 |
| | (SEQ ID NO:20) | (SEQ ID NO:38) | | |
| 5745506 | CTGCAGAGAGCTTTCCATCC | GTCTCTGGCTCATCGTCACA | 134 | phosphoinositide-3-kinase adaptor |
| | (SEQ ID NO:21) | (SEQ ID NO:39) | | protein 1 |
| 325130 | GTGCATTGCAGAAGGTCAGA | CTGGTGATAGGCCATCAGGT | 140 | myxovirus (influenza virus) resistance |
| | (SEQ ID NO:22) | (SEQ ID NO:40) | | 1, interferon-inducible protein p78 |
| | | | | (mouse) |
| 52905 | CCAACCATTTTGAGGGTCAC | ACCCTTCCTCCAGCATTCTT | 130 | dual specificity phosphatase 1 |
| | (SEQ ID NO:23) | (SEQ ID NO:41) | | |
| 127270 | AGCCCCCTGTCTTGGATACT | CGAGAAGGTTGAGGTGGAGA | 133 | activating transcription factor 5 |
| | (SEQ ID NO:24) | (SEQ ID NO:42) | | |
| 487534 | GGTGCCATGGATGTAGCTTT | AGAGAGGCATCCTCCAGACA | 124 | leucine aminopeptidase 3 |
| | (SEQ ID NO:25) | (SEQ ID NO:43) | | |
| 207669 | GCAGGAAGACAGTGGAGAGC | GAGCCAGCACTTCTGGGTAG | 125 | Dll1gp1e-like |
| | (SEQ ID NO:26) | (SEQ ID NO:44) | | |
| 3930678 | AGCGGAAGGAGGAGAAAAAG | GTACTCTTGGGCAGGTGAGC | 121 | eukaryotic translation elongation factor |
| | (SEQ ID NO:27) | (SEQ ID NO:45) | | 1 gamma |
| 231624 | GTTCATCTGATGGGCTTCGT | TTTGTTGTGGTGGTTCTCCA | 132 | syntaxin binding protein 5 (tomosyn) |
| | (SEQ ID NO:28) | (SEQ ID NO:46) | | |

1. A method of determining responsiveness to a therapy for a disease in a subject, said method comprising:
applying an abundance value for each product in a plurality of products to a model, wherein the abundance value for all or a portion of the products in the plurality of products is obtained by measurement of a biological sample from the subject, and
the plurality of products comprises a respective product of each of at least four different genes set forth in table 1; wherein
a first result of said applying is deemed to indicate that said subject is responsive to said therapy for said disease, and
a second result of said applying is deemed to indicate that said subject is nonresponsive to said therapy for said disease, and
wherein either (i) said therapy is a liver disease therapy and said disease is a liver disease, or (ii) said therapy is an immunomodulatory disease therapy and said disease is a disease treatable with an immunomodulatory disease therapy.

2. The method of claim 1, wherein each product in the plurality of products is an abundance value for an RNA transcript of a gene set forth in table 1 in said biological sample.

3. The method of claim 1, wherein each product in the plurality of products is an abundance value for a protein encoded by a gene set forth in table 1 in said biological sample.

4. The method of claim 1, wherein said therapy is a liver disease therapy and said disease is a liver disease.

5. The method of claim 1, wherein said therapy is an immunomodulatory disease therapy and said disease is a disease treatable with an immunomodulatory disease therapy.

6. The method of claim 1, wherein said model is a clustering algorithm and wherein said applying comprises:
clustering (i) the abundance value for each product in the plurality of products from said subject, and (ii) the abundance value for each product in the plurality of products from a plurality of training subjects, wherein said plurality of training subjects comprises subjects that are known to be responsive to said disease therapy and subjects that are known to be nonresponsive to said disease therapy, wherein
the coclustering of the abundance of each product in the plurality of products from said subject with a cluster of said plurality of training subjects that represents those subjects that are known to be responsive to said disease is deemed to indicate that said subject is responsive to said disease therapy, and
the coclustering of the abundance of each product in the plurality of products from said subject with a cluster of said plurality of training subjects that represents those subjects that are known to be nonresponsive to said disease therapy is deemed to indicate that said subject is nonresponsive to said disease therapy.

7. The method of claim 1, wherein said model is a neural network and wherein said applying comprises:
training the neural network with the abundance value for each product in the plurality of products from a plurality of training subjects, wherein said plurality of training subjects comprises subjects that are known to be responsive to said disease therapy and subjects that are known to be nonresponsive to said disease therapy; and
inputting the abundance value for each product in the plurality of products from said subject to the trained neural network, wherein
a first outcome of said neural network upon said inputting is deemed to indicate that said subject is responsive to said disease therapy, and
a second outcome of said neural network upon said inputting is deemed to indicate that said subject is nonresponsive to said disease therapy.

8. The method of claim 1, wherein said model is a regression model and wherein said applying comprises:
forming a regression equation by regressing the abundance of each product in the plurality of products from a plurality of training subjects, wherein said plurality of training subjects comprises subjects that are known to be responsive to said disease therapy and subjects that are known to be nonresponsive to said disease therapy; and
inputting the abundance of each product in the plurality of products from said subject to the regression equation, wherein
a first result of said regression equation is deemed to indicate that said subject is responsive to said disease therapy, and
a second result of said regression equation is deemed to indicate that said subject is nonresponsive to said disease therapy.

9. The method of claim 1, wherein said model is linear discriminant analysis and wherein said applying comprises:
computing a plurality of linear discriminant terms using the abundance of each product in the plurality of products from a plurality of training subjects, wherein said plurality of training subjects comprises subjects that are known to be responsive to said disease therapy and subjects that are known to be nonresponsive to said disease therapy; and
computing values for the plurality of linear discriminant terms for each respective training subject in the plurality of training subjects;
computing values for the plurality of linear discriminant terms for the subject; wherein
the grouping, based on the values for the plurality of linear discriminant term values, of the subject with one or more training subjects that are known to be responsive to said disease therapy is deemed to indicate that said subject is responsive to said disease therapy, and
the grouping, based on the values for the plurality of linear discriminant term values, of the subject with one or more training subjects that are known to be nonresponsive to said disease is deemed to indicate that said subject is nonresponsive to said disease therapy.

10. The method of claim 1, wherein said model is quadratic discriminant analysis and wherein said applying comprises:
computing a plurality of quadratic discriminant terms using the abundance of each product in the plurality of products from a plurality of training subjects, wherein said plurality of training subjects comprises subjects that are known to be responsive to said disease therapy and subjects that are known to be nonresponsive to said disease therapy; and
determining values for the plurality of quadratic discriminant terms for each respective training subject in the plurality of training subjects;
determining values for the plurality of quadratic discriminant terms for the subject; wherein
the grouping, based on the values for the plurality of quadratic discriminant term values, of the subject with one or more training subjects that known to be are responsive to said disease therapy is deemed to indicate that said subject is responsive to said disease therapy, and
the grouping, based on the values for the plurality of quadratic discriminant term values, of the subject with one or more training subjects that are known to be nonresponsive to said disease therapy is deemed to indicate that said subject is nonresponsive to said disease therapy.

11. The method of claim 1, wherein said model is principal component analysis and wherein said applying comprises:
computing a plurality of principal components using the abundance of each product in the plurality of products from a plurality of training subjects, wherein said plurality of training subjects comprises subjects that are known to be responsive to said disease therapy and subjects that are known to be nonresponsive to said disease therapy;
determining the values for the plurality of principal components for each respective training subject in the plurality of training subjects;
determining the values for the plurality of principal components for the subject; wherein
the grouping, based on the values for the plurality of principal components, of the subject with one or more training subjects that are known to be responsive to said disease therapy is deemed to indicate that said subject is responsive to said disease therapy, and
the grouping, based on the values for the plurality of principal components, of the subject with one or more training subjects that are nonresponsive to said disease is deemed to indicate that said subject is nonresponsive to said disease therapy.

12. The method of claim 1, wherein said model is a support vector machine and wherein said applying comprises:
constructing the support vector machine with the abundance of each product in the plurality of products from a plurality of training subjects, wherein said plurality of training subjects comprises subjects that are known to be responsive to said disease therapy and subjects that are known to be nonresponsive to said disease therapy; and
inputting the abundance of each product in the plurality of products from said subject to the support vector machine, wherein
a first outcome of said support vector machine upon said inputting is deemed to indicate that said subject is responsive to said disease therapy, and
a second outcome of said support vector machine upon said inputting is deemed to indicate that said subject is nonresponsive to said disease therapy.

13. The method of claim 1, wherein said model is a decision tree and wherein said applying comprises:
constructing the decision tree with the abundance of each product in the plurality of products from a plurality of training subjects, wherein said plurality of training subjects comprises subjects that are known to be responsive to said immunomodulatory disease therapy and subjects that are known to be nonresponsive to said immunomodulatory disease therapy; and
inputting the abundance of each product in the plurality of products from said subject to the decision tree, wherein
a first outcome of said decision tree upon said inputting is deemed to indicate that said subject is responsive to said disease therapy, and
a second outcome of said decision tree upon said inputting is deemed to indicate that said subject is nonresponsive to said disease therapy.

14. The method of claim 1, wherein said model is a nearest neighbor analysis and wherein said applying comprises:
constructing a neighborhood with the abundance of each product in the plurality of products from a plurality of training subjects, wherein said plurality of training subjects comprises subjects that are known to be responsive to said disease therapy and subjects that are known to be nonresponsive to said disease therapy;
inputting the abundance of each product in the plurality of products from said subject into the neighborhood;
determining whether a predetermined number of neighbors closest to said subject in said neighborhood are responsive to said disease therapy or nonresponsive to said disease therapy, wherein
a majority of said predetermined number of neighbors closest to said subject in said neighborhood that is responsive to said disease therapy is deemed to indicate that said subject is responsive to said disease therapy, and
a majority of said predetermined number of neighbors closest to said subject in said neighborhood that is nonresponsive to said disease therapy is deemed to indicate that said subject is nonresponsive to said disease therapy.

15. The method of claim 1, wherein the plurality of products consists of respective products of a maximum of one hundred genes.

16. The method of claim 1, wherein the plurality of products consists of respective products of a maximum of fifty genes.

17. The method of claim 1, wherein the plurality of products consists of respective products of a maximum of twenty-five genes.

18. The method of claim 1, wherein the plurality of products consists of respective products of a maximum of fifteen genes.

19. The method of claim 1, wherein the plurality of products consists of respective products of a maximum of ten genes.

20. The method of claim 1, wherein the plurality of products consists of respective products of a maximum of eight genes.

21. The method of claim 1, wherein the plurality of products consists of respective products of the genes set forth in table 1.

22. The method of claim 1, wherein the plurality of products consists of respective products of between four and forty genes set forth in table 1.

23. The method of claim 1, wherein the plurality of products consists of respective products of between four and twenty genes set forth in table 1.

24. The method of claim 1, wherein the plurality of products consists of respective products of between four and eight genes set forth in table 1.

25. The method of claim 1, wherein the plurality of products comprises a product of one or more of the group consisting of SEQ ID NO: 1, SEQ ID NO: 3, SEQ ID NO. 5, SEQ ID NO: 7, and SEQ ID NO: 9.

26. The method of claim 1, wherein the plurality of products comprises a product of one or more of the group consisting of SEQ ID NO: 2, SEQ ID NO: 4, SEQ ID NO. 6, SEQ ID NO: 8, and SEQ ID NO: 10.

27. The method of claim 1, wherein the plurality of products consists of products of OAS3, G1P3, DUSP1, IFIT1, MX1, G1P2, LAP3, cig5, LGP1, USP18, RPS28, CEB1, RPLP2, STXBP5, ETEF1, OAS2, ATF5, and PI3KAP1, respectively.

28. The method of claim 1, wherein the plurality of products consists of a product of IFIT1, OAS2, DUSP1, ATF5, LGP1, RPS28, USP18, and STXBP5, respectively.

29. The method of claim 1, wherein said subject is human.

30. The method of claim 1, wherein said subject is a mouse, a rat, a monkey, a hamster, a sheep, a cow, a pig, a horse, a cat or a dog.

31. The method of claim 1, further comprising a step of determining said abundance value for each product in said plurality of products prior to said step (a).

32. The method of claim 31, wherein said determining comprises hybridizing a polynucleotide encoding the product under conditions of high stringency to nucleotides of said genes set forth in table 1, or hybridizing a nucleotide sequence under conditions of high stringency to a polynucleotide that is complementary to nucleotides of said genes.

33. The method of claim 31, wherein said determining comprises hybridizing a polynucleotide encoding the product under conditions of moderate stringency to nucleotides of said genes set forth in table 1, or hybridizing a nucleotide sequence under conditions of moderate stringency to a polynucleotide that is complementary to nucleotides of said genes.

34. The method of claim 1, wherein said disease therapy comprises administration of human interferon to said subject.

35. The method of claim 34, wherein said human interferon is human interferon alpha or human interferon beta.

36. The method of claim 1, wherein said disease is hepatitis C.

37. The method of claim 1, wherein said disease is an immune-related disease.

38. The method of claim 37, wherein said immune-related disease is multiple sclerosis, idiopathic pulmonary fibrosis, Guillain-Barre Syndrome, adult systemic mastocytosis, ulcerative colitis, Crohn's disease, hepatitis C associated cryoglobulinemia, or HTLV-1 associated myelopathy.

39. The method of claim 1, wherein said disease is caused by a viral infection of said subject.

40. The method of claim 1, wherein said disease is a bacterial disease caused by a bacterium.

41. The method of claim 40, wherein said bacterium is cryptococcal meningitis or Tuberculosis.

42. The method of claim 1, wherein said disease is a neoplastic disease.

43. The method of claim 1, wherein said disease is renal cell carcinoma, hepatocellular carcinoma, a malignant carcinoid tumor, a neuroendocrine tumor, lymphoma, acute leukemia, chronic leukemia, chronic myelogenous leukemia, urothelial cancer, prostate cancer, penile cancer, nasopharyngeal cancer, pancreatic cancer, gastric cancer, cervical cancer, colorectal cancer, small cell lung cancer, non small cell lung cancer, malignant mesothelioma, or breast cancer.

44. The method of claim 1, wherein said disease is diabetic retinopathy or Peyronie's disease.

45. A computer program product comprising a computer readable storage medium and a computer program mechanism embedded therein, the computer program mechanism comprising:
a data analysis module for determining a responsiveness to a disease therapy in a subject for a disease, wherein either (i) said therapy is a liver disease therapy and said disease is a liver disease, or (ii) said therapy is an immunomodulatory disease therapy and said disease is a disease treatable with an immunomodulatory disease therapy, the data analysis module comprising:
instructions for applying an abundance of each product in a plurality of products to a model, wherein the abundance of all or a portion of the products in the plurality of products is obtained by measurement of a biological sample from the subject, and
the plurality of products comprises a respective product of each of at least four different genes set forth in table 1; wherein
a first result of said instructions for applying is deemed to indicate that said subject is responsive to said disease therapy for said disease, and
a second result of said instructions for applying is deemed to indicate that said subject is not responsive to said disease therapy for said disease.

46. A computer comprising:
a central processing unit;
a memory, coupled to the central processing unit, the memory storing a data analysis module for determining a responsiveness to a disease therapy in a subject for a disease, wherein either (i) said therapy is a liver disease therapy and said disease is a liver disease, or (ii) said therapy is an immunomodulatory disease therapy and said disease is a disease treatable with an immunomodulatory disease therapy, the data analysis module comprising:
instructions for applying an abundance of each product in a plurality of products to a model, wherein the abundance of all or a portion of the products in the plurality of products is obtained by measurement of a biological sample from the subject, and
the plurality of products comprises a respective product of each of at least four different genes set forth in table 1; wherein
a first result of said instructions for applying is deemed to indicate that said subject is responsive to said disease therapy for said disease, and
a second result of said instructions for applying is deemed to indicate that said subject is not responsive to said disease therapy for said disease.

47. A method for identifying a candidate molecule for use as a liver disease therapy agent or an immunomodulatory disease therapy agent in the treatment of a disease afflicting a subject, the method comprising:
(a) contacting a cell, or recombinantly expressing within the cell, a test molecule; and
(b) determining whether the RNA expression or protein expression in said cell of at least one open reading frame is changed in step (a) relative to the expression of said open reading frame in the absence of the test molecule, each said open reading frame being regulated by a promoter native to a gene in table 1 or a homolog of a gene in table 1, with the proviso that said gene is not USP18,
wherein, when the RNA expression or protein expression of said at least one open reading frame is changed, the test molecule is identified as a candidate molecule for use as a liver disease therapy agent or an immunomodulatory disease therapy agent.

48. The method of claim 47, wherein step (b) comprises determining whether the RNA expression or protein expression of said at least one open reading frame is lowered in step (a) relative to the expression of said open reading frame in the absence of the candidate molecule wherein at least one open reading frame is regulated by a promoter native to ISG15.

49. The method of claim 47, wherein step (b) comprises determining whether RNA expression is changed.

50. The method of claim 47, wherein step (b) comprises determining whether protein expression is changed.

51. The method of claim 47, wherein step (b) comprises determining whether RNA or protein expression of at least two of said open reading frames is changed.

52. The method of claim 47, wherein step (a) comprises contacting the cell with the candidate molecule, and wherein step (a) is carried out in a liquid high throughput-like assay.

53. The method of claim 47, wherein the cell comprises a promoter region of at least one gene selected from the group consisting of SEQ ID NO: 1, SEQ ID NO: 3, SEQ ID NO: 5, SEQ ID NO: 7, and homologs of each of the foregoing, each promoter region being operably linked to a marker gene; and wherein step (b) comprises determining whether the RNA expression or protein expression of the marker gene(s) is changed in step (a) relative to the expression of said marker gene in the absence of the candidate molecule.

54. The method of claim 53, wherein the marker gene is selected from the group consisting of green fluorescent protein, red fluorescent protein, blue fluorescent protein, luciferase, LEU2, LYS2, ADE2, TRP1, CAN1, CYH2, GUS, CUP 1, and chloramphenicol acetyl transferase.

55. The method of claim 47, wherein said subject is human.

56. The method of claim 47, wherein said subject is a mouse, a rat, a monkey, a hamster, a sheep, a cow, a pig, a horse, a cat or a dog.

57. The method of claim 47, wherein said disease is hepatitis C.

58. The method of claim 47, wherein said disease is an immune-related disease.

59. The method of claim 47, wherein said disease is caused by a viral infection of said subject.

60. The method of claim 47, wherein said disease is a bacterial disease caused by a bacterium.

61. The method of claim 47, wherein said bacterium is cryptococcal meningitis or Tuberculosis.

62. The method of claim 47, wherein said disease is a neoplastic disease.

63. A method for identifying a candidate molecule for use as a liver disease therapy agent or an immunomodulatory disease therapy agent in the treatment of a disease afflicting a subject, the method comprising:
determining whether a test molecule specifically binds to a polypeptide, wherein the polypeptide is:
(a) a first polypeptide, the amino acid sequence of which comprises SEQ ID NO: 2, SEQ ID NO: 4, SEQ ID NO: 6, or SEQ ID NO: 8; or
(b) a second polypeptide that comprises a homolog of SEQ ID NO: 2, SEQ ID NO: 4, SEQ ID NO: 6, or SEQ ID NO: 8; or
(c) a third polypeptide that comprises the protein product of a polynucleotide wherein said polynucleotide hybridizes under conditions of high stringency to a nucleic acid consisting of SEQ ID NO: 1, SEQ ID NO: 3, SEQ ID NO: 5, or SEQ ID NO: 7 or the complement of SEQ ID NO: 1, SEQ ID NO: 3, SEQ ID NO: 5, or SEQ ID NO: 7,
wherein said determining comprises contacting the polypeptide with the test molecule under conditions suitable for binding, and detecting a specific binding of the test molecule to the polypeptide, wherein when specific binding is detected, the test molecule is identified as a candidate molecule for use as a liver disease therapy agent or an immunomodulatory disease therapy agent.

64. The process of claim 63, wherein the specific binding of the test molecule to the polypeptide is detected by gel filtration, an affinity column, or a modulation of an enzymatic activity of said polypeptide.

65. The method of claim 63, wherein said disease is hepatitis C.

66. The method of claim 63, wherein said disease is an immune-related disease.

67. The method of claim 63, wherein said disease is multiple sclerosis, idiopathic pulmonary fibrosis, Guillain-Barre Syndrome, adult systemic mastocytosis, ulcerative colitis, Crohn's disease, hepatitis C associated cryoglobulinemia, or HTLV-1 associated myelopathy.

68. The method of claim 63, wherein said disease is inflicted by a viral infection of said subject.

69. The method of claim 63, wherein said disease is a bacterial disease caused by a bacterium.

70. The method of claim 69, wherein said bacterium is cryptococcal meningitis or Tuberculosis.

71. The method of claim 63, wherein said disease is a neoplastic disease.

72. The method of claim 63, wherein said disease is renal cell carcinoma, hepatocellular carcinoma, a malignant carcinoid tumor, a neuroendocrine tumor, lymphoma, acute leukemia, chronic leukemia, chronic myelogenous leukemia, urothelial cancer, prostate cancer, penile cancer, nasopharyngeal cancer, pancreatic cancer, gastric cancer, cervical cancer, colorectal cancer, small cell lung cancer, non small cell lung cancer, malignant mesothelioma, or breast cancer.

73. The method of claim 63, wherein said disease is diabetic retinopathy or Peyronie's disease.

74. A method of administering a liver disease therapy or an immunomodulatory disease therapy comprising:
administering to a subject in which such treatment is desired a therapeutically effective amount of a compound that modulates in the subject an abundance or an activity of a protein comprising a sequence selected from the group consisting of SEQ ID NO: 2, SEQ ID NO: 4, SEQ ID NO: 6, SEQ ID NO: 8, and homologs of each of the foregoing.

75. The method of claim 74, wherein said subject is human.

76. The method of claim 74, wherein said subject is a mouse, a rat, a monkey, a hamster, a sheep, a cow, a pig, a horse, a cat or a dog.

77. A method for identifying a candidate molecule for use as a liver disease therapy agent or an immunomodulatory disease therapy agent, comprising:
contacting a cell, or recombinantly expressing within the cell, a test molecule; and
determining whether the abundance or activity of a protein comprising SEQ ID NO: 2, SEQ ID NO: 4, SEQ ID NO: 6, or SEQ ID NO: 8 in the cell is changed relative to the abundance or activity, respectively, of said protein in the absence of the test molecule, wherein when the abundance or activity of said protein is changed, the test molecule is identified as a candidate molecule for use as a liver disease therapy agent or an immunomodulatory disease therapy agent.

78. A method for identifying a liver disease therapy agent or an immunomodulatory disease therapy agent, comprising:
(i) contacting a polypeptide with a test molecule, wherein said polypeptide is:
(a) a first polypeptide, the amino acid sequence of which comprises SEQ ID NO: 2, SEQ ID NO: 4, SEQ ID NO: 6, or SEQ ID NO: 8; or
(b) a second polypeptide that comprises a homolog of SEQ ID NO: 2, SEQ ID NO: 4, SEQ ID NO: 6, or SEQ ID NO: 8; or
(c) a third polypeptide that comprises the protein product of a polynucleotide wherein said polynucleotide hybridizes under conditions of high stringency to a nucleic acid consisting of SEQ ID NO: 1, SEQ ID NO: 3, SEQ ID NO: 5, or SEQ ID NO: 7 or the complements of SEQ ID NO: 1, SEQ ID NO: 3, SEQ ID NO: 5, or SEQ ID NO: 7; and
(ii) determining whether said test molecule modulates the biological activity of said polypeptide relative to the biological activity of said polypeptide in the absence of the test molecule,
wherein when the abundance or activity of said polypeptide is changed, the test molecule is identified as a candidate molecule for use as a liver disease therapy agent or an immunomodulatory disease therapy agent.

79. A computer system comprising:
a central processing unit; and
a memory, coupled to the central processing unit, the memory storing
(a) a sequence of one or more genes or a sequence of a polypeptide encoded by said one or more genes, wherein said one or more genes are selected from the group consisting of G1P2/ISG15/IFI-15, G1P3/IFI-6-16, OAS3, RPLP2, CEB1, VIPERIN/CIG5, PI3KAP1, MX1, LAP3, ETEF1, IFIT1/IFI56, OAS2, DUSP1, ATF5, LGP-1, RPS28, and STXBP5;
(b) one or more computer programs, wherein said computer programs comprise instructions for executing at least one supervised classifier analysis technique; and
(c) instructions for outputting a predicted response of a subject to a regimen of pegylated interferon alpha and ribavirin in a therapy for hepatitis C viral infection.

80. A method for predicting the response of a subject to a regimen of pegylated interferon alpha and ribavirin in a therapy for a hepatitis C viral infection, the method comprising:
(a) determining the expression levels of the following genes in a tissue sample from the subject: G1P2/ISG15/IFI-15, G1P3/IFI-6-16, OAS3, RPLP2, CEB1, VIPERIN/CIG5, PI3KAP1, MX1, LAP3, ETEF1, IFIT1I/IFI56, OAS2, DUSP1, ATF5, LGP-1, RPS28, USP18/UBP43, and STXBP5;
(b) comparing the levels of expression in (a) to a corresponding control sample from a subject not having a hepatitis C viral infection; and
(c) predicting that the subject will be nonresponsive to a regimen of pegylated interferon alpha and ribavirin in a therapy for hepatitis C if there is an increase in the expression levels of G1P2/ISG15/IFI-15, G1P3/IFI-6-16, OAS3, RPLP2, CEB1, VIPERIN/CIG5, PI3KAP1, MX1, LAP3, IFIT1/IFI56, OAS2, DUSP1, ATF5, LGP-1, RPS28, and USP18/UBP43 in (a) relative to the expression levels of such genes in the control sample, and if there is a decrease in the expression levels of ETEF1 and STXBP5 in (a) relative to the expression levels of such genes in the control sample.

81. A method for predicting the response of a subject to a regimen of PegIFNα and ribavirin in a therapy for a hepatitis C viral infection, the method comprising:
(a) determining the expression levels of the following genes in a tissue sample from the subject: IFIT1/IFI56, OAS2, DUSP1, ATF5, LGP-1, RPS28, USP18/UBP43, and STXBP5;
(b) comparing the levels of expression in (a) to a corresponding control sample from a subject not having a hepatitis C viral infection; and
(c) predicting that the subject will be nonresponsive to a regimen of PegIFNα and ribavirin in a therapy for a hepatitis C viral infection if there is an increase in the expression levels of IFIT1/IFI56, OAS2, DUSP1, ATF5, LGP-1, RPS28, and USP18/UBP43 in (a) relative to the expression levels of such genes in the control sample, and if there is a decrease in the expression levels of STXBP5 in (a) relative to the expression levels of STXBP5 in the control sample.

82. A method for predicting the response of a subject to a regimen of PegIFNα and ribavirin in a therapy for a hepatitis C viral infection, the method comprising:
(a) determining the expression levels of at least one of the following genes in a tissue sample from the subject: G1P2/ISG15/IFI-15, G1P3/IFI-6-16, OAS3, RPLP2, CEB1, VIPERIN/CIG5, PI3KAP1, MX1, LAP3, ETEF1, IFIT1/IFI56, OAS2, DUSP1, ATF5, LGP-1, RPS28, and STXBP5;
(b) comparing the levels of expression in (a) to a corresponding control sample from a subject not having a hepatitis C viral infection; and
(c) predicting that the subject will be nonresponsive to a regimen of PegIFNα and ribavirin in a therapy for said hepatitis C viral infection if there is an increase in the expression levels of the one or more genes measures in step (a) relative to the expression levels of such genes in the control sample, and if there is a decrease in the expression levels of ETEF1 and STXBP5 in (a) relative to the expression levels of such genes in the control sample.

83. A method for predicting the response of a subject to a regimen of PegIFNα and ribavirin in a therapy for a hepatitis C viral infection, the method comprising:
(a) determining the expression levels of at least one of the following genes in a tissue sample from the subject: IFIT1/IFI56, OAS2, DUSP1, ATF5, LGP-1, RPS28, USP18/UBP43, and STXBP5;
(b) comparing the levels of expression in (a) to a corresponding control sample from a subject not having a hepatitis C viral infection; and
(c) predicting that the subject will be nonresponsive to a regimen of PegIFNα and ribavirin in a therapy for hepatitis C if there is an increase in the expression levels of the one or more genes measured in step (a) relative to the expression levels in such genes in the control sample, and if there is a decrease in the expression levels of STXBP5 in (a) relative to the expression levels in such genes in the control sample.

84. A method for predicting the response of a subject to a regimen of PegIFNα and ribavirin in a therapy for a hepatitis C viral infection, the method comprising:
(a) determining the expression levels of two or more of the following genes in a tissue sample from the subject: G1P2/ISG15/IFI-15, G1P3/IFI-6-16, OAS3, RPLP2, CEB1, VIPERIN/CIG5, PI3KAP1, MX1, LAP3, ETEF1, IFIT1/IFI56, OAS2, DUSP1, ATF5, LGP-1, RPS28, USP18/UBP43, and STXBP5;
(b) comparing the levels of expression in (a) to a corresponding control sample from a subject not having a hepatitis C viral infection; and
(c) predicting that a subject will be nonresponsive to a regimen of PegIFNα and ribavirin in a therapy for hepatitis C if there is an increase in the expression levels of the genes measured in step (a) relative to the expression levels of such genes in the control sample, and if there is a decrease in the expression levels of ETEF1 and STXBP5 in (a) relative to the expression levels of such genes in the control sample.

85. A method for predicting the response of a subject to a regimen of PegIFNα and ribavirin in a therapy for a hepatitis C viral infection, the method comprising:
(a) determining the expression levels of two or more of the following genes in a tissue sample from the subject: IFIT1/IFI56, OAS2, DUSP1, ATF5, LGP-1, RPS28, USP18[UBP43, and STXBP5;
(b) comparing the levels of expression in (a) to a corresponding control sample from a subject not having a hepatitis C viral infection; and
(c) predicting that a subject will be nonresponsive to a regimen of PegIFNα and ribavirin in a therapy for hepatitis C if there is an increase in the expression levels of the genes measured in step (a) relative to the expression levels in such genes in the control sample, and if there is a decrease in the expression levels of STXBP5 in (a) relative to the expression levels in such genes in the control sample.

86. A method for predicting the response of a subject to a regimen of PegIFNα and ribavirin in a therapy for a hepatitis C viral infection, the method comprising:
(a) determining the expression levels of at least 1 of the following genes in a tissue sample from the subject: IFI-6-16 (G1P3), LAP3 (luecine aminopeptidase 3) CIG5 (Viperin) and LGP1 (d11lgp1e-like);
(b) comparing the levels of expression in (a) to a corresponding control sample from a subject not infected with a hepatitis C viral infection; and
(c) predicting that the subject will be nonresponsive to a regimen of PegIFNα and ribavirin in a therapy for hepatitis C if there is an increase in the expression levels of such genes in (a) relative to the expression levels of such genes in the control sample.

87. A method of determining responsiveness to a regimen of PegIFNα and ribavirin for a hepatitis C viral infection in a subject, said method comprising:
applying an abundance value for each product in a plurality of products to a model, wherein the abundance value for all or a portion of the products in the plurality of products is obtained by measurement of a tissue sample from the subject, and
the plurality of products comprises a respective product of each of at least four different genes set forth in table 1; wherein
a first result of said applying is deemed to indicate that said subject is responsive to said PegIFNα plus ribavirin therapy for said hepatitis C viral infection, and
a second result of said applying is deemed to indicate that said subject is nonresponsive to said PegIFNα plus ribavirin therapy for said hepatitis C viral infection.

88. A computer program product for use in conjunction with a computer system, the computer program product comprising a computer readable storage medium, the computer readable storage medium comprising a sequence of two or more genes or a sequence of two or more polypeptides encoded by said two or more genes, wherein said two or more genes are G1P2/ISG15/IFI-15, G1P3/IFI-6-16, OAS3, RPLP2, CEB1, VIPERIN/CIG5, PI3KAP1, MX1, LAP3, ETEF1, IFIT1/IFI56, OAS2, DUSP1, ATF5, LGP-1, RPS28, USP18/UBP43, STXBP5 or some combination thereof, and instructions for outputting a predicted response of a subject to a regimen of PegIFNα and ribavirin in a therapy for hepatitis C viral infection.


SEQUENCE LISTING (10 sequences)

SEQ ID NO: 1 - DNA, 2853 bp, Homo sapiens
gctctgctccaggcatctgccacaatgtgggtgcttacacctgctgcttttgctgggaagctcttgagtgtgttcaggcaacctctgagctctctgtggaggagcctggtcccgctgttctgctggctgagggcaaccttctggctgctagctaccaagaggagaaagcagcagctggtcctgagagggccagatgagaccaaagaggaggaagaggaccctcctctgcccaccaccccaaccagcgtcaactatcacttcactcgccagtgcaactacaaatgcggcttctgtttccacacagccaaaacatcctttgtgctgccccttgaggaagcaaagagaggattgcttttgcttaaggaagctggtatggagaagatcaacttttcaggtggagagccatttcttcaagaccggggagaatacctgggcaagttggtgaggttctgcaaagtagagttgcggctgcccagcgtgagcatcgtgagcaatggaagcctgatccgggagaggtggttccagaattatggtgagtatttggacattctcgctatctcctgtgacagctttgacgaggaagtcaatgtccttattggccgtggccaaggaaagaagaaccatgtggaaaaccttcaaaagctgaggaggtggtgtagggattatagagtcgctttcaagataaattctgtcattaatcgtttcaacgtggaagaggacatgacggaacagatcaaagcactaaaccctgtccgctggaaagtgttccagtgcctcttaattgagggtgagaattgtggagaagatgctctaagagaagcagaaagatttgttattggtgatgaagaatttgaaagattcttggagcgccacaaagaagtgtcctgcttggtgcctgaatctaaccagaagatgaaagactcctaccttattctggatgaatatatgcgctttctgaactgtagaaagggacggaaggacccttccaagtccatcctggatgttggtgtagaagaagctataaaattcagtggatttgatgaaaagatgtttctgaagcgaggaggaaaatacatatggagtaaggctgatctgaagctggattggtagagcggaaagtggaacgagacttcaacacaccagtgggaaaactcctagagtaactgccattgtctgcaatactatcccgttggtatttcccagtggctgaaaacctgattttctgctgcacgtggcatctgattacctgtggtcactgaacacacgaataacttggatagcaaatcctgagacaatggaaaaccattaactttacttcattggcttataaccttgttgttattgaaacagcacttctgtttttgagtttgttttagctaaaaagaaggaatacacacaggaataatgaccccaaaaatgcttagataaggcccctatacacaggacctgacatttagctcaatgatgcgtttgtaagaaataagctctagtgatatctgtgggggcaaaatttaatttggatttgattttttaaaacaatgtttactgcgatttctatatttccattttgaaactatttcttgttccaggtttgttcatttgacagagtcagtattttttgccaaatatccagataaccagttttcacatctgagacattacaaagtatctgcctcaattatttctgctggttataatgcttttttttttttgcctttatgccattgcagtcttgtactttttactgtgatgtacagaaatagtcaacagatgtttccaagaacatatgatatgataatcctaccaattttcaagaagtctctagaaagagataacacatggaaagacggcgtggtgcagcccagcccacggtgcctgttccatgaatgctggctacctatgtgtgtggtacctgttgtgtccctttctcttcaaagatccctgagcaaaacaaagatacgctttccatttgatgatggagttgacatggaggcagtgcttgcattgctttgttcgcctatcatctggccacatgaggctgtcaagcaaaagaataggagtgtagttgagtagctggttggccctacatttctgagaagtgacgttacactgggttggcataagatatcctaaaatcacgctggaaccttgggcaaggaagaatgtgagcaagagtagagagagtgcctggatttcatgtcagtgaagccatgtcaccatatcatatttttgaatgaactctgagtcagttgaaatagggtaccatctaggtcagtttaagaagagtcagctcagagaaagcaagcataagggaaaatgtcacgtaaactagatcagggaacaaaatcctctccttgtggaaatatcccatgcagtttgttgatacaacttagtatcttattgcctaaaaaaaaatttcttatcattgtttcaaaaaagcaaaatcatggaaaatttttgttgtccaggcaaataaaaggtcattttaatttaaaaaaaaaaaaaaaaaaaaaaaaaaaaaaaaggccaaggaaaaaaaatattcctacttaaattttaagtctataattcaatttaaatatgtgtgtgtctcatccaggataggataggttgtcttctattttccattttacctatttactttttttgtaagaaaagagaagaatgaattctaaagatgttccccatgggttttgattgtgtctaagctatgatgaccttcatataatcagcataaacataaaacaaattttttacttaacatgagtgcactttactaatcctcatggcacagtggctcacgcctgtaatcccagcacttggggaggacaatgtggggtggatcacgaggtc

SEQ ID NO: 2 - protein, 361 aa, Homo sapiens
MWVLTPAAFAGKLLSVFRQPLSSLWRSLVPLFCWLRATFWLLATKRRKQQLVLRGPDETKEEEEDPPLPTTPTSVNYHFTRQCNYKCGFCFHTAKTSFVLPLEEAKRGLLLLKEAGMEKINFSGGEPFLQDRGEYLGKLVRFCKVELRLPSVSIVSNGSLIRERWFQNYGEYLDILAISCDSFDEEVNVLIGRGQGKKNHVENLQKLRRWCRDYRVAFKINSVINRFNVEEDMTEQIKALNPVRWKVFQCLLIEGENCGEDALREAERFVIGDEEFERFLERHKEVSCLVPESNQKMKDSYLILDEYMRFLNCRKGRKDPSKSILDVGVEEAIKFSGFDEKMFLKRGGKYIWSKADLKLDW

SEQ ID NO: 3 - DNA, 2649 bp, Homo sapiens
cttttcttttttttttgacagggtctcactctgttgcccaagctggggtgcagtggcacgatcttggctcactgtagccttgacatcctgggctcaagggatcctcccatctcagcctcccaagtagctgcgactatgggtgtgacaccacgctgggctagtttttcaattttttgtagagatggagtctccctatgttgctcaggccggttgcgaactcctgggctcaagtgattctcctgcctcagcctcccaaagtgctgggattacagatgatagccacctcacccggcccacccctaccttctgaaagaggcattcttattcttattcccattttgcagatcaggaaacagagctcagtgcagcccactaaattgctcagggccctacagctaacaagcggcagaggcaggatctgcactcaggagctgcttggagatgctgctgtggccactgctgctgctgctgctgctgctgccaacattggccctgctcaggcagcagcggtcccaggatgccaggctgtcctggcttgctggcctccagcaccgagtggcatggggggccctggtctgggcagccacctggcagcgccggaggctggagcagagcacgctccatgtgcaccagagccagcagcaggccctgaggtggtgtctacagggagcccagcgcccccactgttccctcagaaggagcacagacataagcaccttccggaatcatctccctctgaccaaggccagccagacccagcaggaagacagtggagagcagccactgcccccgacctcaaaccaggaccttggggaggcctctctgcaggccaccttgctgggtctggcagccctaaacaaggcctacccagaagtgctggctcagggacgcactgcccgtgtgacgctcacatccccttggccccgacccctgccttggcctgggaataccctgggccaggtgggcacccctggaaccaaggaccctagggccctgctgctggacgcactgaggtccccagggctgagggcactggaggctgggacggctgtcgaacttctggatgttttcttgggcctggagactgatggtgaagagctagctggggcgatagctgccgggaaccctggagcgcctctccgtgaacgggcagctgagctccgggaggccctagagcaggggccacggggactggcccttcggctctggccaaagctgcaggtggtggtgactctggatgcaggaggccaggccgaggctgtggctgccctcggggccttgtggtgccaaggactagccttcttctctcctgcttatgctgcctcgggaggggtgctgggcctaaacctacagccagagcagccccatgggctctaccttctgccccctggggccccctttatcgagctgctcccagtcaaggaaggcacccaggaggaagctgcctccaccctccttttggccgaggcccagcagggcaaggagtatgagctggtgctgacggaccgcgccagcctcaccaggtgccgcctgggtgatgtggtgcgagtggttggtgcctacaatcagtgtccagtcgtcaggttcatctgcaggctggaccagaccctgagtgtgcgaggggaagatattggtgaagacctgttctctgaggccctgggccgggcagtggggcagtgggcgggggccaagctgctggaccatggctgtgtggagagcagcattctggattcctctgcgggctctgctccccactacgaggtgtttgtggcgctgagggggctgaggaatctgtcagaggaaaatcgagacaagctggaccactgccttcaggaagcctctccccgctacaagtccctgcggttctggggcagcgtgggccctgccagagtccacctggtggggcagggagccttccgagcactccgggcagccctcgctgcctgcccctcctcccccttcccccctgcgatgccccgggtccttcggcacaggcacctggcccagtgtctgcaggagagggtggtgtcctgagtcaagtcctgccccaccgcccagctccccccagaggccacctcgcccctccctctgggacctctccggatggggagtccttggccagggtctctgactctgtgtcacctgacatttgcccatgagagccgctgggccttagagaggccttggcccagctgaccggttctgaagtatgggcctccggggttagcagatgccagcagtgcctgcccgtgtccccatgtcccggcatgaaggacactgctagagagttaccatgcacaccgatggtttcctgtatcacagcccaaagaggttctctggtggccacagctgtgtgctcagtcagtgcactgggcaagctagaagtgttggggggttaatgtccccaggagcagcaaccctgagtcaataaggagcaggacctcagcttcattgtccttgagcaggacaattctgaagtgtattctacataaactctcagaggatgcccagcaggatggagtcccagttgcccgcagcagtaacccactcattcatgtacttcctgcgggggctctcccttccctctcttccccactcccccgccttgggcttcctgggatggctcccaaataaacctcttgcacccag

SEQ ID NO: 4 - protein, 530 aa, Homo sapiens
MLLWPLLLLLLLLPTLALLRQQRSQDARLSWLAGLQHRVAWGALVWAATWQRRRLEQSTLHVHQSQQQALRWCLQGAQRPHCSLRRSTDISTFRNHLPLTKASQTQQEDSGEQPLPPTSNQDLGEASLQATLLGLAALNKAYPEVLAQGRTARVTLTSPWPRPLPWPGNTLGQVGTPGTKDPRALLLDALRSPGLRALEAGTAVELLDVFLGLETDGEELAGAIAAGNPGAPLRERAAELREALEQGPRGLALRLWPKLQVVVTLDAGGQAEAVAALGALWCQGLAFFSPAYAASGGVLGLNLQPEQPHGLYLLPPGAPFIELLPVKEGTQEEAASTLLLAEAQQGKEYELVLTDRASLTRCRLGDVVRVVGAYNQCPVVRFICRLDQTLSVRGEDIGEDLFSEALGRAVGQWAGAKLLDHGCVESSILDSSAGSAPHYEVFVALRGLRNLSEENRDKLDHCLQEASPRYKSLRFWGSVGPARVHLVGQGAFRALRAALAACPSSPFPPAMPRVLRHRHLAQCLQERVVS

SEQ ID NO: 5 - DNA, 817 bp, Homo sapiens
gaaccgtttactcgctgctgtgcccatctatcagcaggctccgggctgaagattgcttctcttctctcctccaaggtctagtgacggagcccgcgcgcggcgccaccatgcggcagaaggcggtatcgcttttcttgtgctacctgctgctcttcacttgcagtggggtggaggcaggtaagaaaaagtgctcggagagctcggacagcggctccgggttctggaaggccctgaccttcatggccgtcggaggaggactcgcagtcgccgggctgcccgcgctgggcttcaccggcgccggcatcgcggccaactcggtggctgcctcgctgatgagctggtctgcgatcctgaatgggggcggcgtgcccgccggggggctagtggccacgctgcagagcctcggggctggtggcagcagcgtcgtcataggtaatattggtgccctgatgggctacgccacccacaagtatctcgatagtgaggaggatgaggagtagccagcagctcccagaacctcttcttccttcttggcctaactcttccagttaggatctagaactttgcctttttttttttttttttttttttttgagatgggttctcactatattgtccaggctagagtgcagtggctattcacagatgcgaacatagtacactgcagcctccaactcctagcctcaagtgatcctcctgtctcaacctcccaagtaggattacaagcatgcgccgacgatgcccagaatccagaactttgtctatcactctccccaacaacctagatgtgaaaacagaataaacttcacccagaaaa

SEQ ID NO: 6 - protein, 130 aa, Homo sapiens
MRQKAVSLFLCYLLLFTCSGVEAGKKKCSESSDSGSGFWKALTFMAVGGGLAVAGLPALGFTGAGIAANSVAASLMSWSAILNGGGVPAGGLVATLQSLGAGGSSVVIGNIGALMGYATHKYLDSEEDEE

SEQ ID NO: 7 - DNA, 1973 bp, Homo sapiens
ggccgagccgacaagatgttcttgctgcctcttccggctgcggggcgagtagtcgtccgacgtctggccgtgagacgtttcgggagccggagtctctccaccgcagacatgacgaagggccttgttttaggaatctattccaaagaaaaagaagatgatgtgccacagttcacaagtgcaggagagaattttgataaattgttagctggaaagctgagagagactttgaacatatctggaccacctctgaaggcagggaagactcgaaccttttatggtctgcatcaggacttccccagcgtggtgctagttggcctcggcaaaaaggcagctggaatcgacgaacaggaaaactggcatgaaggcaaagaaaacatcagagctgctgttgcagcggggtgcaggcagattcaagacctggagctctcgtctgtggaggtggatccctgtggagacgctcaggctgctgcggagggagcggtgcttggtctctatgaatacgatgacctaaagcaaaaaaagaagatggctgtgtcggcaaagctctatggaagtggggatcaggaggcctggcagaaaggagtcctgtttgcttctgggcagaacttggcacgccaattgatggagacgccagccaatgagatgacgccaaccagatttgccgaaattattgagaagaatctcaaaagtgctagtagtaaaaccgaggtccatatcagacccaagtcttggattgaggaacaggcaatgggatcattcctcagtgtggccaaaggatctgacgagcccccagtcttcttggaaattcactacaaaggcagccccaatgcaaacgaaccacccctggtgtttgttgggaaaggaattacctttgacagtggtggtatctccatcaaggcttctgcaaatatggacctcatgagggctgacatgggaggagctgcaactatatgctcagccatcgtgtctgctgcaaagcttaatttgcccattaatattataggtctggcccctctttgtgaaaatatgcccagcggcaaggccaacaagccgggggatgttgttagagccaaaaacgggaagaccatccaggttgataacactgatgctgaggggaggctcatactggctgatgcgctctgttacgcacacacgtttaacccgaaggtcatcctcaatgccgccaccttaacaggtgccatggatgtagctttgggatcaggtgccactggggtctttaccaattcatcctggctctggaacaaactcttcgaggccagcattgaaacaggggaccgtgtctggaggatgcctctcttcgaacattatacaagacaggttgtagattgccagcttgctgatgttaacaacattggaaaatacagatctgcaggagcatgtacagctgcagcattcctgaaagaattcgtaactcatcctaagtgggcacatttagacatagcaggcgtgatgaccaacaaagatgaagttccctatctacggaaaggcatgactgggaggcccacaaggactctcattgagttcttacttcgtttcagtcaagacaatgcttagttcagatactcaaaaatgtcttcactctgtcttaaattggacagttgaacttaaaaggtttttgaataaatggatgaaaatcttttaacggagacaaaggatggtatttaaaaatgtagaacacaatgaaatttgtatgccttgatttttttttcatttcacacaaagatttataaaggtaaagttaatatcttacttgataaggatttttaagatactctataaatgattaaaatttttagaacttcctaatcacttttcagagtatatgtttttcattgagaagcaaaattgtaactcagatttgtgatgctaggaacatgagcaaactgaaaattactatgcacttgtcagaaacaataaatgcaacttgttgtgcaaaaaaaaaaaaaaaaaaa

SEQ ID NO: 8 - protein, 519 aa, Homo sapiens
MFLLPLPAAGRVVVRRLAVRRFGSRSLSTADMTKGLVLGIYSKEKEDDVPQFTSAGENFDKLLAGKLRETLNISGPPLKAGKTRTFYGLHQDFPSVVLVGLGKKAAGIDEQENWHEGKENIRAAVAAGCRQIQDLELSSVEVDPCGDAQAAAEGAVLGLYEYDDLKQKKKMAVSAKLYGSGDQEAWQKGVLFASGQNLARQLMETPANEMTPTRFAEIIEKNLKSASSKTEVHIRPKSWIEEQAMGSFLSVAKGSDEPPVFLEIHYKGSPNANEPPLVFVGKGITFDSGGISIKASANMDLMRADMGGAATICSAIVSAAKLNLPINIIGLAPLCENMPSGKANKPGDVVRAKNGKTIQVDNTDAEGRLILADALCYAHTFNPKVILNAATLTGAMDVALGSGATGVFTNSSWLWNKLFEASIETGDRVWRMPLFEHYTRQVVDCQLADVNNIGKYRSAGACTAAAFLKEFVTHPKWAHLDIAGVMTNKDEVPYLRKGMTGRPTRTLIEFLLRFSQDNA

SEQ ID NO: 9 - skipped

SEQ ID NO: 10 - protein, 372 aa, Homo sapiens
MSKAFGLLRQICQSILAESSQSPADLEEKKEEDSNMKREQPRERPRAWDYPHGLVGLHNIGQTCCLNSLIQVFVMNVDFTRILKRITVPRGADEQRRSVPFQMLLLLEKMQDSRQKAVRPLELAYCLQKCNVPLFVQHDAAQLYLKLWNLIKDQITDVHLVERLQALYTIRVKDSLICVDCAMESSRNSSMLTLPLSLFDVDSKPLKTLEDALHCFFQPRELSSKSKCFCENCGKKTRGKQVLKLTHLPQTLTIHLMRFSIRNSQTRKICHSLYFPQSLDFSQILPMKRESCDAEEQSGGQYELFAVIAHVGMADSGHYCVYIRNAVDGKWFCFNDSNICLVSWEDIQCTYGNPNYHWQETAYLLVYMKMEC